(19) United States
(12) Patent Application Publication
(10) Pub. No.: US 2023/0127951 A1
(43) Pub. Date: Apr. 27, 2023

(54) ORGANIC ELECTROLUMINESCENCE DEVICE AND POLYCYCLIC COMPOUND FOR ORGANIC ELECTROLUMINESCENCE DEVICE

(71) Applicant: Samsung Display Co., LTD., Yongin-si (KR)

(72) Inventor: Takuya UNO, Yokohama (JP)

(21) Appl. No.: 18/071,473

(22) Filed: Nov. 29, 2022

Related U.S. Application Data
(62) Division of application No. 16/702,357, filed on Dec. 3, 2019.

(30) Foreign Application Priority Data
Mar. 5, 2019 (KR) 10-2019-0025425
Jun. 28, 2019 (KR) 10-2019-0077607

(51) Int. Cl. C07D 409/12; H01L 51/00; C07D 413/10; C07D 405/10; C07D 405/12; C07D 407/14; C07D 407/12; C07D 407/04; C07D 409/14; C07D 417/10; C07D 333/54; C07D 409/10; C07D 409/04; C07D 405/04; C07D 307/79

(52) U.S. Cl. CPC C07D 409/12; H01L 51/006; C07D 413/10; C07D 405/10; C07D 405/12; C07D 407/14; C07D 407/12; C07D 407/04; C07D 409/14; C07D 417/10; C07D 333/54; C07D 409/10; C07D 409/04; C07D 405/04; H01L 51/0094; H01L 51/0052; H01L 51/0073; H01L 51/0074; C07D 307/79

(57) ABSTRACT

An organic electroluminescence device includes a first electrode, a hole transport region disposed on the first electrode, an emission layer disposed on the hole transport region, an electron transport region disposed on the emission layer, and a second electrode disposed on the electron transport region, wherein the hole transport region includes a polycyclic compound represented by Formula 5 or Formula 6, thereby showing high emission efficiency.

### CROSS REFERENCE TO RELATED APPLICATIONS

This application is a Divisional of U.S. patent application Ser. No. 16/702,357, filed on Dec. 3, 2019, which claims priority from and the benefit of Korean Patent Application Nos. 10-2019-0025425, filed on Mar. 5, 2019 and 10-2019-0077607, filed on Jun. 28, 2019, which are hereby incorporated by reference for all purposes as if fully set forth herein.

### BACKGROUND

### Field

Exemplary embodiments of the invention relate generally to an organic electroluminescence device and a polycyclic compound for the organic electroluminescence device.

### Discussion of the Background

Recently, the development of an organic electroluminescence display device as an image display device is being actively conducted. Different from a liquid crystal display device, the organic electroluminescence display device is a so-called self-luminescent display device in which holes and electrons injected from a first electrode and a second electrode recombine in an emission layer, and a light-emitting material including an organic compound in the emission layer emits light to be used for a display.

In the application of an organic electroluminescence device to a display device, the decrease of the driving voltage, and the increase of the emission efficiency and the life of the organic electroluminescence device are required, and developments on materials for an organic electroluminescence device stably attaining the requirements are continuously being pursued.

The above information disclosed in this Background section is only for understanding of the background of the inventive concepts, and, therefore, it may contain information that does not constitute prior art.

### SUMMARY

Devices constructed according to exemplary embodiments of the invention are capable of providing an organic electroluminescence device and a polycyclic compound for the organic electroluminescence device, and more particularly, to an organic electroluminescence device having high efficiency, and a polycyclic compound included in a hole transport region of an organic electroluminescence device.

Additional features of the inventive concepts will be set forth in the description which follows, and in part will be apparent from the description, or may be learned by practice of is the inventive concepts.

An exemplary embodiment of the inventive concepts provides an organic electroluminescence device including a first electrode, a hole transport region on the first electrode, an emission layer on the hole transport region, an electron transport region on the emission layer, and a second electrode on the electron transport region, wherein the hole transport region includes a polycyclic compound represented by the following Formula 1:

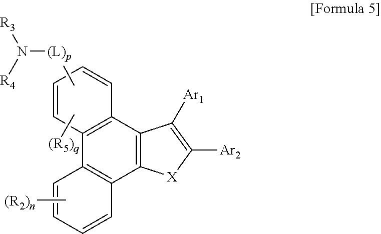

In Formula 1, X is 0 or S, Ar₁ and Ar₂ are each independently a substituted or unsubstituted aryl group of 6 to 30 carbon atoms for forming a ring, or a substituted or unsubstituted heteroaryl group of 2 to 30 carbon atoms for forming a ring, R₁ and R₂ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, “m” and “n” are each independently an integer of 0 to 4, and any one among Ar₁, Ar₂, R₁ and R₂ is represented by the following Formula 2:

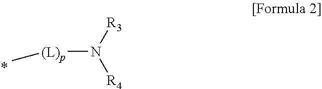

In Formula 2, L is a direct linkage, a substituted or unsubstituted arylene group of 6 to 30 carbon atoms for forming a ring, or a substituted or unsubstituted heteroarylene group of 2 to 30 carbon atoms for forming a ring, “p” is an integer of 0 to 3, and R₃ and R₄ are each is independently a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, or combined with an adjacent group to form a ring, where if Ar₁ or Ar₂ in Formula 1 are represented by Formula 2, L is not the direct linkage.

In an embodiment, Formula 1 may be represented by the following Formula 3 or Formula 4:

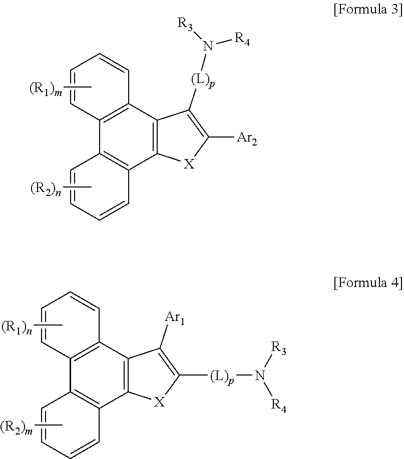

In Formula 3 and Formula 4, X, Ar₁, Ar₂, R₁ to R₄, L, “m”, “n” and “p” are the same as defined in Formula 1 and Formula 2.

In an embodiment, Formula 1 may be represented by the following Formula 5 or

Formula 6:

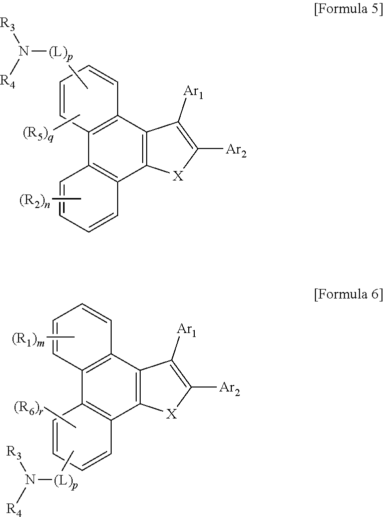

In an embodiment, in Formula 5 and Formula 6, R₅ and R₆ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, “q” and “r” are each independently an integer of 0 to 3, and X, Ar₁, Ar₂, R₁ to R₄, L, “m”, “n” and “p” are the same as defined in Formula 1 and Formula 2.

In an embodiment, L may be a substituted or unsubstituted arylene group of 6 to 12 carbon atoms for forming a ring.

In an embodiment, L may be a substituted or unsubstituted phenylene group.

In an embodiment, Ar₁ and Ar₂ may be each independently a substituted or unsubstituted aryl group of 6 to 20 carbon atoms for forming a ring.

In an embodiment, X may be O.

In an embodiment, Formula 5 may be represented by the following Formula 7 or Formula 8:

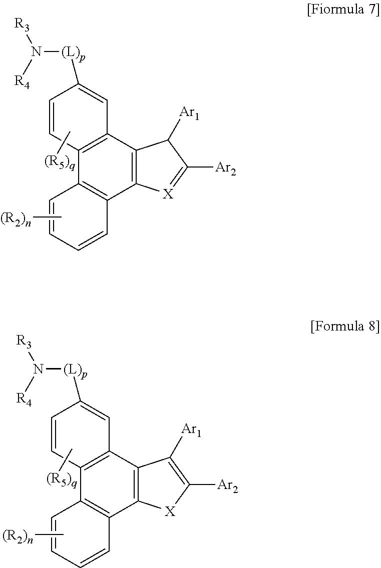

In Formula 7 and Formula 8, X, Ar₁, Ar₂, R₂ to R₅, L, “n”, “p” and “q” are the same as defined in Formula 5.

In an embodiment, Formula 6 may be represented by the following Formula 9 or Formula 10:

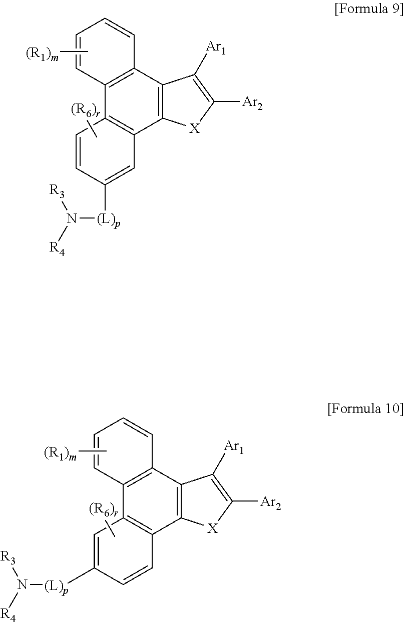

In Formula 9 and Formula 10, X, Ar₁, Ar₂, R₁, R₃, R₄, R₆, L, “m”, “p” and “r” are the same as defined in Formula 6.

In an embodiment, the hole transport region may include a hole injection layer on the first electrode, and a hole transport layer on the hole injection layer, wherein the hole transport layer may include the polycyclic compound represented by Formula 1.

In an embodiment, the hole transport region may further include an electron blocking layer on the hole transport layer.

In an embodiment, the polycyclic compound represented by Formula 1 may be any one selected among compounds represented in Compound Group 1 and Compound Group 2.

In an embodiment, the polycyclic compound represented by Formula 1 may be any one selected among compounds represented in Compound Group 3 to Compound Group 6.

In an exemplary embodiment of the inventive concepts, there is provided a polycyclic compound represented by Formula 1.

It is to be understood that both the foregoing general description and the following detailed description are exemplary and explanatory and are intended to provide further explanation of the invention as claimed.

### BRIEF DESCRIPTION OF THE FIGURES

The accompanying drawings, which are included to provide a further understanding of the invention and are incorporated in and constitute a part of this specification, illustrate exemplary embodiments of the invention, and together with the description serve to explain the inventive concepts.

FIG. 1 is a cross-sectional view schematically illustrating an organic electroluminescence device according to an embodiment of the inventive concepts;

FIG. 2 is a cross-sectional view schematically illustrating an organic electroluminescence device according to an embodiment of the inventive concepts; and

FIG. 3 is a cross-sectional view schematically illustrating an organic electroluminescence device according to an embodiment of the inventive concepts.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z—axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

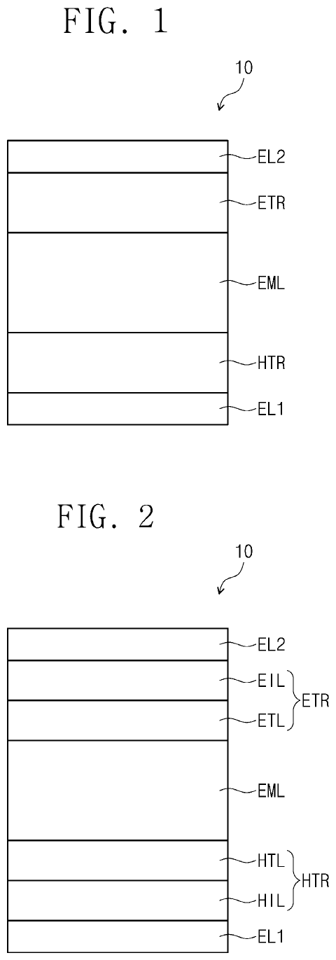

icFirst, the organic electroluminescence device according to an embodiment of the inventive concepts will be explained with reference to FIGS. 1 to 3.

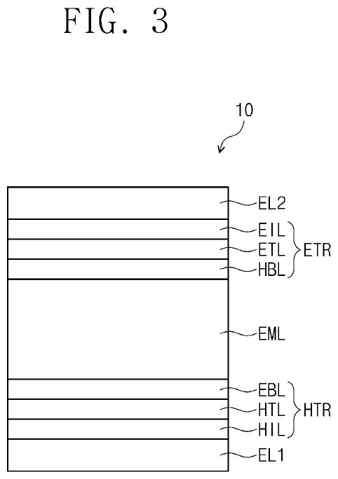

FIG. 1 is a cross-sectional view schematically illustrating an organic electroluminescence device according to an embodiment of the inventive concepts. FIG. 2 is a cross-sectional view schematically illustrating an organic electroluminescence device according to an embodiment of the inventive concepts. FIG. 3 is a cross-sectional view schematically is illustrating an organic electroluminescence device according to an embodiment of the inventive concepts.

Referring to FIGS. 1 to 3, an organic electroluminescence device 10 according to an embodiment includes a first electrode ELL a hole transport region HTR, an emission layer EML, an electron transport region ETR and a second electrode EL2, laminated one by one.

The hole transport region HTR includes the polycyclic compound according to an embodiment of the inventive concepts. Hereinafter, the polycyclic compound according to an embodiment of the inventive concepts will be explained in detail, and then, each layer of the organic electroluminescence device 10 will be explained.

In the description, [symbol] means a connecting position.

In the description, the term “substituted or unsubstituted” corresponds to substituted or unsubstituted with at least one substituent selected from the group consisting of a deuterium atom, a halogen atom, a cyano group, a nitro group, an amino group, a silyl group, a boron group, a phosphine oxide group, a phosphine sulfide group, an alkyl group, an alkenyl group, an aryl group, and a heterocyclic group. In addition, each of the exemplified substituents may be substituted or unsubstituted. For example, a biphenyl group may be interpreted as an aryl group, or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The ring formed by the combination with an adjacent group may be a monocyclic ring or a polycyclic ring. In addition, the ring formed via the combination with an adjacent group may be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other.

In the description, examples of the halogen atom include a fluorine atom, a chlorine atom, a bromine atom or an iodine atom.

In the description, the alkyl may be a linear, branched or cyclic type. The carbon number of the alkyl may be 1 to 50, 1 to 30, 1 to 20, 1 to 10, or 1 to 6. Examples of the alkyl may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, the aryl group means an optional functional group or substituent derived from an aromatic hydrocarbon ring. The aryl group may be a monocyclic aryl group or a polycyclic aryl group. The carbon number for forming a ring in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinqphenyl, sexiphenyl, triphenylenyl, pyrenyl, benzofluoranthenyl, chrysenyl, etc., without limitation.

In the description, the fluorenyl group may be substituted, and two substituents may be combined with each other to form a Spiro structure. Examples of a substituted fluorenyl group are as follows. However, an embodiment of the inventive concepts is not limited thereto.

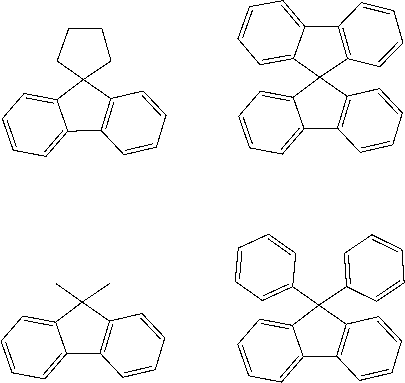

In the description, the heteroaryl may be a heteroaryl including at least one of O, N, P, Si or S as a heteroatom. The carbon number for forming a ring of the heteroaryl may be 2 to 30, or 2 to 20. The heteroaryl may be monocyclic heteroaryl or polycyclic heteroaryl. Examples of the polycyclic heteroaryl may have a dicyclic or tricyclic structure. Examples of the heteroaryl may include thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phenoxazyl, phthalazinyl, pyrido pyrimidinyl, pyrido pyrazinyl, pyrazino pyrazinyl, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophenyl, thienothiophene, benzofuranyl, phenanthroline, thiazolyl, isooxazolyl, oxadiazolyl, thiadiazolyl, benzothiazolyl, phenothiazinyl, dibenzosilole, dibenzofuran, etc., without limitation.

In the description, the silyl group includes an alkyl silyl group and an aryl silyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, an embodiment of the inventive concepts is not limited thereto.

In the description, the explanation on the aryl group may be applied to the arylene group except that the arylene group is a divalent group.

In the description, the explanation on the heteroaryl group may be applied to the heteroarylene group except that the heteroarylene group is a divalent group.

The polycyclic compound according to an embodiment of the inventive concepts is represented by the following Formula 1:

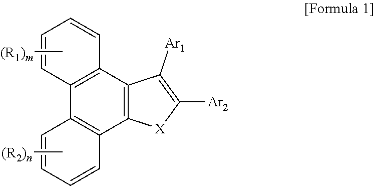

In Formula 1, X is O or S.

In Formula 1, Ar₁ and Ar₂ are each independently a substituted or unsubstituted aryl group of 6 to 30 carbon atoms for forming a ring, or a substituted or unsubstituted heteroaryl group of 2 to 30 carbon atoms for forming a ring.

In Formula 1, R₁ and R₂ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring.

In Formula 1, “m” is an integer of 0 to 4. Meanwhile, if “m” is 2 or more, a plurality of R₁ groups are the same or different.

In Formula 1, “n” is an integer of 0 to 4. Meanwhile, if “m” is 2 or more, a plurality of R₂ groups are the same or different.

In Formula 1, any one among Ar₁, Ar₂, R₁ and R₂ is represented by the following

Formula 2:

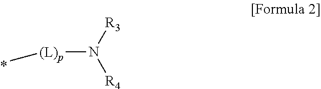

In Formula 2, L is a substituted or unsubstituted arylene group of 6 to 30 carbon atoms for forming a ring, or a substituted or unsubstituted heteroarylene group of 2 to 30 carbon atoms for forming a ring.

In Formula 2, “p” is an integer of 0 to 3. Meanwhile, if “p” is 2 or more, a plurality of L groups are the same or different.

In Formula 2, R₃ and R₄ are each independently a substituted or unsubstituted alkyl group of 1 to 20 carbon atoms, a substituted or unsubstituted aryl group of 6 to 30 carbon atoms for forming a ring, a substituted or unsubstituted heteroaryl group of 2 to 30 carbon atoms for forming a ring, or combined with an adjacent group to form a ring.

Meanwhile, if Ar₁ or Ar₂ in Formula 1 are represented by Formula 2, L in Formula 2 is not the direct linkage. In the polycyclic compound according to the inventive concepts, Ar₁ and Ar₂ are definitely substituted or unsubstituted aryl groups or substituted or unsubstituted heteroaryl groups. That is, the polycyclic compound has a structure in which an aryl group or a heteroaryl group, which has a large volume, is substituted at the α position and βposition of a highly reactive furan ring and thiophene ring of phenanthrofuran and phenanthrothiophene.

In an embodiment, Ar₁ in Formula 1 may be represented by Formula 2. In this case, Formula 1 may be represented by Formula 3.

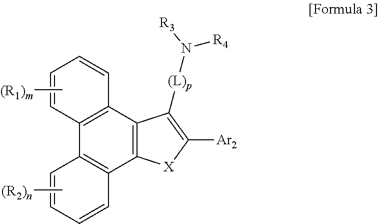

In Formula 3, X, Ar₂, R₁ to R₄, L, “m”, “n” and “p” are the same as defined in Formula 1 and Formula 2.

In an embodiment, Ar₂ in Formula 1 may be represented by Formula 2. In this case, Formula 1 may be represented by Formula 4.

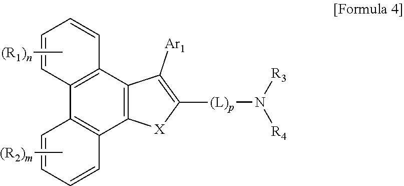

In Formula 4, X, Ar₁, R₁ to R₄, L, “m”, “n” and “p” are the same as defined in Formula 1 and Formula 2.

In an embodiment, R₁ in Formula 1 may be represented by Formula 2. In this case, Formula 1 may be represented by the following Formula 5:

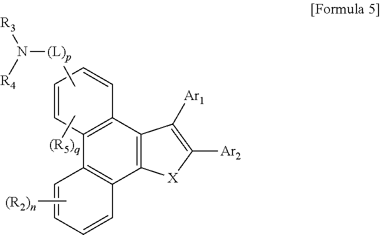

In Formula 5, R₅ is a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring.

In Formula 5, “q” may be an integer of 0 to 3. Meanwhile, if “q” is 2 or more, a plurality of R₅ groups are the same or different.

In Formula 5, X, Ar₁, Ar₂, R₂ to R₄, L, “n” and “p” are the same as defined in Formula 1 and Formula 2.

In an embodiment, Formula 5 may be represented by the following Formula 7 or Formula 8:

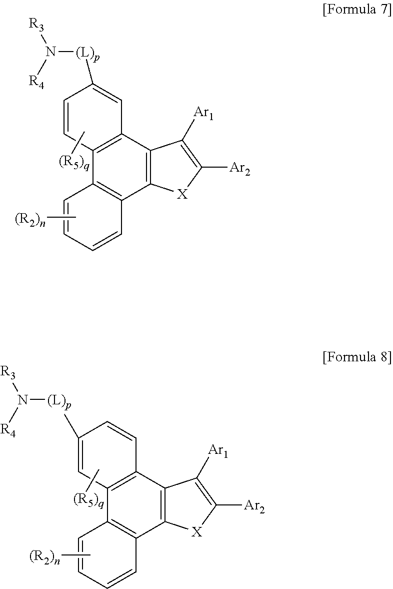

In Formula 7 and Formula 8, X, Ar₁, Ar₂, R₂ to R₅, L, “n”, “p” and “q” are the same as defined in Formula 5.

In an embodiment, R₂ in Formula 1 may be represented by Formula 2. In this case, Formula 1 may be represented by the following Formula 6:

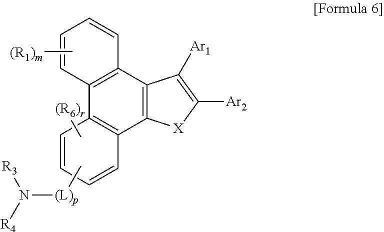

In Formula 6, R₆ may be a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring.

In Formula 6, “r” may be an integer of 0 to 3. Meanwhile, if “r” is 2 or more, a plurality of R₆ groups are the same or different.

In Formula 6, X, Ar₁, Ar₂, R₁, R₃, R₄, L, “m” and “p” are the same as defined in Formula 1 and Formula 2.

In an embodiment, Formula 6 may be represented by the following Formula 9 and Formula 10:

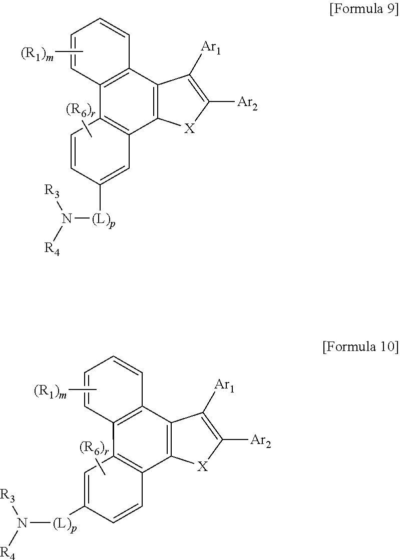

In Formula 9 and Formula 10, X, Ar₁, Ar₂, R₁, R₃, R₄, R₆, L, “m”, “p” and “r” are the same as defined in Formula 6.

In Formula 1, L may be a substituted or unsubstituted arylene group of 6 to 12 carbon atoms for forming a ring. L may be, for example, a substituted or unsubstituted phenylene group. However, an embodiment of the inventive concepts is not limited thereto. In this case, “m” may be 1. However, an embodiment of the inventive concepts is not limited thereto.

In Formula 1, Ar₁ and Ar₂ may be each independently a substituted or unsubstituted aryl group of 6 to 20 carbon atoms for forming a ring. For example, Ar₁ and Ar₂ may be each independently a substituted or unsubstituted phenyl group, a substituted or unsubstituted biphenyl group, a substituted or unsubstituted naphthyl group, or a substituted or unsubstituted fluorenyl group. However, an embodiment of the inventive concepts is not limited thereto.

In an embodiment, X in Formula 1 may be 0.

The polycyclic compound represented by Formula 1 according to an embodiment of the inventive concepts may be any one selected among the compounds represented in the following Compound Group 1 and Compound Group 2, without limitation:

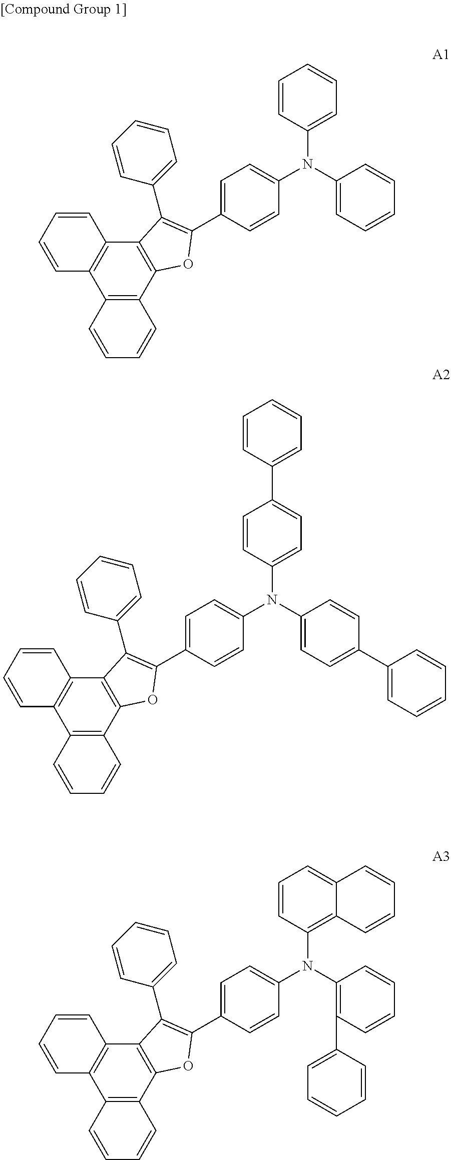 [SMILES: c1ccc(-c2c(-c3ccc(N(c4ccccc4)c4ccccc4)cc3)oc3c4ccccc4c4ccccc4c23)cc1 ; c1ccc(-c2ccc(N(c3ccc(-c4ccccc4)cc3)c3ccc(-c4oc5c6ccccc6c6ccccc6c5c4-c4ccccc4)cc3)cc2)cc1 ; c1ccc(-c2ccccc2N(c2ccc(-c3oc4c5ccccc5c5ccccc5c4c3-c3ccccc3)cc2)c2cccc3ccccc23)cc1]

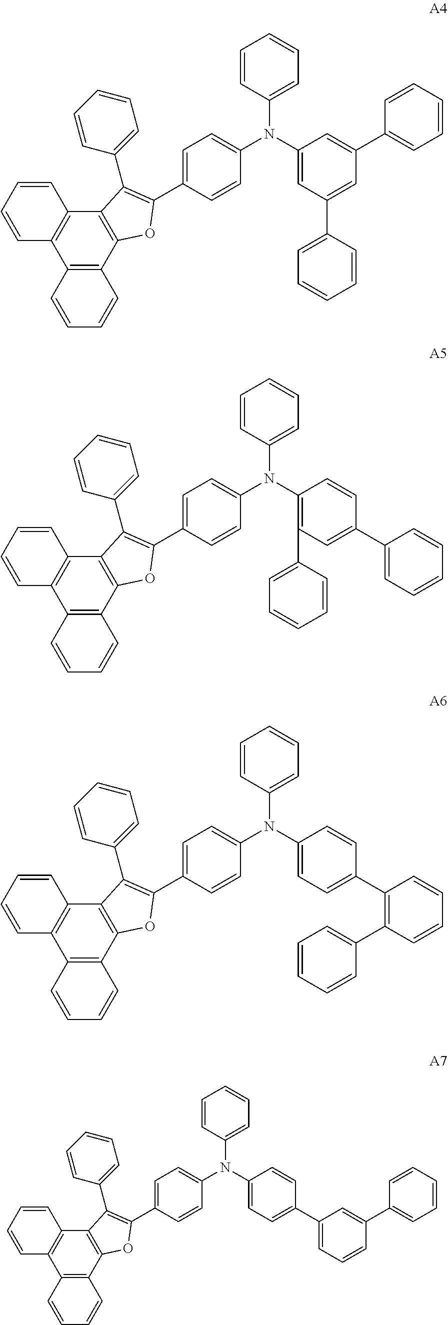 [SMILES: c1ccc(-c2cc(-c3ccccc3)cc(N(c3ccccc3)c3ccc(-c4oc5c6ccccc6c6ccccc6c5c4-c4ccccc4)cc3)c2)cc1 ; c1ccc(-c2ccc(N(c3ccccc3)c3ccc(-c4oc5c6ccccc6c6ccccc6c5c4-c4ccccc4)cc3)c(-c3ccccc3)c2)cc1 ; c1ccc(-c2ccccc2-c2ccc(N(c3ccccc3)c3ccc(-c4oc5c6ccccc6c6ccccc6c5c4-c4ccccc4)cc3)cc2)cc1 ; c1ccc(-c2cccc(-c3ccc(N(c4ccccc4)c4ccc(-c5oc6c7ccccc7c7ccccc7c6c5-c5ccccc5)cc4)cc3)c2)cc1]

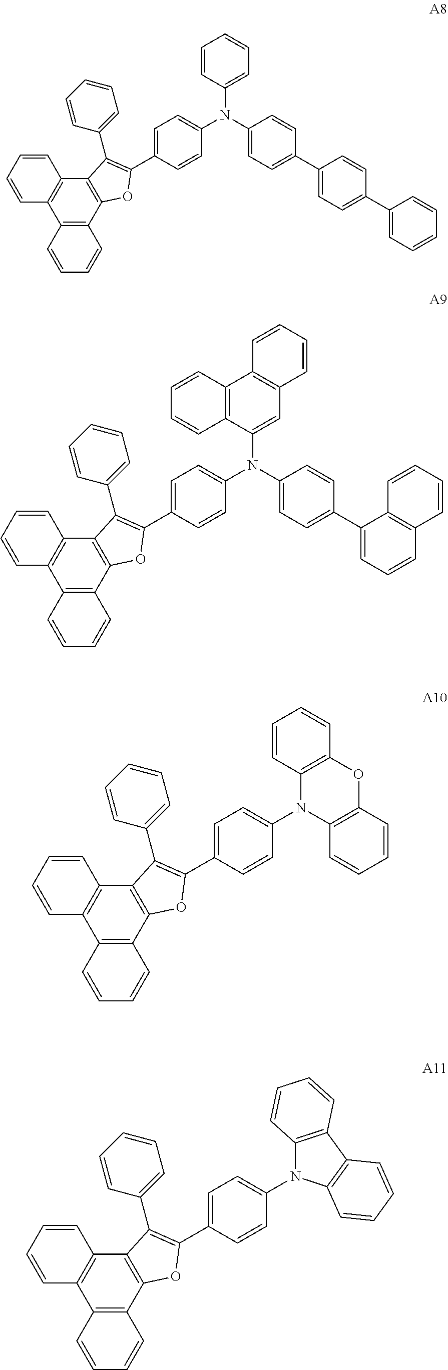 [SMILES: c1ccc(-c2ccc(-c3ccc(N(c4ccccc4)c4ccc(-c5oc6c7ccccc7c7ccccc7c6c5-c5ccccc5)cc4)cc3)cc2)cc1 ; c1ccc(-c2c(-c3ccc(N(c4ccc(-c5cccc6ccccc56)cc4)c4cc5ccccc5c5ccccc45)cc3)oc3c4ccccc4c4ccccc4c23)cc1 ; c1ccc(-c2c(-c3ccc(N4c5ccccc5Oc5ccccc54)cc3)oc3c4ccccc4c4ccccc4c23)cc1 ; c1ccc(-c2c(-c3ccc(-n4c5ccccc5c5ccccc54)cc3)oc3c4ccccc4c4ccccc4c23)cc1]

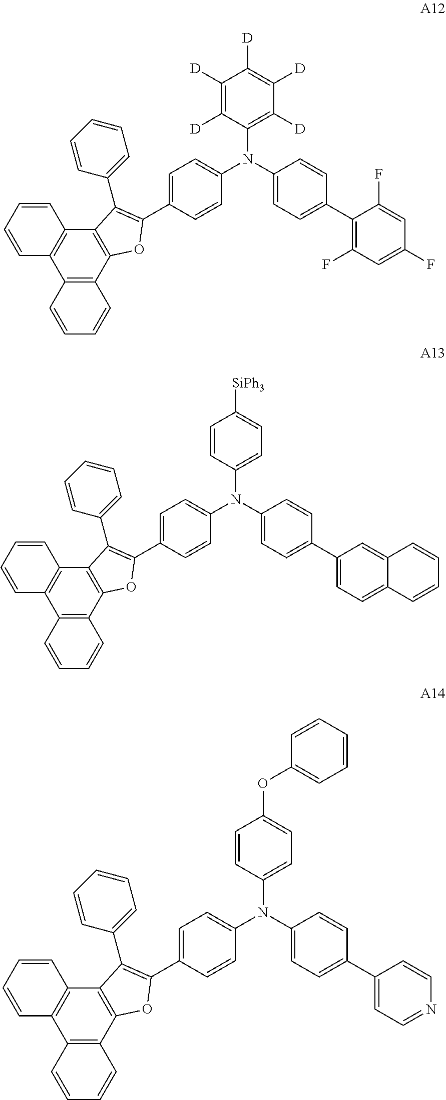

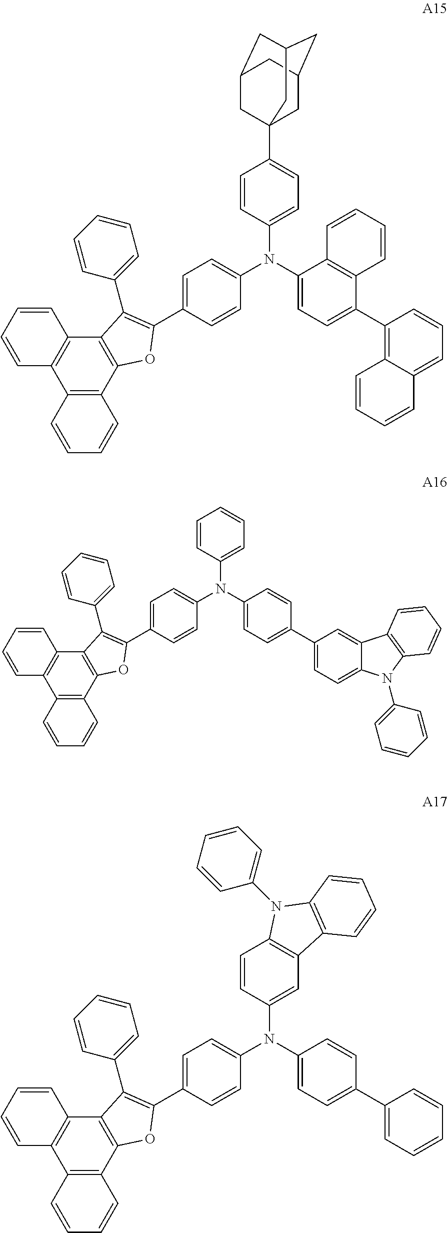 [SMILES: c1ccc(-c2c(-c3ccc(N(c4ccc(C56CC7CC(CC(C7)C5)C6)cc4)c4ccc(-c5cccc6ccccc56)c5ccccc45)cc3)oc3c4ccccc4c4ccccc4c23)cc1 ; c1ccc(-c2c(-c3ccc(N(c4ccccc4)c4ccc(-c5ccc6c(c5)c5ccccc5n6-c5ccccc5)cc4)cc3)oc3c4ccccc4c4ccccc4c23)cc1 ; c1ccc(-c2ccc(N(c3ccc(-c4oc5c6ccccc6c6ccccc6c5c4-c4ccccc4)cc3)c3ccc4c(c3)c3ccccc3n4-c3ccccc3)cc2)cc1]

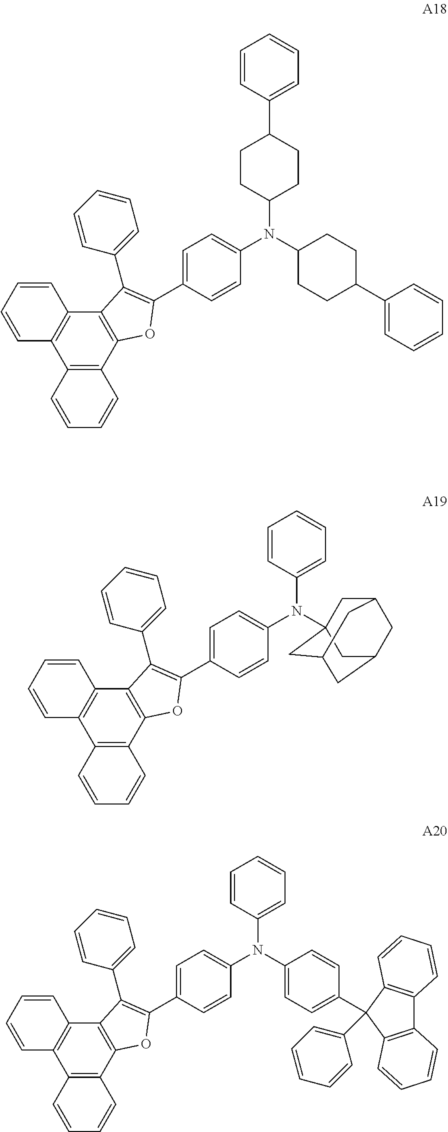 [SMILES: c1ccc(-c2c(-c3ccc(N(C4CCC(c5ccccc5)CC4)C4CCC(c5ccccc5)CC4)cc3)oc3c4ccccc4c4ccccc4c23)cc1 ; c1ccc(-c2c(-c3ccc(N(c4ccccc4)C45CC6CC(CC(C6)C4)C5)cc3)oc3c4ccccc4c4ccccc4c23)cc1 ; c1ccc(-c2c(-c3ccc(N(c4ccccc4)c4ccc(C5(c6ccccc6)c6ccccc6-c6ccccc65)cc4)cc3)oc3c4ccccc4c4ccccc4c23)cc1]

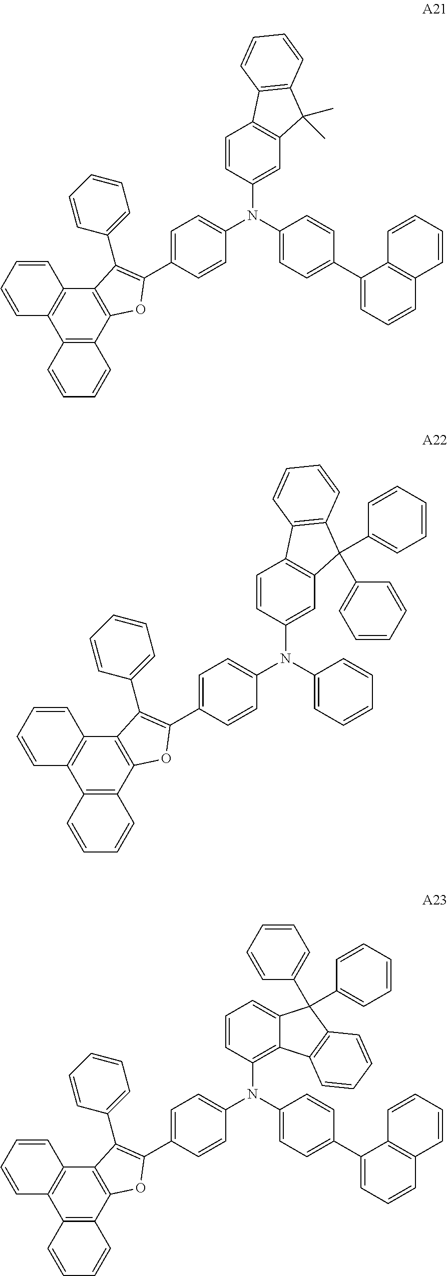 [SMILES: CC1(C)c2ccccc2-c2ccc(N(c3ccc(-c4oc5c6ccccc6c6ccccc6c5c4-c4ccccc4)cc3)c3ccc(-c4cccc5ccccc45)cc3)cc21 ; c1ccc(-c2c(-c3ccc(N(c4ccccc4)c4ccc5c(c4)C(c4ccccc4)(c4ccccc4)c4ccccc4-5)cc3)oc3c4ccccc4c4ccccc4c23)cc1 ; c1ccc(-c2c(-c3ccc(N(c4ccc(-c5cccc6ccccc56)cc4)c4cccc5c4-c4ccccc4C5(c4ccccc4)c4ccccc4)cc3)oc3c4ccccc4c4ccccc4c23)cc1]

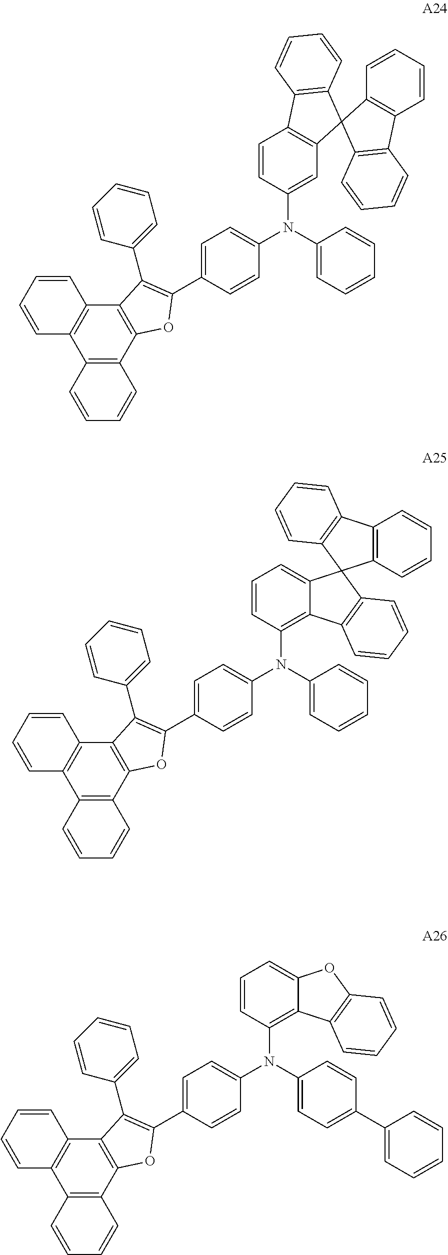 [SMILES: c1ccc(-c2c(-c3ccc(N(c4ccccc4)c4ccc5c(c4)C4(c6ccccc6-c6ccccc64)c4ccccc4-5)cc3)oc3c4ccccc4c4ccccc4c23)cc1 ; c1ccc(-c2c(-c3ccc(N(c4ccccc4)c4cccc5c4-c4ccccc4C54c5ccccc5-c5ccccc54)cc3)oc3c4ccccc4c4ccccc4c23)cc1 ; c1ccc(-c2ccc(N(c3ccc(-c4oc5c6ccccc6c6ccccc6c5c4-c4ccccc4)cc3)c3cccc4oc5ccccc5c34)cc2)cc1]

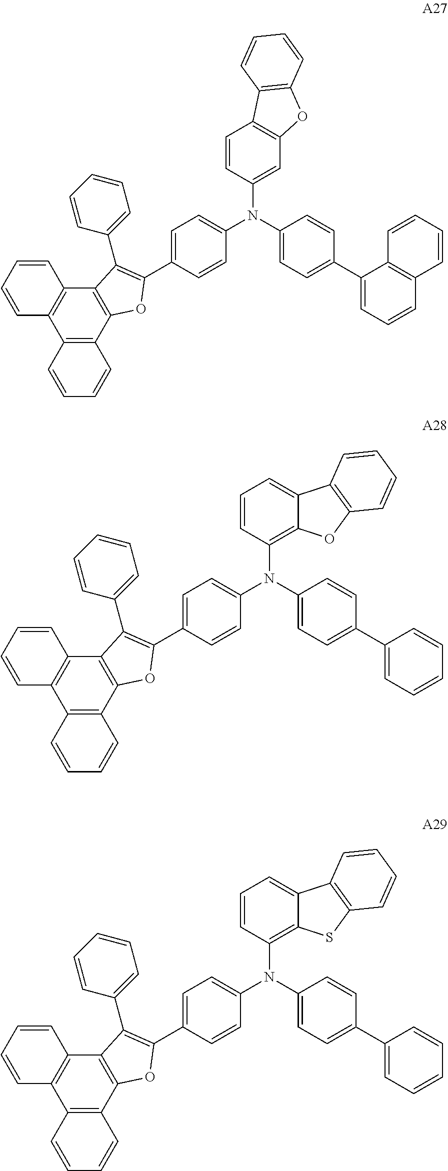 [SMILES: c1ccc(-c2c(-c3ccc(N(c4ccc(-c5cccc6ccccc56)cc4)c4ccc5c(c4)oc4ccccc45)cc3)oc3c4ccccc4c4ccccc4c23)cc1 ; c1ccc(-c2ccc(N(c3ccc(-c4oc5c6ccccc6c6ccccc6c5c4-c4ccccc4)cc3)c3cccc4c3oc3ccccc34)cc2)cc1 ; c1ccc(-c2ccc(N(c3ccc(-c4oc5c6ccccc6c6ccccc6c5c4-c4ccccc4)cc3)c3cccc4c3sc3ccccc34)cc2)cc1]

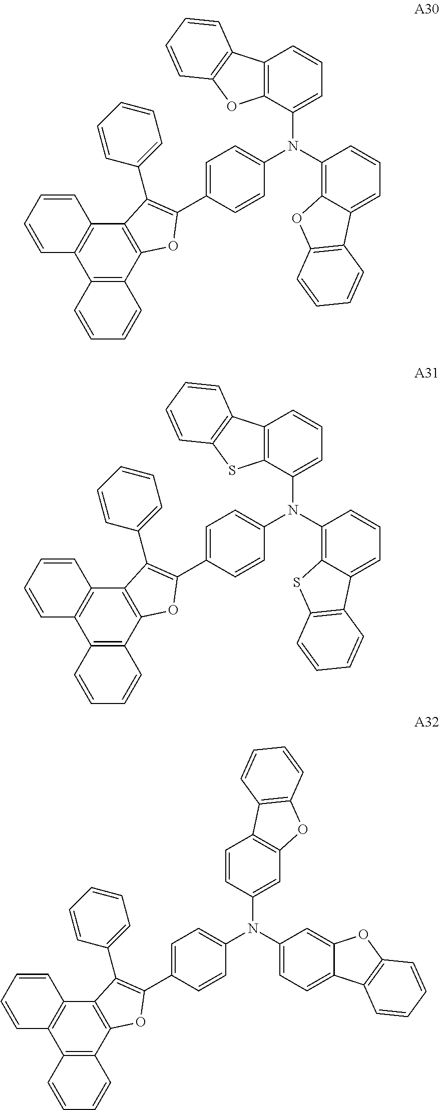 [SMILES: c1ccc(-c2c(-c3ccc(N(c4cccc5c4oc4ccccc45)c4cccc5c4oc4ccccc45)cc3)oc3c4ccccc4c4ccccc4c23)cc1 ; c1ccc(-c2c(-c3ccc(N(c4cccc5c4sc4ccccc45)c4cccc5c4sc4ccccc45)cc3)oc3c4ccccc4c4ccccc4c23)cc1 ; c1ccc(-c2c(-c3ccc(N(c4ccc5c(c4)oc4ccccc45)c4ccc5c(c4)oc4ccccc45)cc3)oc3c4ccccc4c4ccccc4c23)cc1]

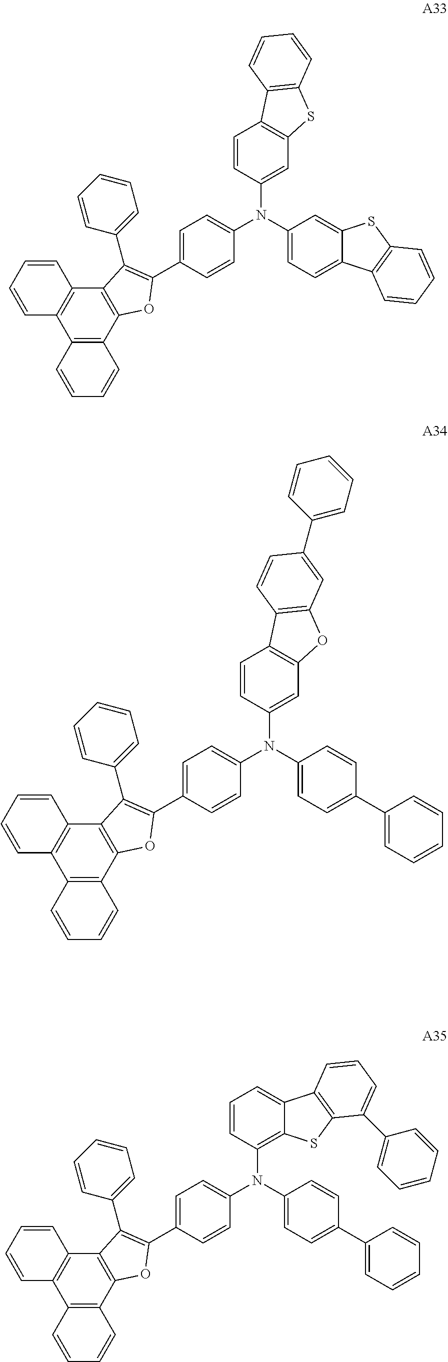 [SMILES: c1ccc(-c2c(-c3ccc(N(c4ccc5c(c4)sc4ccccc45)c4ccc5c(c4)sc4ccccc45)cc3)oc3c4ccccc4c4ccccc4c23)cc1 ; c1ccc(-c2ccc(N(c3ccc(-c4oc5c6ccccc6c6ccccc6c5c4-c4ccccc4)cc3)c3ccc4c(c3)oc3cc(-c5ccccc5)ccc34)cc2)cc1 ; c1ccc(-c2ccc(N(c3ccc(-c4oc5c6ccccc6c6ccccc6c5c4-c4ccccc4)cc3)c3cccc4c3sc3c(-c5ccccc5)cccc34)cc2)cc1]

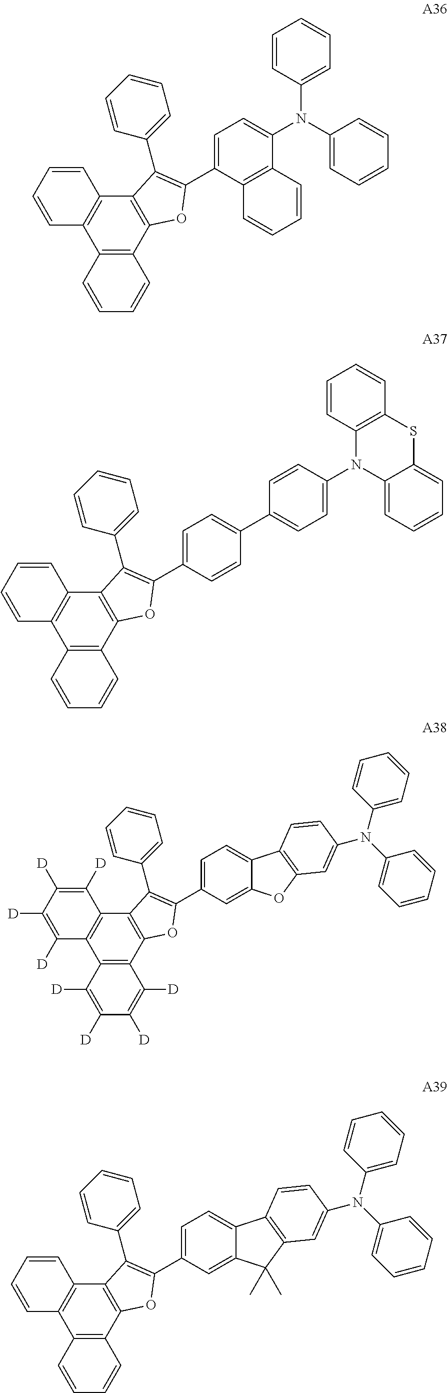 [SMILES: c1ccc(-c2c(-c3ccc(N(c4ccccc4)c4ccccc4)c4ccccc34)oc3c4ccccc4c4ccccc4c23)cc1 ; c1ccc(-c2c(-c3ccc(-c4ccc(N5c6ccccc6Sc6ccccc65)cc4)cc3)oc3c4ccccc4c4ccccc4c23)cc1 ; [2H]c1c([2H])c([2H])c2c(c1[2H])c1oc(-c3ccc4c(c3)oc3cc(N(c5ccccc5)c5ccccc5)ccc34)c(-c3ccccc3)c1c1c([2H])c([2H])c([2H])c([2H])c21 ; CC1(C)c2cc(-c3oc4c5ccccc5c5ccccc5c4c3-c3ccccc3)ccc2-c2ccc(N(c3ccccc3)c3ccccc3)cc21]

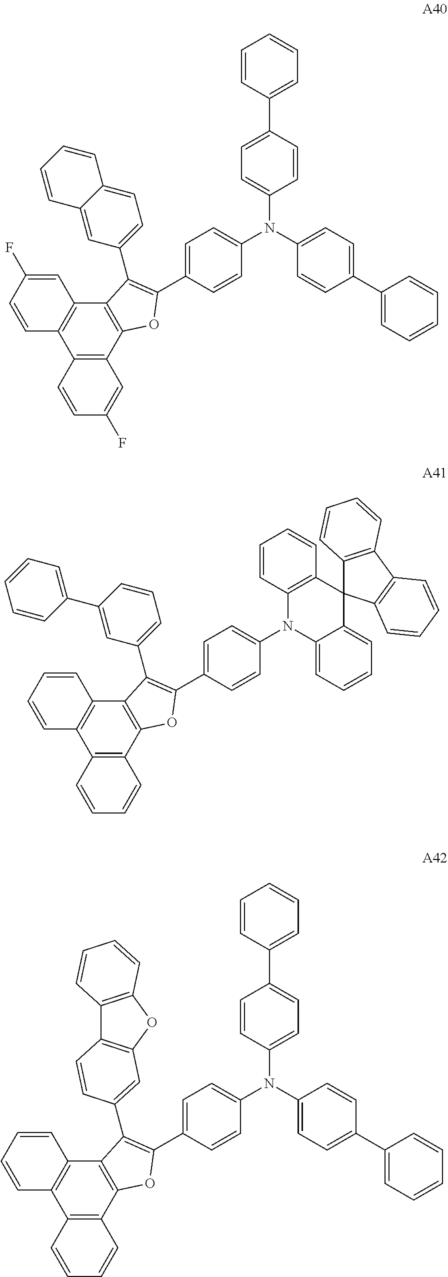 [SMILES: Fc1ccc2c3ccc(F)cc3c3c(-c4ccc5ccccc5c4)c(-c4ccc(N(c5ccc(-c6ccccc6)cc5)c5ccc(-c6ccccc6)cc5)cc4)oc3c2c1 ; c1ccc(-c2cccc(-c3c(-c4ccc(N5c6ccccc6C6(c7ccccc7-c7ccccc76)c6ccccc65)cc4)oc4c5ccccc5c5ccccc5c34)c2)cc1 ; c1ccc(-c2ccc(N(c3ccc(-c4ccccc4)cc3)c3ccc(-c4oc5c6ccccc6c6ccccc6c5c4-c4ccc5c(c4)oc4ccccc45)cc3)cc2)cc1]

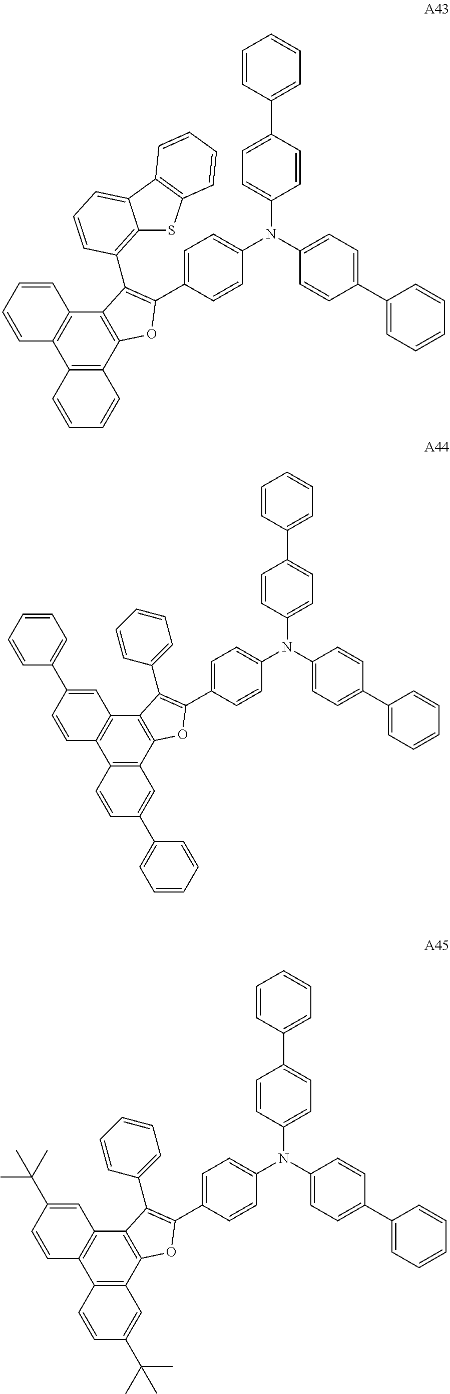 [SMILES: c1ccc(-c2ccc(N(c3ccc(-c4ccccc4)cc3)c3ccc(-c4oc5c6ccccc6c6ccccc6c5c4-c4cccc5c4sc4ccccc45)cc3)cc2)cc1 ; c1ccc(-c2ccc(N(c3ccc(-c4ccccc4)cc3)c3ccc(-c4oc5c6cc(-c7ccccc7)ccc6c6ccc(-c7ccccc7)cc6c5c4-c4ccccc4)cc3)cc2)cc1 ; CC(C)(C)c1ccc2c3ccc(C(C)(C)C)cc3c3c(-c4ccccc4)c(-c4ccc(N(c5ccc(-c6ccccc6)cc5)c5ccc(-c6ccccc6)cc5)cc4)oc3c2c1]

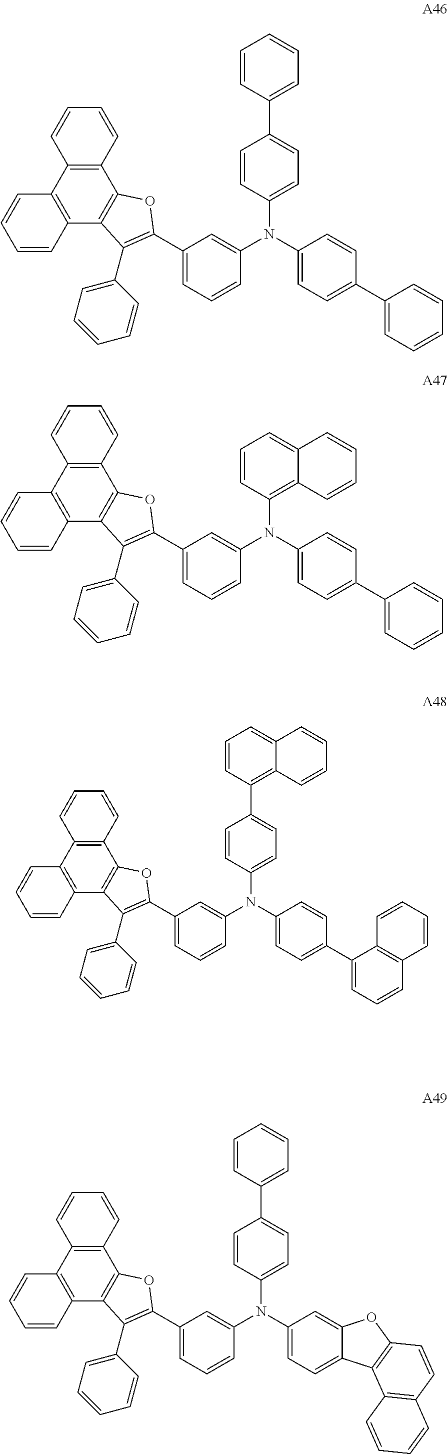 [SMILES: c1ccc(-c2ccc(N(c3ccc(-c4ccccc4)cc3)c3cccc(-c4oc5c6ccccc6c6ccccc6c5c4-c4ccccc4)c3)cc2)cc1 ; c1ccc(-c2ccc(N(c3cccc(-c4oc5c6ccccc6c6ccccc6c5c4-c4ccccc4)c3)c3cccc4ccccc34)cc2)cc1 ; c1ccc(-c2c(-c3cccc(N(c4ccc(-c5cccc6ccccc56)cc4)c4ccc(-c5cccc6ccccc56)cc4)c3)oc3c4ccccc4c4ccccc4c23)cc1 ; c1ccc(-c2ccc(N(c3cccc(-c4oc5c6ccccc6c6ccccc6c5c4-c4ccccc4)c3)c3ccc4c(c3)oc3ccc5ccccc5c34)cc2)cc1]

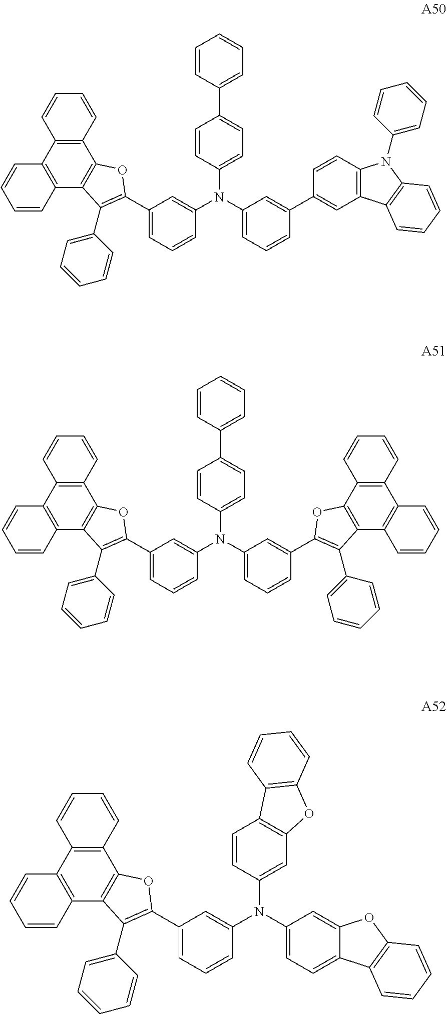 [SMILES: c1ccc(-c2ccc(N(c3cccc(-c4ccc5c(c4)c4ccccc4n5-c4ccccc4)c3)c3cccc(-c4oc5c6ccccc6c6ccccc6c5c4-c4ccccc4)c3)cc2)cc1 ; c1ccc(-c2ccc(N(c3cccc(-c4oc5c6ccccc6c6ccccc6c5c4-c4ccccc4)c3)c3cccc(-c4oc5c6ccccc6c6ccccc6c5c4-c4ccccc4)c3)cc2)cc1 ; c1ccc(-c2c(-c3cccc(N(c4ccc5c(c4)oc4ccccc45)c4ccc5c(c4)oc4ccccc45)c3)oc3c4ccccc4c4ccccc4c23)cc1]

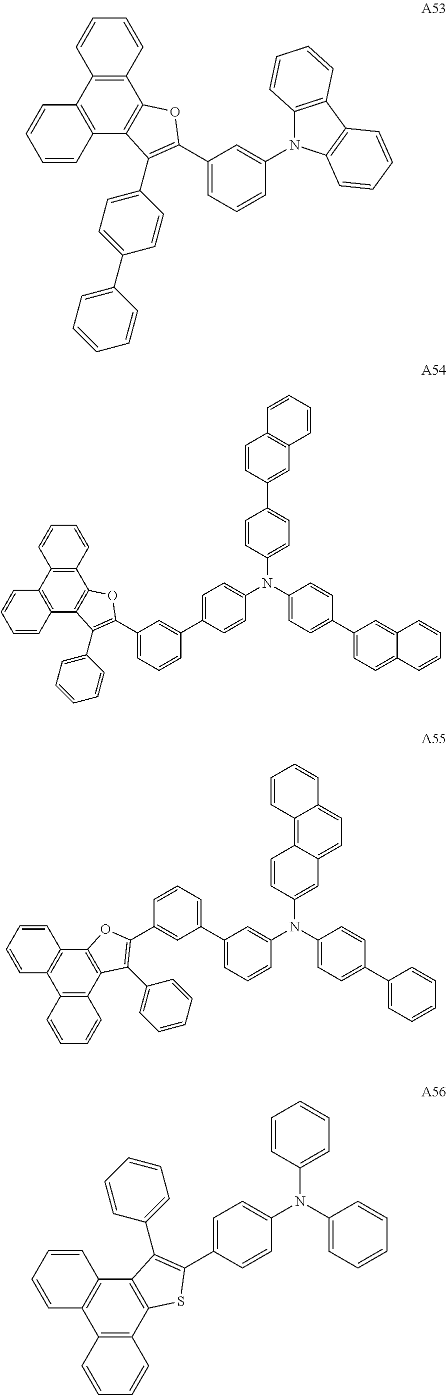 [SMILES: c1ccc(-c2ccc(-c3c(-c4cccc(-n5c6ccccc6c6ccccc65)c4)oc4c5ccccc5c5ccccc5c34)cc2)cc1 ; c1ccc(-c2c(-c3cccc(-c4ccc(N(c5ccc(-c6ccc7ccccc7c6)cc5)c5ccc(-c6ccc7ccccc7c6)cc5)cc4)c3)oc3c4ccccc4c4ccccc4c23)cc1 ; c1ccc(-c2ccc(N(c3cccc(-c4cccc(-c5oc6c7ccccc7c7ccccc7c6c5-c5ccccc5)c4)c3)c3ccc4c(ccc5ccccc54)c3)cc2)cc1 ; c1ccc(-c2c(-c3ccc(N(c4ccccc4)c4ccccc4)cc3)sc3c4ccccc4c4ccccc4c23)cc1]

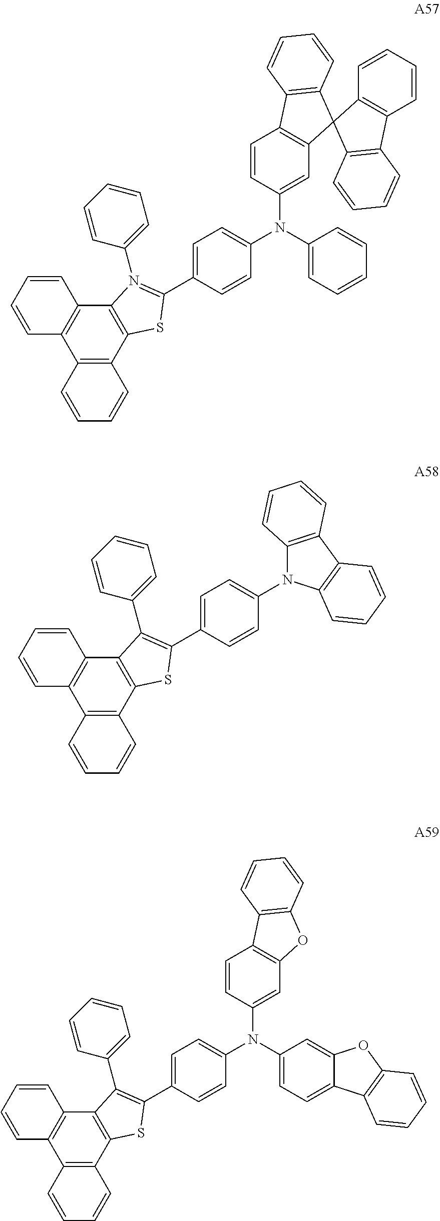

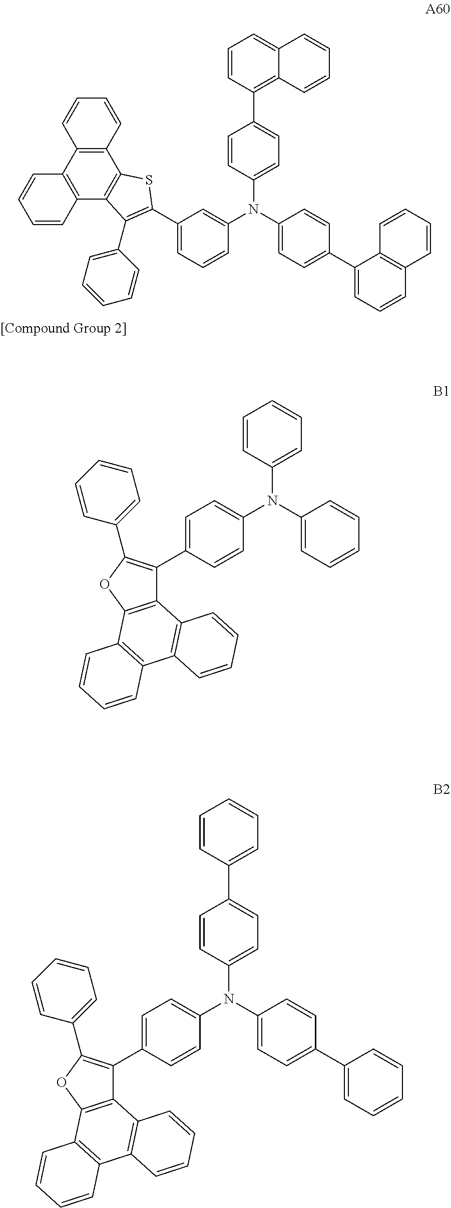 [SMILES: c1ccc(-c2c(-c3cccc(N(c4ccc(-c5cccc6ccccc56)cc4)c4ccc(-c5cccc6ccccc56)cc4)c3)sc3c4ccccc4c4ccccc4c23)cc1 ; c1ccc(-c2oc3c4ccccc4c4ccccc4c3c2-c2ccc(N(c3ccccc3)c3ccccc3)cc2)cc1 ; c1ccc(-c2ccc(N(c3ccc(-c4ccccc4)cc3)c3ccc(-c4c(-c5ccccc5)oc5c6ccccc6c6ccccc6c45)cc3)cc2)cc1]

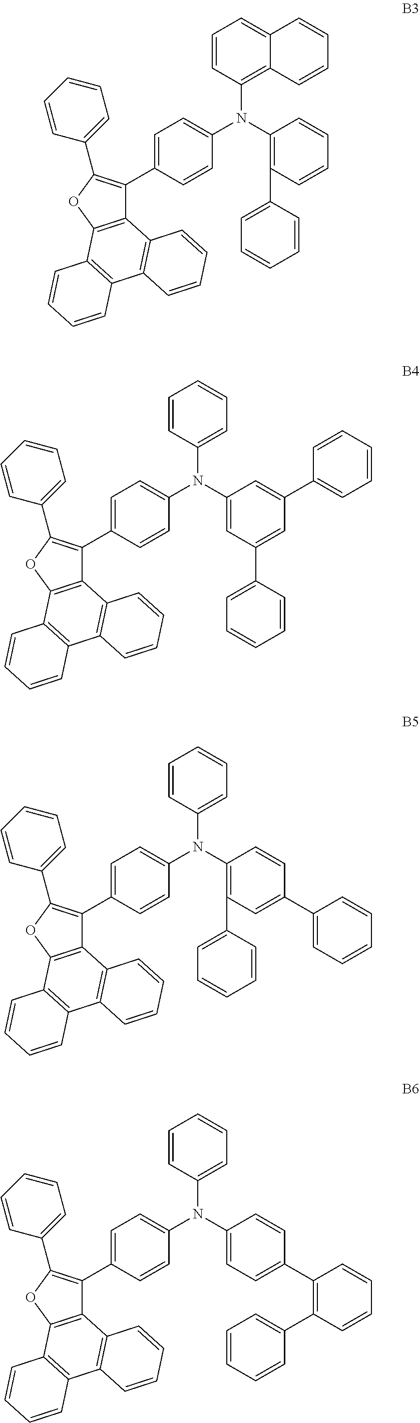 [SMILES: c1ccc(-c2ccccc2N(c2ccc(-c3c(-c4ccccc4)oc4c5ccccc5c5ccccc5c34)cc2)c2cccc3ccccc23)cc1 ; c1ccc(-c2cc(-c3ccccc3)cc(N(c3ccccc3)c3ccc(-c4c(-c5ccccc5)oc5c6ccccc6c6ccccc6c45)cc3)c2)cc1 ; c1ccc(-c2ccc(N(c3ccccc3)c3ccc(-c4c(-c5ccccc5)oc5c6ccccc6c6ccccc6c45)cc3)c(-c3ccccc3)c2)cc1 ; c1ccc(-c2ccccc2-c2ccc(N(c3ccccc3)c3ccc(-c4c(-c5ccccc5)oc5c6ccccc6c6ccccc6c45)cc3)cc2)cc1]

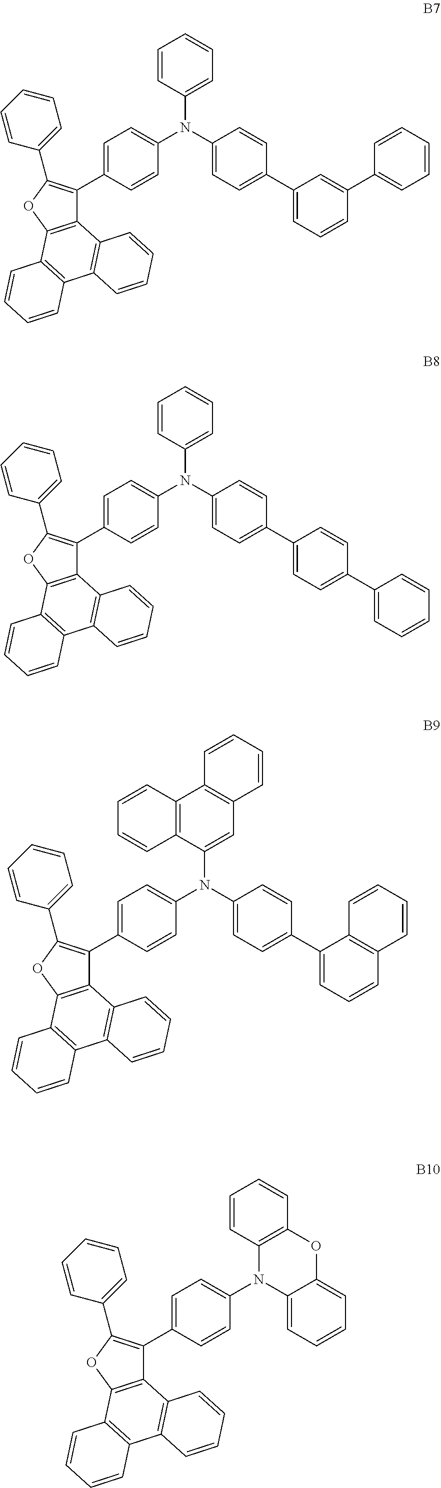 [SMILES: c1ccc(-c2cccc(-c3ccc(N(c4ccccc4)c4ccc(-c5c(-c6ccccc6)oc6c7ccccc7c7ccccc7c56)cc4)cc3)c2)cc1 ; c1ccc(-c2ccc(-c3ccc(N(c4ccccc4)c4ccc(-c5c(-c6ccccc6)oc6c7ccccc7c7ccccc7c56)cc4)cc3)cc2)cc1 ; c1ccc(-c2oc3c4ccccc4c4ccccc4c3c2-c2ccc(N(c3ccc(-c4cccc5ccccc45)cc3)c3cc4ccccc4c4ccccc34)cc2)cc1 ; c1ccc(-c2oc3c4ccccc4c4ccccc4c3c2-c2ccc(N3c4ccccc4Oc4ccccc43)cc2)cc1]

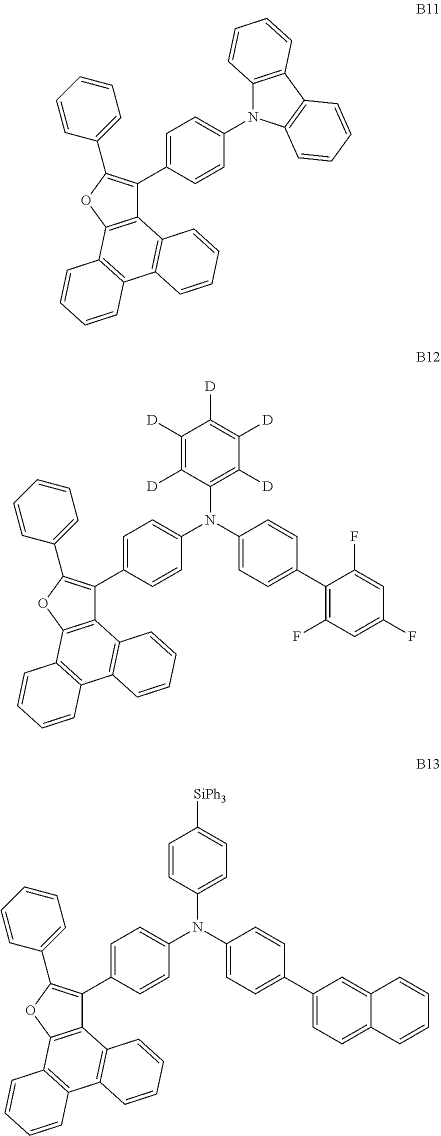

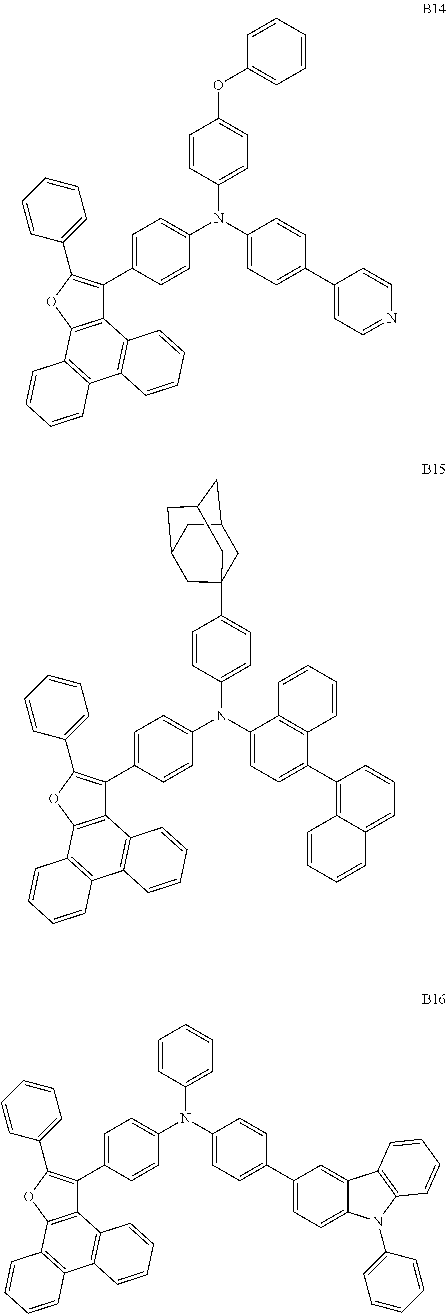 [SMILES: c1ccc(Oc2ccc(N(c3ccc(-c4ccncc4)cc3)c3ccc(-c4c(-c5ccccc5)oc5c6ccccc6c6ccccc6c45)cc3)cc2)cc1 ; c1ccc(-c2oc3c4ccccc4c4ccccc4c3c2-c2ccc(N(c3ccc(C45CC6CC(CC(C6)C4)C5)cc3)c3ccc(-c4cccc5ccccc45)c4ccccc34)cc2)cc1 ; c1ccc(-c2oc3c4ccccc4c4ccccc4c3c2-c2ccc(N(c3ccccc3)c3ccc(-c4ccc5c(c4)c4ccccc4n5-c4ccccc4)cc3)cc2)cc1]

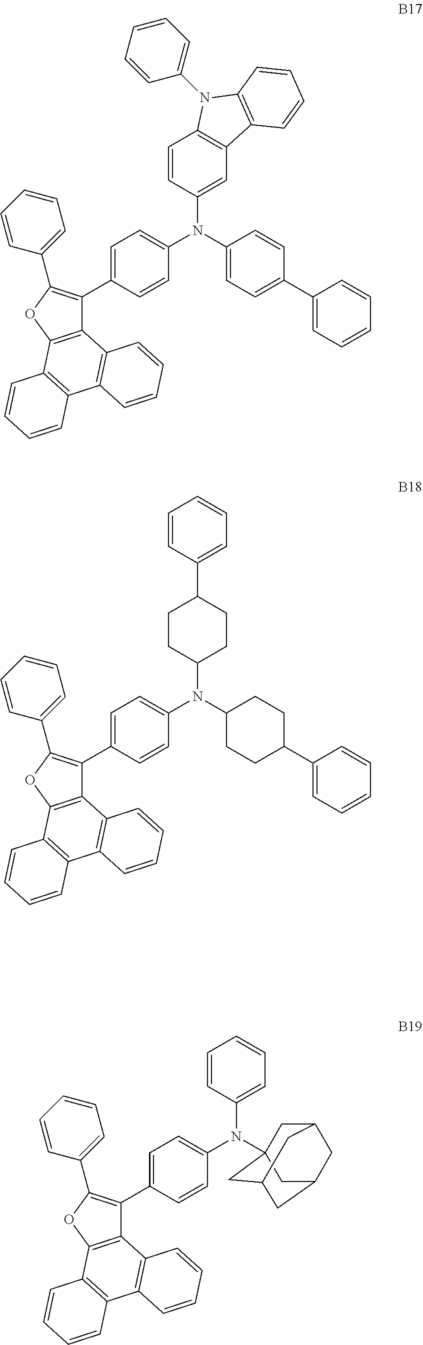 [SMILES: c1ccc(-c2ccc(N(c3ccc(-c4c(-c5ccccc5)oc5c6ccccc6c6ccccc6c45)cc3)c3ccc4c(c3)c3ccccc3n4-c3ccccc3)cc2)cc1 ; c1ccc(-c2oc3c4ccccc4c4ccccc4c3c2-c2ccc(N(C3CCC(c4ccccc4)CC3)C3CCC(c4ccccc4)CC3)cc2)cc1 ; c1ccc(-c2oc3c4ccccc4c4ccccc4c3c2-c2ccc(N(c3ccccc3)C34CC5CC(CC(C5)C3)C4)cc2)cc1]

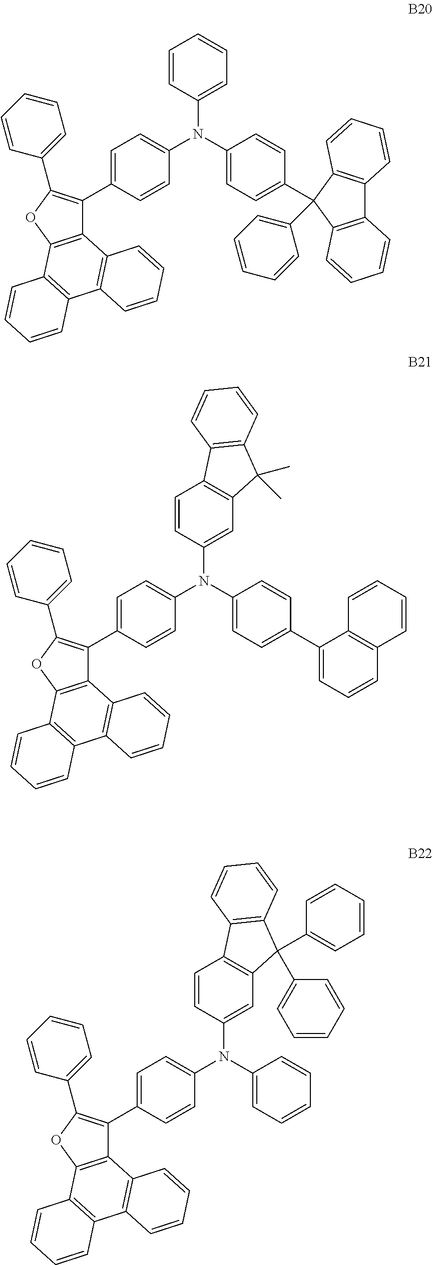 [SMILES: c1ccc(-c2oc3c4ccccc4c4ccccc4c3c2-c2ccc(N(c3ccccc3)c3ccc(C4(c5ccccc5)c5ccccc5-c5ccccc54)cc3)cc2)cc1 ; CC1(C)c2ccccc2-c2ccc(N(c3ccc(-c4cccc5ccccc45)cc3)c3ccc(-c4c(-c5ccccc5)oc5c6ccccc6c6ccccc6c45)cc3)cc21 ; c1ccc(-c2oc3c4ccccc4c4ccccc4c3c2-c2ccc(N(c3ccccc3)c3ccc4c(c3)C(c3ccccc3)(c3ccccc3)c3ccccc3-4)cc2)cc1]

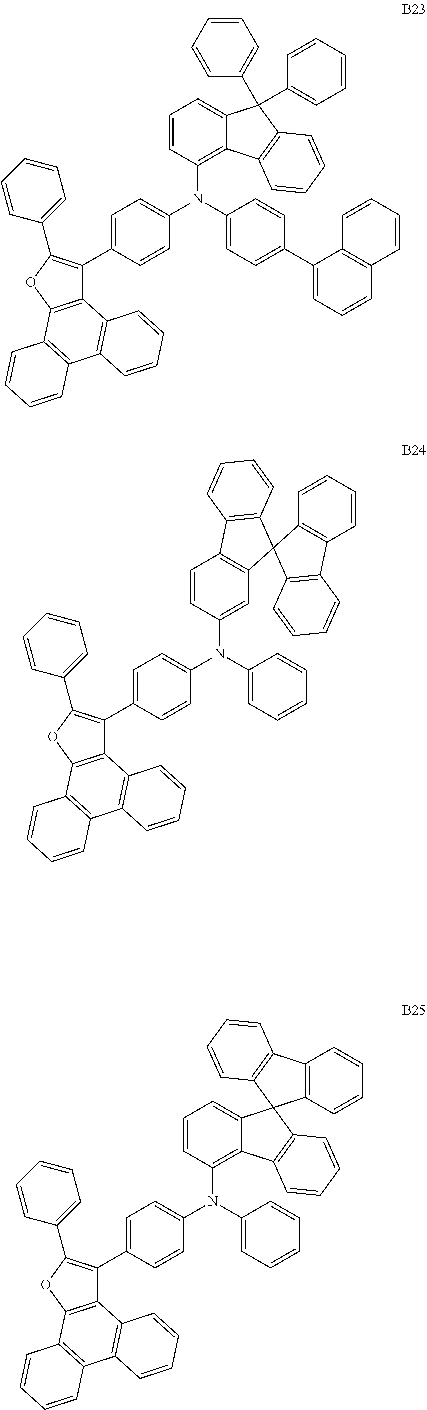 [SMILES: c1ccc(-c2oc3c4ccccc4c4ccccc4c3c2-c2ccc(N(c3ccc(-c4cccc5ccccc45)cc3)c3cccc4c3-c3ccccc3C4(c3ccccc3)c3ccccc3)cc2)cc1 ; c1ccc(-c2oc3c4ccccc4c4ccccc4c3c2-c2ccc(N(c3ccccc3)c3ccc4c(c3)C3(c5ccccc5-c5ccccc53)c3ccccc3-4)cc2)cc1 ; c1ccc(-c2oc3c4ccccc4c4ccccc4c3c2-c2ccc(N(c3ccccc3)c3cccc4c3-c3ccccc3C43c4ccccc4-c4ccccc43)cc2)cc1]

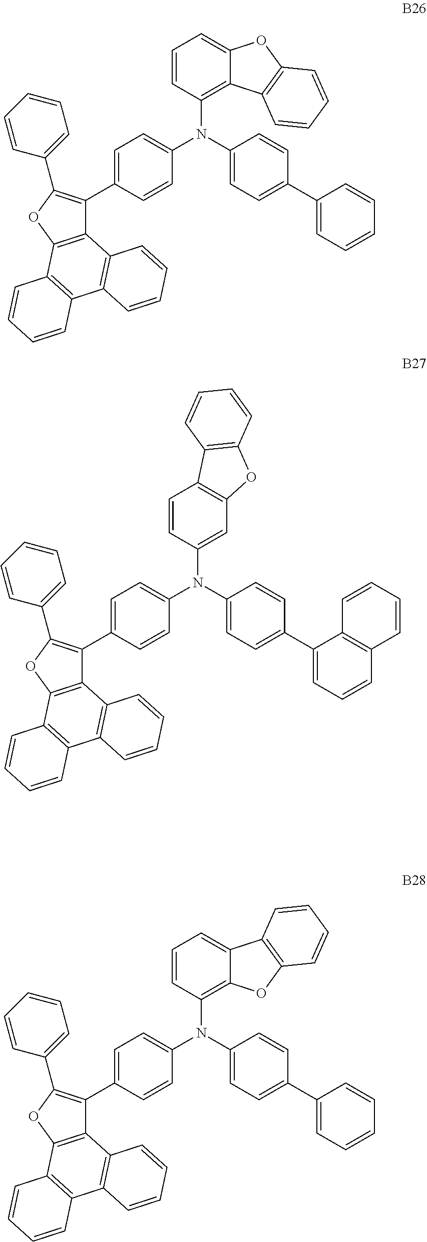 [SMILES: c1ccc(-c2ccc(N(c3ccc(-c4c(-c5ccccc5)oc5c6ccccc6c6ccccc6c45)cc3)c3cccc4oc5ccccc5c34)cc2)cc1 ; c1ccc(-c2oc3c4ccccc4c4ccccc4c3c2-c2ccc(N(c3ccc(-c4cccc5ccccc45)cc3)c3ccc4c(c3)oc3ccccc34)cc2)cc1 ; c1ccc(-c2ccc(N(c3ccc(-c4c(-c5ccccc5)oc5c6ccccc6c6ccccc6c45)cc3)c3cccc4c3oc3ccccc34)cc2)cc1]

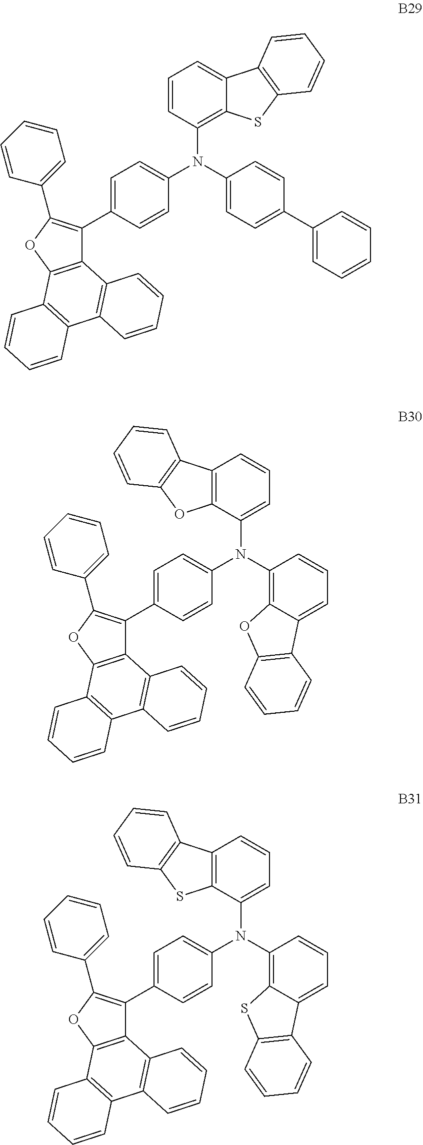 [SMILES: c1ccc(-c2ccc(N(c3ccc(-c4c(-c5ccccc5)oc5c6ccccc6c6ccccc6c45)cc3)c3cccc4c3sc3ccccc34)cc2)cc1 ; c1ccc(-c2oc3c4ccccc4c4ccccc4c3c2-c2ccc(N(c3cccc4c3oc3ccccc34)c3cccc4c3oc3ccccc34)cc2)cc1 ; c1ccc(-c2oc3c4ccccc4c4ccccc4c3c2-c2ccc(N(c3cccc4c3sc3ccccc34)c3cccc4c3sc3ccccc34)cc2)cc1]

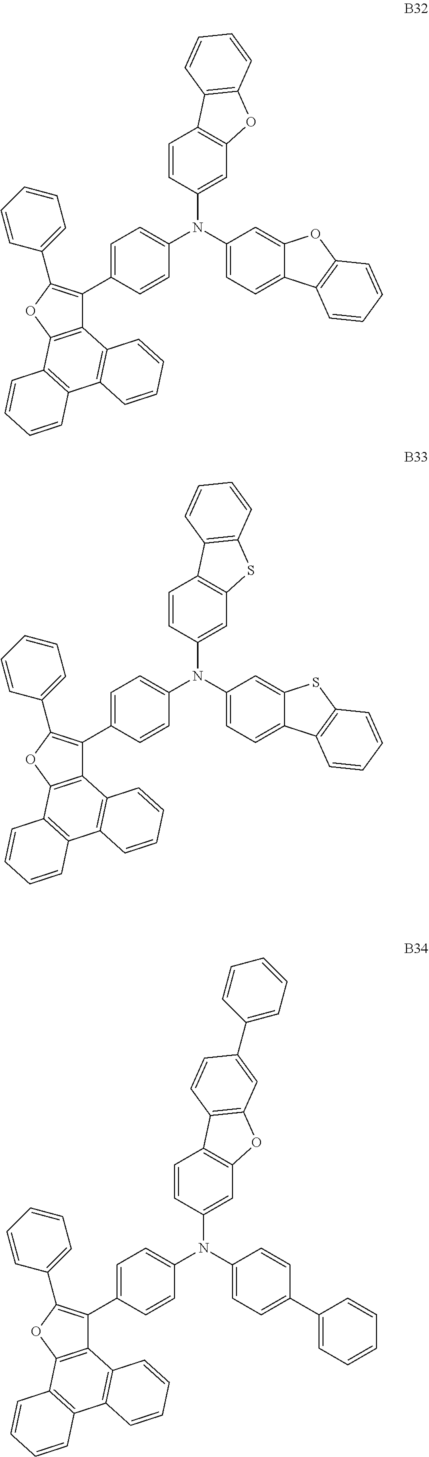 [SMILES: c1ccc(-c2oc3c4ccccc4c4ccccc4c3c2-c2ccc(N(c3ccc4c(c3)oc3ccccc34)c3ccc4c(c3)oc3ccccc34)cc2)cc1 ; c1ccc(-c2oc3c4ccccc4c4ccccc4c3c2-c2ccc(N(c3ccc4c(c3)sc3ccccc34)c3ccc4c(c3)sc3ccccc34)cc2)cc1 ; c1ccc(-c2ccc(N(c3ccc(-c4c(-c5ccccc5)oc5c6ccccc6c6ccccc6c45)cc3)c3ccc4c(c3)oc3cc(-c5ccccc5)ccc34)cc2)cc1]

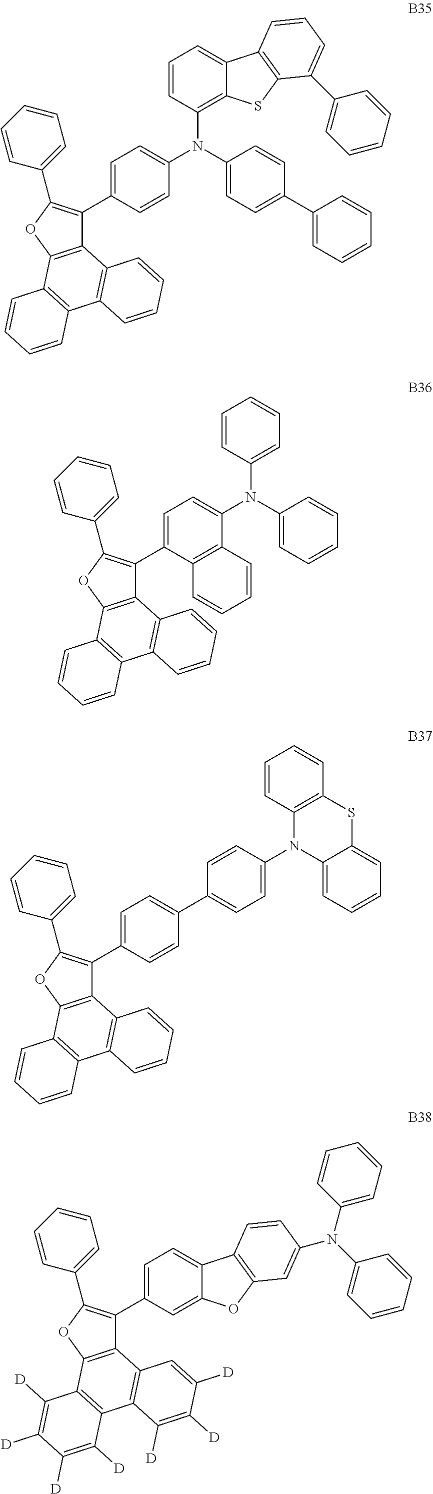 [SMILES: c1ccc(-c2ccc(N(c3ccc(-c4c(-c5ccccc5)oc5c6ccccc6c6ccccc6c45)cc3)c3cccc4c3sc3c(-c5ccccc5)cccc34)cc2)cc1 ; c1ccc(-c2oc3c4ccccc4c4ccccc4c3c2-c2ccc(N(c3ccccc3)c3ccccc3)c3ccccc23)cc1 ; c1ccc(-c2oc3c4ccccc4c4ccccc4c3c2-c2ccc(-c3ccc(N4c5ccccc5Sc5ccccc54)cc3)cc2)cc1 ; [2H]c1cc2c3c(-c4ccc5c(c4)oc4cc(N(c6ccccc6)c6ccccc6)ccc45)c(-c4ccccc4)oc3c3c([2H])c([2H])c([2H])c([2H])c3c2c([2H])c1[2H]]

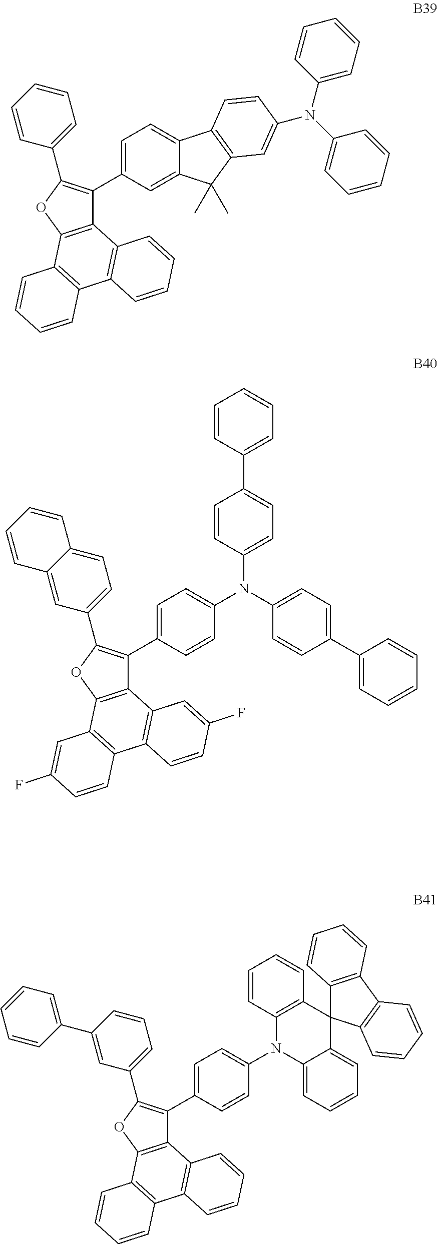 [SMILES: CC1(C)c2cc(-c3c(-c4ccccc4)oc4c5ccccc5c5ccccc5c34)ccc2-c2ccc(N(c3ccccc3)c3ccccc3)cc21 ; Fc1ccc2c3ccc(F)cc3c3c(-c4ccc(N(c5ccc(-c6ccccc6)cc5)c5ccc(-c6ccccc6)cc5)cc4)c(-c4ccc5ccccc5c4)oc3c2c1 ; c1ccc(-c2cccc(-c3oc4c5ccccc5c5ccccc5c4c3-c3ccc(N4c5ccccc5C5(c6ccccc6-c6ccccc65)c5ccccc54)cc3)c2)cc1]

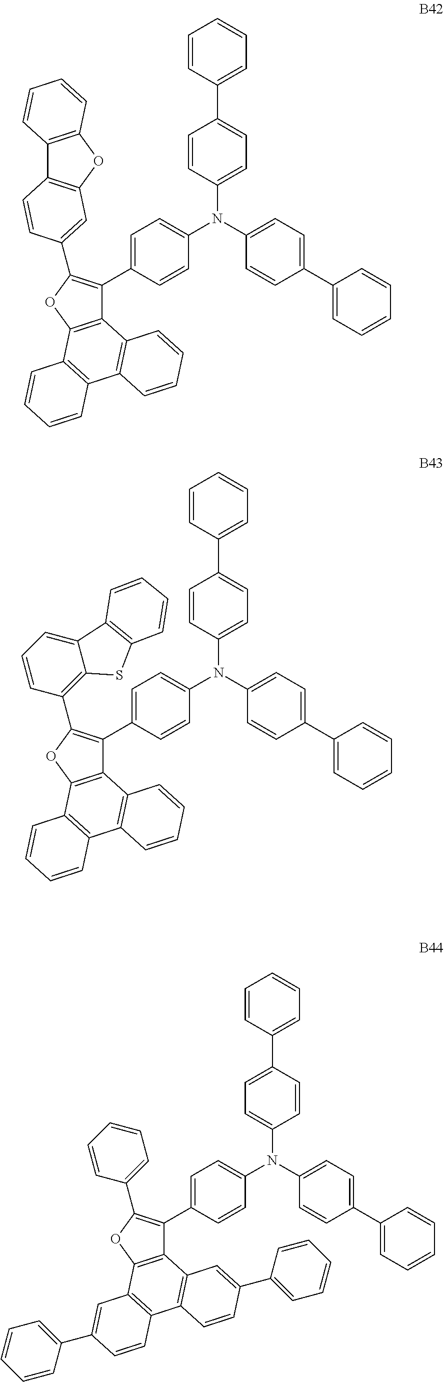 [SMILES: c1ccc(-c2ccc(N(c3ccc(-c4ccccc4)cc3)c3ccc(-c4c(-c5ccc6c(c5)oc5ccccc56)oc5c6ccccc6c6ccccc6c45)cc3)cc2)cc1 ; c1ccc(-c2ccc(N(c3ccc(-c4ccccc4)cc3)c3ccc(-c4c(-c5cccc6c5sc5ccccc56)oc5c6ccccc6c6ccccc6c45)cc3)cc2)cc1 ; c1ccc(-c2ccc(N(c3ccc(-c4ccccc4)cc3)c3ccc(-c4c(-c5ccccc5)oc5c6cc(-c7ccccc7)ccc6c6ccc(-c7ccccc7)cc6c45)cc3)cc2)cc1]

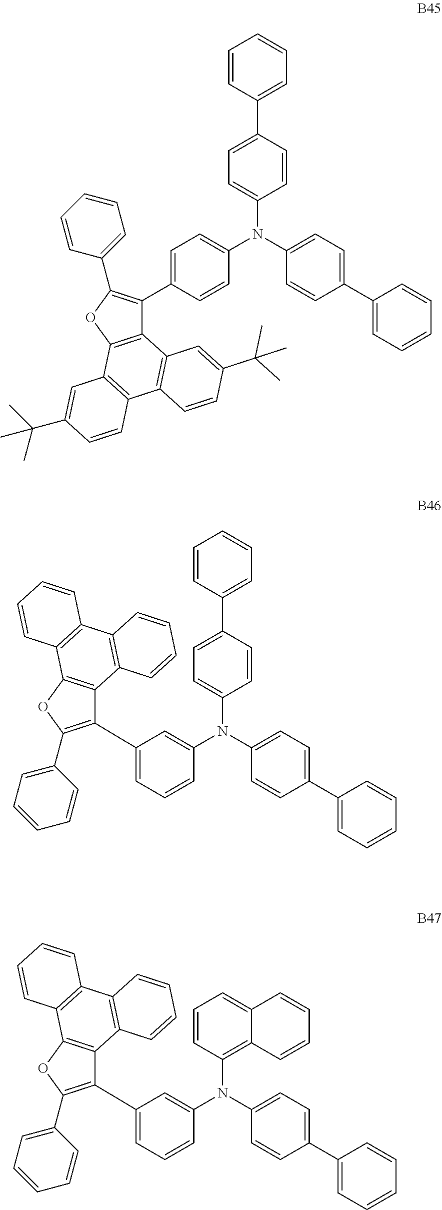 [SMILES: CC(C)(C)c1ccc2c3ccc(C(C)(C)C)cc3c3c(-c4ccc(N(c5ccc(-c6ccccc6)cc5)c5ccc(-c6ccccc6)cc5)cc4)c(-c4ccccc4)oc3c2c1 ; c1ccc(-c2ccc(N(c3ccc(-c4ccccc4)cc3)c3cccc(-c4c(-c5ccccc5)oc5c6ccccc6c6ccccc6c45)c3)cc2)cc1 ; c1ccc(-c2ccc(N(c3cccc(-c4c(-c5ccccc5)oc5c6ccccc6c6ccccc6c45)c3)c3cccc4ccccc34)cc2)cc1]

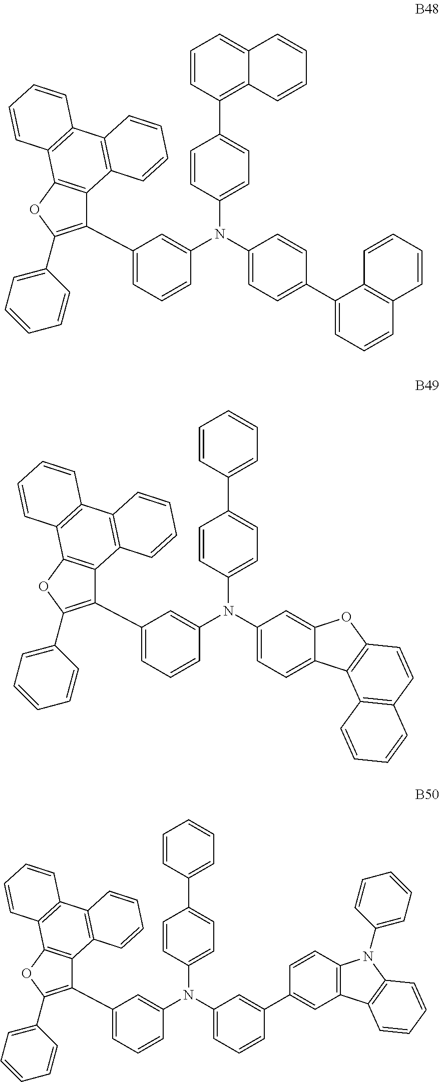 [SMILES: c1ccc(-c2oc3c4ccccc4c4ccccc4c3c2-c2cccc(N(c3ccc(-c4cccc5ccccc45)cc3)c3ccc(-c4cccc5ccccc45)cc3)c2)cc1 ; c1ccc(-c2ccc(N(c3cccc(-c4c(-c5ccccc5)oc5c6ccccc6c6ccccc6c45)c3)c3ccc4c(c3)oc3ccc5ccccc5c34)cc2)cc1 ; c1ccc(-c2ccc(N(c3cccc(-c4ccc5c(c4)c4ccccc4n5-c4ccccc4)c3)c3cccc(-c4c(-c5ccccc5)oc5c6ccccc6c6ccccc6c45)c3)cc2)cc1]

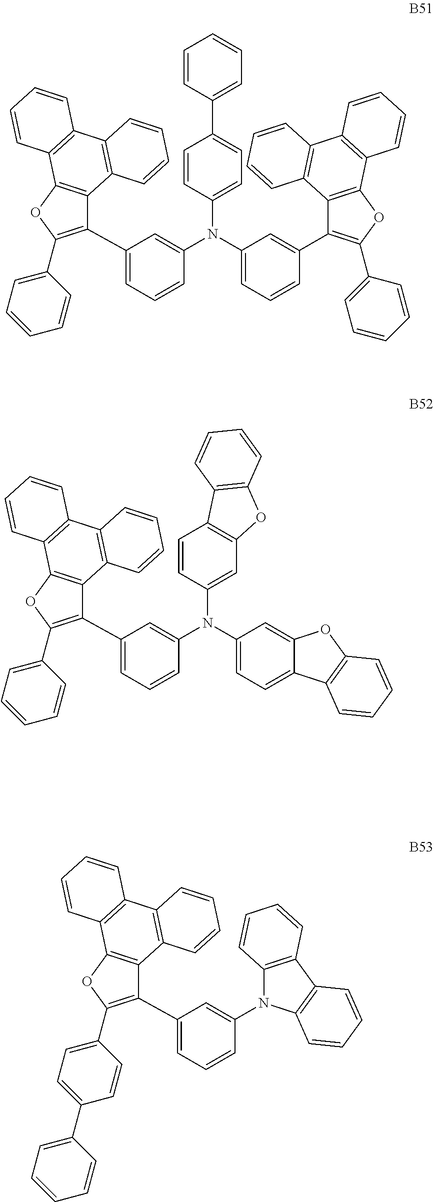 [SMILES: c1ccc(-c2ccc(N(c3cccc(-c4c(-c5ccccc5)oc5c6ccccc6c6ccccc6c45)c3)c3cccc(-c4c(-c5ccccc5)oc5c6ccccc6c6ccccc6c45)c3)cc2)cc1 ; c1ccc(-c2oc3c4ccccc4c4ccccc4c3c2-c2cccc(N(c3ccc4c(c3)oc3ccccc34)c3ccc4c(c3)oc3ccccc34)c2)cc1 ; c1ccc(-c2ccc(-c3oc4c5ccccc5c5ccccc5c4c3-c3cccc(-n4c5ccccc5c5ccccc54)c3)cc2)cc1]

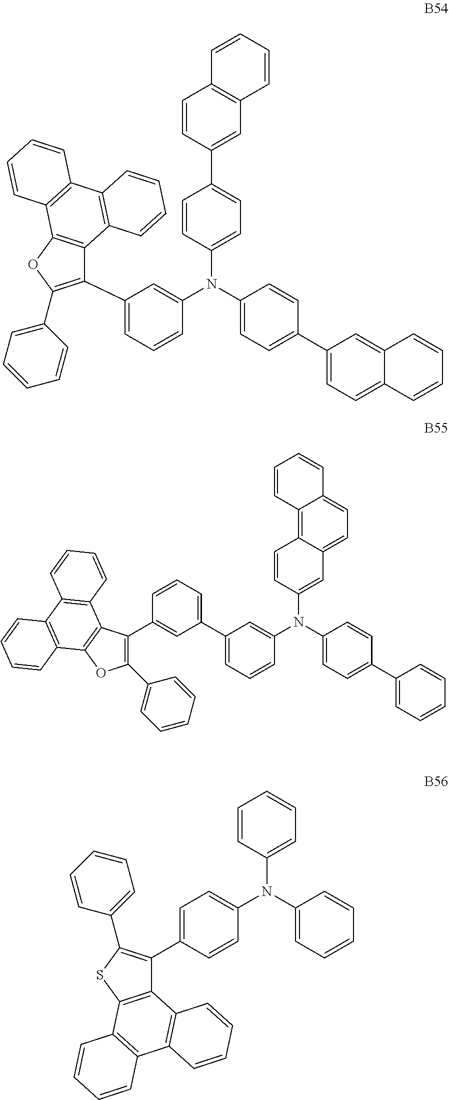 [SMILES: c1ccc(-c2oc3c4ccccc4c4ccccc4c3c2-c2cccc(N(c3ccc(-c4ccc5ccccc5c4)cc3)c3ccc(-c4ccc5ccccc5c4)cc3)c2)cc1 ; c1ccc(-c2ccc(N(c3cccc(-c4cccc(-c5c(-c6ccccc6)oc6c7ccccc7c7ccccc7c56)c4)c3)c3ccc4c(ccc5ccccc54)c3)cc2)cc1 ; c1ccc(-c2sc3c4ccccc4c4ccccc4c3c2-c2ccc(N(c3ccccc3)c3ccccc3)cc2)cc1]

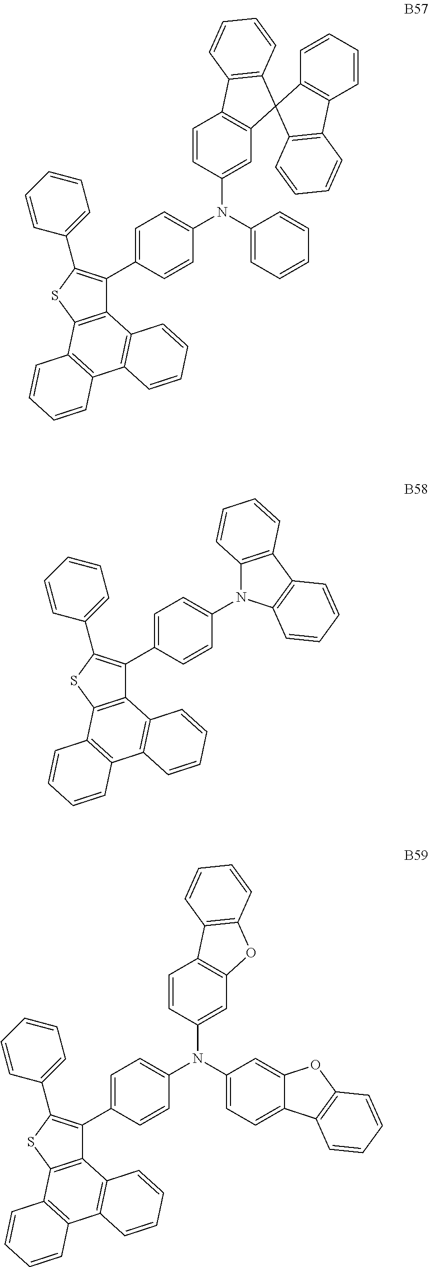 [SMILES: c1ccc(-c2sc3c4ccccc4c4ccccc4c3c2-c2ccc(N(c3ccccc3)c3ccc4c(c3)C3(c5ccccc5-c5ccccc53)c3ccccc3-4)cc2)cc1 ; c1ccc(-c2sc3c4ccccc4c4ccccc4c3c2-c2ccc(-n3c4ccccc4c4ccccc43)cc2)cc1 ; c1ccc(-c2sc3c4ccccc4c4ccccc4c3c2-c2ccc(N(c3ccc4c(c3)oc3ccccc34)c3ccc4c(c3)oc3ccccc34)cc2)cc1]

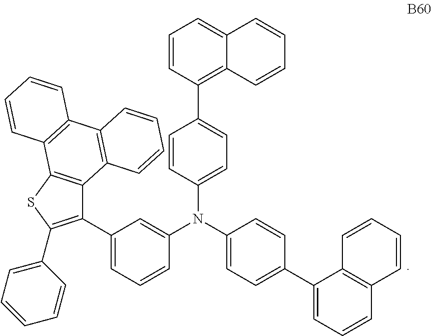 [SMILES: c1ccc(-c2sc3c4ccccc4c4ccccc4c3c2-c2cccc(N(c3ccc(-c4cccc5ccccc45)cc3)c3ccc(-c4cccc5ccccc45)cc3)c2)cc1]

The polycyclic compound represented by Formula 1 according to an embodiment of the inventive concepts may be any one selected among the compounds represented in the following Compound Group 3 to Compound Group 6, without limitation:

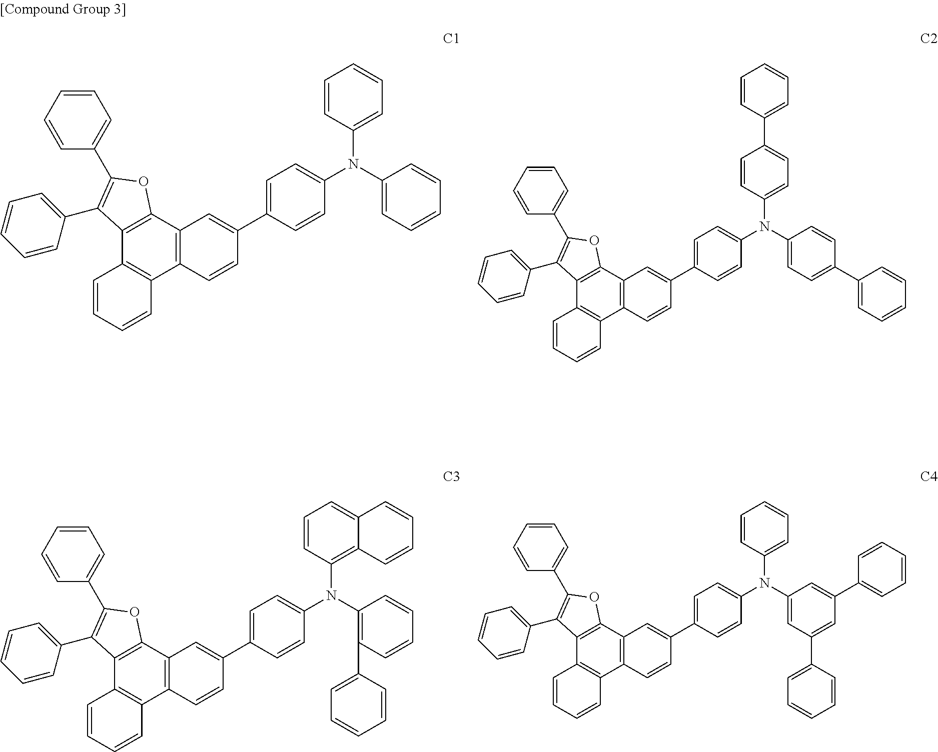 [SMILES: c1ccc(-c2oc3c4cc(-c5ccc(N(c6ccccc6)c6ccccc6)cc5)ccc4c4ccccc4c3c2-c2ccccc2)cc1 ; c1ccc(-c2ccc(N(c3ccc(-c4ccccc4)cc3)c3ccc(-c4ccc5c6ccccc6c6c(-c7ccccc7)c(-c7ccccc7)oc6c5c4)cc3)cc2)cc1 ; c1ccc(-c2ccccc2N(c2ccc(-c3ccc4c5ccccc5c5c(-c6ccccc6)c(-c6ccccc6)oc5c4c3)cc2)c2cccc3ccccc23)cc1 ; c1ccc(-c2cc(-c3ccccc3)cc(N(c3ccccc3)c3ccc(-c4ccc5c6ccccc6c6c(-c7ccccc7)c(-c7ccccc7)oc6c5c4)cc3)c2)cc1]

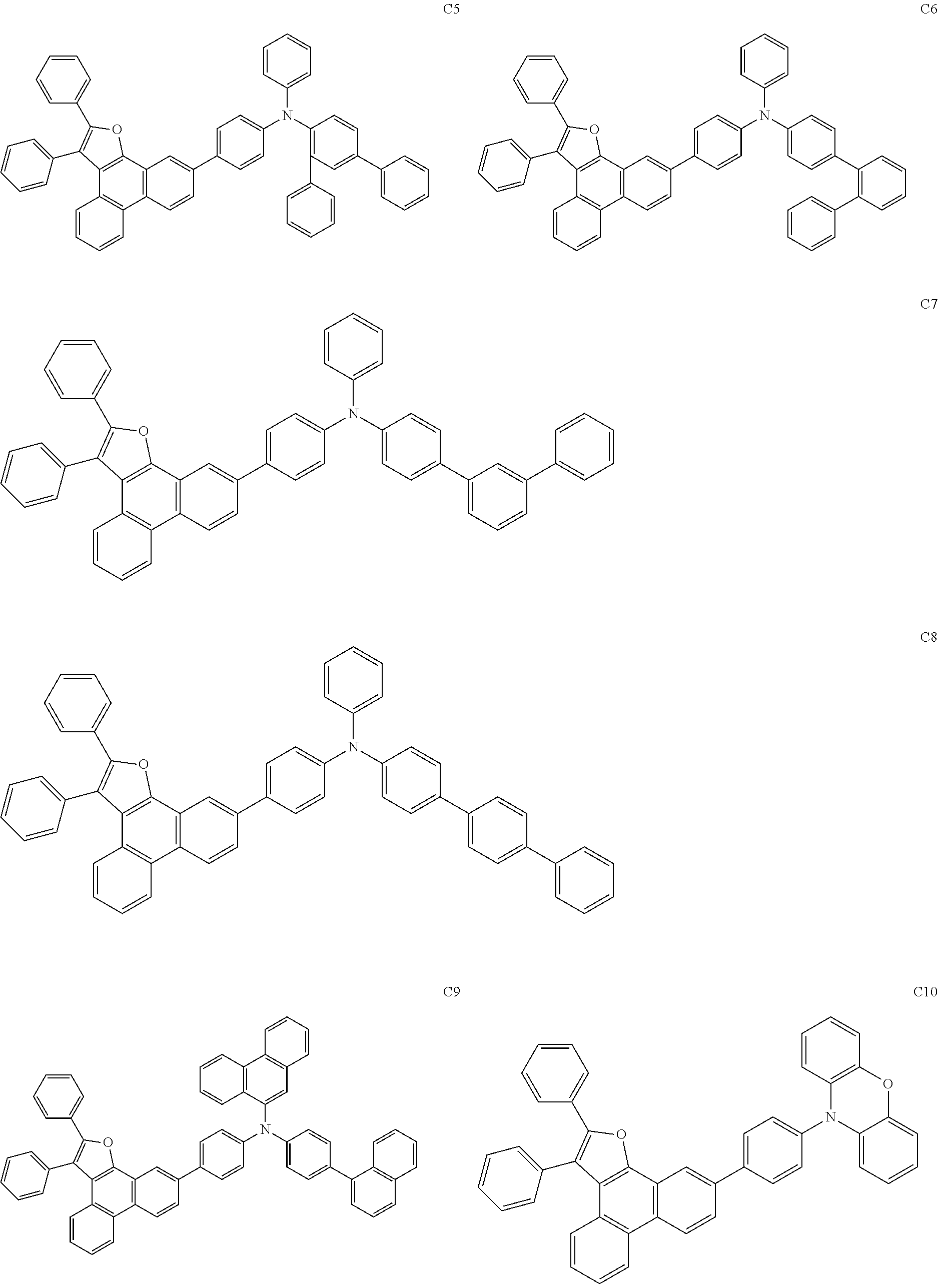

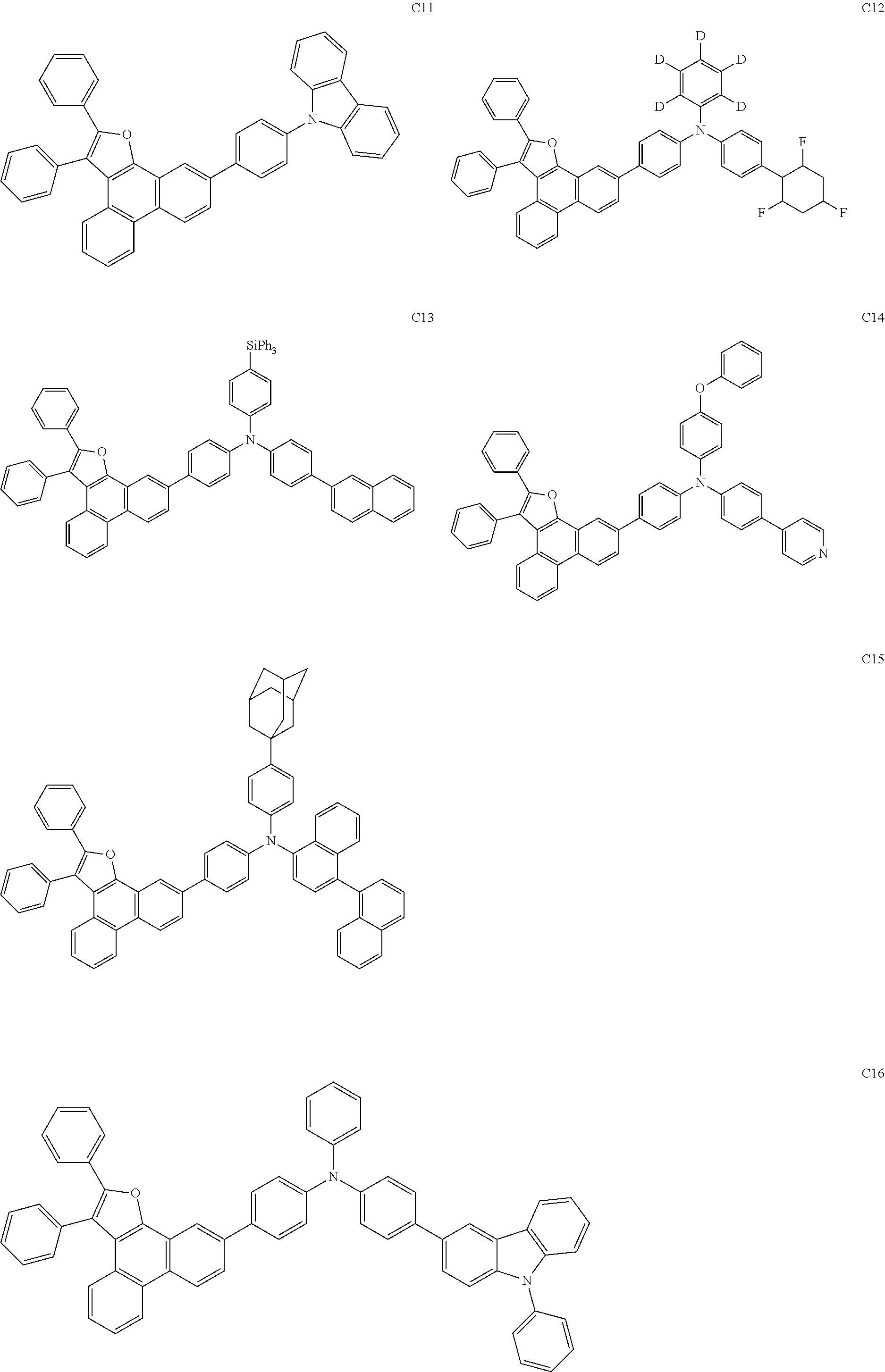

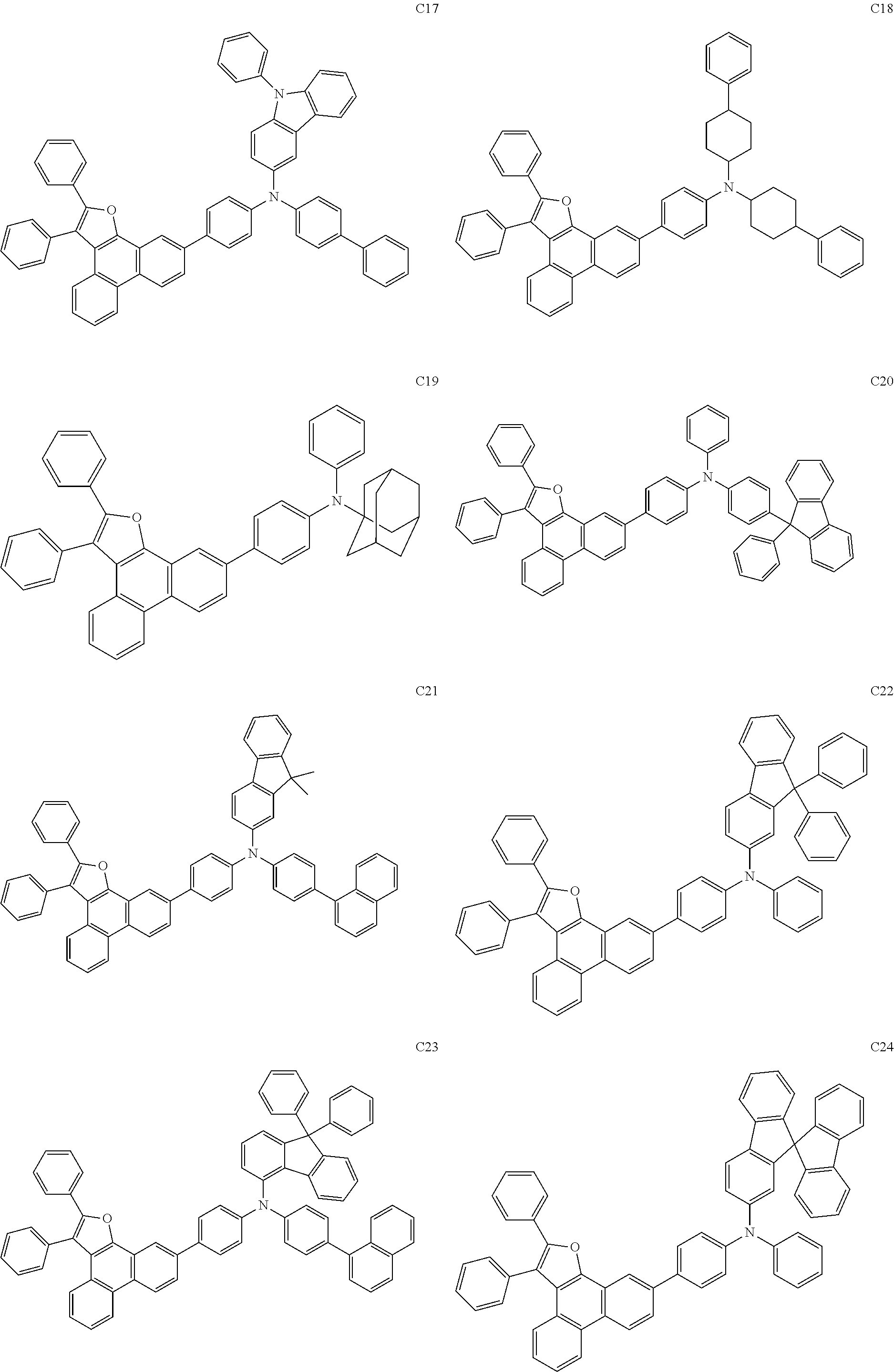

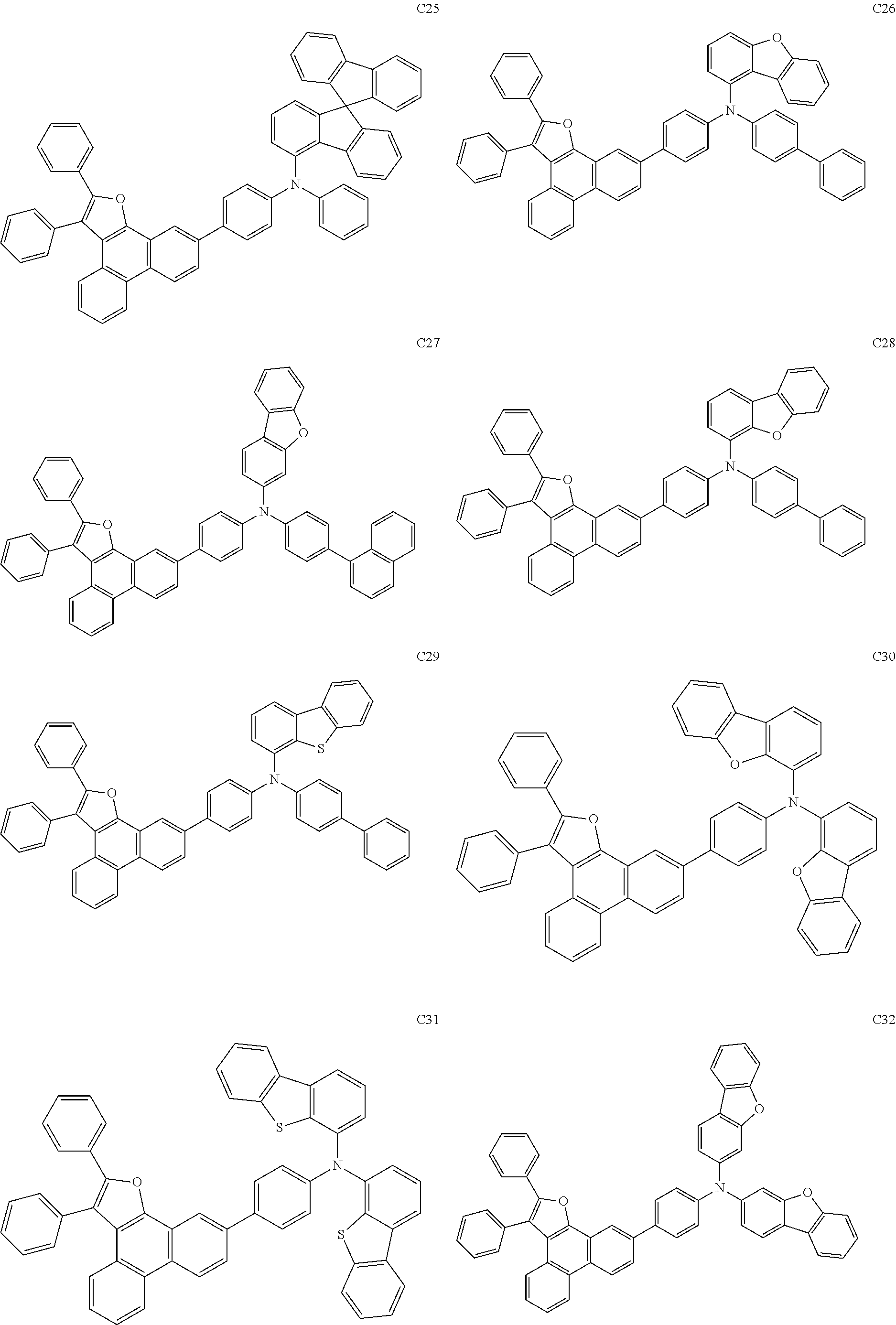

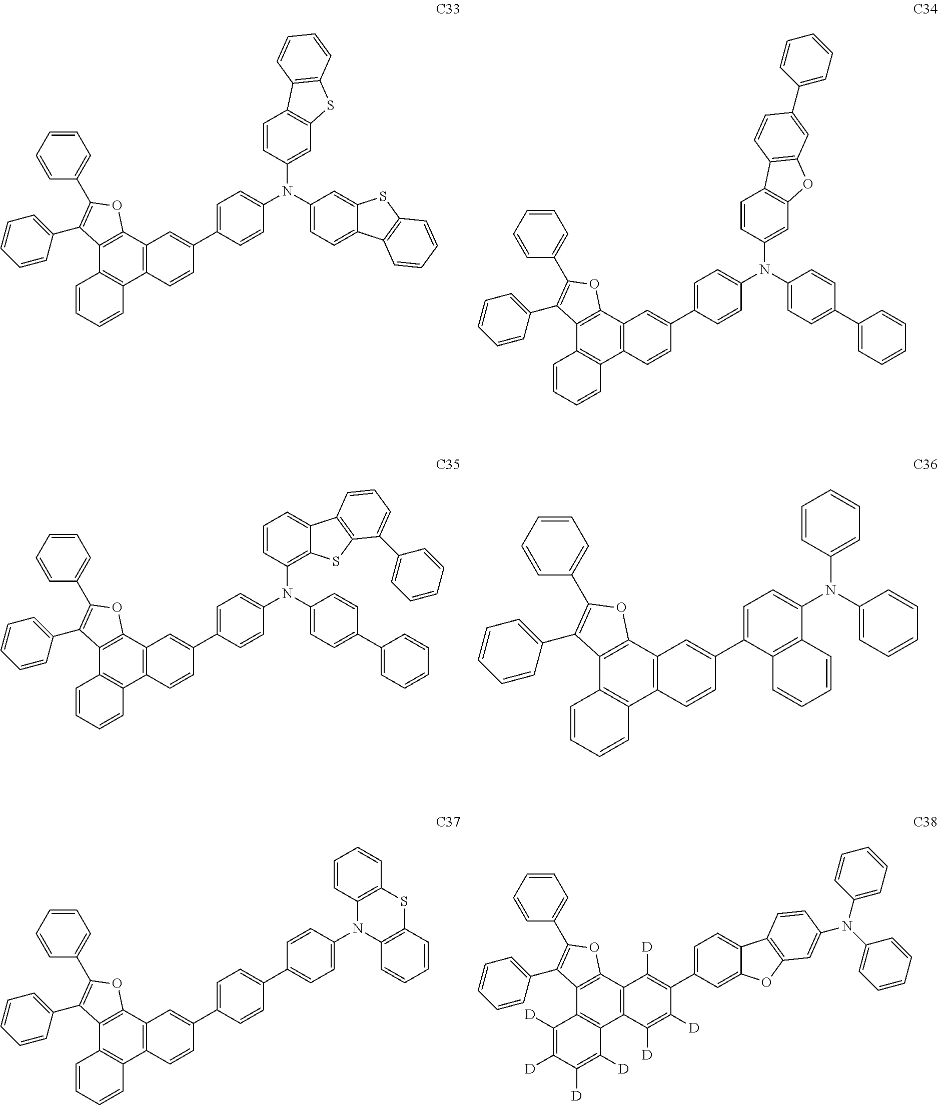

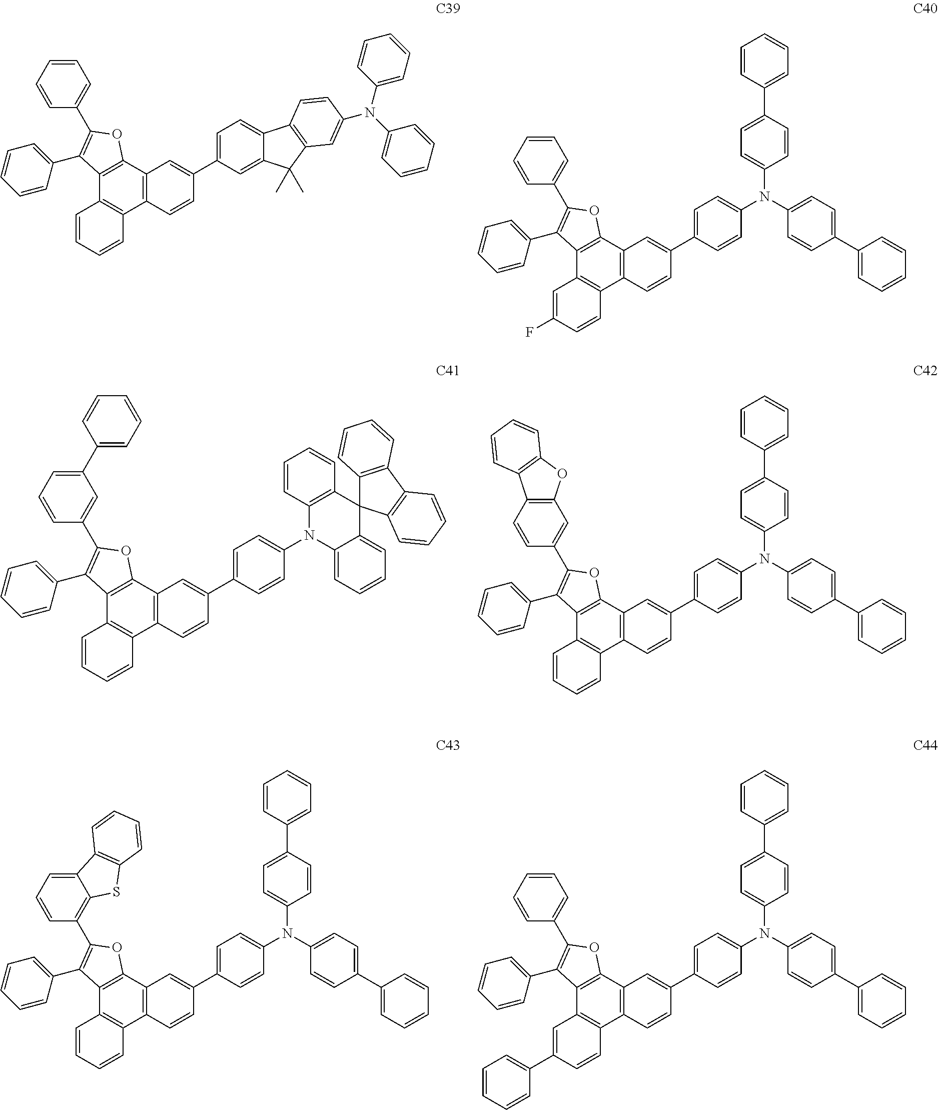

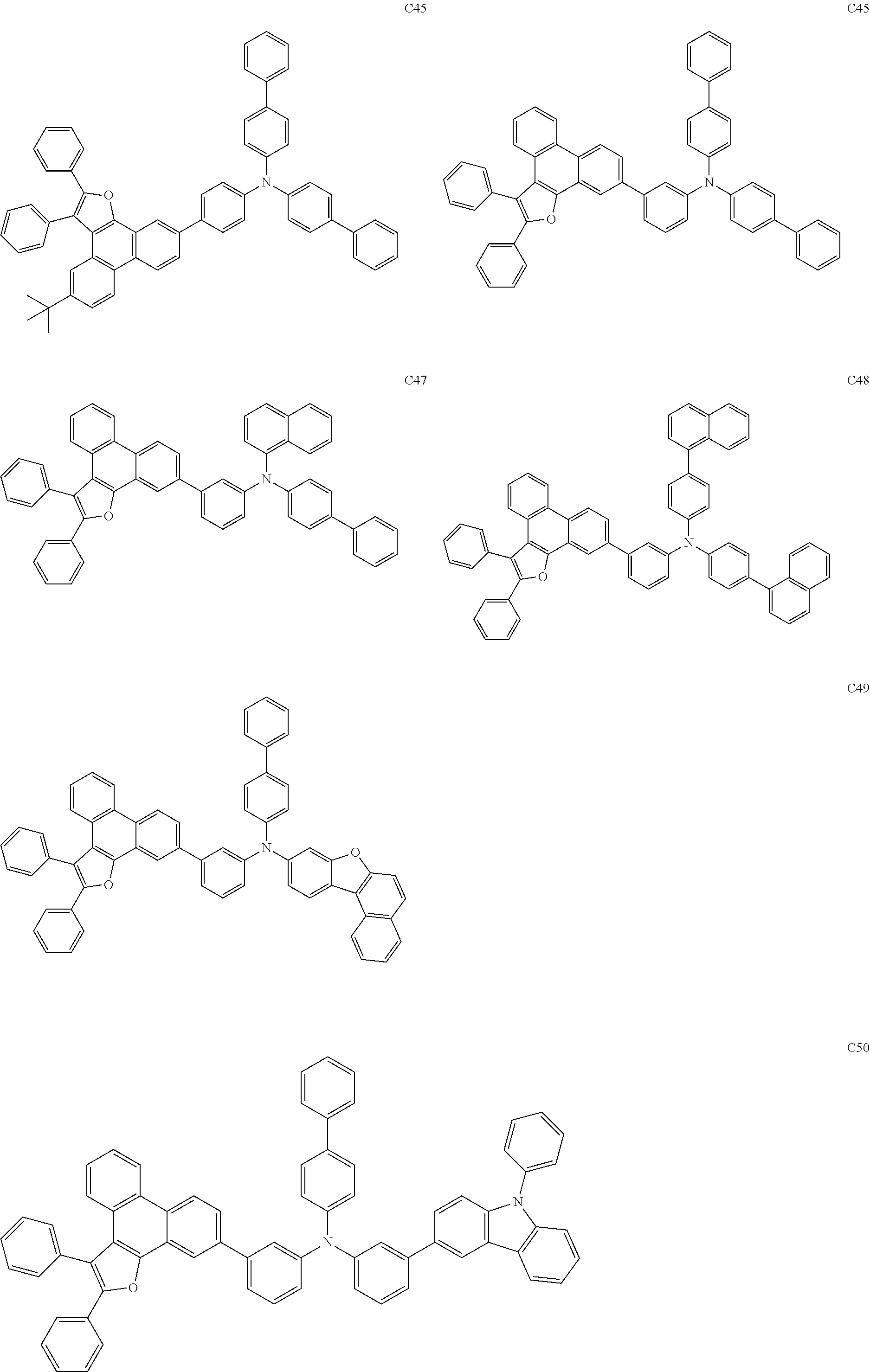

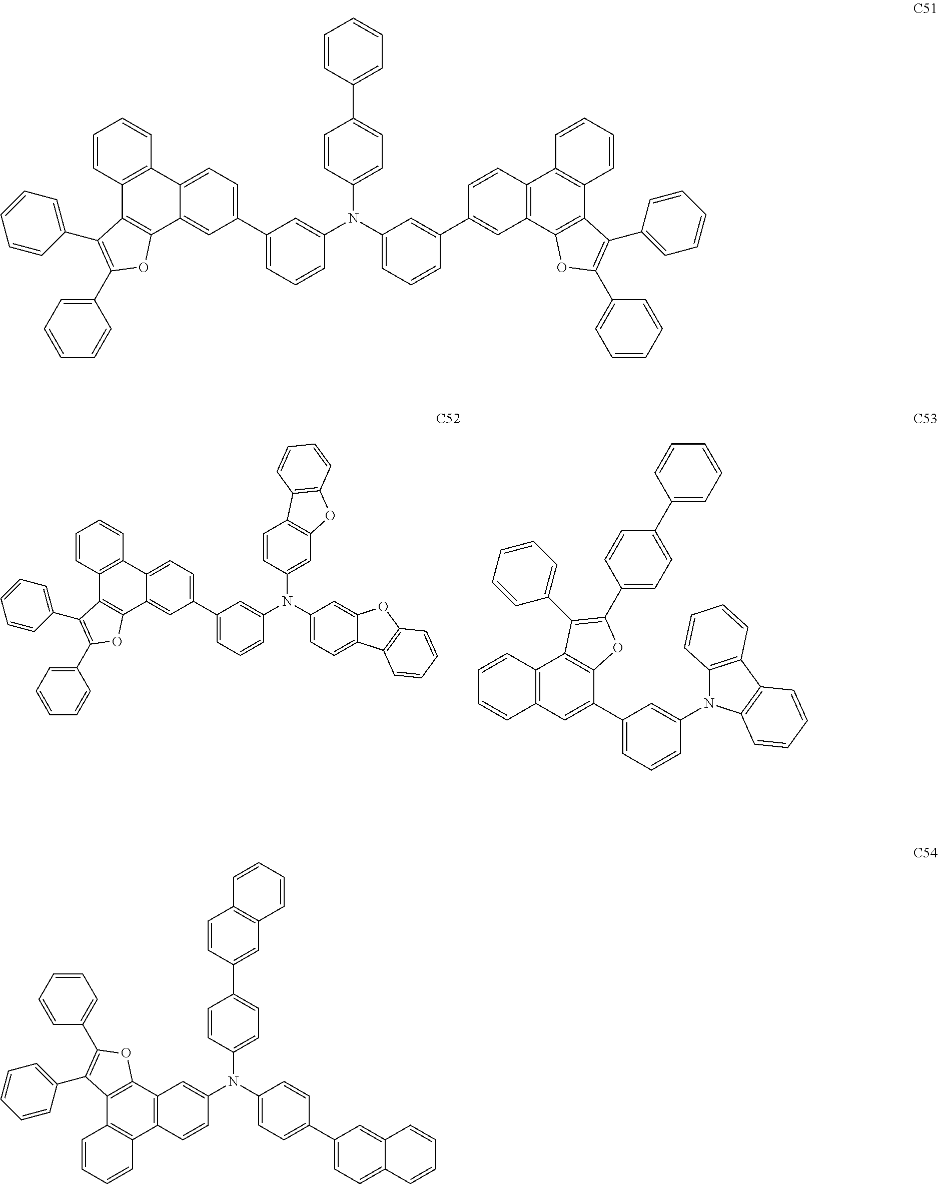

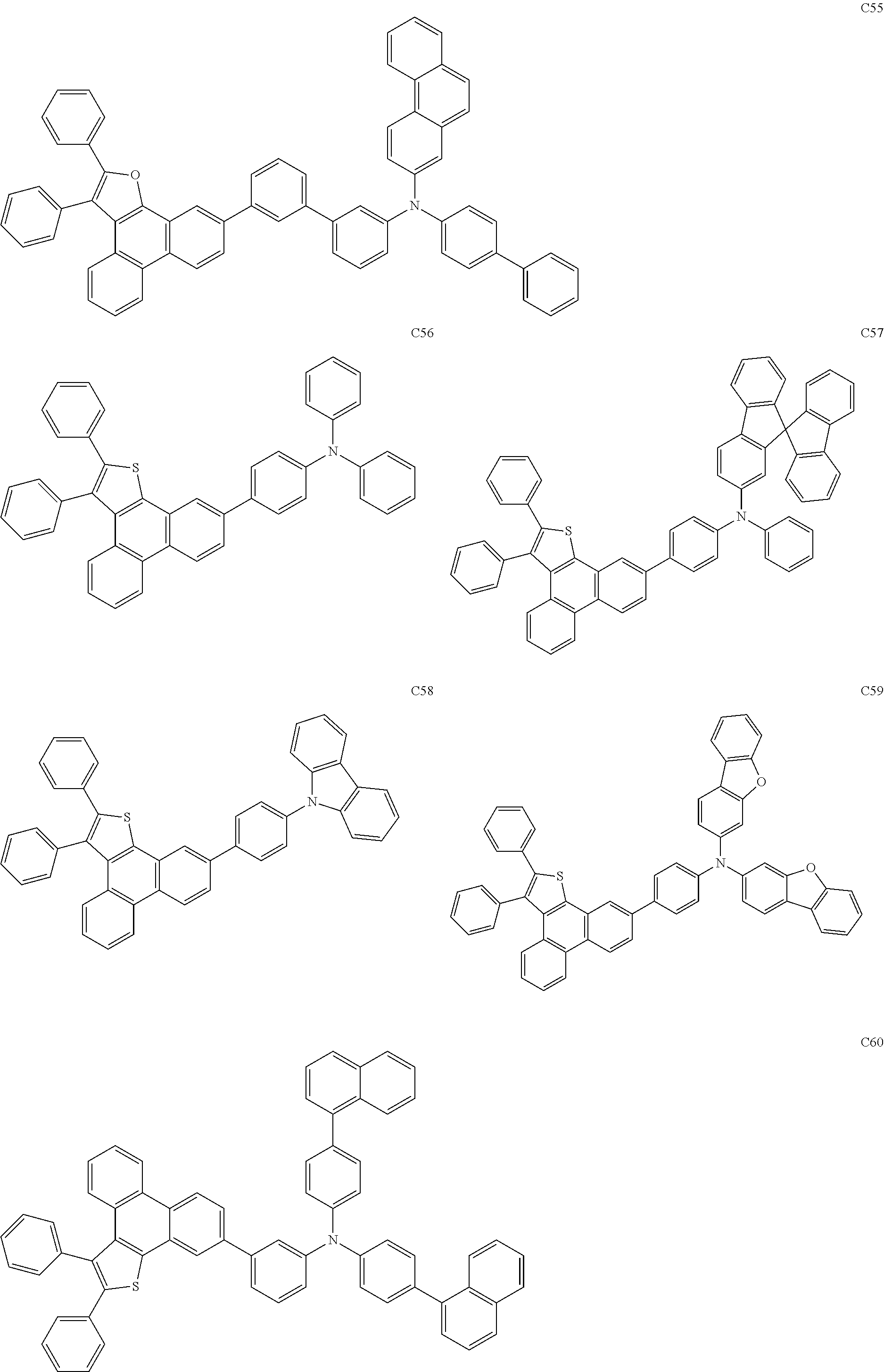

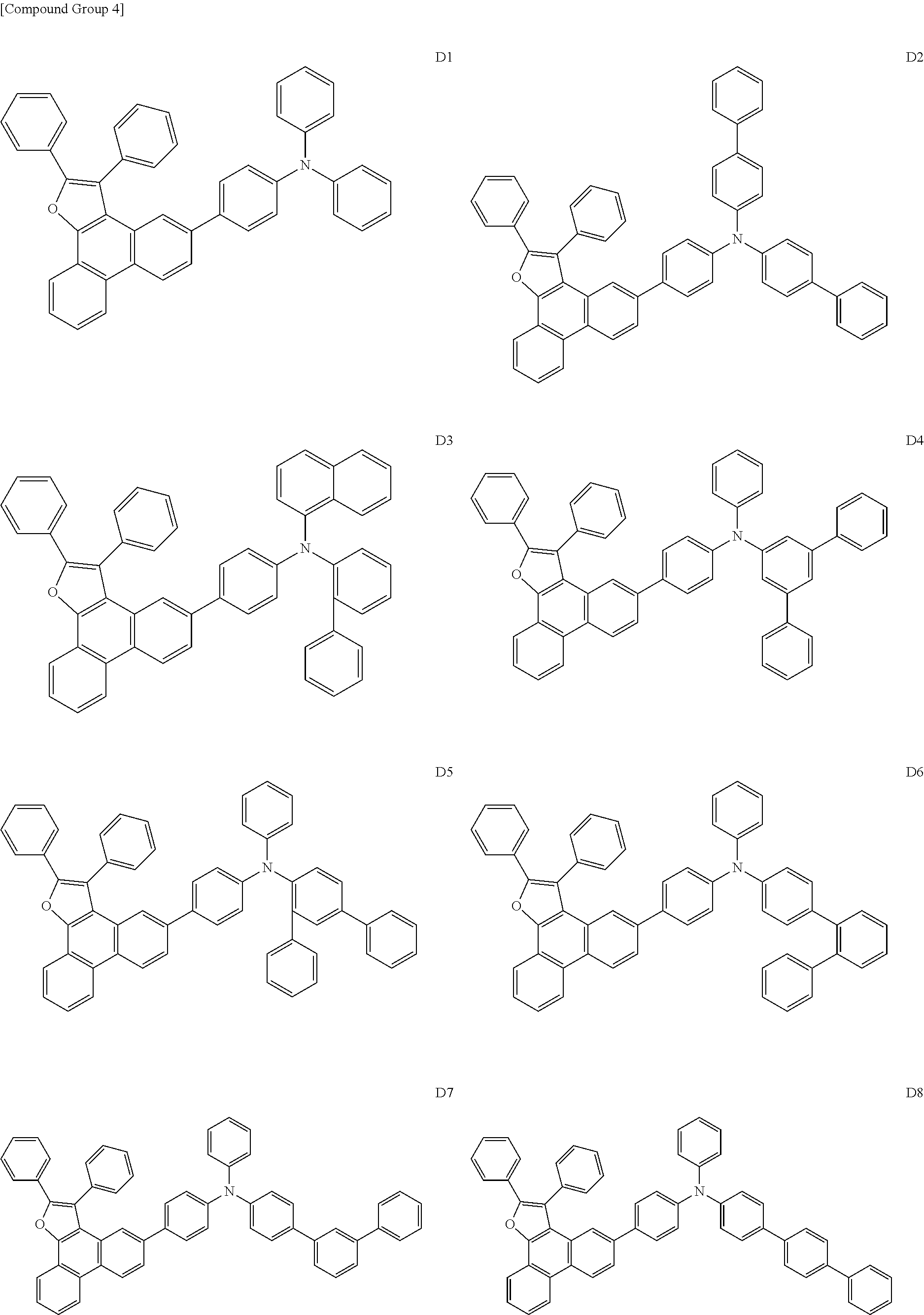

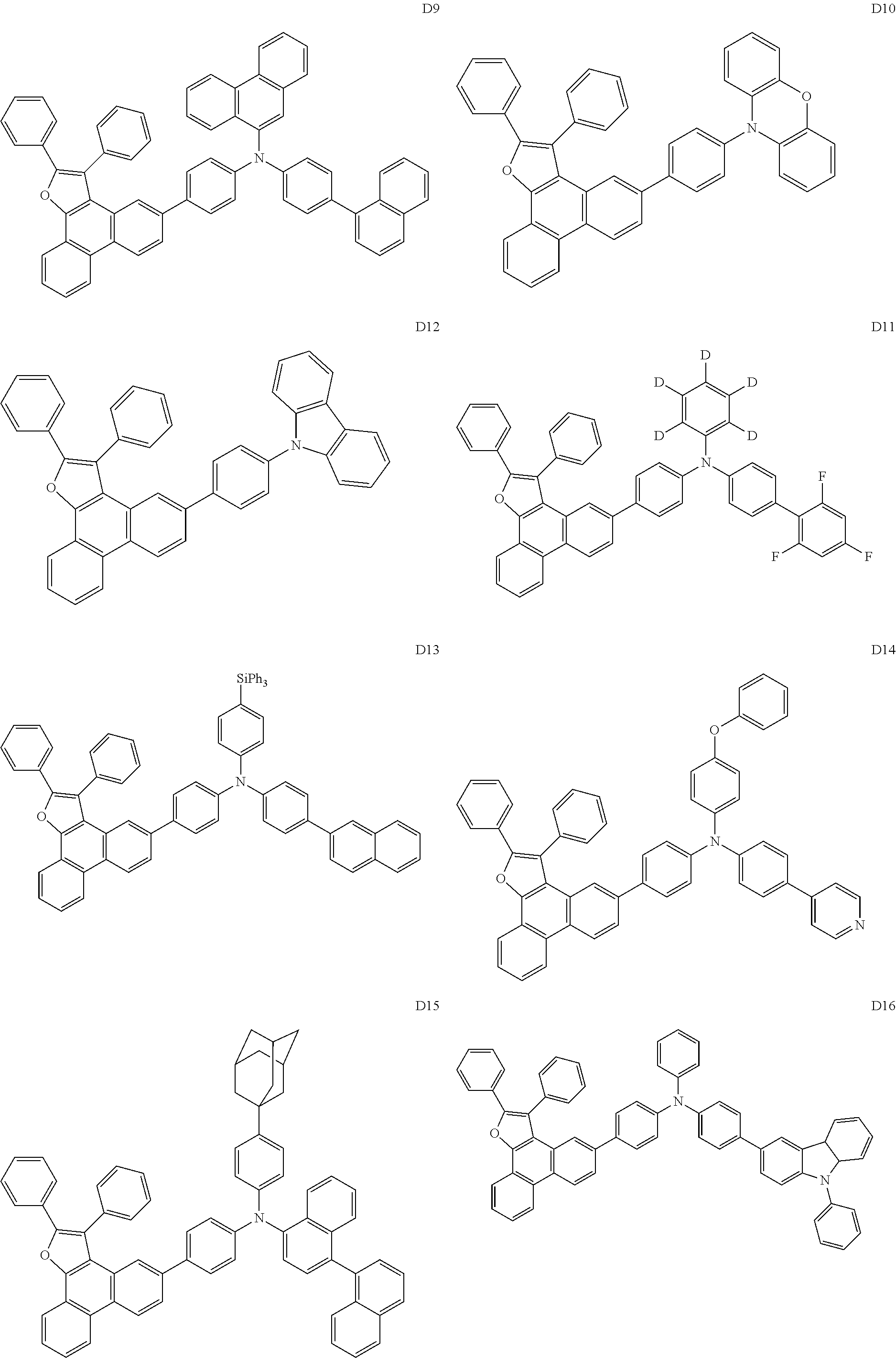

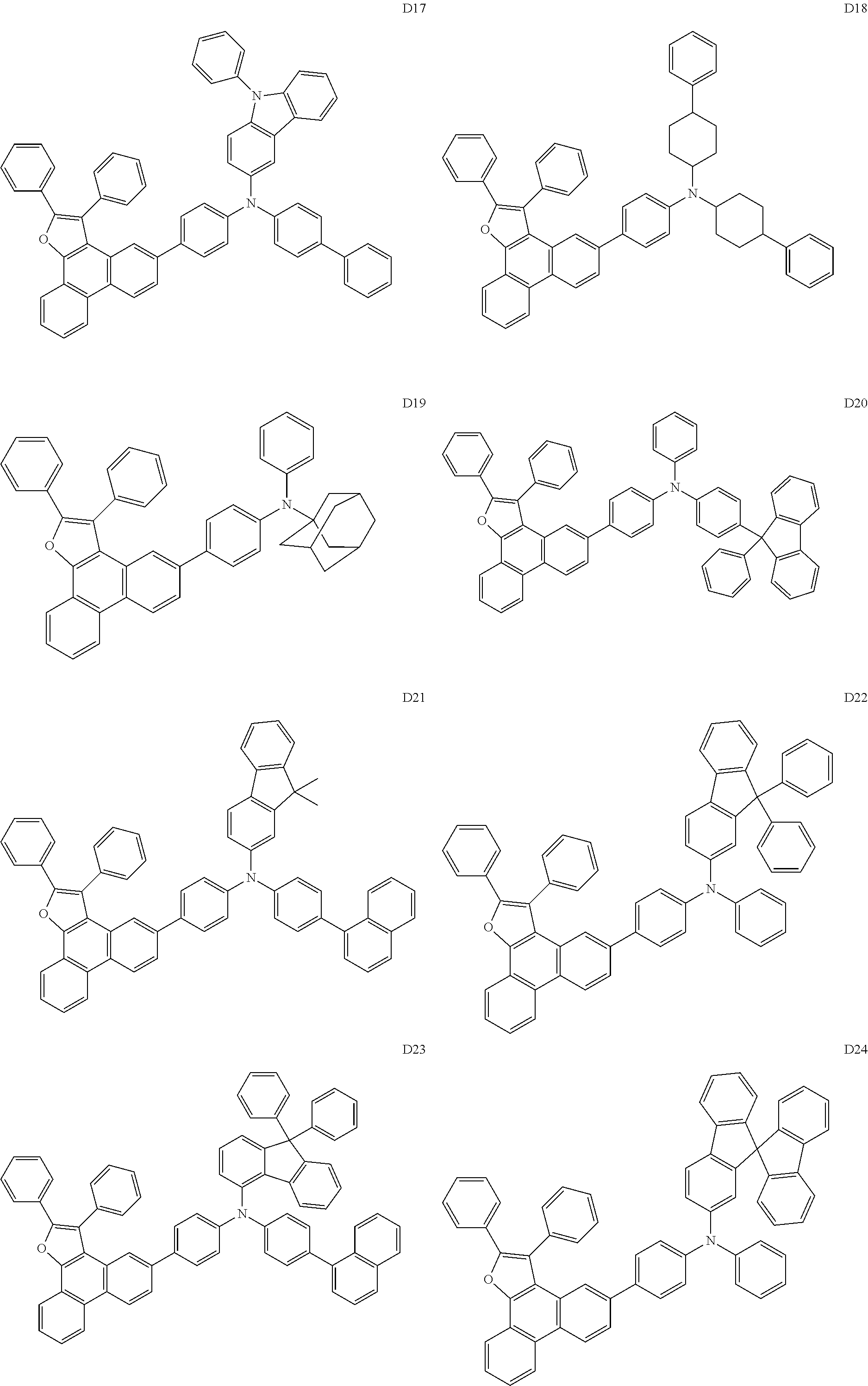

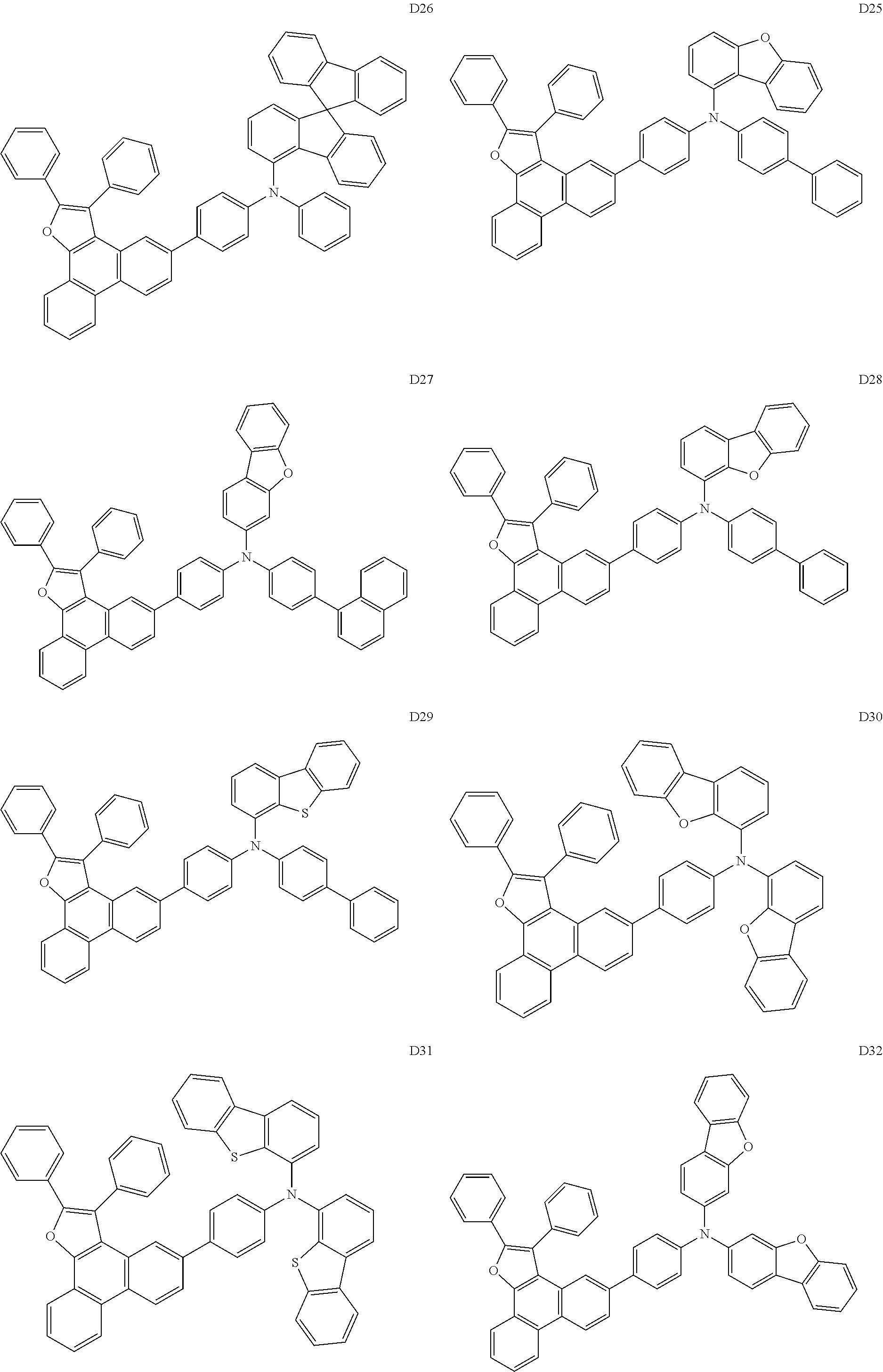

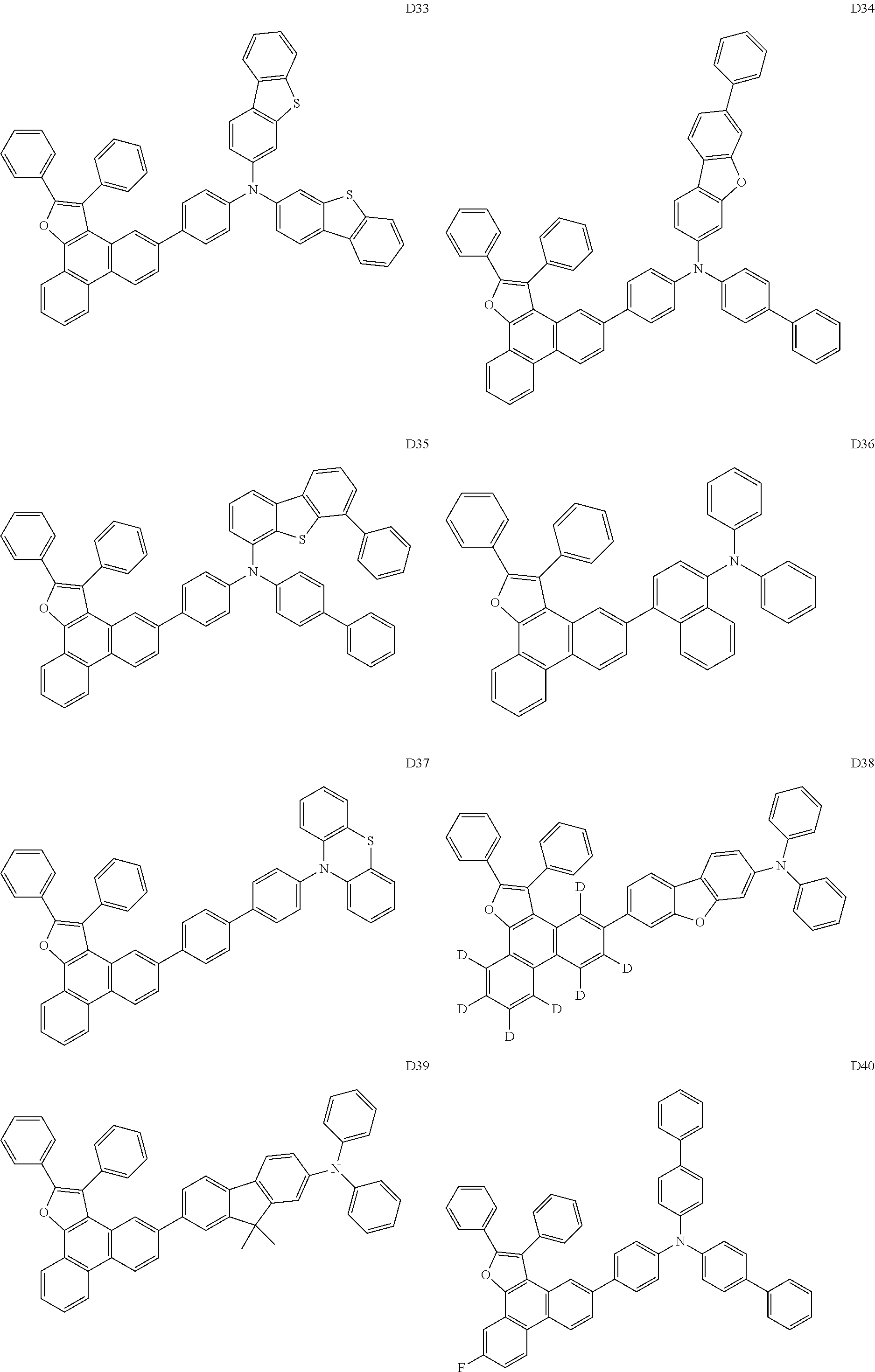

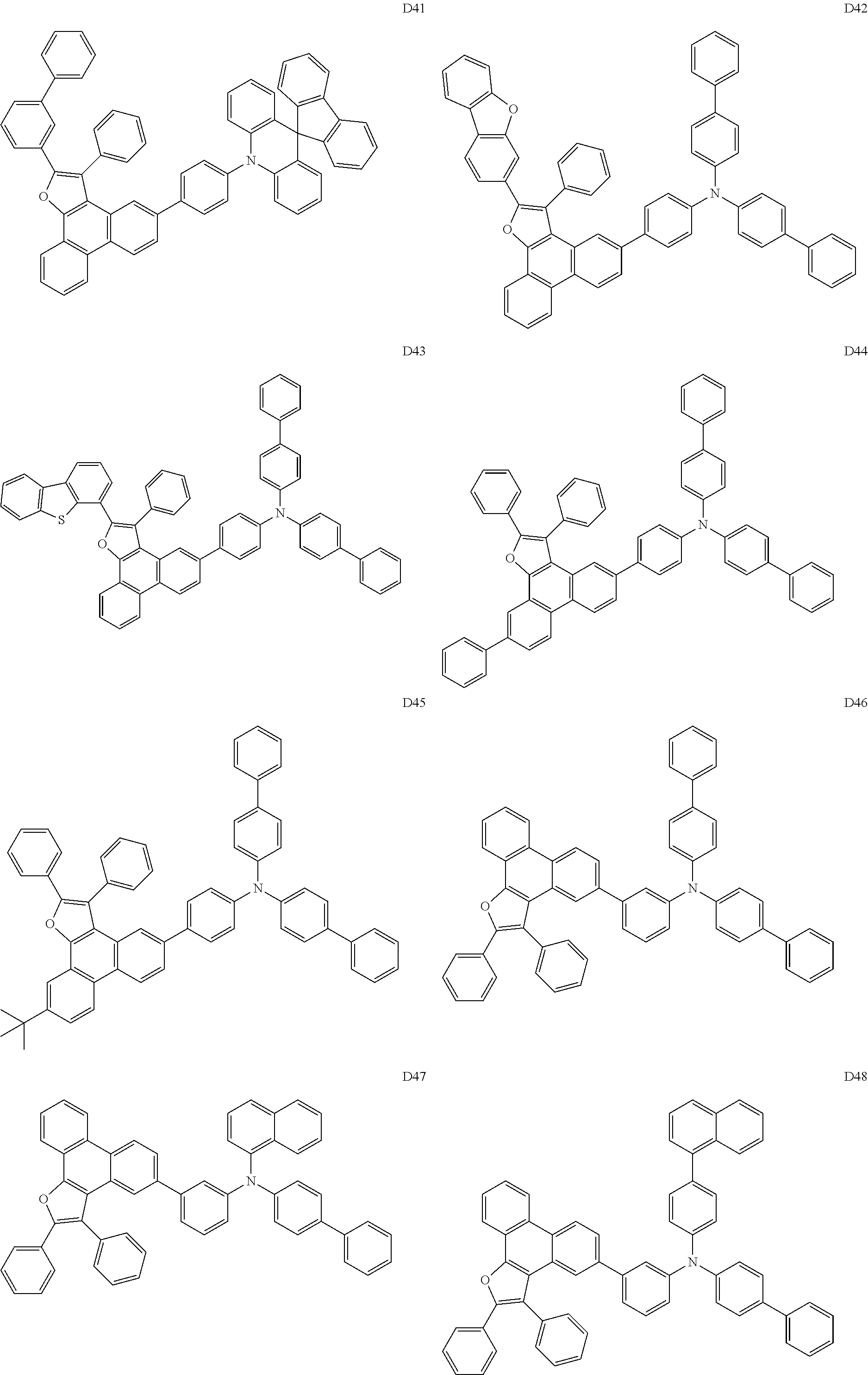

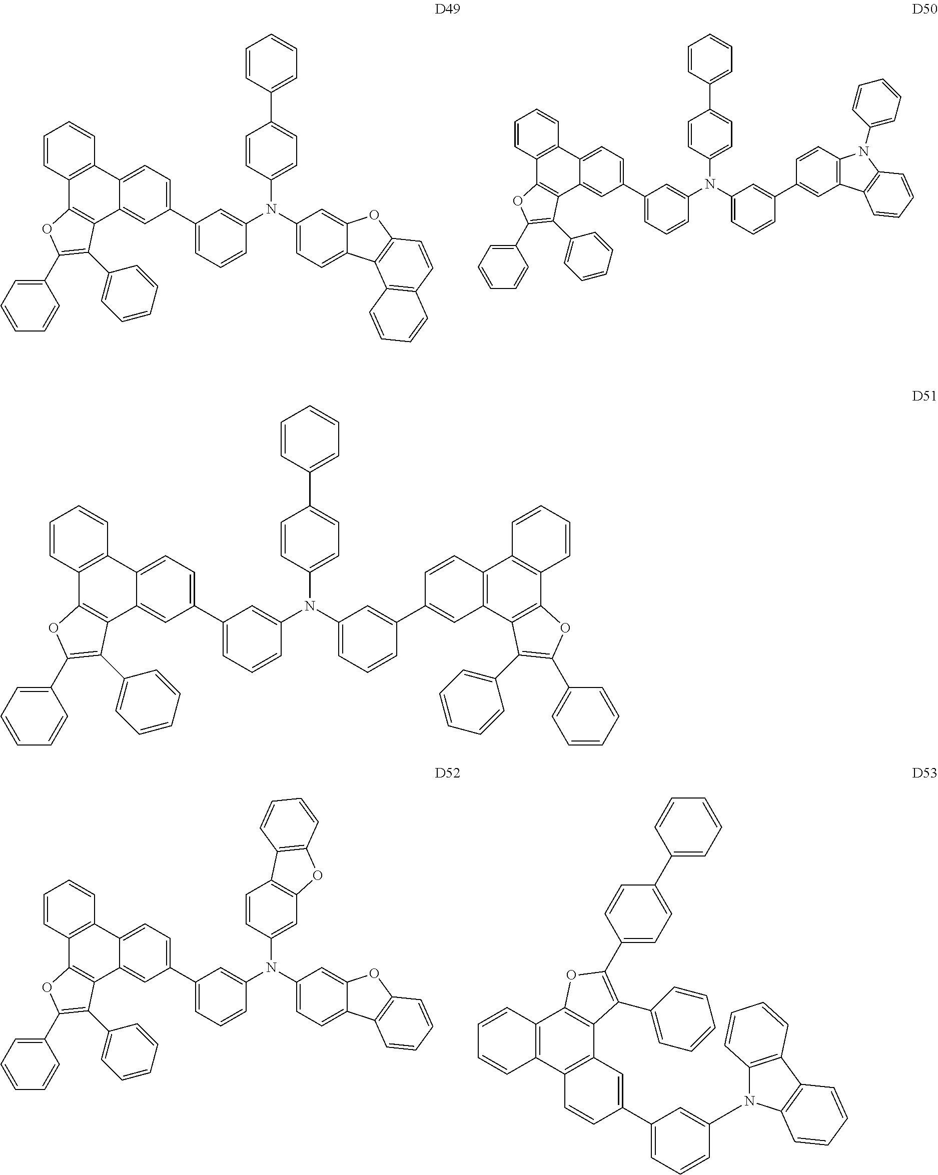

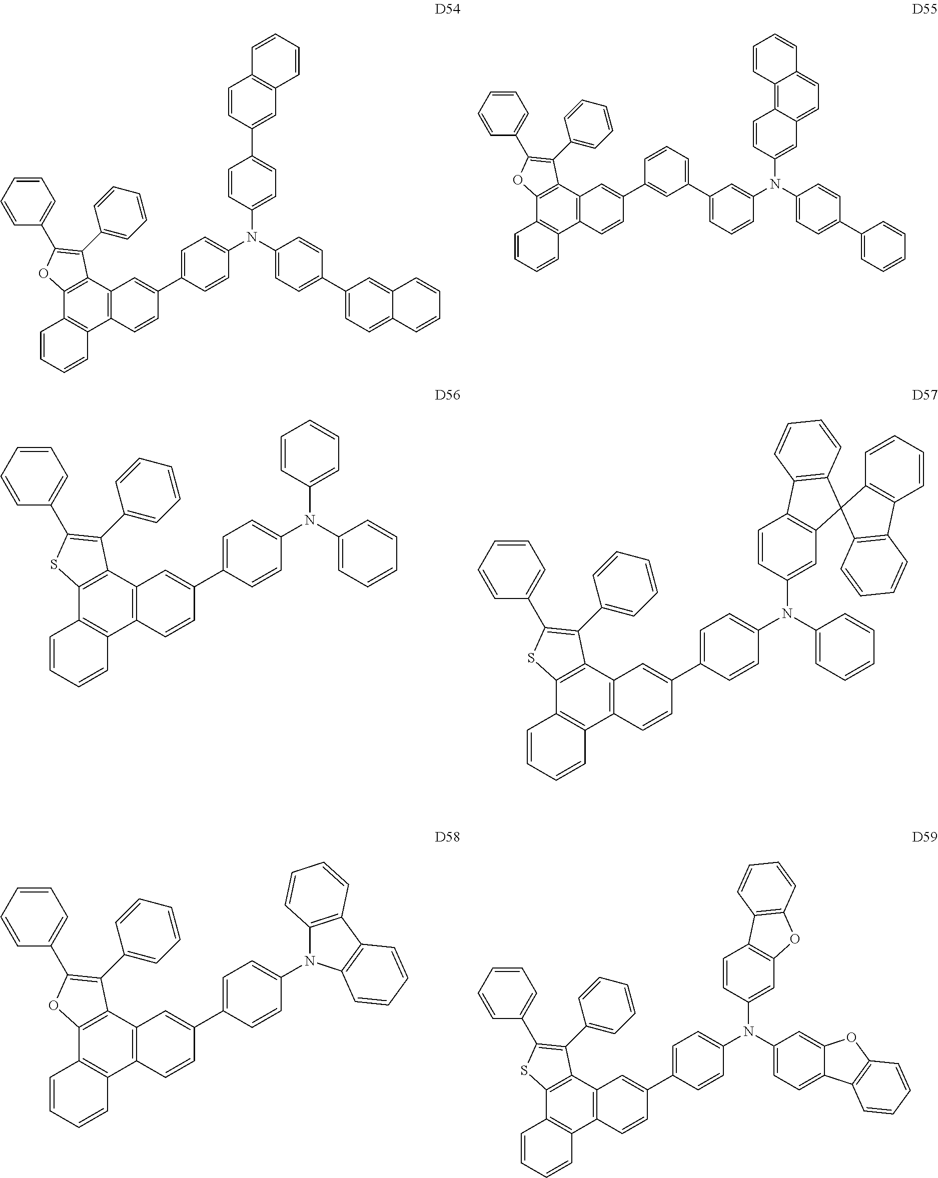

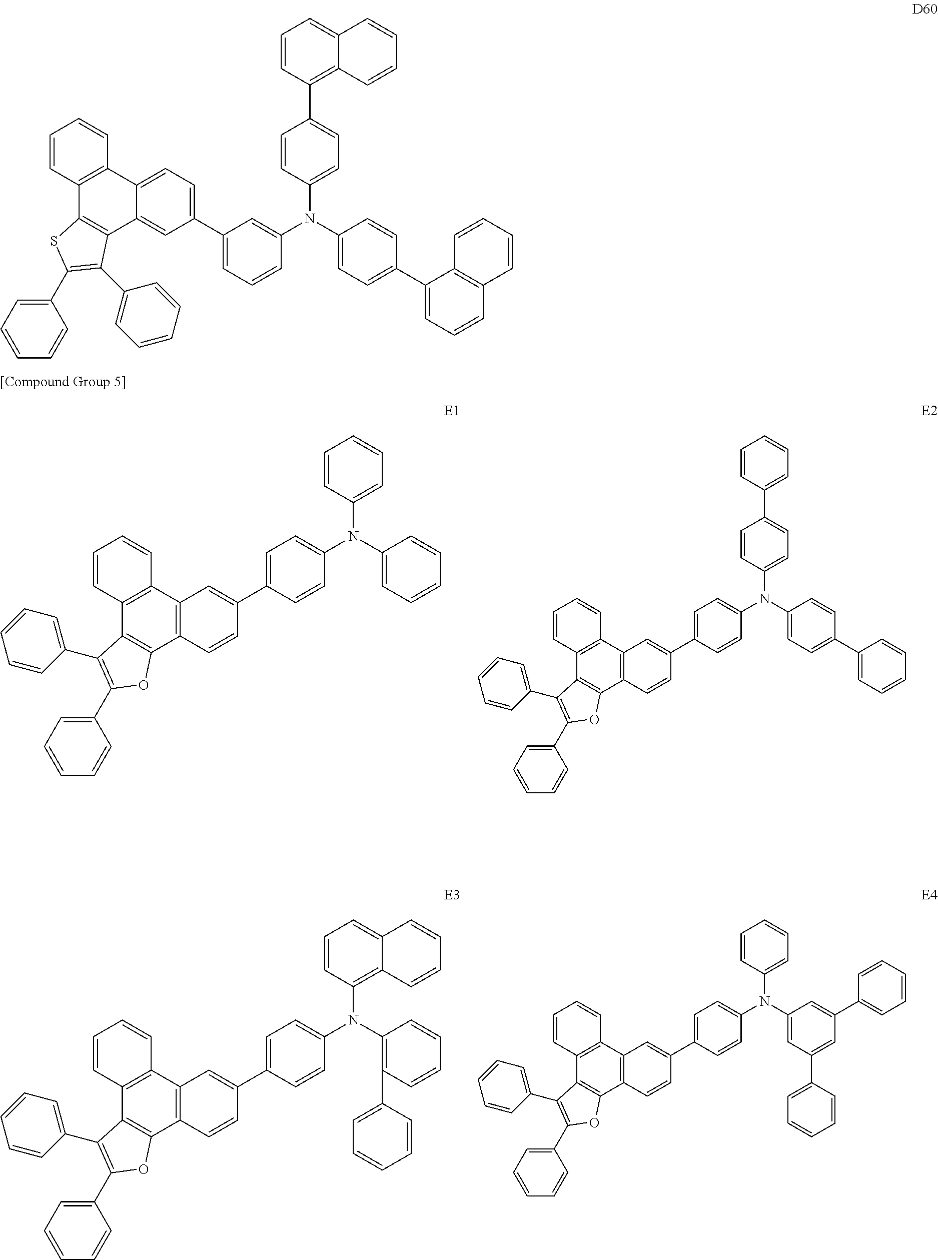

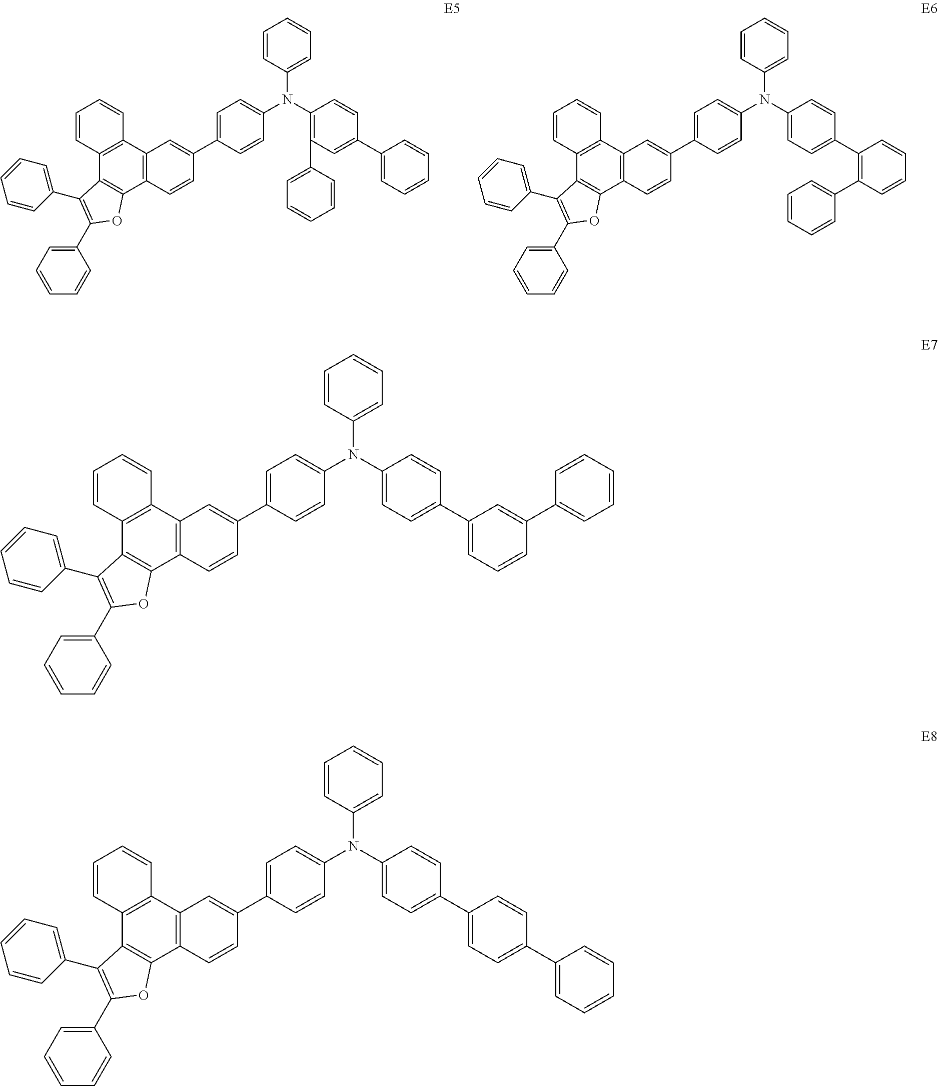 [SMILES: c1ccc(-c2ccc(N(c3ccccc3)c3ccc(-c4ccc5c(c4)c4ccccc4c4c(-c6ccccc6)c(-c6ccccc6)oc54)cc3)c(-c3ccccc3)c2)cc1 ; c1ccc(-c2ccccc2-c2ccc(N(c3ccccc3)c3ccc(-c4ccc5c(c4)c4ccccc4c4c(-c6ccccc6)c(-c6ccccc6)oc54)cc3)cc2)cc1 ; c1ccc(-c2cccc(-c3ccc(N(c4ccccc4)c4ccc(-c5ccc6c(c5)c5ccccc5c5c(-c7ccccc7)c(-c7ccccc7)oc65)cc4)cc3)c2)cc1 ; c1ccc(-c2ccc(-c3ccc(N(c4ccccc4)c4ccc(-c5ccc6c(c5)c5ccccc5c5c(-c7ccccc7)c(-c7ccccc7)oc65)cc4)cc3)cc2)cc1]

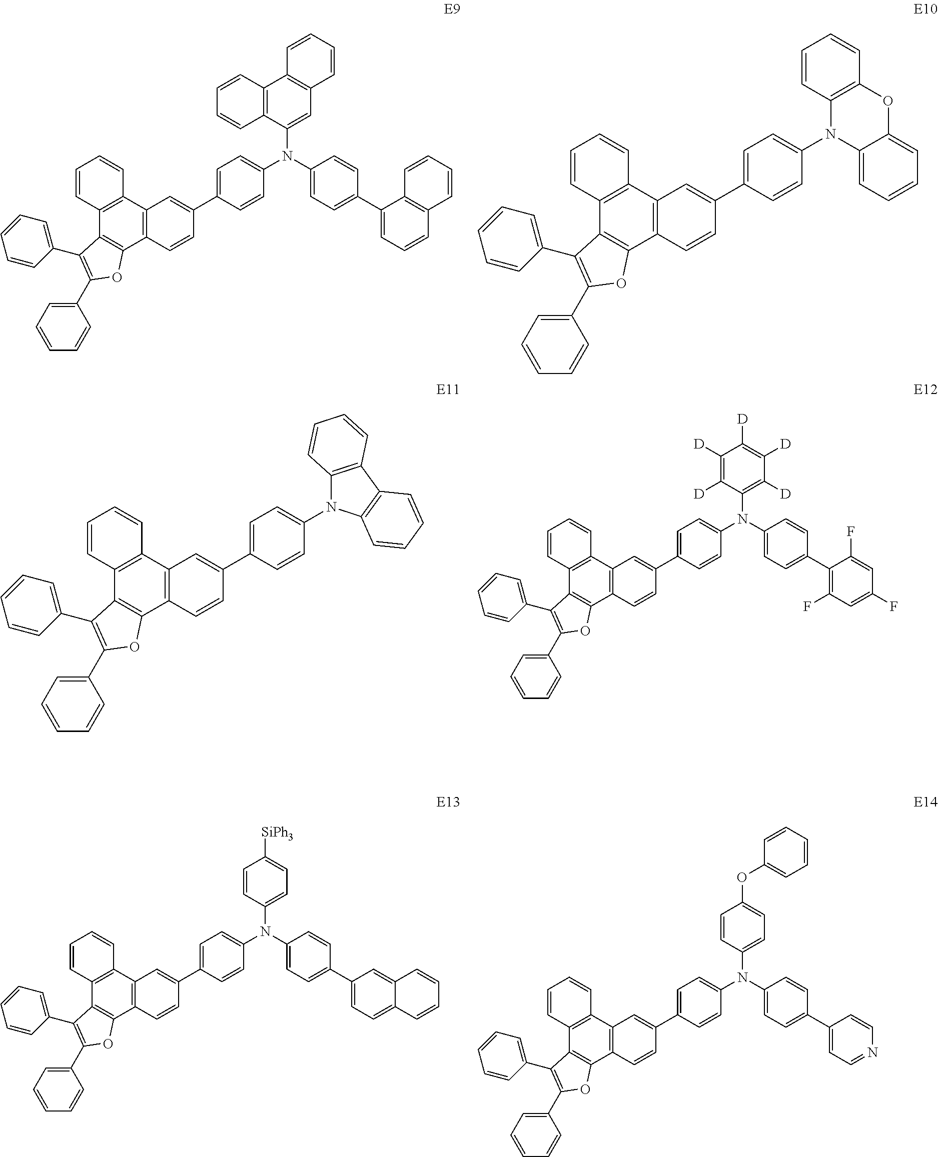

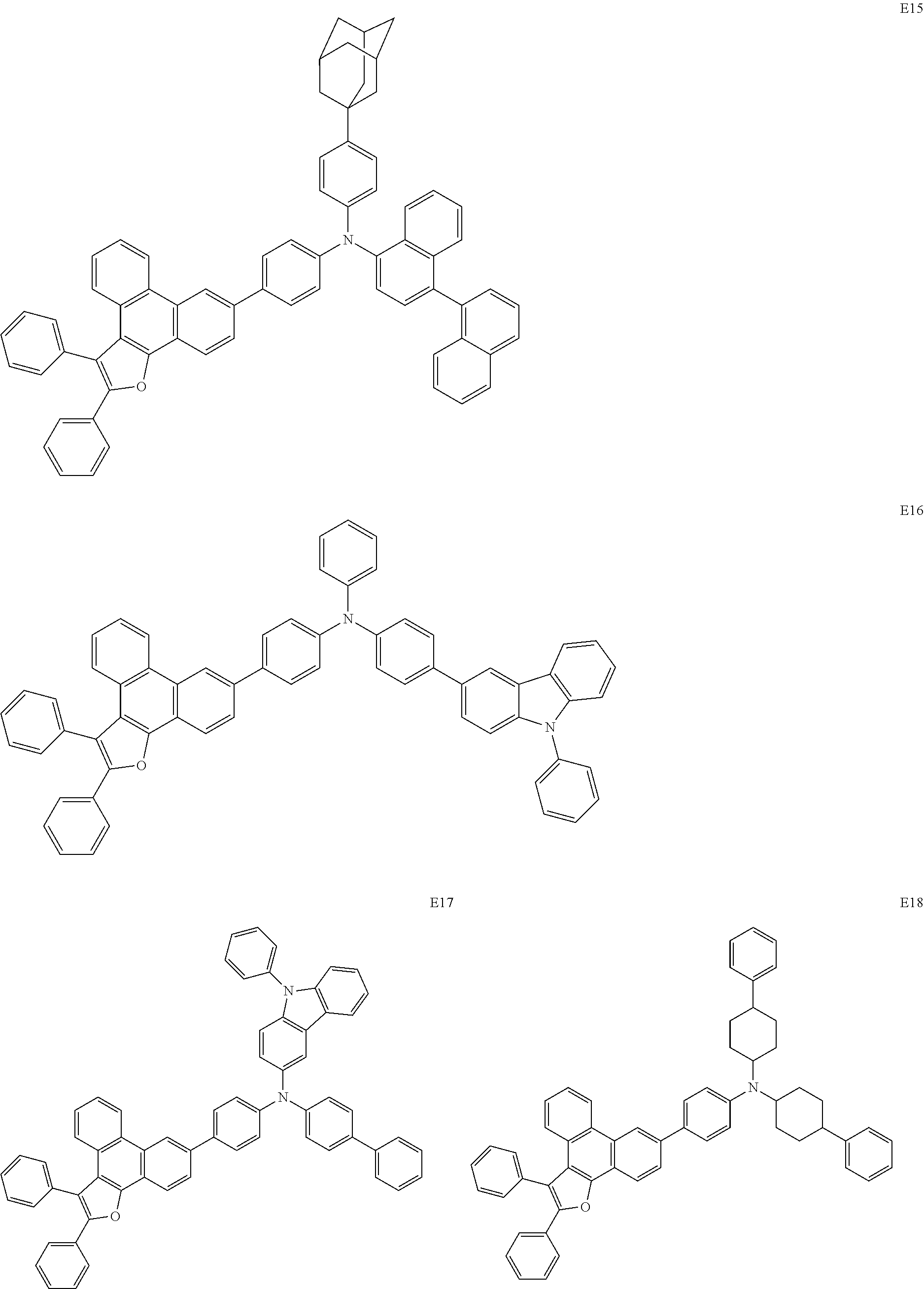 [SMILES: c1ccc(-c2oc3c4ccc(-c5ccc(N(c6ccc(C78CC9CC(CC(C9)C7)C8)cc6)c6ccc(-c7cccc8ccccc78)c7ccccc67)cc5)cc4c4ccccc4c3c2-c2ccccc2)cc1 ; c1ccc(-c2oc3c4ccc(-c5ccc(N(c6ccccc6)c6ccc(-c7ccc8c(c7)c7ccccc7n8-c7ccccc7)cc6)cc5)cc4c4ccccc4c3c2-c2ccccc2)cc1 ; c1ccc(-c2ccc(N(c3ccc(-c4ccc5c(c4)c4ccccc4c4c(-c6ccccc6)c(-c6ccccc6)oc54)cc3)c3ccc4c(c3)c3ccccc3n4-c3ccccc3)cc2)cc1 ; c1ccc(-c2oc3c4ccc(-c5ccc(N(C6CCC(c7ccccc7)CC6)C6CCC(c7ccccc7)CC6)cc5)cc4c4ccccc4c3c2-c2ccccc2)cc1]

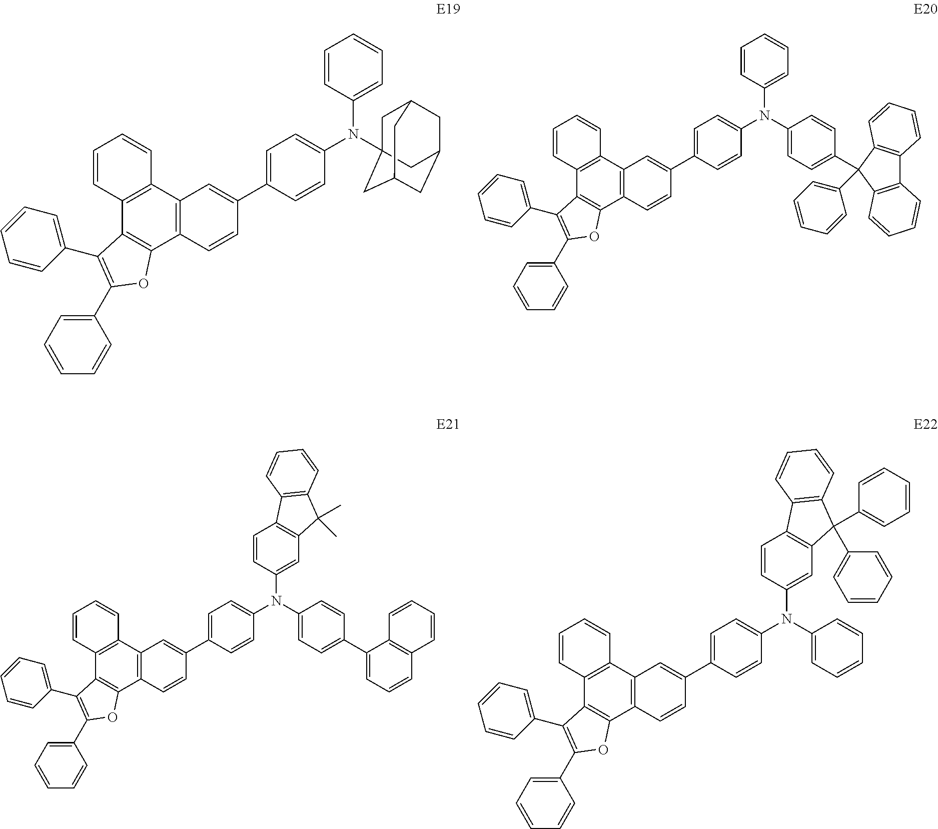 [SMILES: c1ccc(-c2oc3c4ccc(-c5ccc(N(c6ccccc6)C67CC8CC(CC(C8)C6)C7)cc5)cc4c4ccccc4c3c2-c2ccccc2)cc1 ; c1ccc(-c2oc3c4ccc(-c5ccc(N(c6ccccc6)c6ccc(C7(c8ccccc8)c8ccccc8-c8ccccc87)cc6)cc5)cc4c4ccccc4c3c2-c2ccccc2)cc1 ; CC1(C)c2ccccc2-c2ccc(N(c3ccc(-c4ccc5c(c4)c4ccccc4c4c(-c6ccccc6)c(-c6ccccc6)oc54)cc3)c3ccc(-c4cccc5ccccc45)cc3)cc21 ; c1ccc(-c2oc3c4ccc(-c5ccc(N(c6ccccc6)c6ccc7c(c6)C(c6ccccc6)(c6ccccc6)c6ccccc6-7)cc5)cc4c4ccccc4c3c2-c2ccccc2)cc1]

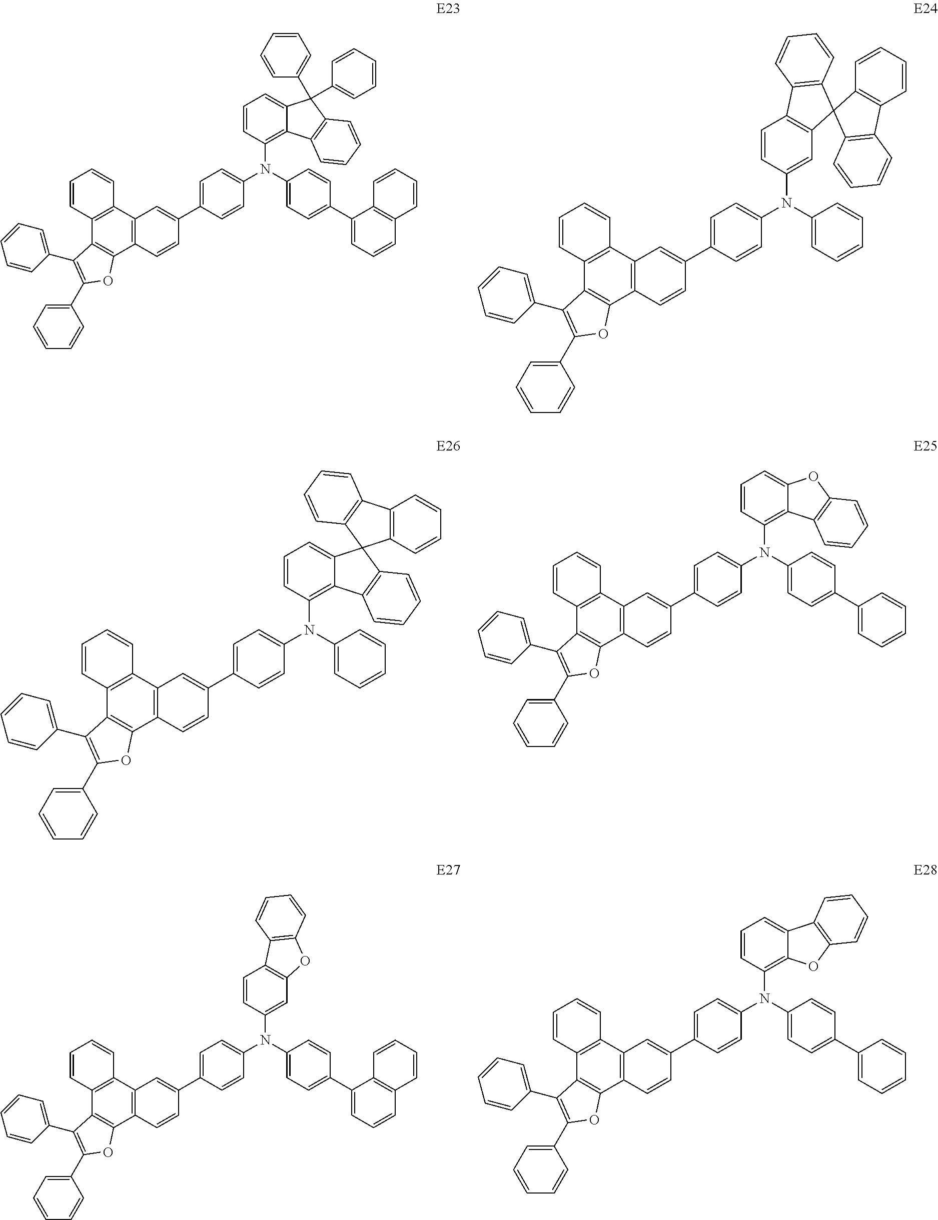

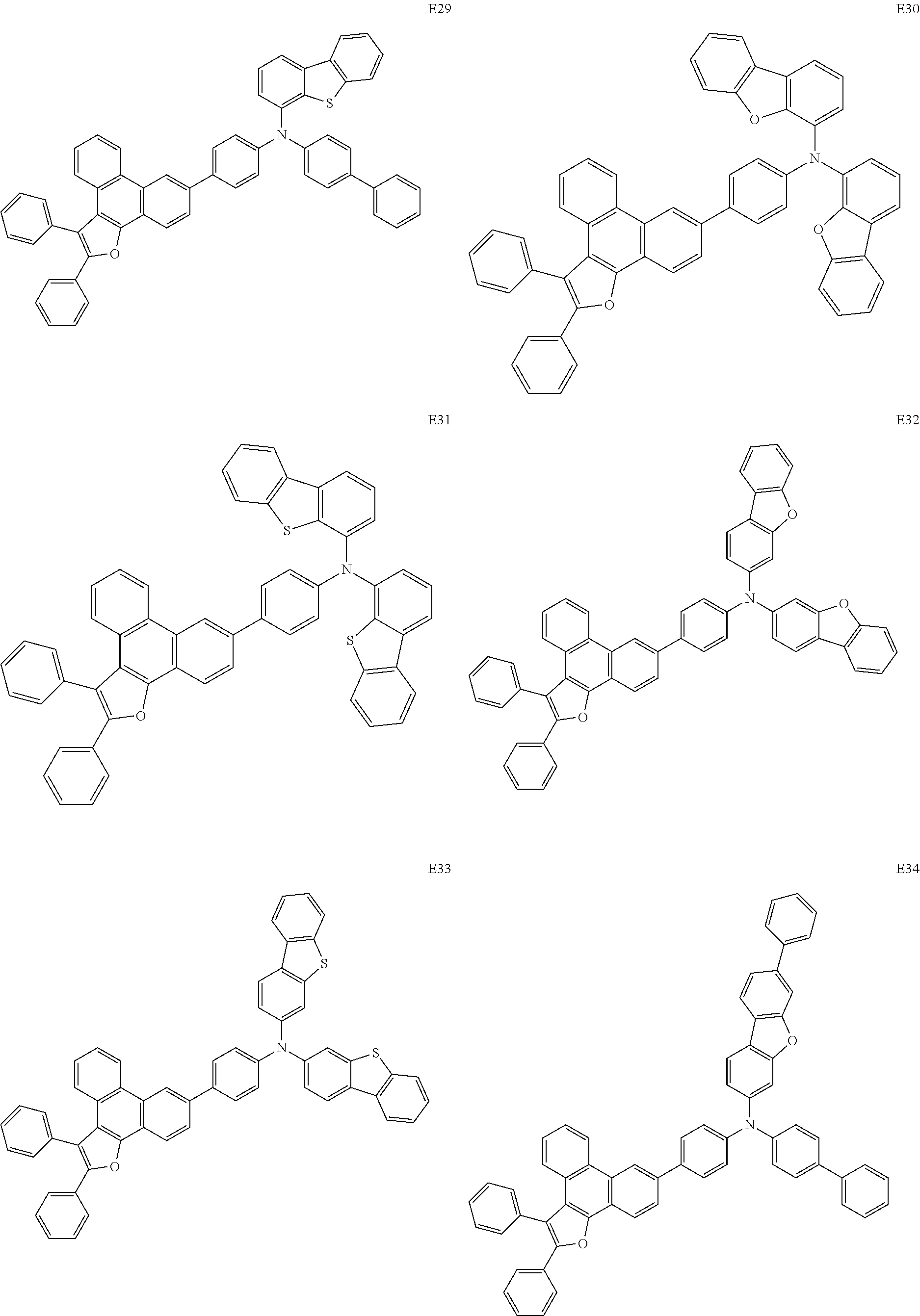

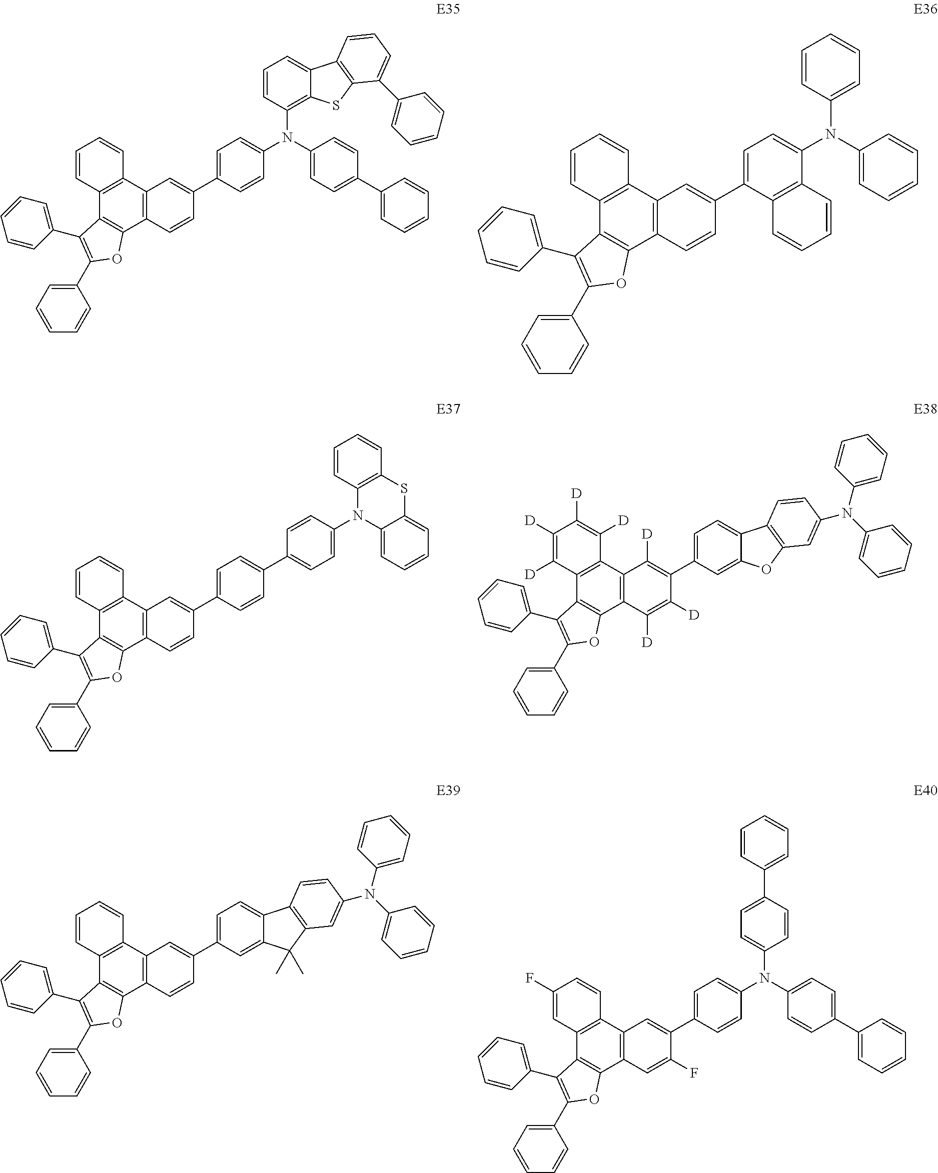

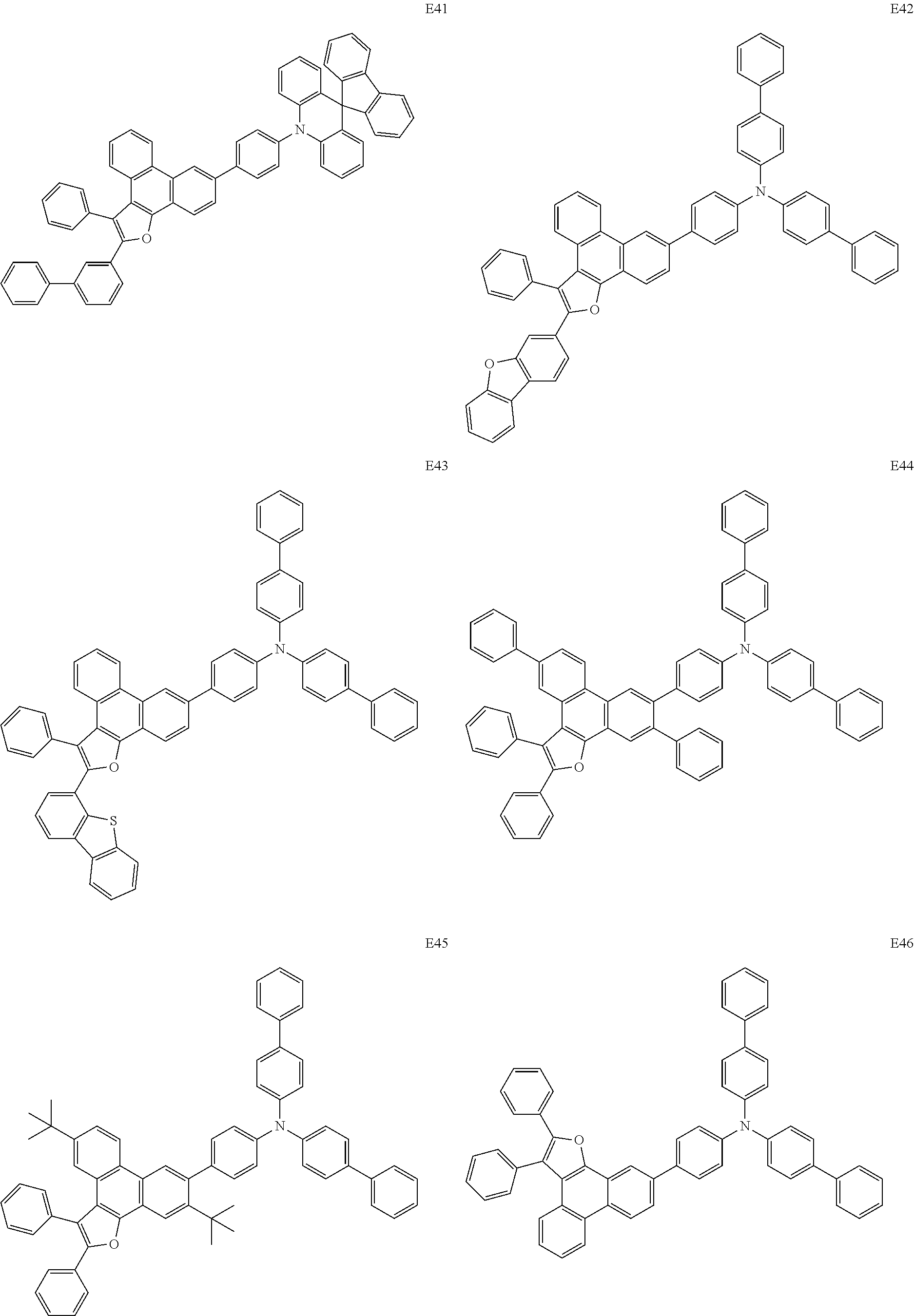

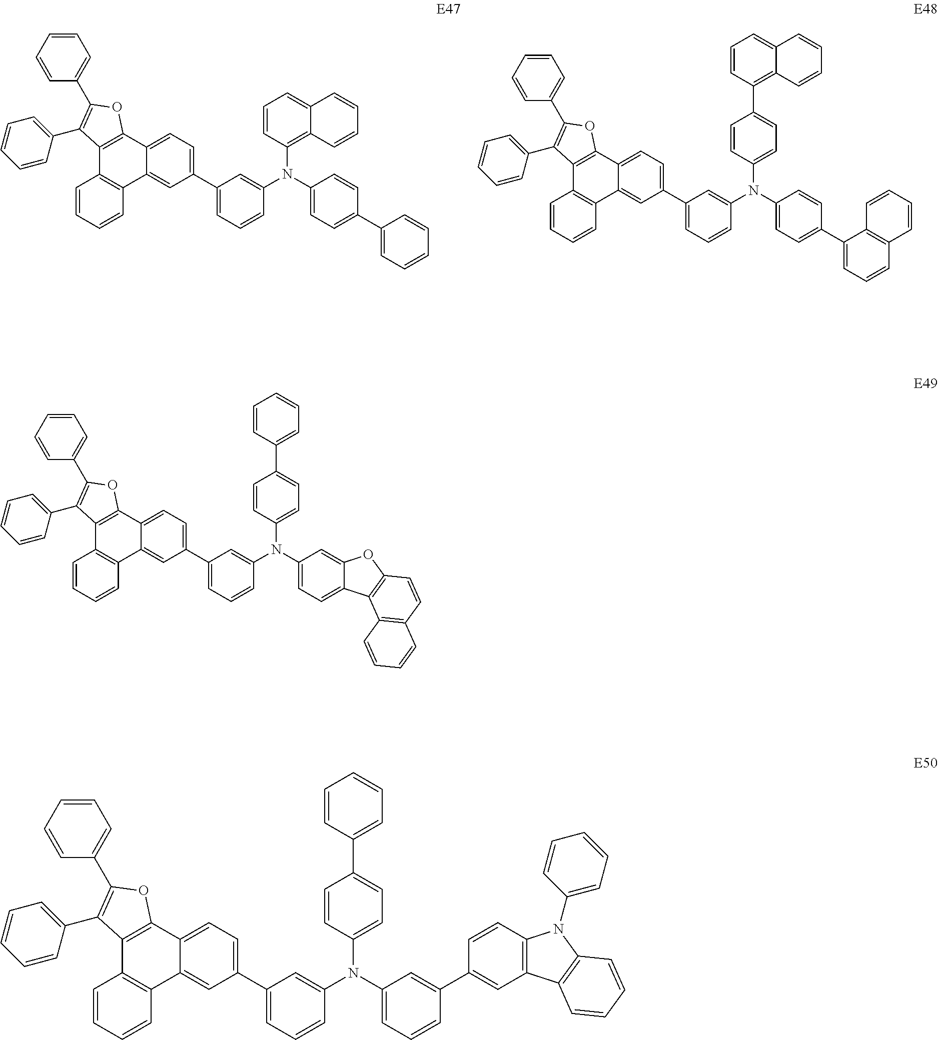 [SMILES: c1ccc(-c2ccc(N(c3cccc(-c4ccc5c(c4)c4ccccc4c4c(-c6ccccc6)c(-c6ccccc6)oc54)c3)c3cccc4ccccc34)cc2)cc1 ; c1ccc(-c2oc3c4ccc(-c5cccc(N(c6ccc(-c7cccc8ccccc78)cc6)c6ccc(-c7cccc8ccccc78)cc6)c5)cc4c4ccccc4c3c2-c2ccccc2)cc1 ; c1ccc(-c2ccc(N(c3cccc(-c4ccc5c(c4)c4ccccc4c4c(-c6ccccc6)c(-c6ccccc6)oc54)c3)c3ccc4c(c3)oc3ccc5ccccc5c34)cc2)cc1 ; c1ccc(-c2ccc(N(c3cccc(-c4ccc5c(c4)c4ccccc4c4c(-c6ccccc6)c(-c6ccccc6)oc54)c3)c3cccc(-c4ccc5c(c4)c4ccccc4n5-c4ccccc4)c3)cc2)cc1]

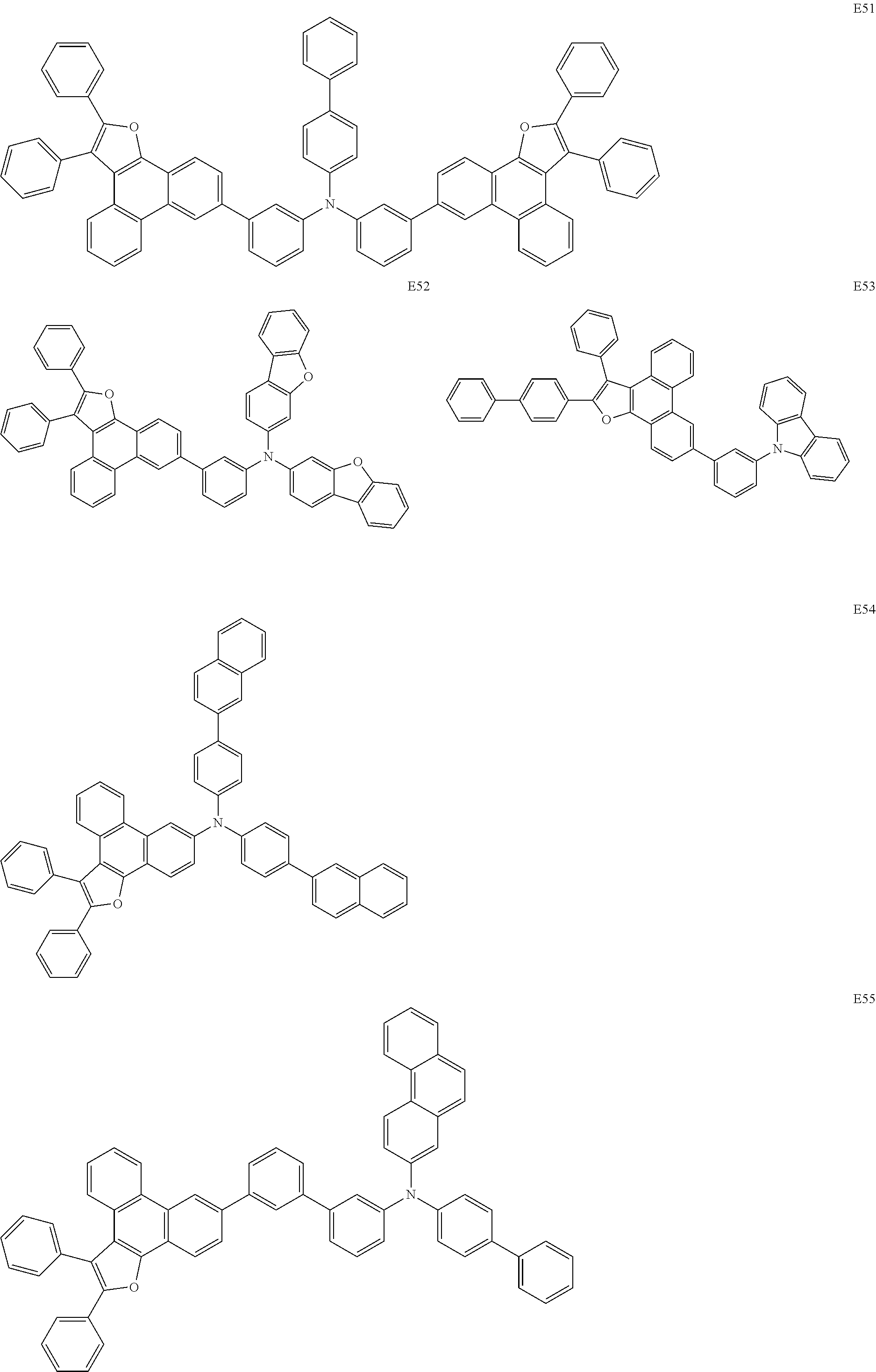

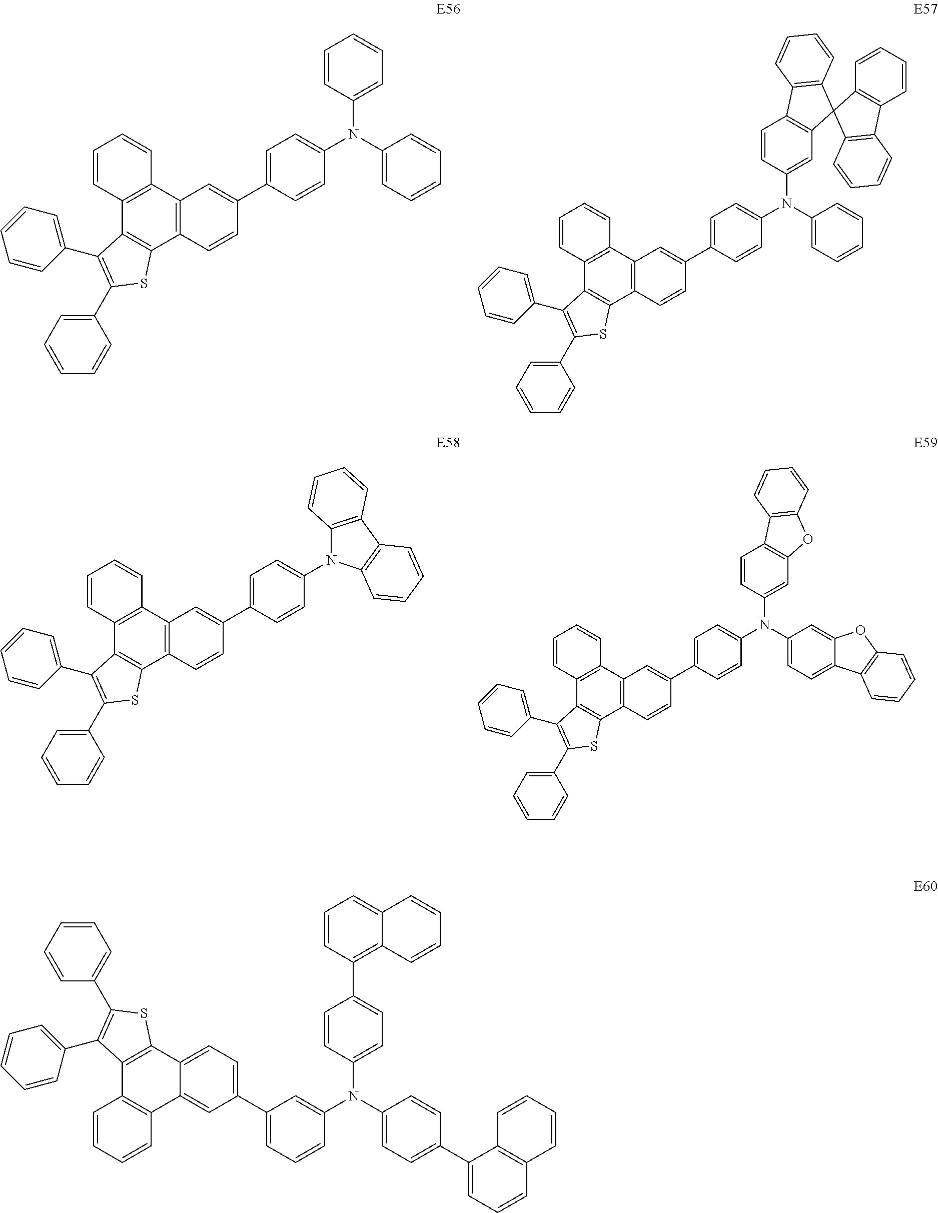 [SMILES: c1ccc(-c2sc3c4ccc(-c5ccc(N(c6ccccc6)c6ccccc6)cc5)cc4c4ccccc4c3c2-c2ccccc2)cc1 ; c1ccc(-c2sc3c4ccc(-c5ccc(N(c6ccccc6)c6ccc7c(c6)C6(c8ccccc8-c8ccccc86)c6ccccc6-7)cc5)cc4c4ccccc4c3c2-c2ccccc2)cc1 ; c1ccc(-c2sc3c4ccc(-c5ccc(-n6c7ccccc7c7ccccc76)cc5)cc4c4ccccc4c3c2-c2ccccc2)cc1 ; c1ccc(-c2sc3c4ccc(-c5ccc(N(c6ccc7c(c6)oc6ccccc67)c6ccc7c(c6)oc6ccccc67)cc5)cc4c4ccccc4c3c2-c2ccccc2)cc1 ; c1ccc(-c2sc3c4ccc(-c5cccc(N(c6ccc(-c7cccc8ccccc78)cc6)c6ccc(-c7cccc8ccccc78)cc6)c5)cc4c4ccccc4c3c2-c2ccccc2)cc1]

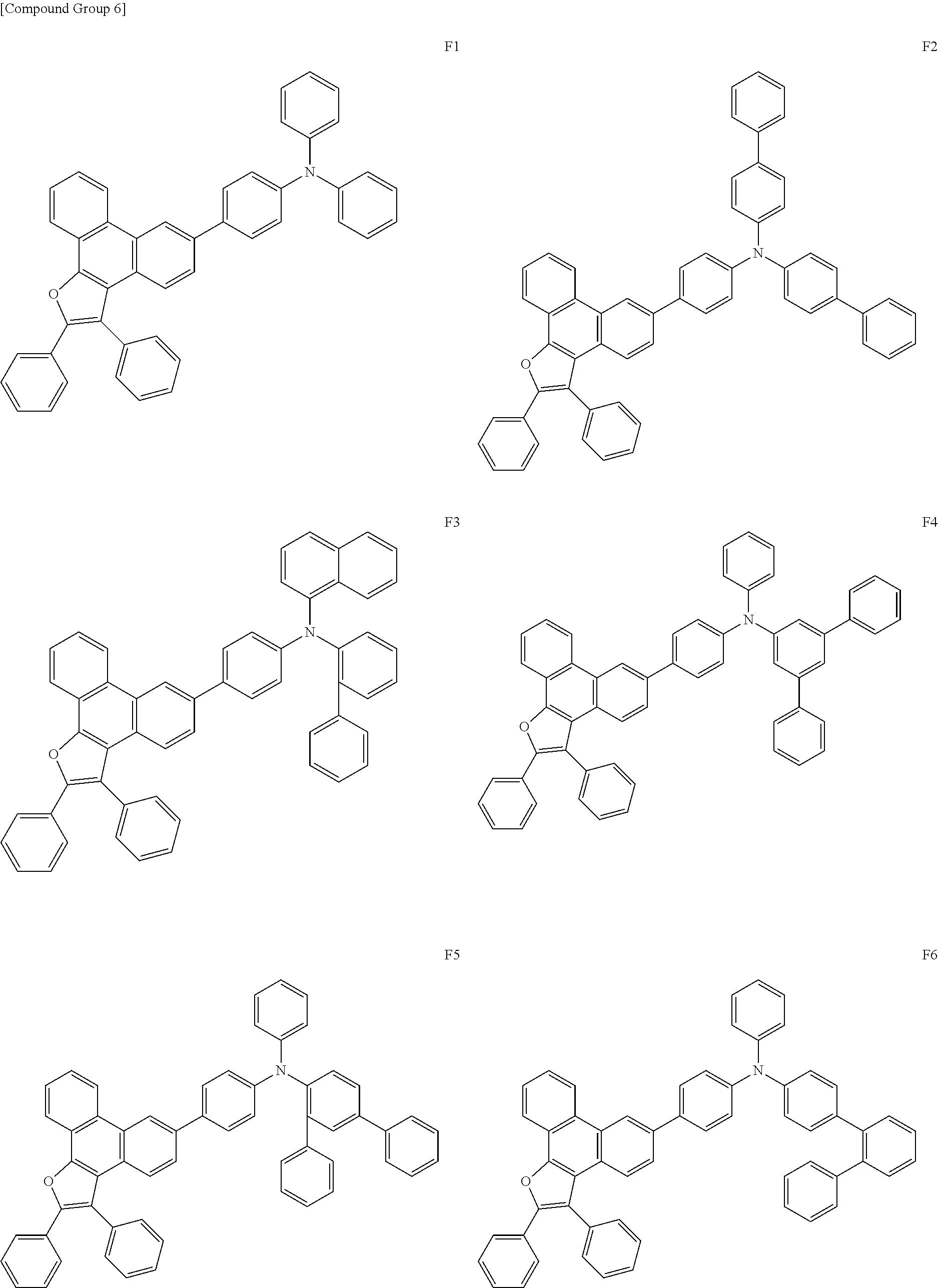

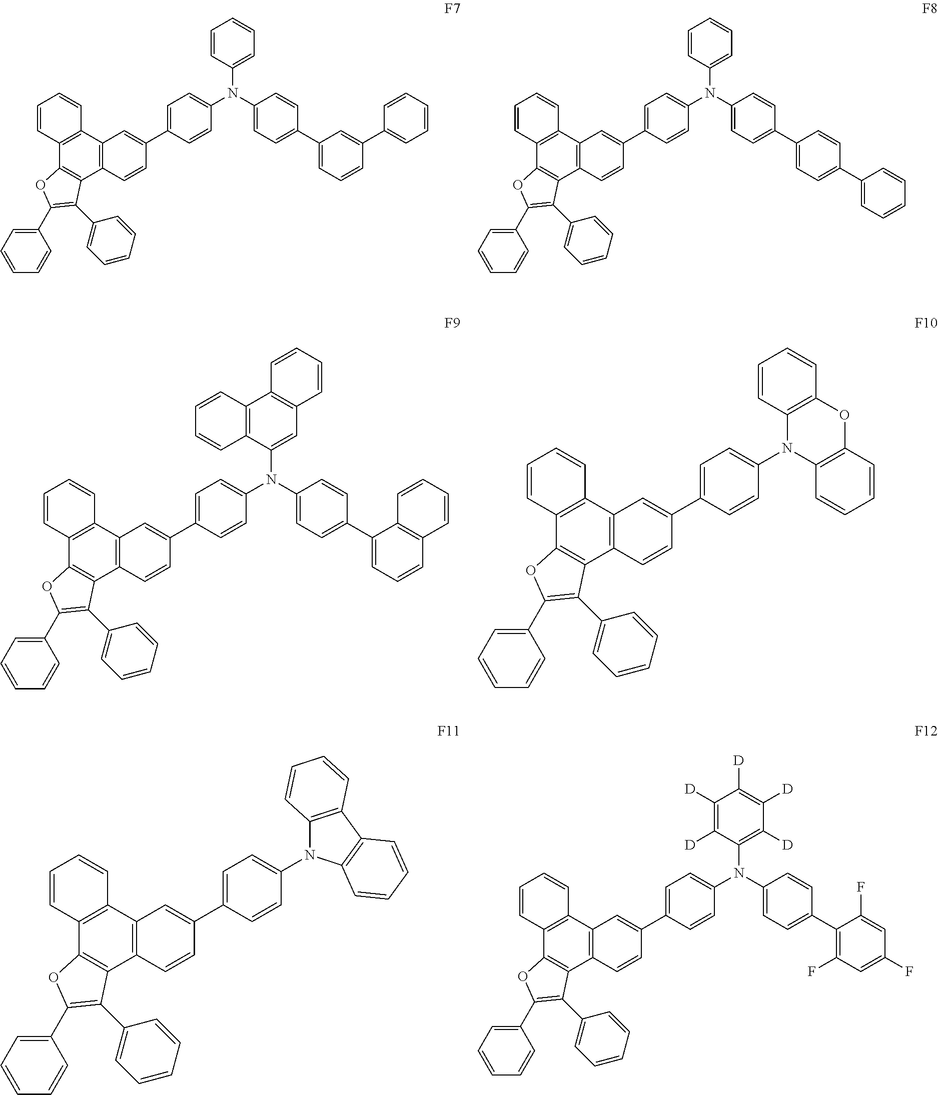

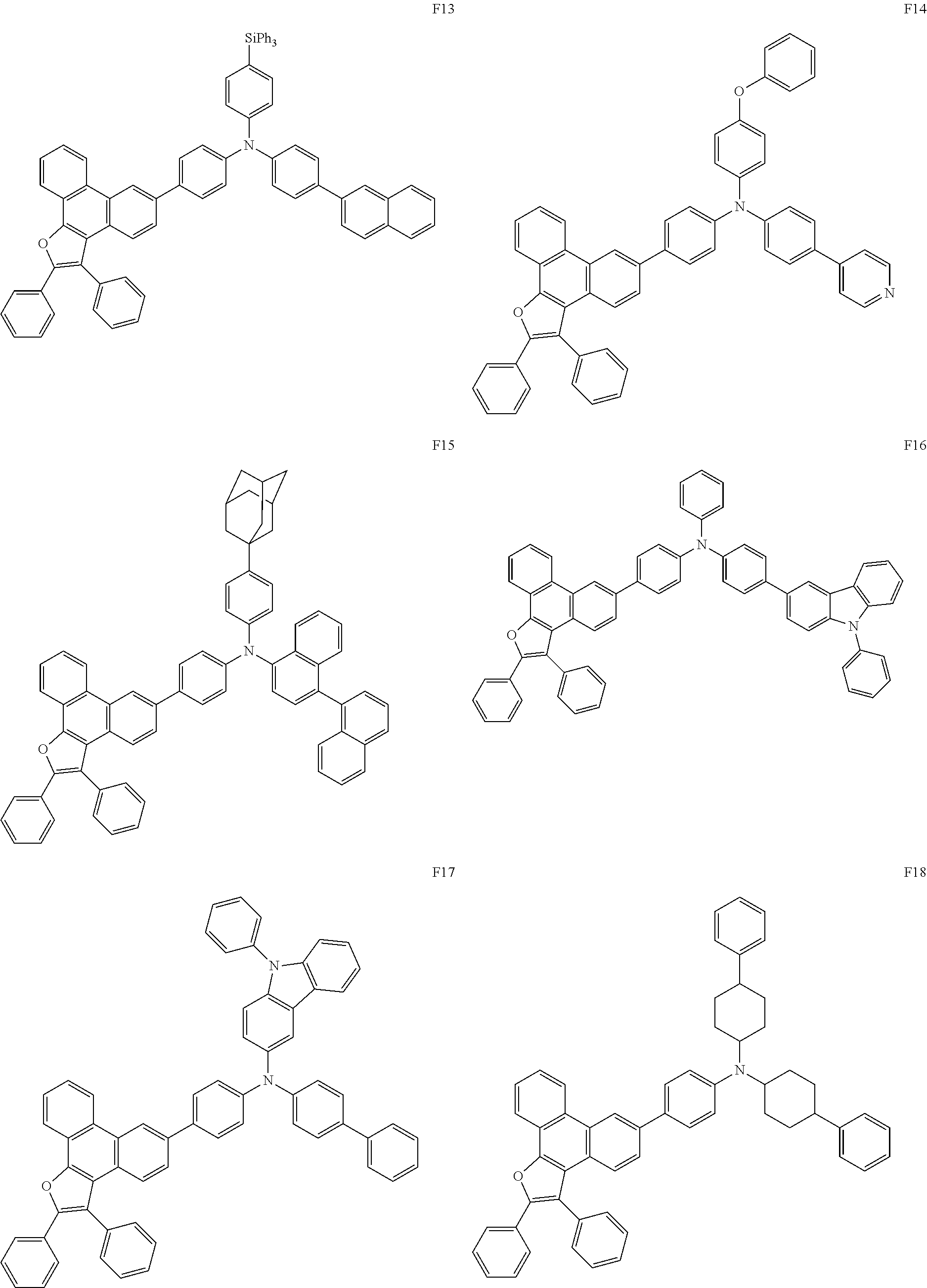

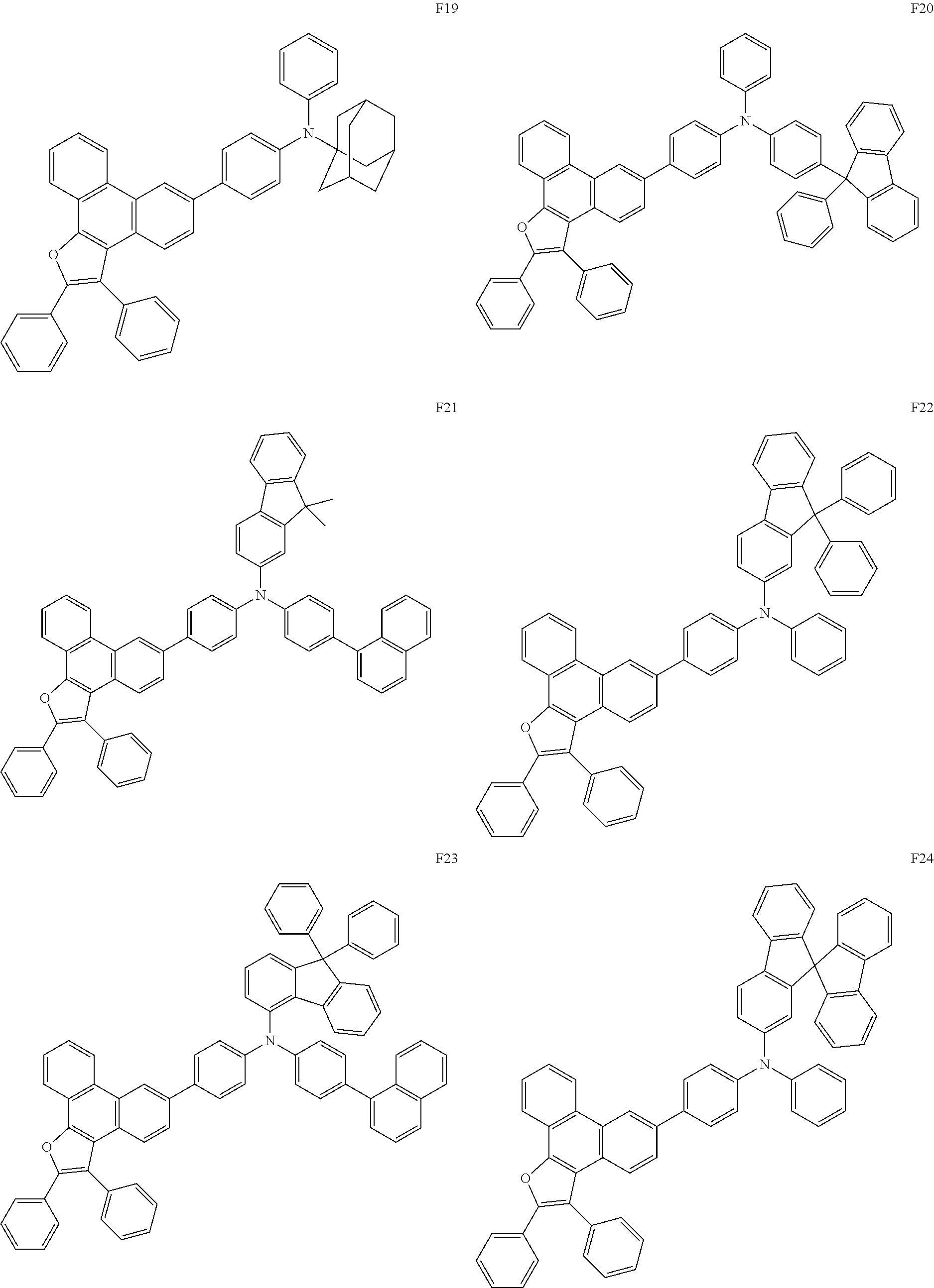

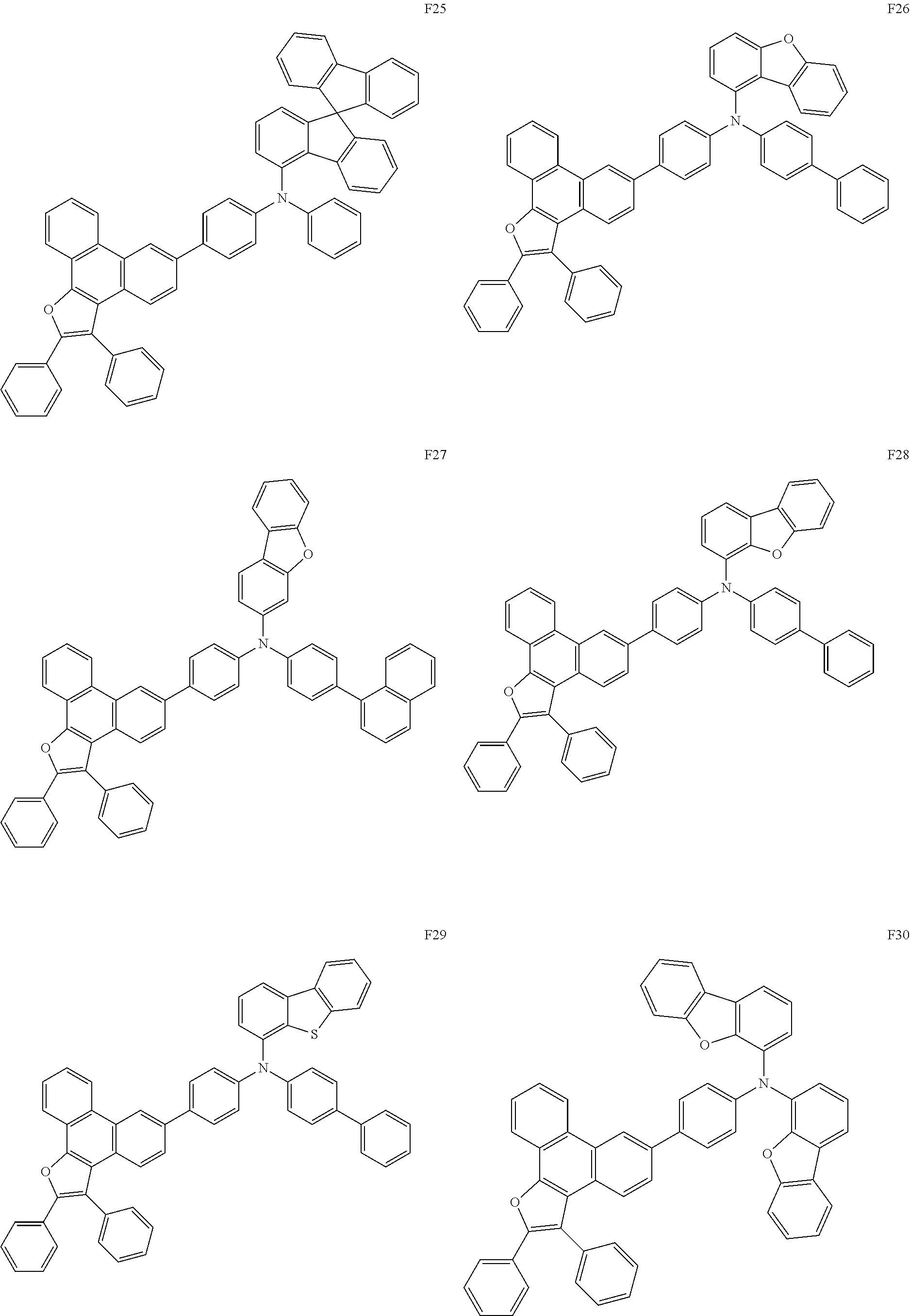

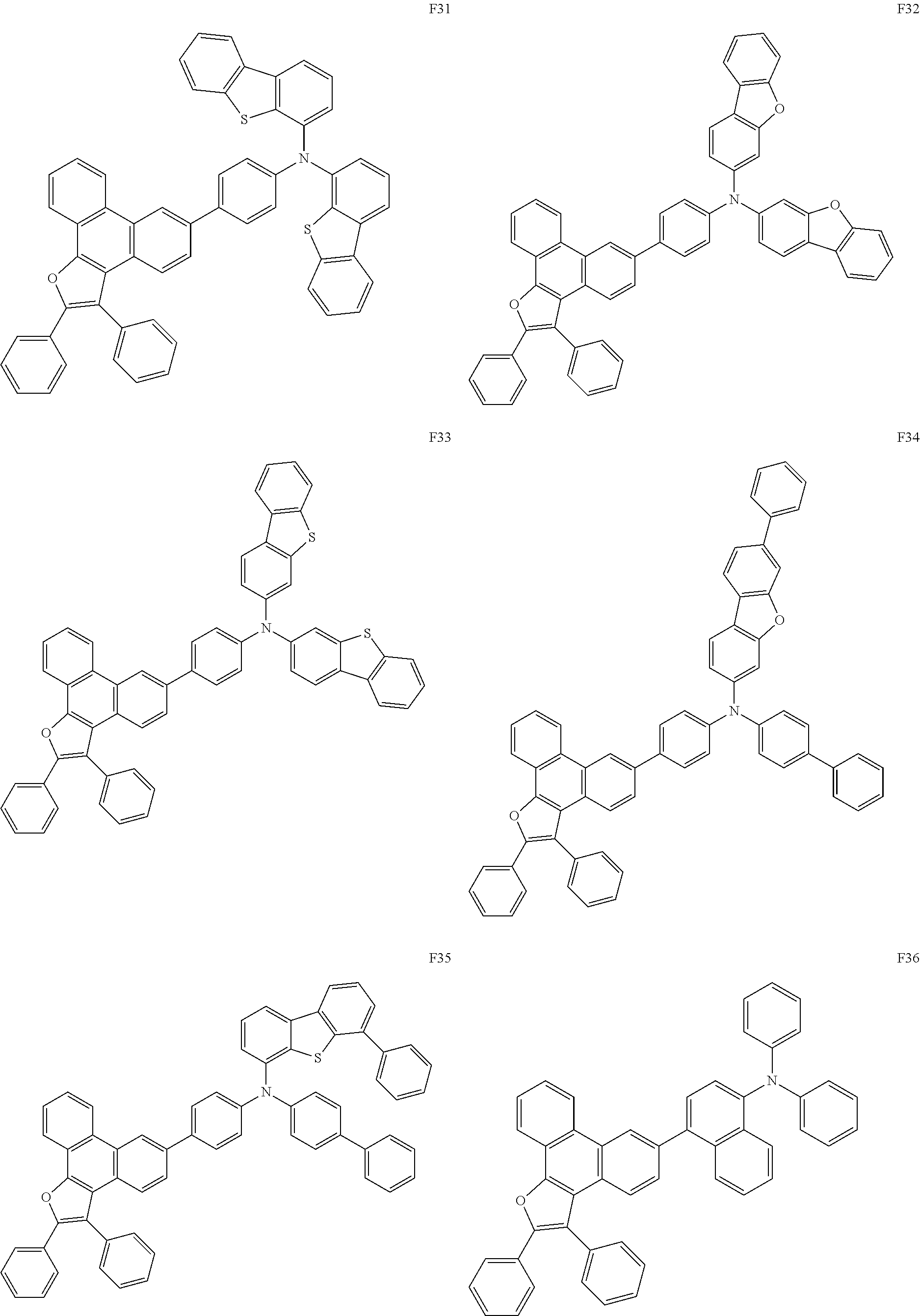

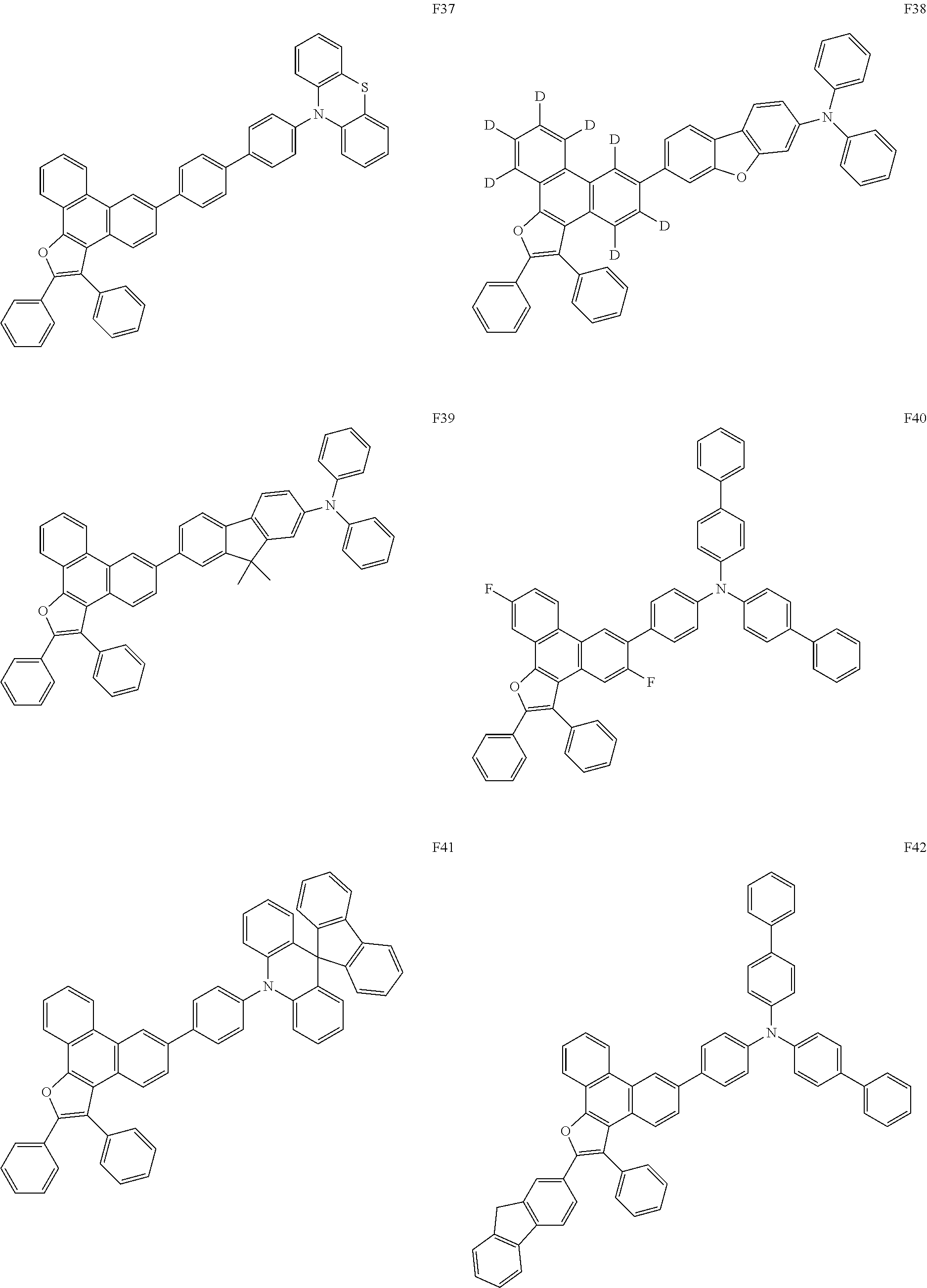

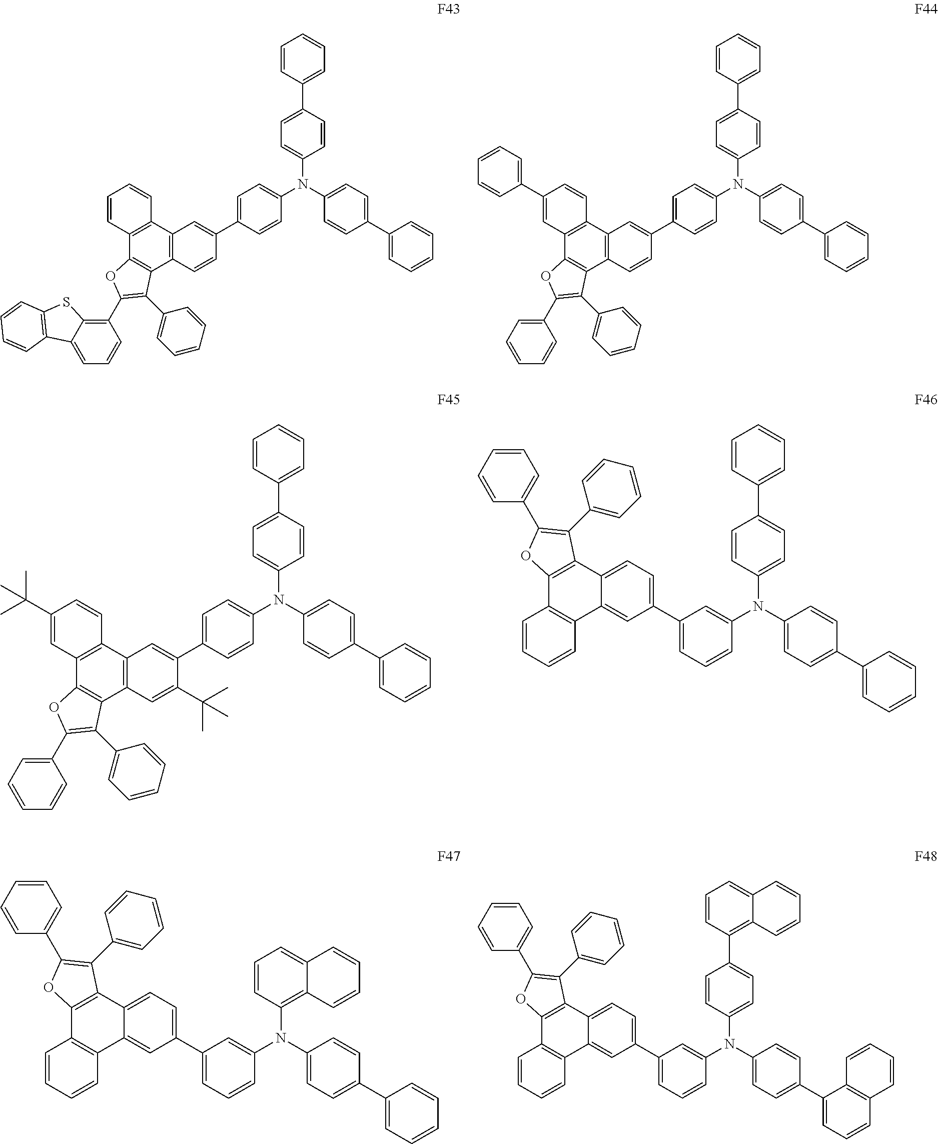

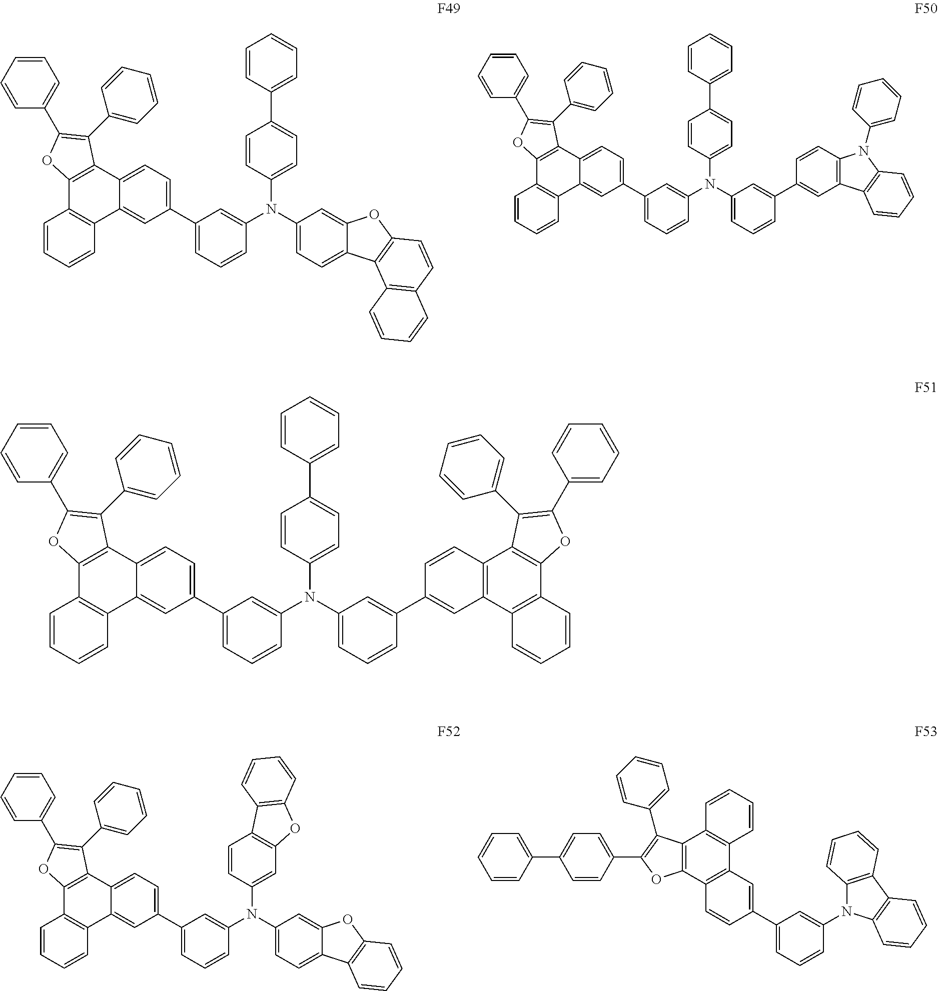

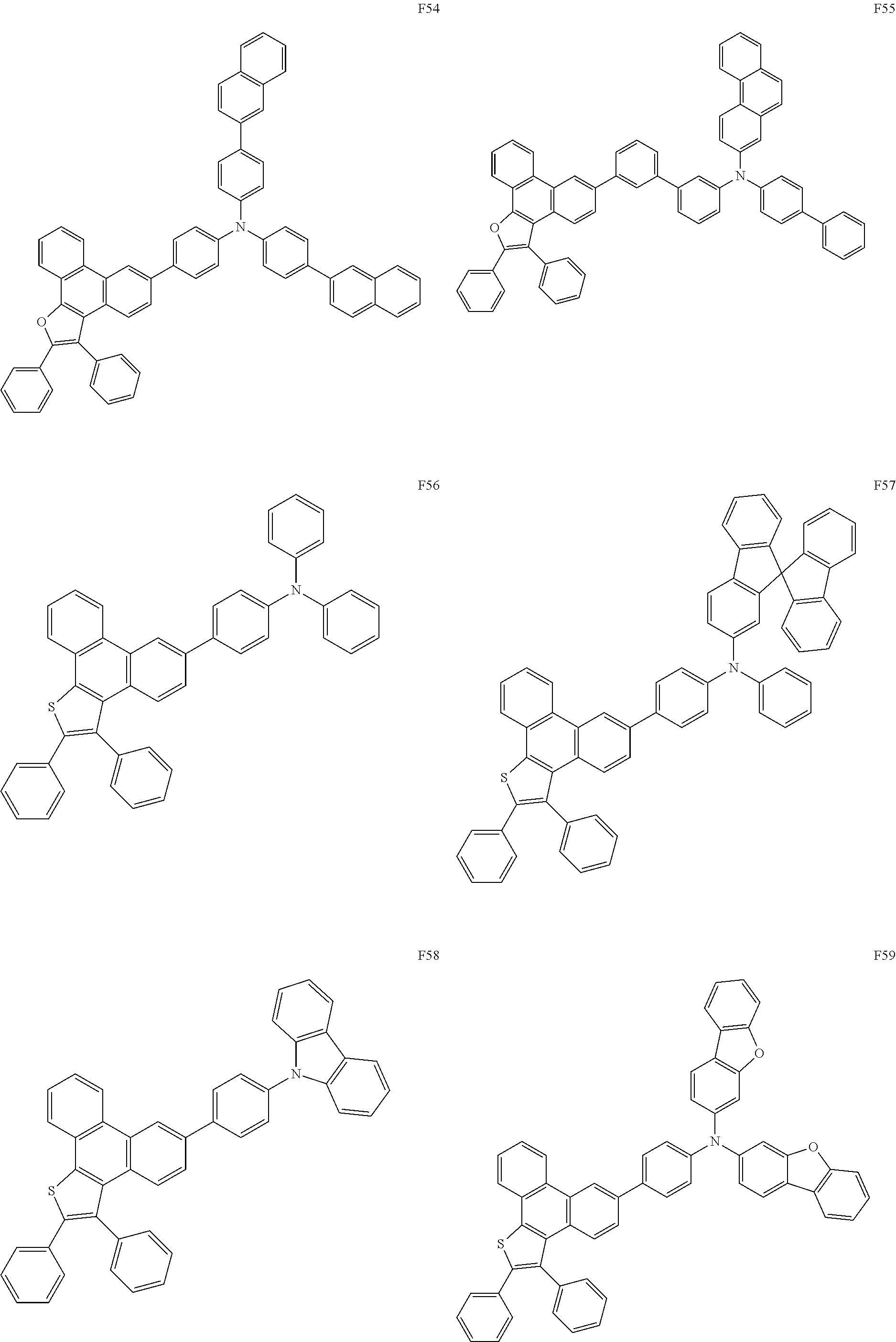

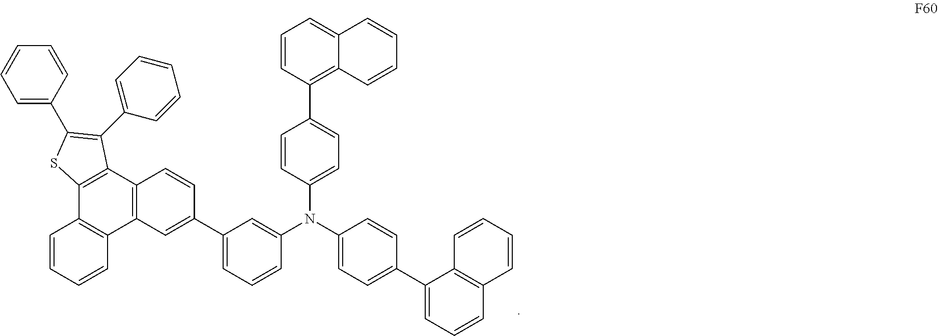 [SMILES: c1ccc(-c2sc3c4ccccc4c4cc(-c5cccc(N(c6ccc(-c7cccc8ccccc78)cc6)c6ccc(-c7cccc8ccccc78)cc6)c5)ccc4c3c2-c2ccccc2)cc1]

Referring to FIGS. 1 to 3 again, an organic electroluminescence device according to an embodiment of the inventive concepts will be explained. The hole transport region HTR includes the polycyclic compound according to an embodiment of the inventive concepts. For example, the hole transport region HTR includes the polycyclic compound represented by Formula 1.

Hereinafter, particular explanation will be given mainly with the difference from the polycyclic compound according to an embodiment of the inventive concepts, and unexplained parts will follow the polycyclic compound according to an embodiment of the inventive concepts.

In the organic electroluminescence devices 10 of an embodiment, the first electrode EL1 has conductivity. The first electrode EL1 may be formed using a metal alloy or a conductive compound. The first electrode EL1 may be an anode.

The first electrode EL1 may be a transmissive electrode, a transflective electrode, or a reflective electrode. If the first electrode EL1 is the transmissive electrode, the first electrode EL1 may be formed using a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). Also, the first electrode EL1 may have a structure including a plurality of layers including a reflective layer or a transflective layer formed using the above materials, and a transmissive conductive layer formed using ITO, IZO, ZnO, or ITZO. For example, the first electrode EL1 may include a plurality of layers of ITO/Ag/ITO.

The thickness of the first electrode EL1 may be from about 1,000 Å to about 10,000 Å, for example, from about 1,000 Å to about 3,000 Å.

The hole transport region HTR is provided on the first electrode EL1. The hole transport region HTR may include at least one of a hole injection layer HIL, a hole transport layer HTL, a hole buffer layer, or an electron blocking layer EBL.

The hole transport region HTR may include the polycyclic compound according to an embodiment of the inventive concepts as described above.

The hole transport region HTR may have a single layer formed using a single material, a single layer formed using a plurality of different materials, or a multilayer structure including a plurality of layers formed using a plurality of different materials.

For example, the hole transport region HTR may have the structure of a single layer of a hole injection layer HIL, or a hole transport layer HTL, and may have a structure of a single layer formed using a hole injection material and a hole transport material. Alternatively, the hole transport region HTR may have a structure of a single layer formed using a plurality of different materials, or a structure laminated from the first electrode EL1 of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL, without limitation.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

As described above, the hole transport region HTR may have a multilayer structure having a plurality of layers, and any one layer among the plurality of the layers may include the polycyclic compound represented by Formula 1. For example, the hole transport region HTR may include a hole injection layer HIL disposed on the first electrode EL1, and a hole transport layer HTL disposed on the hole injection layer HIL, and the hole transport layer HTL may include the polycyclic compound represented by Formula 1. However, an embodiment of the inventive concepts is not limited thereto, for example, the hole injection layer HIL may include the polycyclic compound represented by Formula 1. In addition, the hole transport region HTR may further include an electron blocking layer EBL disposed on the hole transport layer HTL.

The hole transport region HTR may include one or two or more kinds of the polycyclic compounds represented by Formula 1. For example, the hole transport region HTR may include at least one selected from the compounds represented in Compound Group 1 to Compound Group 6.

However, the hole transport region may further include the materials listed below in each layer.

The hole injection layer HIL may include, for example, a phthalocyanine compound such as copper phthalocyanine, N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino)triphenylamine (m-MTDATA), 4,4′,4″-tris(N,N-diphenylamino)triphenylamine (TDATA), 4,4′,4″-tris(N,-(2-naphthyl)-N-phenylamino)triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), (polyaniline)/poly(4-styrenesulfonate) (PANI/PSS), N,N′-di(naphthylene-1-yl)-N,N′-diphenyl-benzidine (NPD), triphenylamine-containing polyetherketone (TPAPEK), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (HAT-CN), etc. However, an embodiment of the inventive concepts is not limited thereto.

The hole transport layer HTL may include the polycyclic compound represented by Formula 1 as described above. However, an embodiment of the inventive concepts is not limited thereto, and common materials well-known in the art may be included. For example, the hole transport layer HTL may include carbazole derivatives such as N-phenyl carbazole and polyvinyl carbazole, fluorine-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(1-naphthlene-1-yl)-N,N′-diphenyl-benzidine (NPD), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), etc.

Meanwhile, the hole transport region HTR may further include an electron blocking layer EBL, and the electron blocking layer EBL may be disposed between the hole transport layer HTL and the emission layer EML. The electron blocking layer EBL plays the role of preventing electron injection from an electron transport region ETR to a hole transport region HTR.

The electron blocking layer EBL may include, for example, carbazole derivatives such as N-phenyl carbazole, and polyvinyl carbazole, fluorine-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(naphthalene-1-yl)-N,N′-diphenyl-benzidine (NPD), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), mCP, etc. In addition, the electron blocking layer EBL may include the polycyclic compound according to an embodiment of the inventive concepts.

The thickness of the hole transport region HTR may be from about 100 Å to about 10,000 Å, for example, from about 100 Å to about 5,000 Å. The thickness of the hole injection layer HIL may be, for example, from about 30 Å to about 1,000 Å, and the thickness of the hole transport layer HTL may be from about 30 Å to about 1,000 Å. For example, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material in addition to the above-described materials to improve conductivity. The charge generating material may be dispersed uniformly or non-uniformly in the hole transport region HTR. The charge generating material may be, for example, a p-dopant. The p-dopant may be one of quinone derivatives, metal oxides, or cyano group-containing compounds, without limitation. For example, non-limiting examples of the p-dopant may include quinone derivatives such as tetracyanoquinodimethane (TCNQ) and 2,3,5,6-tetrafluoro-tetracyanoquinodimethane (F4-TCNQ), metal oxides such as tungsten oxide and molybdenum oxide, without limitation.

As described above, the hole transport region HTR may further include at least one of a hole buffer layer or an electron blocking layer EBL in addition to the hole injection layer HIL and the hole transport layer HTL. The hole buffer layer may compensate a resonance distance according to the wavelength of light emitted from the emission layer EML and increase light emission efficiency. Materials included in the hole transport region HTR may be used as materials included in the hole buffer layer.

The emission layer EML is provided on the hole transport region HTR. The emission layer EML may have a thickness of, for example, about 100 Å to about 600 Å. The emission layer EML may have a single layer formed using a single material, a single layer formed using a plurality of different materials, or a multilayer structure having a plurality of layers formed using a plurality of different materials.

The emission layer EML may be formed using various methods such as a vacuum deposition method, a spin coating method, a cast method, a Langmuir-Blodgett (LB) method, an inkjet printing method, a laser printing method, and a laser induced thermal imaging (LITI) method.

The emission layer EML may emit one of red light, green light, blue light, white light, yellow light, or cyan light. The emission layer EML may include a fluorescence emitting material or a phosphorescence emitting material.

As the material of the emission layer EML, well-known light-emitting materials may be used, and may be selected from fluoranthene derivatives, pyrene derivatives, arylacetylene derivatives, anthracene derivatives, fluorene derivatives, perylene derivatives, chrysene derivatives, etc., without specific limitation. Preferably, pyrene derivatives, perylene derivatives, and anthracene derivatives may be used. For example, as the host material of the emission layer EML, an anthracene derivative represented by Formula 11 below may be used.

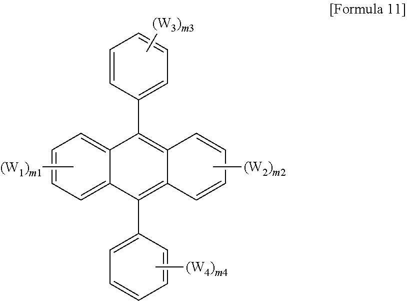

In Formula 11, W₁ to W₄ are each independently a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, or may be combined with an adjacent group to form a ring, m1 and m2 are each independently an integer of 0 to 4, and m3 and m4 are each independently an integer of 0 to 5.

If m1 is 1, W₁ may not be a hydrogen atom, if m2 is 1, W₂ may not be a hydrogen atom, if m3 is 1, W₃ may not be a hydrogen atom, and if m4 is 1, W₄ may not be a hydrogen atom.

If m1 is 2 or more, a plurality of W₁ groups are the same or different. If m2 is 2 or more, a plurality of W₂ groups are the same or different. If m3 is 2 or more, a plurality of W₃ groups are the same or different. If m4 is 2 or more, a plurality of W₄ groups are the same or different.

The compound represented by Formula 11 is an embodiment, and may include compounds represented by the structures below. However, an embodiment of the compound represented by Formula 11 is not limited thereto.

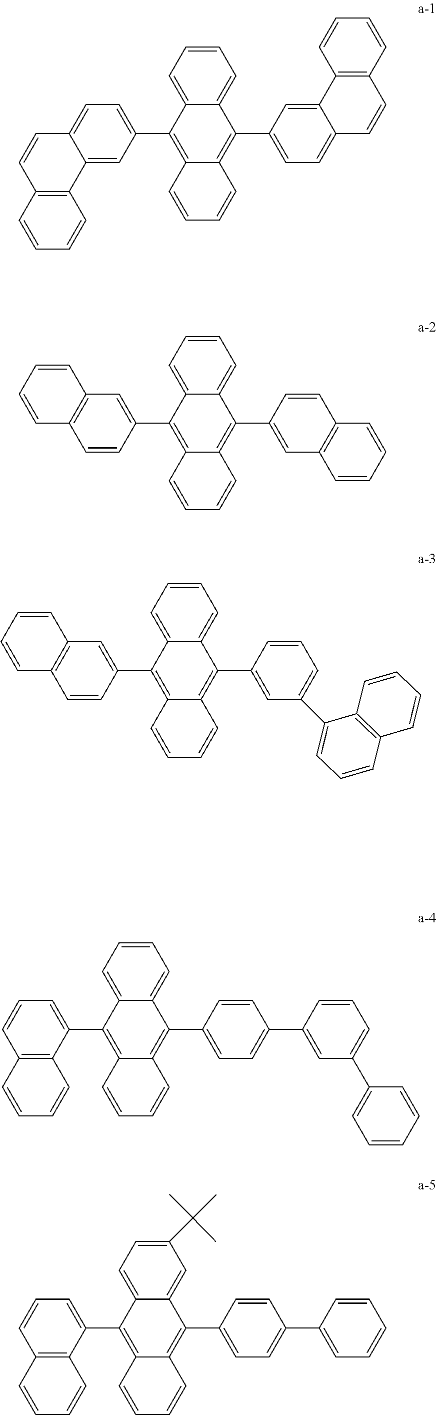

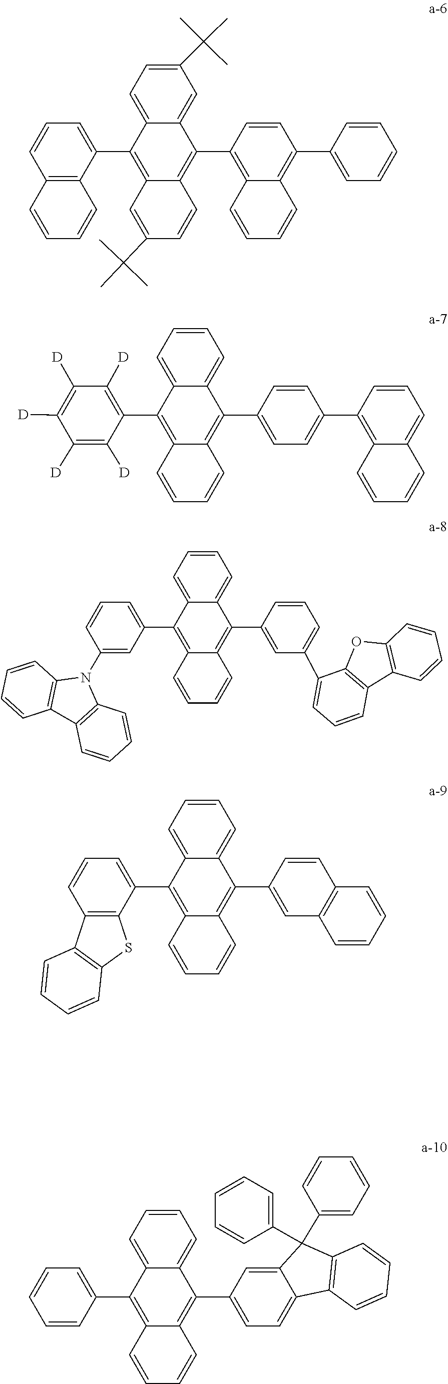

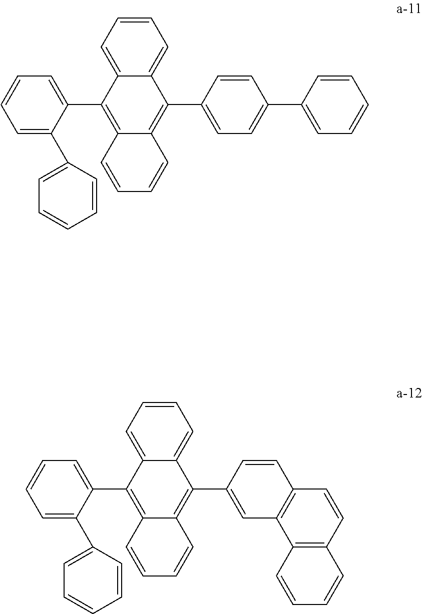

The emission layer EML may include, for example, a fluorescence material including any one selected from the group consisting of spiro-DPVBi, 2,2′,7,7′-tetrakis(biphenyl-4-yl)-9,9′-spirobifluorene(spiro-sexiphenyl) (spiro-6P), distyryl-benzene (DSB), distyryl-arylene (DSA), a polyfluorene (PFO)-based polymer and a poly(p-phenylene vinylene (PPV)-based polymer.

The emission layer EML may further include a dopant and the dopant may use known materials. For example, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E) (diphenylamino)styryl)naphthalen-2-yl)vinyl)phenyl)-N-phenylbenzenamine (N-BDAVBi), perylene and the derivatives thereof (for example, 2,5,8,11-tetra-t-butylperylene (TBPe)), pyrene and the derivatives thereof (for example, 1,1-dipyrene, 1,4-dipyrenylbenzene, 1,4-bis(N,N-diphenylamino)pyrene), 1,6-bis(N,N-diphenylamino)pyrene, 2,5,8,11-tetra-t-butylperylene (TPB), 1,3,5-tris(N-phenylbenzimidazole-2-yl)benzene) (TPBi), etc. may be used as the dopant.

The emission layer EML may include, for example, tris(8-hydroxyquinolino)aluminum (Alq₃), 4,4′-bis(N-carbazolyl)-1,1′-biphenyl (CBP), poly(N-vinylcarbazole) (PVK), 9,10-di(naphthalene-2-yl)anthracene (ADN), 4,4′,4″-tris(carbazol-9-yl)-triphenylamine (TCTA), 1,3,5-tris(N-phenylbenzimidazole-2-yl)benzene (TPBi), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), bis[2-(diphenylphosphino)phenyl]ether oxide (DPEPO), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), 2,8-bis(diphenylphosphoryl)dibenzofuran (PPF), etc.

The electron transport region ETR is provided on the emission layer EML. The electron transport region ETR may include at least one of an electron blocking layer EBL, an electron transport layer ETL or an electron injection layer EIL. However, an embodiment of the inventive concepts is not limited thereto.

The electron transport region ETR may have a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure having a plurality of different materials, or a structure laminated from the first electrode EL1 of electron transport layer ETL/electron injection layer EIL, or hole blocking layer/electron transport layer ETL/electron injection layer EIL, without limitation. The thickness of the electron transport region ETR may be, for example, from about 100 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

If the electron transport region ETR includes an electron transport layer ETL, the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate (Bebq2), 9,10-di(naphthalene-2-yl)anthracene (ADN), or a mixture thereof, without limitation.

If the electron transport region ETR includes the electron transport layer ETL, the thickness of the electron transport layer ETL may be from about 100 Å to about 1,000 Å and may be, for example, from about 150 Å to about 500 Å. If the thickness of the electron transport layer ETL satisfies the above-described range, satisfactory electron transport properties may be obtained without substantial increase of a driving voltage.

If the electron transport region ETR includes the electron injection layer EIL, the electron transport region ETR may include, for example, a metal halide such as LiF, NaCl, CsF, RbCl, RbI, KI, a metal in lanthanoides such as Yb, a metal oxide such as Li₂O, BaO, or lithium quinolate (LiQ). However, an embodiment of the inventive concepts is not limited thereto. The electron injection layer EIL also may be formed using a mixture material of an electron transport material and an insulating organo metal salt. The organo metal salt may be a material having an energy band gap of about 4 eV or more. Particularly, the organo metal salt may include, for example, metal acetates, metal benzoates, metal acetoacetates, metal acetylacetonates, or metal stearates.

If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, satisfactory electron injection properties may be obtained without inducing substantial increase of a driving voltage.

The electron transport region ETR may include a hole blocking layer HBL as described above. The hole blocking layer HBL may include, for example, at least one of 2,9-dimethyl-4,7-diphenyl-1,10-phenanthroline (BCP), or 4,7-diphenyl-1,10-phenanthroline (Bphen). However, an embodiment of the inventive concepts is not limited thereto.

The second electrode EL2 is provided on the electron transport region ETR. The second electrode EL2 has conductivity. The second electrode EL2 may be formed using a metal alloy or a conductive compound. The second electrode EL2 may be a cathode. The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). The second electrode EL2 may have a multilayered structure including a reflective layer or a transflective layer formed using the above-described materials and a transparent conductive layer formed using ITO, IZO, ZnO, ITZO, etc.

Though not shown, the second electrode EL2 may be connected with an auxiliary electrode. If the second electrode EL2 is connected with the auxiliary electrode, the resistance of the second electrode EL2 may decrease.

In the organic electroluminescence device 10, according to the application of a voltage to each of the first electrode EL1 and second electrode EL2, holes injected from the first electrode EL1 may move via the hole transport region HTR to the emission layer EML, and electrons injected from the second electrode EL2 may move via the electron transport region ETR to the emission layer EML. The electrons and the holes are recombined in the emission layer EML to produce excitons, and the excitons may emit light via transition from an excited state to a ground state.

If the organic electroluminescence device 10 is a top emission type, the first electrode EL1 may be a reflective electrode and the second electrode EL2 may be a transmissive electrode or a transflective electrode. If the organic electroluminescence device 10 is a bottom emission type, the first electrode EL1 may be a transmissive electrode or a transflective electrode and the second electrode EL2 may be a reflective electrode.

The organic electroluminescence device 10 according to an embodiment of the inventive concepts is characterized in including the polycyclic compound represented by Formula 1, and thus may achieve high efficiency and the increase of life. In addition, effects of decreasing a driving voltage may be achieved.

Hereinafter, the inventive concepts will be more particularly explained referring to particular embodiments and comparative embodiments. The following embodiments are only illustrations to assist the understanding of the inventive concepts, and the scope of the inventive concepts is not limited thereto.

### Synthetic Examples

The polycyclic compound according to an embodiment of the inventive concepts may be synthesized by, for example, the following. However, the synthetic method of the polycyclic compound according to an embodiment of the inventive concepts is not limited thereto.

1. Synthesis of Compound A2

(Synthesis of Intermediate IM-1)

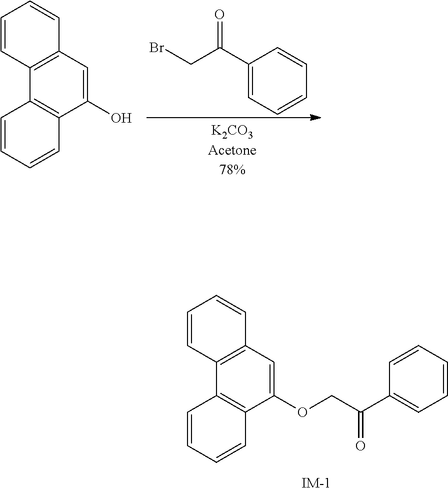

Under an Ar atmosphere, 20.00 g (103.0 mmol) of 9-phenanthrol, 42.69 g (3.0 eq, 308.9 mmol) of K₂CO₃, 30.74 g (1.5 eq, 154.5 mmol) of phenacyl bromide, and 343 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-1 (25.09 g, yield 78%).

FAB-MS was measured, mass number m/z=312 was observed as a molecular ion peak, and Intermediate IM-1 was identified.

(Synthesis of Intermediate IM-2)

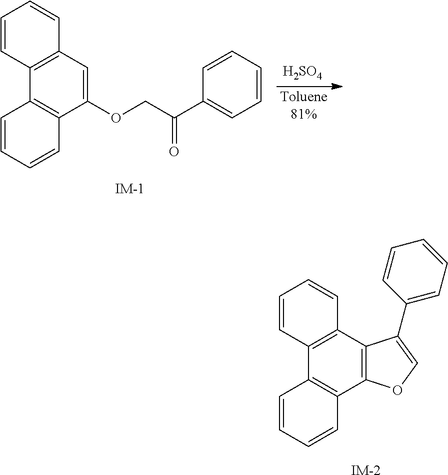

Under an Ar atmosphere, 20.00 g (64.0 mmol) of IM-1, 213 m1 (0.3 M) of toluene and 0.68 m1 (0.2 eq, 12.8 mmol) of H₂SO₄ were added one by one to a 500 m1, three-neck flask, followed by heating and refluxing while stirring while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-2 (15.27 g, yield 81%). FAB-MS was measured, mass number m/z=294 was observed as a molecular ion peak, and Intermediate IM-2 was identified.

(Synthesis of Intermediate IM-3)

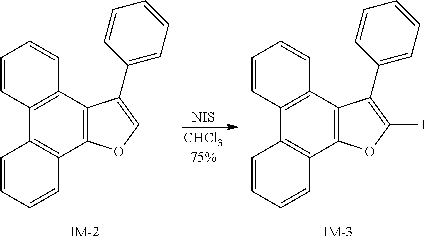

Under an Ar atmosphere, 12.0 g (40.8 mmol) of IM-2, 10.09 g (1.1 eq, 44.8 mmol) of NIS, and 204 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-3 (12.85 g, yield 75%). FAB-MS was measured, mass number m/z=420 was observed as a molecular ion peak, and Intermediate IM-3 was identified.

(Synthesis of Intermediate IM-4)

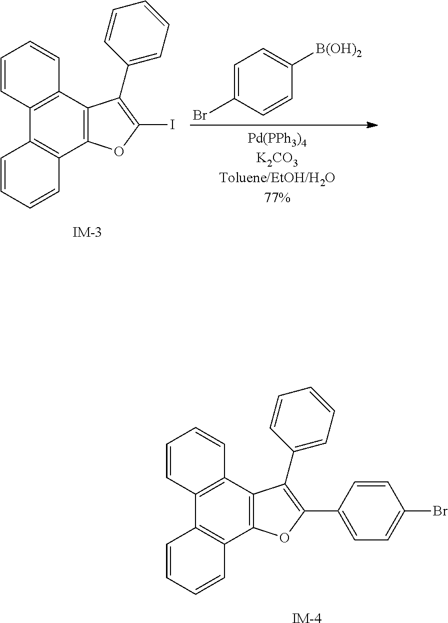

Under an Ar atmosphere, 10.00 g (23.8 mmol) of IM-3, 5.26 g (1.1 eq, 26.2 mmol) of 4-bromophenylboronic acid, 9.87 g (3.0 eq, 71.4 mmol) of K₂CO₃, 1.37 g (0.05 eq, 1.2 mmol) of Pd(PPh₃)₄, and 167 m1 of a mixture solution of toluene/EtOH/H₂O were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-4 (8.23 g, yield 77%). FAB-MS was measured, mass number m/z=449 was observed as a molecular ion peak, and Intermediate IM-4 was identified.

(Synthesis of Compound A2)

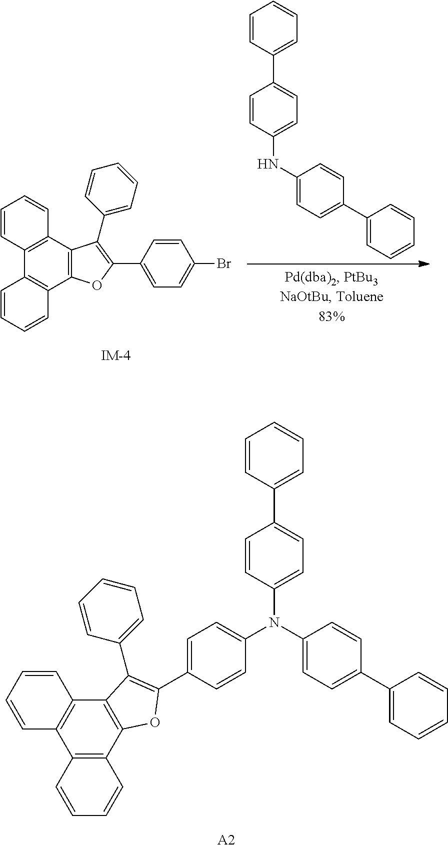

Under an Ar atmosphere, 5.00 g (11.1 mmol) of IM-4, 0.19 g (0.03 eq, 0.3 mmol) of Pd(dba)₂, 2.14 g (2.0 eq, 22.3 mmol) of NaOtBu, 56 m1 of toluene, 3.93 g (1.1 eq, 12.2 mmol) of bis(4-biphenyl)amine and 0.23 g (0.1 eq, 1.1 mmol) of PtBu₃ were added one by one to a 200 ml, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound A2 as a solid (6.37 g, yield 83%). FAB-MS was measured, mass number m/z=689 was observed as a molecular ion peak, and Compound A2 was identified.

2. Synthesis of Compound A48

(Synthesis of Intermediate IM-5)

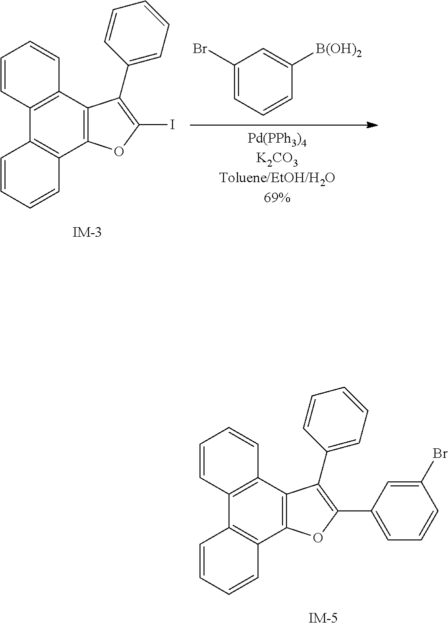

Under an Ar atmosphere, 10.00 g (23.8 mmol) of IM-3, 5.26 g (1.1 eq, 26.2 mmol) of 3-bromophenylboronic acid, 9.87 g (3.0 eq, 71.4 mmol) of K₂CO₃, 1.37 g (0.05 eq, 1.2 mmol) of Pd(PPh₃)₄, and 167 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-5 (7.38 g, yield 69%). FAB-MS was measured, mass number m/z=449 was observed as a molecular ion peak, and Intermediate IM-5 was identified.

(Synthesis of Compound A48)

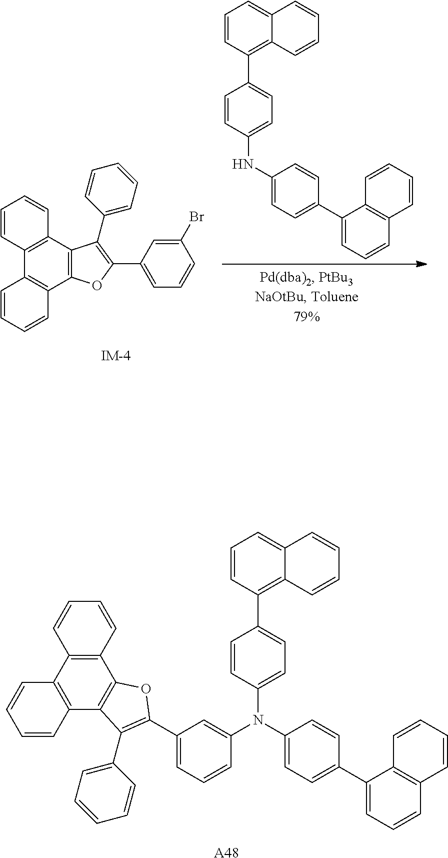

Under an Ar atmosphere, 5.00 g (11.1 mmol) of IM-5, 0.19 g (0.03 eq, 0.3 mmol) of Pd(dba)₂, 2.14 g (2.0 eq, 22.3 mmol) of NaOtBu, 56 m1 of toluene, 5.16 g (1.1 eq, 12.2 mmol) of bis[4-naphthalen-1-yl)phenyl]amine and 0.23 g (0.1 eq, 1.1 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound A48 as a solid (6.94 g, yield 79%). FAB-MS was measured, mass number m/z=789 was observed as a molecular ion peak, and Compound A48 was identified.

3. Synthesis of Compound A57

(Synthesis of Intermediate IM-6)

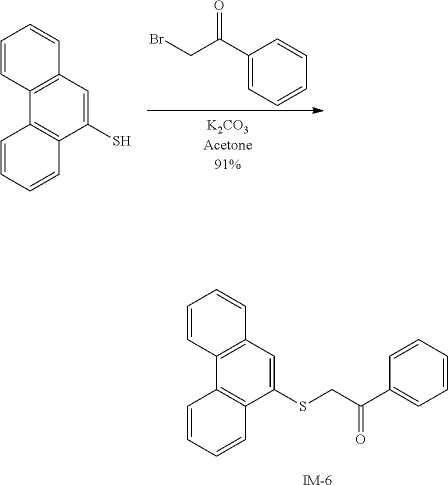

Under an Ar atmosphere, 20.00 g (95.11 mmol) of 9-phenanthrothiol, 39.43 g (3.0 eq, 285.3 mmol) of K₂CO₃, 28.40 g (1.5 eq, 142.7 mmol) of phenacyl bromide and 317 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-6 (28.42 g, yield 91%). FAB-MS was measured, mass number m/z=328 was observed as a molecular ion peak, and Intermediate IM-6 was identified.

(Synthesis of Intermediate IM-7)

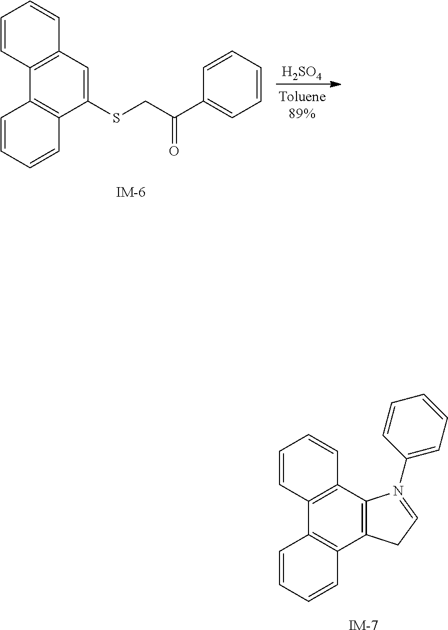

Under an Ar atmosphere, 20.00 g (60.9 mmol) of IM-6, 203 m1 (0.3 M) of toluene and 0.65 m1 (0.2 eq, 12.2 mmol) of H₂SO₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-7 (16.82 g, yield 89%). FAB-MS was measured, mass number m/z=310 was observed as a molecular ion peak, and Intermediate IM-7 was identified.

(Synthesis of Intermediate IM-8)

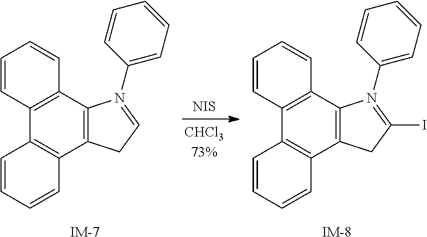

Under an Ar atmosphere, 12.0 g (38.7 mmol) of IM-7, 9.57 g (1.1 eq, 42.5 mmol) of NIS, and 194 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-8 (12.31 g, yield 73%). FAB-MS was measured, mass number m/z=436 was observed as a molecular ion peak, and Intermediate IM-8 was identified.

(Synthesis of Intermediate IM-9)

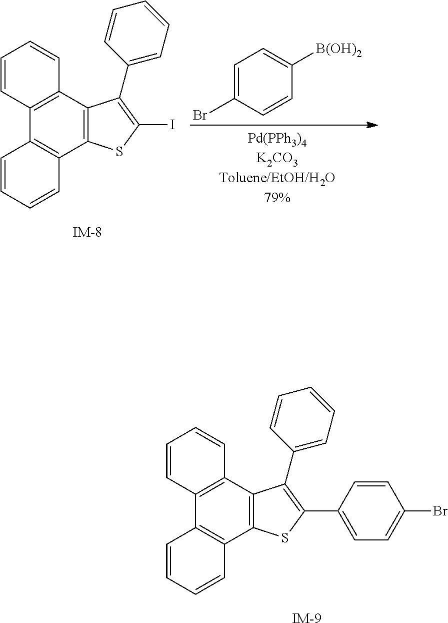

Under an Ar atmosphere, 10.00 g (22.9 mmol) of IM-8, 5.06 g (1.1 eq, 25.2 mmol) of 4-bromophenylboronic acid, 9.50 g (3.0 eq, 71.4 mmol) of K₂CO₃, 1.15 g (0.05 eq, 1.1 mmol) of Pd(PPh₃)₄, and 160 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-9 (8.42 g, yield 79%). FAB-MS was measured, mass number m/z=465 was observed as a molecular ion peak, and Intermediate IM-9 was identified.

(Synthesis of Compound A57)

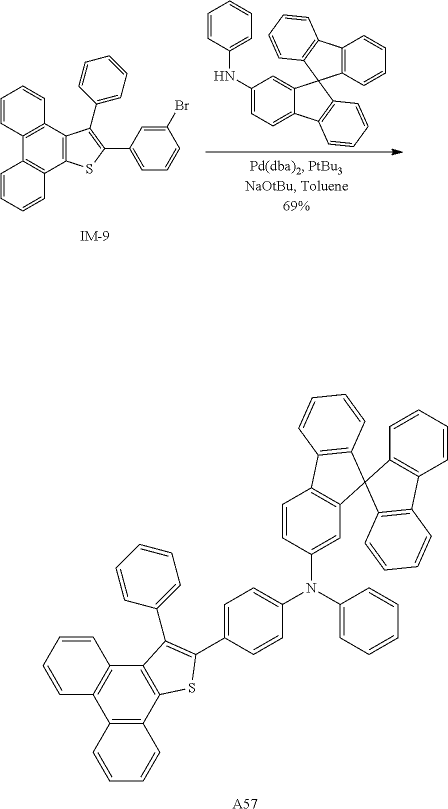

Under an Ar atmosphere, 5.00 g (10.7 mmol) of IM-9, 0.19 g (0.03 eq, 0.3 mmol) of Pd(dba)₂, 2.06 g (2.0 eq, 21.5 mmol) of NaOtBu, 54 m1 of toluene, 4.82 g (1.1 eq, 11.8 mmol) of N-phenyl-9,9′-spirobi[fluoren]-2-amine and 0.22 g (0.1 eq, 1.1 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound A57 as a solid (5.87 g, yield 69%). FAB-MS was measured, mass number m/z=792 was observed as a molecular ion peak, and Compound A57 was identified.

4. Synthesis of Compound B22

(Synthesis of Intermediate IM-10)

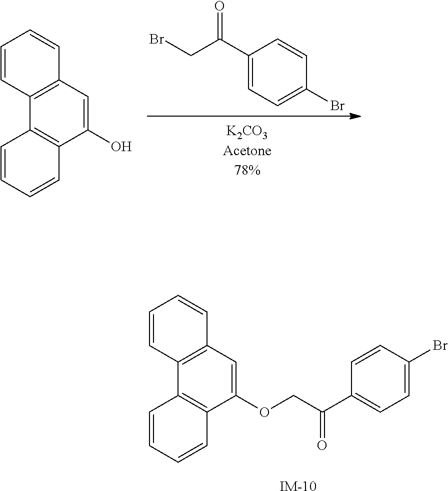

Under an Ar atmosphere, 20.00 g (95.11 mmol) of 9-phenanthrol, 42.70 g (3.0 eq, 308.9 mmol) of K₂CO₃, 42.93 g (1.5 eq, 154.5 mmol) of 4-bromophenacyl bromide and 343 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-10 (31.42 g, yield 78%). FAB-MS was measured, mass number m/z=391 was observed as a molecular ion peak, and

Intermediate IM-10 was identified.

(Synthesis of Intermediate IM-11)

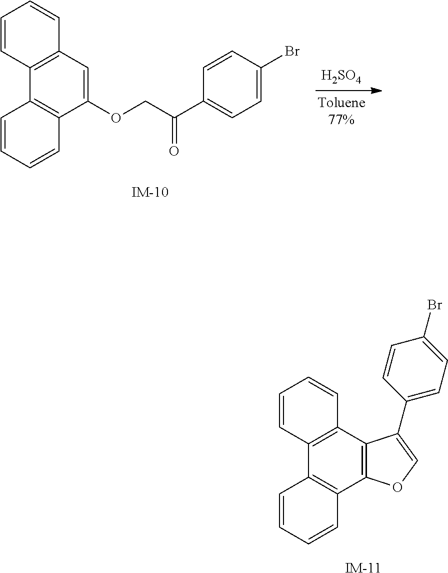

Under an Ar atmosphere, 20.00 g (51.1 mmol) of IM-10, 170 m1 (0.3 M) of toluene and 0.54 m1 (0.2 eq, 10.2 mmol) of H₂SO₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-11 (14.69 g, yield 77%). FAB-MS was measured, mass number m/z=373 was observed as a molecular ion peak, and Intermediate IM-11 was identified.

(Synthesis of Intermediate IM-12)

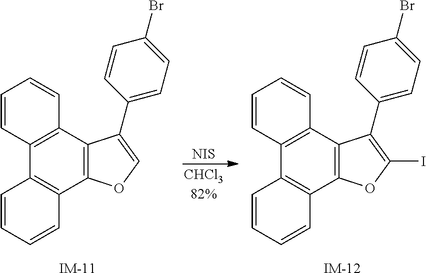

Under an Ar atmosphere, 12.0 g (32.2 mmol) of IM-11, 7.96 g (1.1 eq, 35.4 mmol) of NIS, and 160 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-12 (13.16 g, yield 82%). FAB-MS was measured, mass number m/z=499 was observed as a molecular ion peak, and

Intermediate IM-12 was identified.

(Synthesis of Intermediate IM-13)

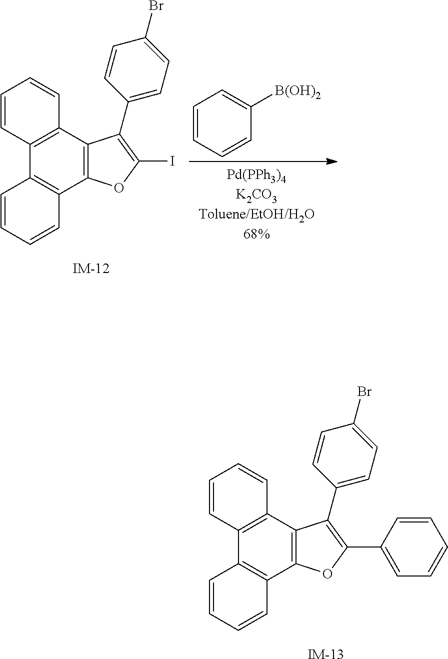

Under an Ar atmosphere, 10.00 g (20.0 mmol) of IM-12, 2.69 g (1.1 eq, 22.0 mmol) of phenylboronic acid, 8.31 g (3.0 eq, 60.1 mmol) of K₂CO₃, 1.16 g (0.05 eq, 1.0 mmol) of Pd(PPh₃)₄, and 140 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-13 (6.12 g, yield 68%). FAB-MS was measured, mass number m/z=449 was observed as a molecular ion peak, and Intermediate IM-13 was identified.

(Synthesis of Compound B22)

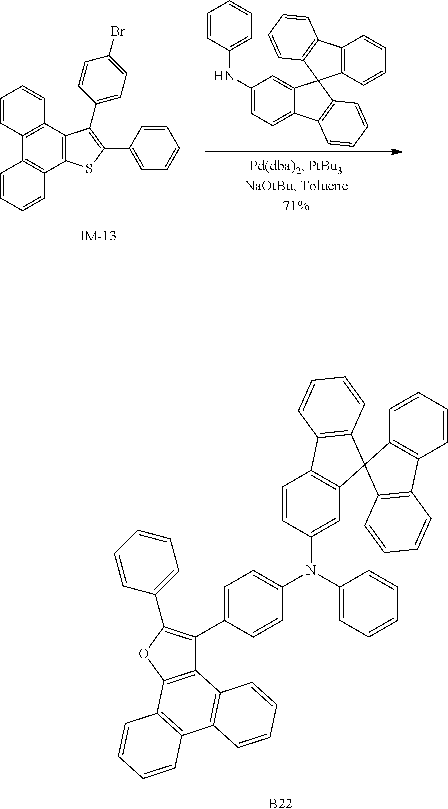

Under an Ar atmosphere, 5.00 g (11.1 mmol) of IM-13, 0.19 g (0.03 eq, 0.3 mmol) of Pd(dba)₂, 2.14 g (2.0 eq, 22.3 mmol) of NaOtBu, 54 m1 of toluene, 5.01 g (1.1 eq, 12.2 mmol) of N,9,9-triphenyl-9H-fluoren-2-amine and 0.23 g (0.1 eq, 1.1 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound B22 as a solid (6.15 g, yield 71%). FAB-MS was measured, mass number m/z=777 was observed as a molecular ion peak, and Compound B22 was identified.

5. Synthesis of Compound B42

(Synthesis of Intermediate IM-14)

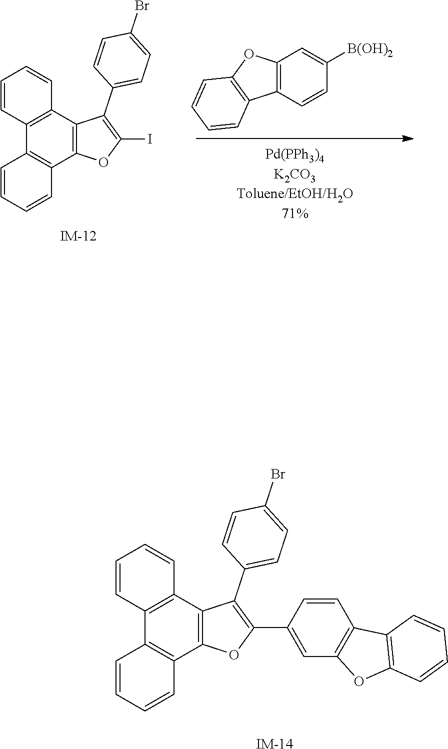

Under an Ar atmosphere, 10.00 g (20.0 mmol) of IM-12, 4.67 g (1.1 eq, 22.0 mmol) of dibenzo[b,d]furan-3-ylboronic acid, 8.31 g (3.0 eq, 60.1 mmol) of K₂CO₃, 1.16 g (0.05 eq, 1.0 mmol) of Pd(PPh₃)₄, and 140 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-14 (7.67 g, yield 71%). FAB-MS was measured, mass number m/z=539 was observed as a molecular ion peak, and Intermediate IM-14 was identified.

(Synthesis of Compound B42)

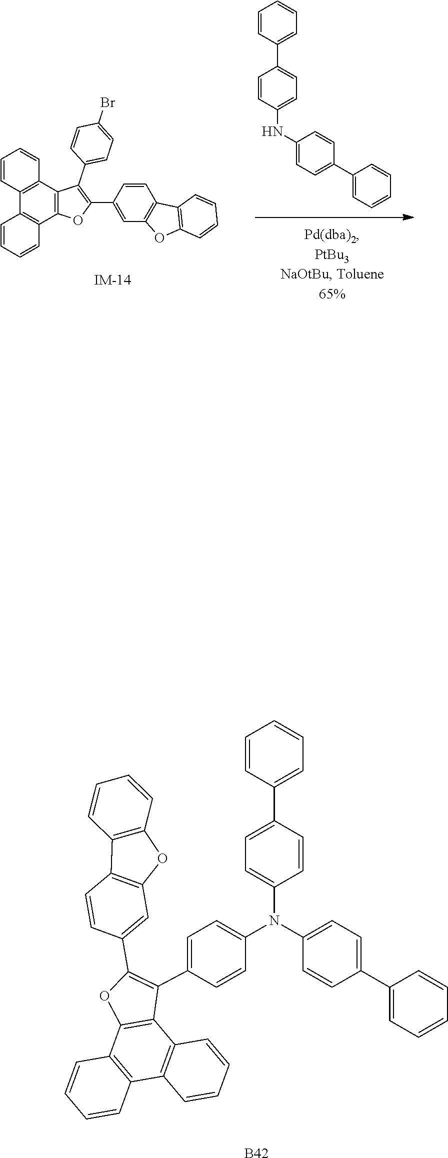

Under an Ar atmosphere, 5.00 g (9.3 mmol) of IM-14, 0.16 g (0.03 eq, 0.3 mmol) of Pd(dba)₂, 1.78 g (2.0 eq, 18.5 mmol) of NaOtBu, 46 m1 of toluene, 3.28 g (1.1 eq, 10.2 mmol) of bis(4-biphenyl)amine and 0.19 g (0.1 eq, 0.9 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound B42 as a solid (4.70 g, yield 69%). FAB-MS was measured, mass number m/z=779 was observed as a molecular ion peak, and Compound B42 was identified.

6. Synthesis of Compound B44

(Synthesis of Intermediate IM-15)

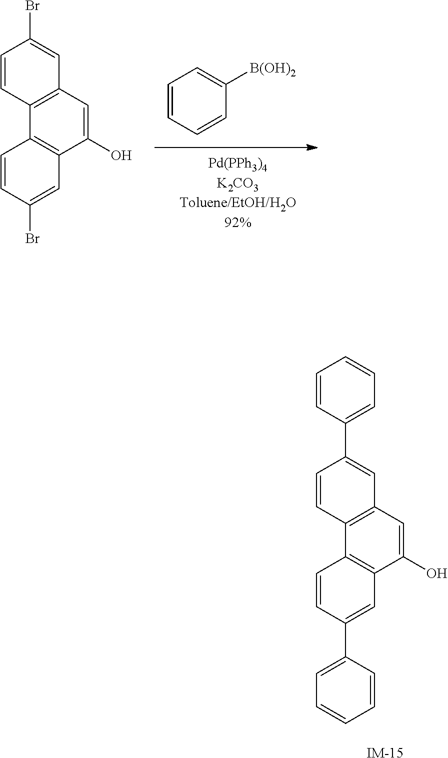

Under an Ar atmosphere, 25.00 g (71.0 mmol) of 2,7-dibromophenanthren-9-ol, 19.05 g (2.2 eq, 156.2 mmol) of phenylboronic acid, 58.89 g (6.0 eq, 426.1 mmol) of K₂CO₃, 8.21 g (0.1 eq, 7.1 mmol) of Pd(PPh₃)₄, and 497 m1 of a mixture solution of toluene/EtOH/H₂O were added one by one to a 1000 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-15 (28.51 g, yield 92%). FAB-MS was measured, mass number m/z=436 was observed as a molecular ion peak, and Intermediate IM-15 was identified.

(Synthesis of Intermediate IM-16)

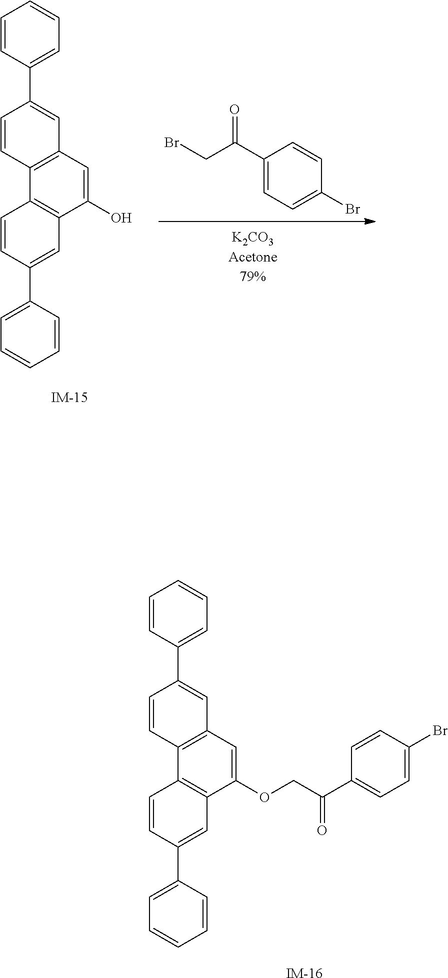

Under an Ar atmosphere, 25.00 g (57.3 mmol) of IM-15, 29.92 g (3.0 eq, 216.49 mmol) of K₂CO₃, 30.09 g (1.5 eq, 108.2 mmol) of 4-bromophenacyl bromide and 240 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-16 (30.98 g, yield 79%). FAB-MS was measured, mass number m/z=543 was observed as a molecular ion peak, and Intermediate IM-16 was identified.

(Synthesis of Intermediate IM-17)

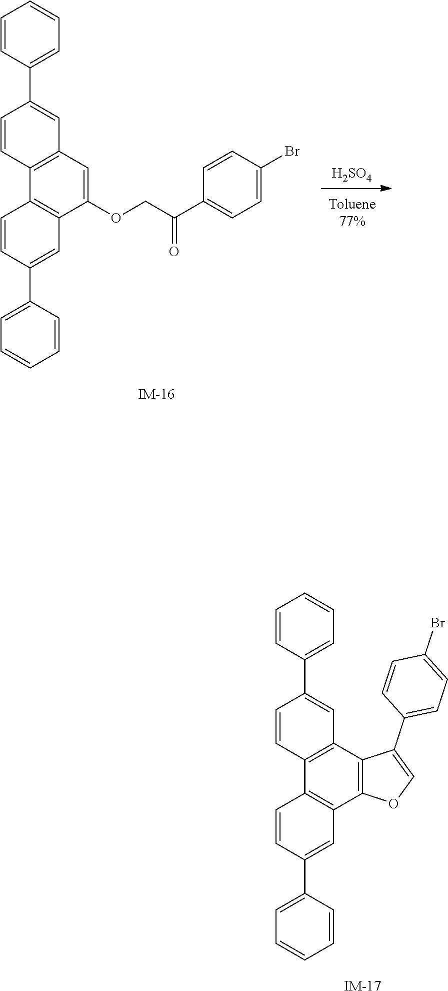

Under an Ar atmosphere, 20.00 g (36.8 mmol) of IM-16, 123 m1 (0.3 M) of toluene and 0.39 m1 (0.2 eq, 7.4 mmol) of H₂SO₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-17 (14.89 g, yield 77%). FAB-MS was measured, mass number m/z=525 was observed as a molecular ion peak, and Intermediate IM-17 was identified.

(Synthesis of Intermediate IM-18)

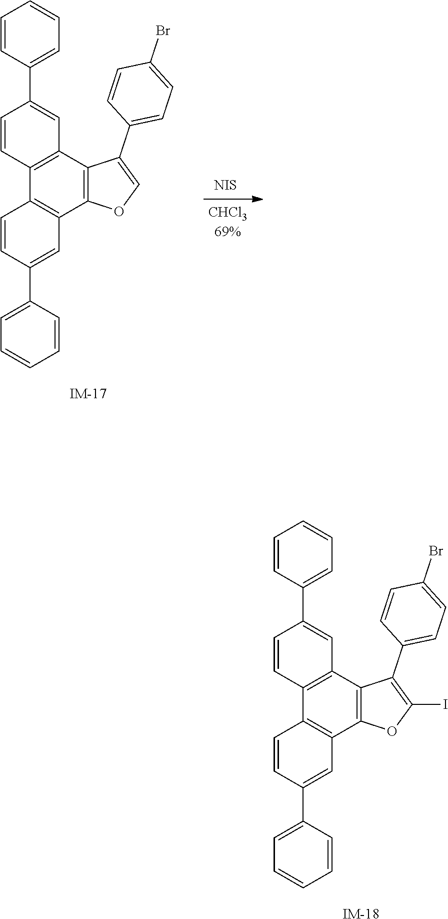

Under an Ar atmosphere, 12.0 g (22.8 mmol) of IM-17, 5.65 g (1.1 eq, 25.1 mmol) of NIS, and 114 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-18 (10.26 g, yield 69%). FAB-MS was measured, mass number m/z=651 was observed as a molecular ion peak, and Intermediate IM-18 was identified.

(Synthesis of Intermediate IM-19)

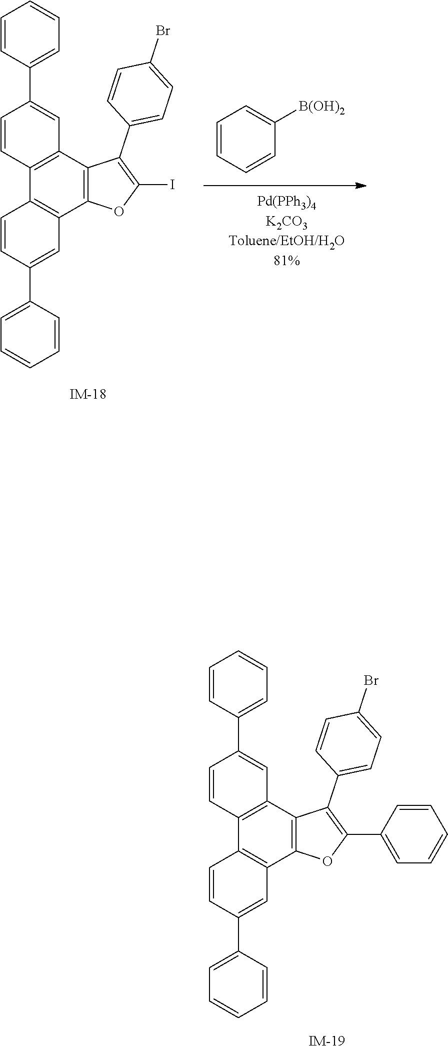

Under an Ar atmosphere, 10.00 g (15.3 mmol) of IM-18, 2.06 g (1.1 eq, 16.9 mmol) of phenylboronic acid, 6.36 g (3.0 eq, 46.1 mmol) of K₂CO₃, 0.89 g (0.05 eq, 0.77 mmol) of Pd(PPh₃)₄, and 180 m1 of a mixture solution of toluene/EtOH/H₂O (4/2/1) were added one by one to a 300 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-19 (7.48 g, yield 81%). FAB-MS was measured, mass number m/z=601 was observed as a molecular ion peak, and

Intermediate IM-19 was identified.

(Synthesis of Compound B44)

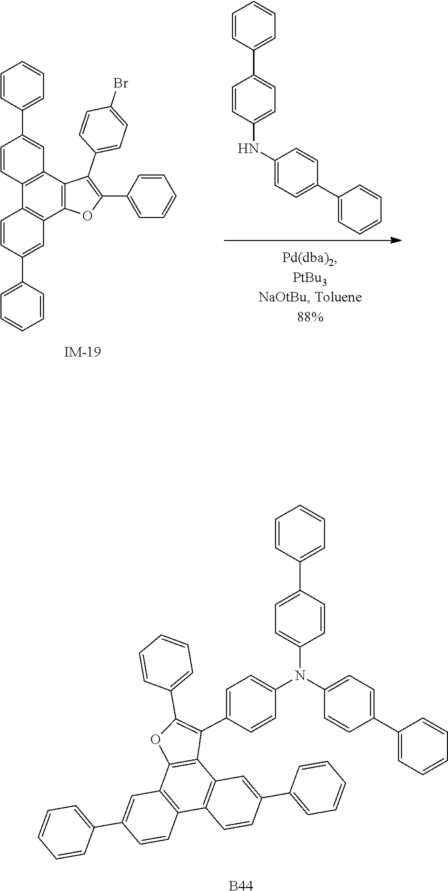

Under an Ar atmosphere, 5.00 g (8.3 mmol) of IM-19, 0.14 g (0.03 eq, 0.2 mmol) of Pd(dba)₂, 1.60 g (2.0 eq, 16.6 mmol) of NaOtBu, 42 m1 of toluene, 2.94 g (1.1 eq, 9.1 mmol) of bis(4-biphenyl)amine and 0.17 g (0.1 eq, 0.8 mmol) of PtBu₃ were added one by one to a 200 ml, three-neck flask, followed by heating and refluxing while stirring at about 120° C. After cooling to room temperature, water was added to the reaction solution and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound B44 as a solid (6.6 g, yield 88%). FAB-MS was measured, mass number m/z=842 was observed as a molecular ion peak, and Compound B42 was identified.

7. Synthesis of Compound C11

(Synthesis of Intermediate IM-20)

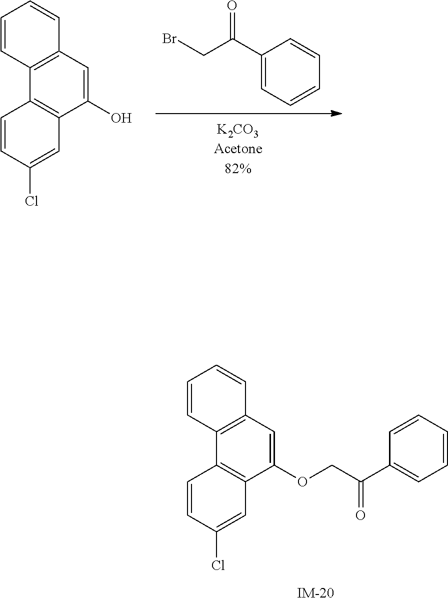

Under an Ar atmosphere, 20.00 g (87.5 mmol) of 7-chlorophenanthren-9-ol, 36.26 g (3.0 eq, 262.4 mmol) of K₂CO₃, 26.11 g (1.5 eq, 131.2 mmol) of phenacyl bromide and 292 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-20 (24.87 g, yield 82%). FAB-MS was measured, mass number m/z=346 was observed as a molecular ion peak, and Intermediate IM-20 was identified.

(Synthesis of Intermediate IM-21)

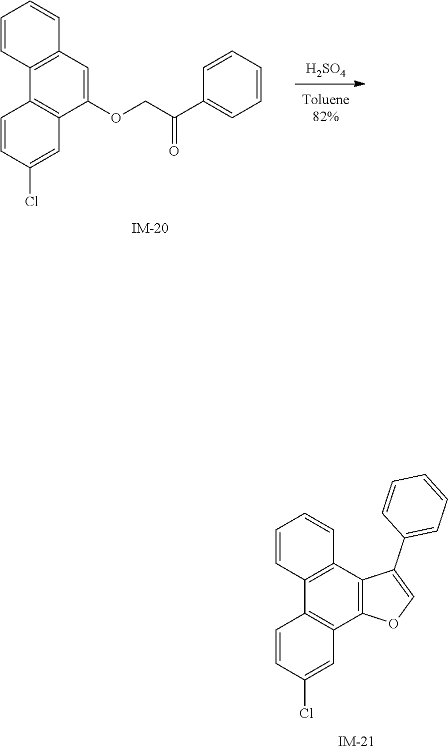

Under an Ar atmosphere, 20.00 g (57.7 mmol) of IM-20, 192 m1 (0.3 M) of toluene and 0.61 m1 (0.2 eq, 11.5 mmol) of H₂SO₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-21 (15.55 g, yield 82%). FAB-MS was measured, mass number m/z=328 was observed as a molecular ion peak, and Intermediate IM-21 was identified.

(Synthesis of Intermediate IM-22)

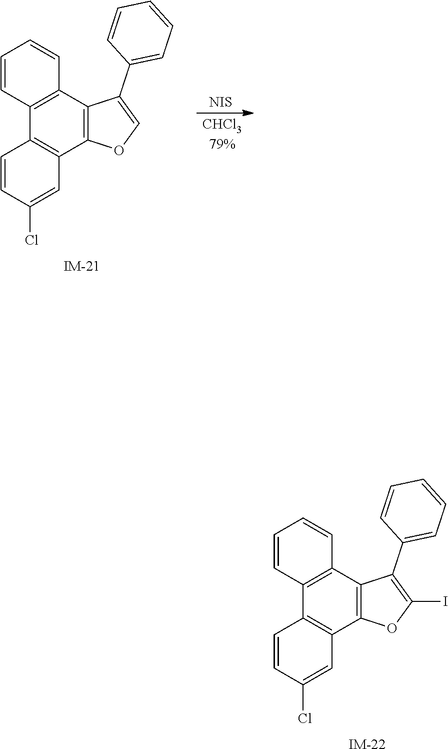

Under an Ar atmosphere, 12.0 g (36.5 mmol) of IM-21, 9.03 g (1.1 eq, 40.1 mmol) of NIS, and 182 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-22 (13.11 g, yield 79%). FAB-MS was measured, mass number m/z=454 was observed as a molecular ion peak, and Intermediate IM-22 was identified.

(Synthesis of Intermediate IM-23)

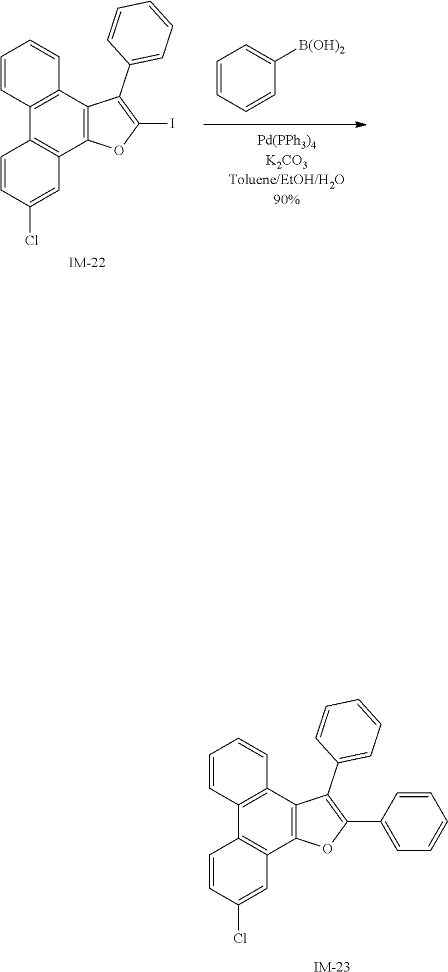

Under an Ar atmosphere, 10.00 g (22.0 mmol) of IM-22, 2.95 g (1.1 eq, 24.2 mmol) of phenylboronic acid, 9.12 g (3.0 eq, 66.0 mmol) of K₂CO₃, 1.27 g (0.05 eq, 1.1 mmol) of Pd(PPh₃)₄, and 154 m1 of a mixture solution of toluene/EtOH/H₂O (4/2/1) were added one by one to a 300 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-23 (8.01 g, yield 90%).

FAB-MS was measured, mass number m/z=404 was observed as a molecular ion peak, and Intermediate IM-23 was identified.

(Synthesis of Compound C11)

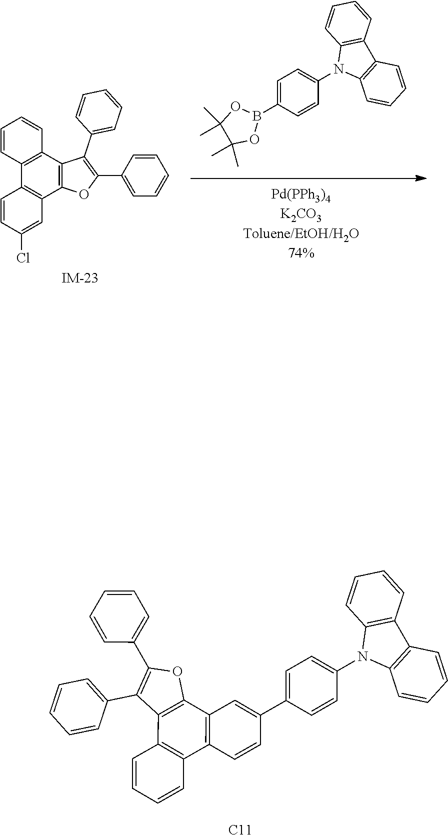

Under an Ar atmosphere, 5.00 g (12.3 mmol) of IM-23, 5.02 g (1.1 eq, 13.6 mmol) of 9-[4-(4,4,5,5-tetramethyl-1,3,2-dioxaborolan-2-yl)phenyl]-9H-carbazole, 5.12 g (3.0 eq, 37.0 mmol) of K₂CO₃, 0.71 g (0.05 eq, 0.62 mmol) of Pd(PPh₃)₄, and 86 m1 of a mixture solution of toluene/EtOH/H₂O (4/2/1) were added one by one to a 300 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound C11 as a solid (6.87 g, yield 88%).

FAB-MS was measured, mass number m/z=611 was observed as a molecular ion peak, and Compound C11 was identified.

8. Synthesis of Compound D32

(Synthesis of Intermediate IM-24)

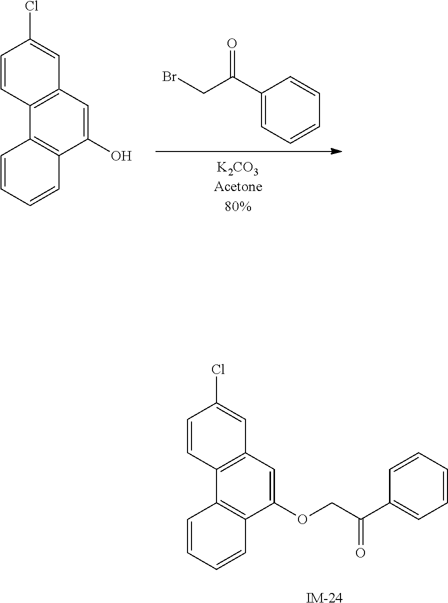

Under an Ar atmosphere, 20.00 g (87.5 mmol) of 2-chlorophenanthren-9-ol, 36.26 g (3.0 eq, 262.4 mmol) of K₂CO₃, 26.11 g (1.5 eq, 131.2 mmol) of phenacyl bromide and 292 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-24 (24.27 g, yield 80%). FAB-MS was measured, mass number m/z=346 was observed as a molecular ion peak, and Intermediate IM-24 was identified.

(Synthesis of Intermediate IM-25)

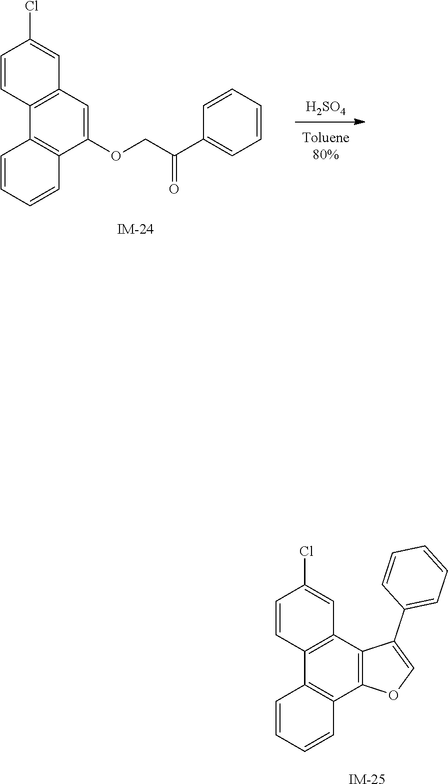

Under an Ar atmosphere, 20.00 g (57.7 mmol) of IM-24, 192 m1 (0.3 M) of toluene and 0.61 m1 (0.2 eq, 11.5 mmol) of H₂SO₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-25 (15.17 g, yield 80%). FAB-MS was measured, mass number m/z=328 was observed as a molecular ion peak, and Intermediate IM-25 was identified.

(Synthesis of Intermediate IM-26)

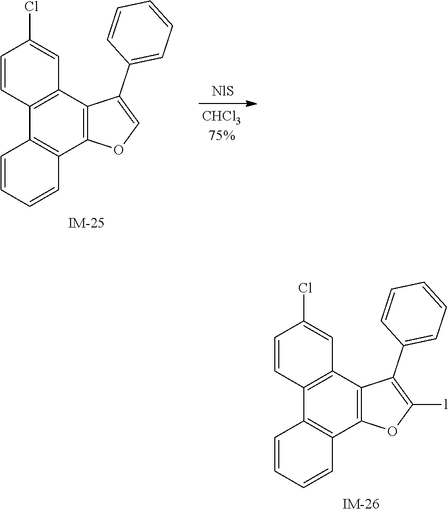

Under an Ar atmosphere, 12.0 g (36.5 mmol) of IM-25, 9.03 g (1.1 eq, 40.1 mmol) of NIS, and 182 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-26 (12.45 g, yield 75%).

FAB-MS was measured, mass number m/z=454 was observed as a molecular ion peak, and Intermediate IM-26 was identified.

(Synthesis of Intermediate IM-27)

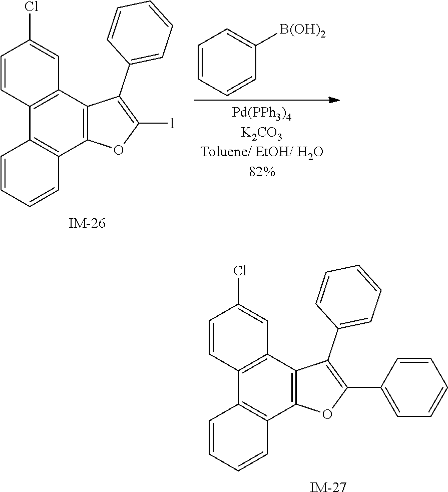

Under an Ar atmosphere, 10.00 g (22.0 mmol) of IM-26, 2.95 g (1.1 eq, 24.2 mmol) of phenylboronic acid, 9.12 g (3.0 eq, 66.0 mmol) of K₂CO₃, 1.27 g (0.05 eq, 1.1 mmol) of Pd(PPh₃)₄, and 154 m1 of a mixture solution of toluene/EtOH/H₂O (4/2/1) were added one by one to a 300 m1, three-neck flask, followed by heating and refluxing while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-27 (7.30 g, yield 82%). FAB-MS was measured, mass number m/z=404 was observed as a molecular ion peak, and

Intermediate IM-27 was identified.

(Synthesis of Compound D32)

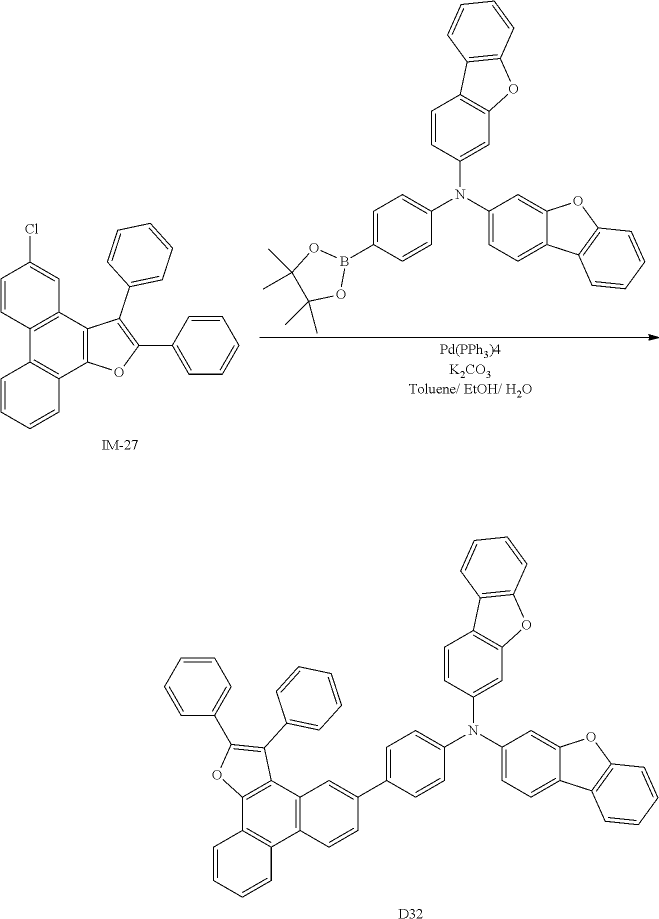

Under an Ar atmosphere, 5.00 g (12.3 mmol) of IM-27, 7.49 g (1.1 eq, 13.6 mmol) of N-(dibenzo[b,d]furan-3-yl)-N-[4-(4,4,5,5-tetramethyl-1,3,2-dioxaborolan-2-yl)phenyl]dibenzo[b,d]furan-3-amine, 5.12 g (3.0 eq, 37.0 mmol) of K₂CO₃, 0.71 g (0.05 eq, 0.62 mmol) of Pd(PPh₃)₄, and 86 m1 of a mixture solution of toluene/EtOH/H₂O (4/2/1) were added one by one to a 300 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound D32 as a solid (6.96 g, yield 71%). FAB-MS was measured, mass number m/z=793 was observed as a molecular ion peak, and Compound D32 was identified.

9. Synthesis of Compound E54

(Synthesis of Intermediate IM-28)

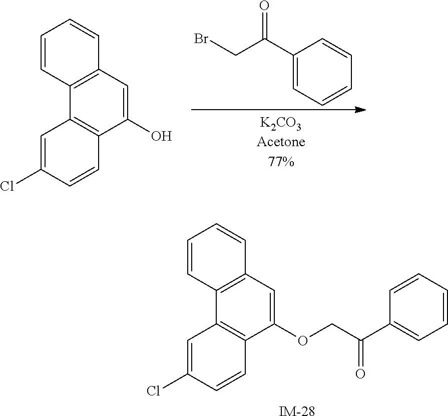

Under an Ar atmosphere, 20.00 g (87.5 mmol) of 6-chlorophenanthren-9-ol, 36.26 g (3.0 eq, 262.4 mmol) of K₂CO_{3, 26.11} g (1.5 eq, 131.2 mmol) of phenacyl bromide and 292 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-28 (23.26 g, yield 77%). FAB-MS was measured, mass number m/z=346 was observed as a molecular ion peak, and Intermediate IM-28 was identified.

(Synthesis of Intermediate IM-29)

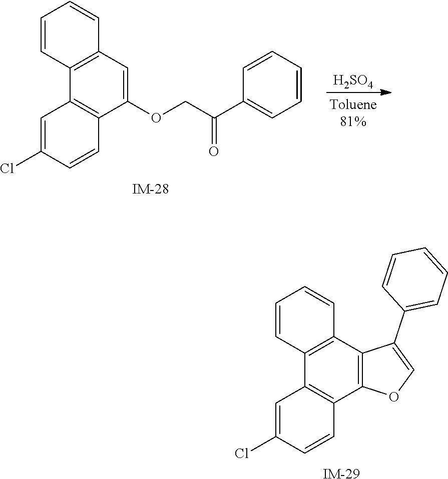

Under an Ar atmosphere, 20.00 g (57.7 mmol) of IM-28, 192 m1 (0.3 M) of toluene and 0.61 m1 (0.2 eq, 11.5 mmol) of H₂₅₀₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-29 (15.36 g, yield 81%). FAB-MS was measured, mass number m/z=328 was observed as a molecular ion peak, and Intermediate IM-29 was identified.

(Synthesis of Intermediate IM-30)

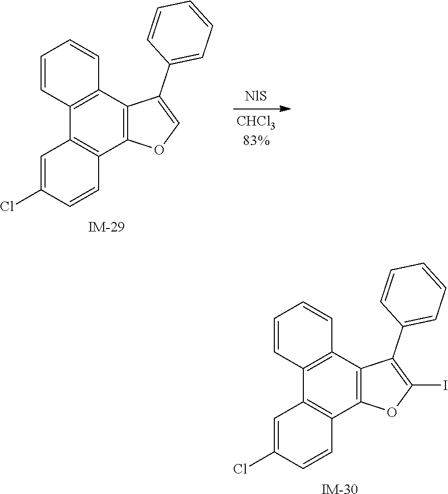

Under an Ar atmosphere, 12.0 g (36.5 mmol) of IM-29, 9.03 g (1.1 eq, 40.1 mmol) of NIS, and 182 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-30 (13.77 g, yield 83%). FAB-MS was measured, mass number m/z=454 was observed as a molecular ion peak, and Intermediate IM-30 was identified.

(Synthesis of Intermediate IM-31)

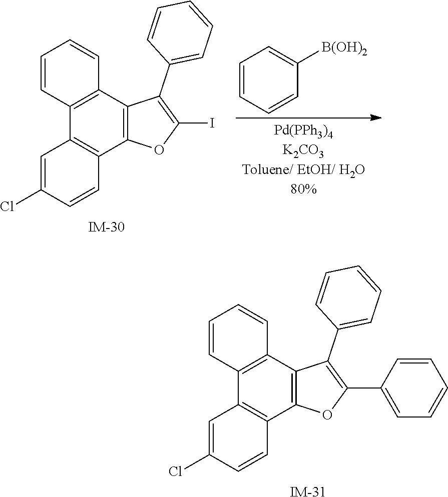

Under an Ar atmosphere, 10.00 g (22.0 mmol) of IM-30, 2.95 g (1.1 eq, 24.2 mmol) of phenylboronic acid, 9.12 g (3.0 eq, 66.0 mmol) of K₂CO₃, 1.27 g (0.05 eq, 1.1 mmol) of Pd(PPh₃)₄, and 154 m1 of a mixture solution of toluene/EtOH/H₂O (4/2/1) were added one by one to a 300 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-31 (7.12 g, yield 80%).

FAB-MS was measured, mass number m/z=404 was observed as a molecular ion peak, and Intermediate IM-31 was identified.

(Synthesis of Compound E54)

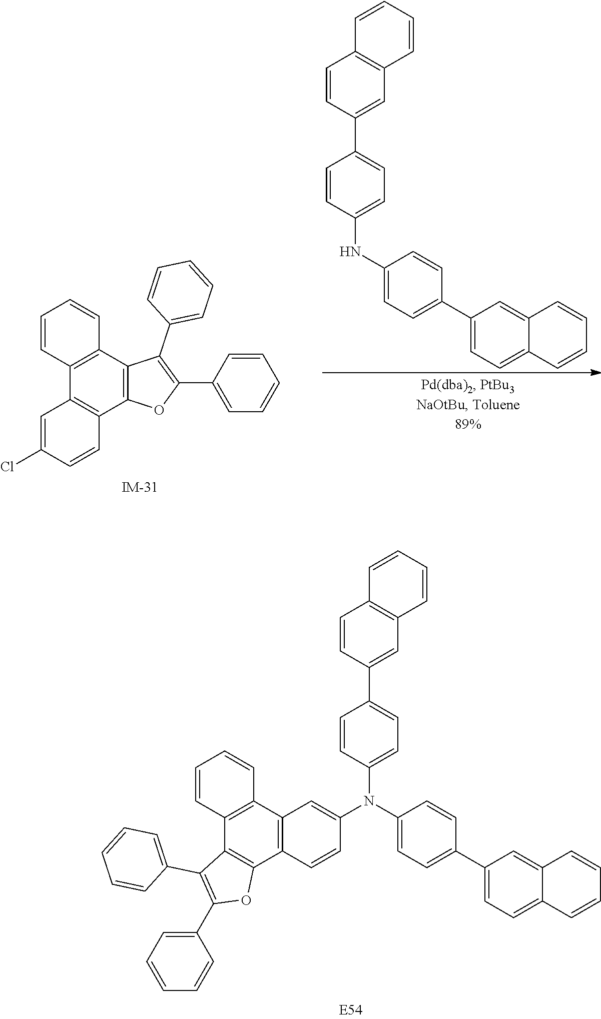

Under an Ar atmosphere, 5.00 g (12.3 mmol) of IM-31, 0.21 g (0.03 eq, 0.4 mmol) of Pd(dba)₂, 2.37 g (2.0 eq, 24.7 mmol) of NaOtBu, 62 m1 of toluene, 5.73 g (1.1 eq, 13.6 mmol) of bis[4-(naphthalen-2-yl)phenyl]amine and 0.25 g (0.1 eq, 1.2 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound E54 as a solid (8.68 g, yield 89%). FAB-MS was measured, mass number m/z=789 was observed as a molecular ion peak, and Compound E54 was identified.

10. Synthesis of Compound F51

(Synthesis of Intermediate IM-32)

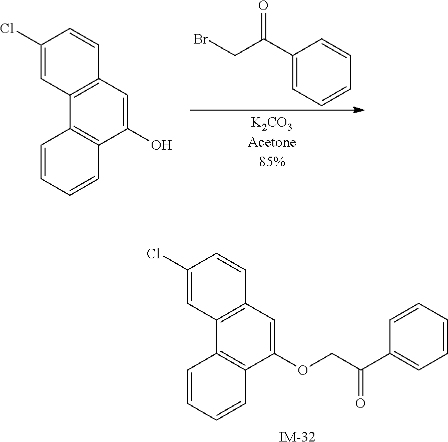

Under an Ar atmosphere, 25.00 g (109.3 mmol) of 3-chlorophenanthren-9-ol, 45.33 g (3.0 eq, 328.0 mmol) of K₂CO₃, 32.64 g (1.5 eq, 164.0 mmol) of phenacyl bromide and 364 m1 (0.3 M) of acetone were added one by one to a 500 m1, three-neck flask, followed by heating and refluxing while stirring at about 70° C. After cooling to room temperature, the reaction solution was filtered by a celite, and an organic layer was concentrated under reduced pressure. The crude product thus obtained was separated by silica gel column chromatography (using a mixture solvent of hexane and toluene as a developer) to obtain Intermediate IM-32 (29.20 g, yield 85%). FAB-MS was measured, mass number m/z=346 was observed as a molecular ion peak, and Intermediate IM-32 was identified.

(Synthesis of Intermediate IM-33)

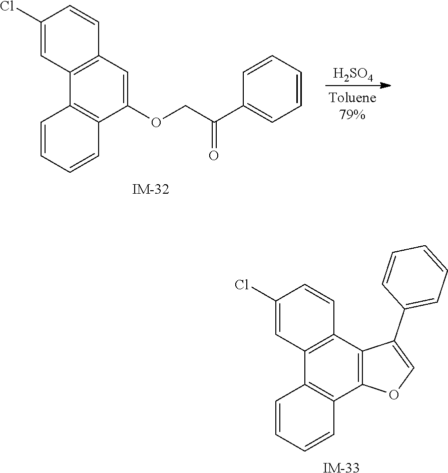

Under an Ar atmosphere, 27.00 g (77.9 mmol) of IM-32, 260 m1 (0.3 M) of toluene and 0.83 m1 (0.2 eq, 15.6 mmol) of H₂₅₀₄ were added one by one to a 500 m1, three-neck flask, followed by heating and refluxing while stirring at about 120° C. After cooling to room temperature, water was added to the reaction solution, and an organic layer was separately taken. Toluene was added to an aqueous layer, and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-33 (20.22 g, yield 79%). FAB-MS was measured, mass number m/z=328 was observed as a molecular ion peak, and Intermediate IM-33 was identified.

(Synthesis of Intermediate IM-34)

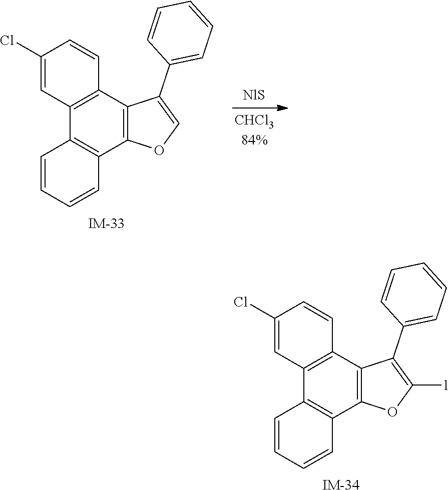

Under an Ar atmosphere, 20.00 g (60.8 mmol) of IM-33, 15.05 g (1.1 eq, 66.9 mmol) of NIS, and 304 m1 (0.2 M) of CHCl₃ were added one by one to a 500 m1, three-neck flask, followed by heating and refluxing while stirring at about 60° C. After cooling to room temperature, the reaction solution was concentrated under reduced pressure and the crude product thus obtained was separated by silica gel column chromatography (using a mixture solvent of hexane and toluene as a developer) to obtain Intermediate IM-34 (23.23 g, yield 84%). FAB-MS was measured, mass number m/z=454 was observed as a molecular ion peak, and Intermediate IM-34 was identified.

(Synthesis of Intermediate IM-35)

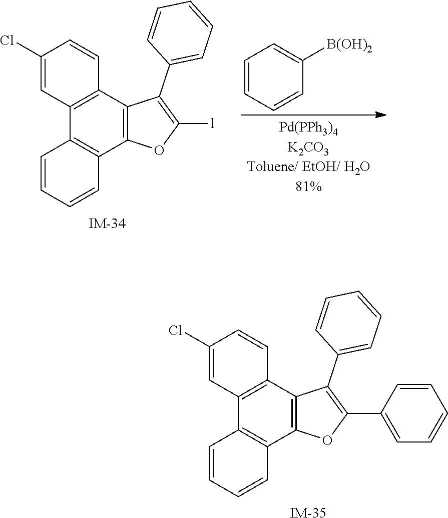

Under an Ar atmosphere, 20.00 g (44.0 mmol) of IM-34, 5.90 g (1.1 eq, 48.4 mmol) of phenylboronic acid, 18.24 g (3.0 eq, 132.0 mmol) of K₂CO₃, 2.54 g (0.05 eq, 2.2 mmol) of Pd(PPh₃)₄, and 308 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-35 (14.43 g, yield 81%). FAB-MS was measured, mass number m/z=404 was observed as a molecular ion peak, and Intermediate IM-35 was identified.

(Synthesis of IM-36)

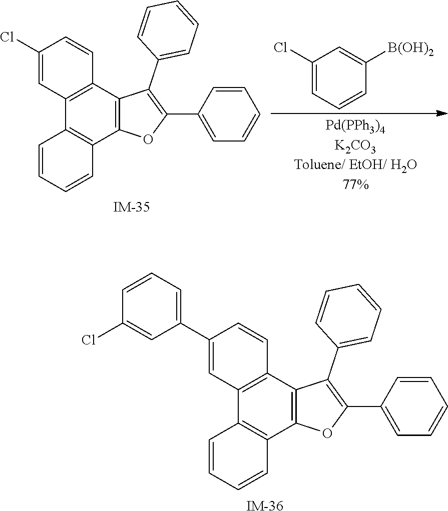

Under an Ar atmosphere, 13.00 g (32.1 mmol) of IM-37, 5.52 g (1.1 eq, 35.3 mmol) of 3-chlorophenylboronic acid, 13.31 g (3.0 eq, 96.3 mmol) of K₂CO₃, 1.86 g (0.05 eq, 1.6 mmol) of Pd(PPh₃)₄, and 225 m1 of a mixture solution of toluene/EtOH/H₂O (4/2/1) were added one by one to a 500 m1, three-neck flask, followed by heating while stirring at about 80° C. After cooling to room temperature, the reaction solution was extracted with toluene. An aqueous layer was removed, and an organic layer was washed with a saturated saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Intermediate IM-36 (11.89 g, yield 77%). FAB-MS was measured, mass number m/z=480 was observed as a molecular ion peak, and Intermediate IM-36 was identified.

(Synthesis of Compound F51)

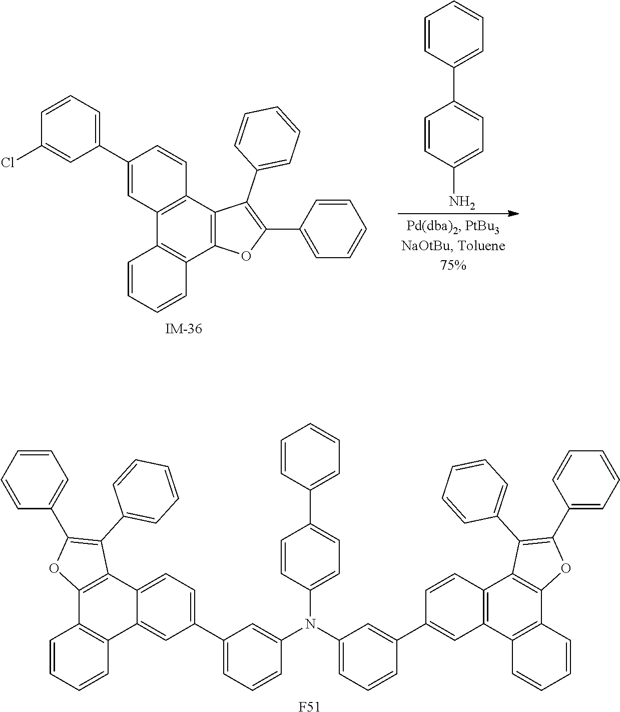

Under an Ar atmosphere, 1.50 g (8.9 mmol) of 4-biphenylamine, 9.38 g (2.2 eq, 19.5 mmol) of IM-36, 0.31 g (0.06 eq, 0.5 mmol) of Pd(dba)₂, 3.41 g (4.0 eq, 35.5 mmol) of NaOtBu, 44 m1 of toluene, 0.36 g (0.2 eq, 1.8 mmol) of PtBu₃ were added one by one to a 200 m1, three-neck flask, followed by heating and refluxing while stirring at about 120° C. After cooling to room temperature, water was added to the reaction solution and an organic layer was separately taken. Toluene was added to an aqueous layer and an organic layer was additionally extracted. The organic layers were combined, washed with a saline solution and dried with MgSO₄. MgSO₄ was separated by filtering and an organic layer was concentrated. The crude product thus obtained was separated by silica gel column chromatography (using a mixture solvent of hexane and toluene as a developer) to obtain Compound F51 as a solid (7.04 g, yield 75%). FAB-MS was measured, mass number m/z=1058 was observed as a molecular ion peak, and Compound F51 was identified.

(Device manufacturing examples)

Organic electroluminescence devices of Examples 1 to 7 were manufactured using the above-described Compounds A1, A5, A12, A17, A33, A73 and A41 as materials for a hole transport region.

### Example Compounds

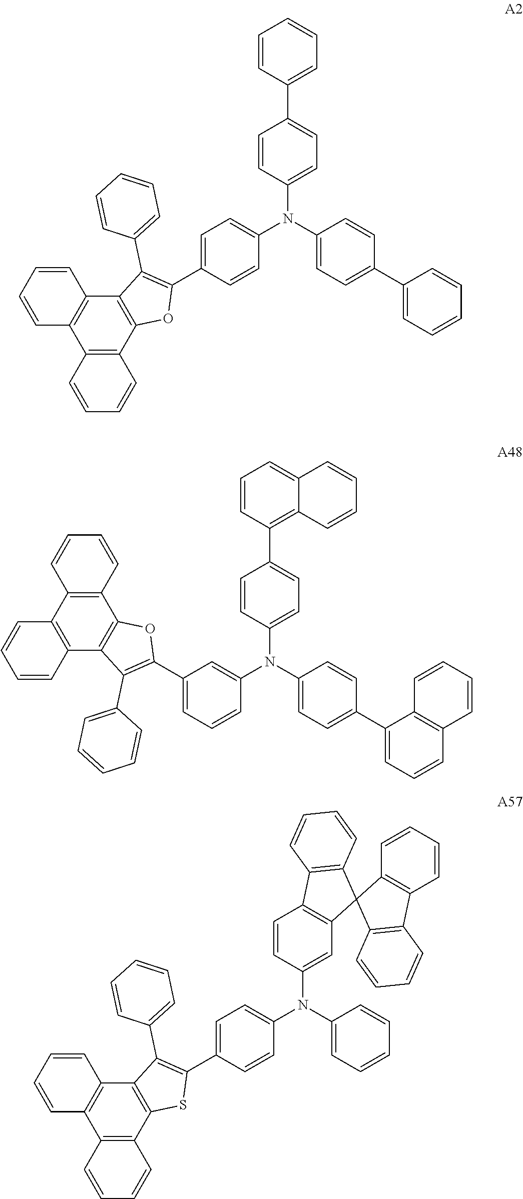 [SMILES: c1ccc(-c2ccc(N(c3ccc(-c4ccccc4)cc3)c3ccc(-c4oc5c6ccccc6c6ccccc6c5c4-c4ccccc4)cc3)cc2)cc1 ; c1ccc(-c2c(-c3cccc(N(c4ccc(-c5cccc6ccccc56)cc4)c4ccc(-c5cccc6ccccc56)cc4)c3)oc3c4ccccc4c4ccccc4c23)cc1 ; c1ccc(-c2c(-c3ccc(N(c4ccccc4)c4ccc5c(c4)C4(c6ccccc6-c6ccccc64)c4ccccc4-5)cc3)sc3c4ccccc4c4ccccc4c23)cc1]

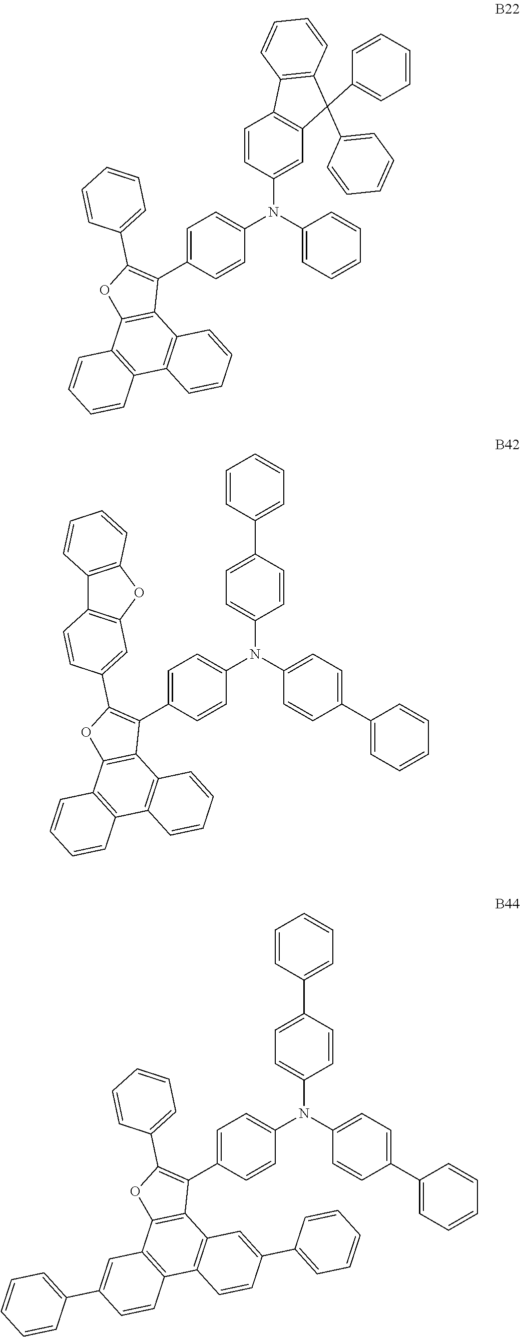 [SMILES: c1ccc(-c2oc3c4ccccc4c4ccccc4c3c2-c2ccc(N(c3ccccc3)c3ccc4c(c3)C(c3ccccc3)(c3ccccc3)c3ccccc3-4)cc2)cc1 ; c1ccc(-c2ccc(N(c3ccc(-c4ccccc4)cc3)c3ccc(-c4c(-c5ccc6c(c5)oc5ccccc56)oc5c6ccccc6c6ccccc6c45)cc3)cc2)cc1 ; c1ccc(-c2ccc(N(c3ccc(-c4ccccc4)cc3)c3ccc(-c4c(-c5ccccc5)oc5c6cc(-c7ccccc7)ccc6c6ccc(-c7ccccc7)cc6c45)cc3)cc2)cc1]

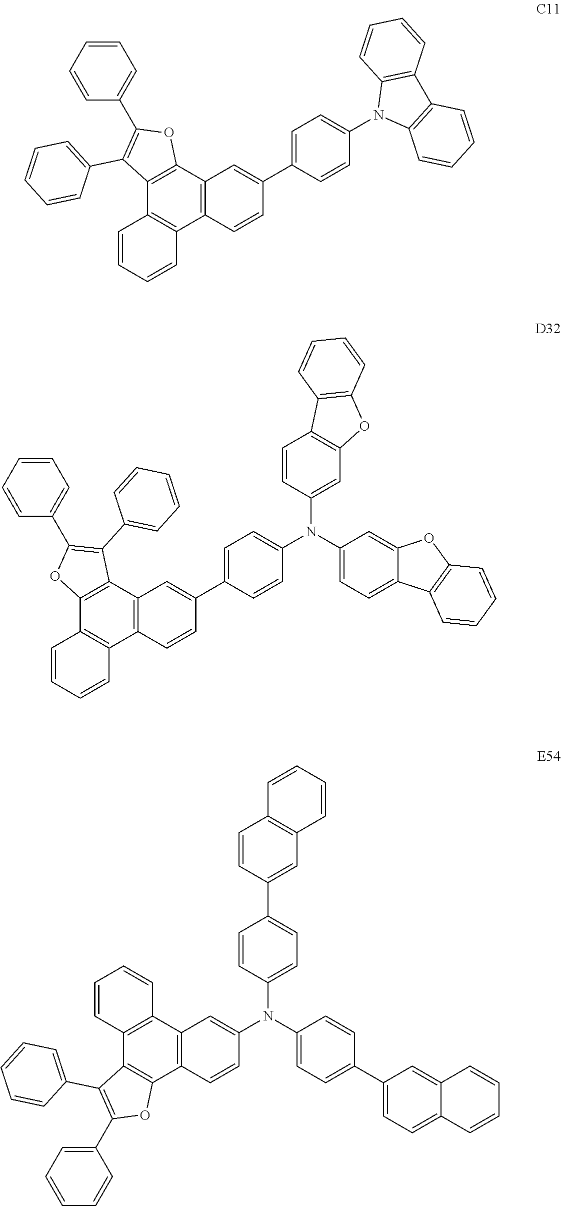 [SMILES: c1ccc(-c2oc3c4cc(-c5ccc(-n6c7ccccc7c7ccccc76)cc5)ccc4c4ccccc4c3c2-c2ccccc2)cc1 ; c1ccc(-c2oc3c4ccccc4c4ccc(-c5ccc(N(c6ccc7c(c6)oc6ccccc67)c6ccc7c(c6)oc6ccccc67)cc5)cc4c3c2-c2ccccc2)cc1 ; c1ccc(-c2oc3c4ccc(N(c5ccc(-c6ccc7ccccc7c6)cc5)c5ccc(-c6ccc7ccccc7c6)cc5)cc4c4ccccc4c3c2-c2ccccc2)cc1]

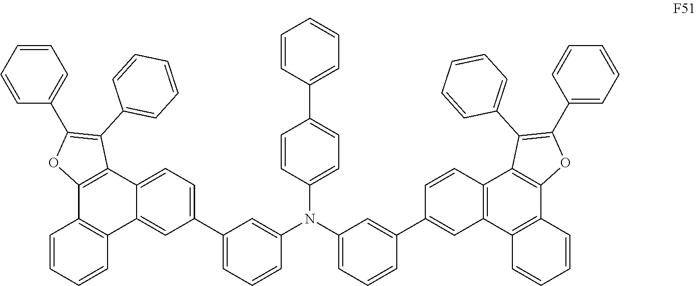 [SMILES: c1ccc(-c2ccc(N(c3cccc(-c4ccc5c(c4)c4ccccc4c4oc(-c6ccccc6)c(-c6ccccc6)c54)c3)c3cccc(-c4ccc5c(c4)c4ccccc4c4oc(-c6ccccc6)c(-c6ccccc6)c54)c3)cc2)cc1]

Organic electroluminescence devices of Comparative Examples 1 to 4 were manufactured using Comparative Compounds R₁ to R₄ below as materials for a hole transport region.

[Comparative Compounds]

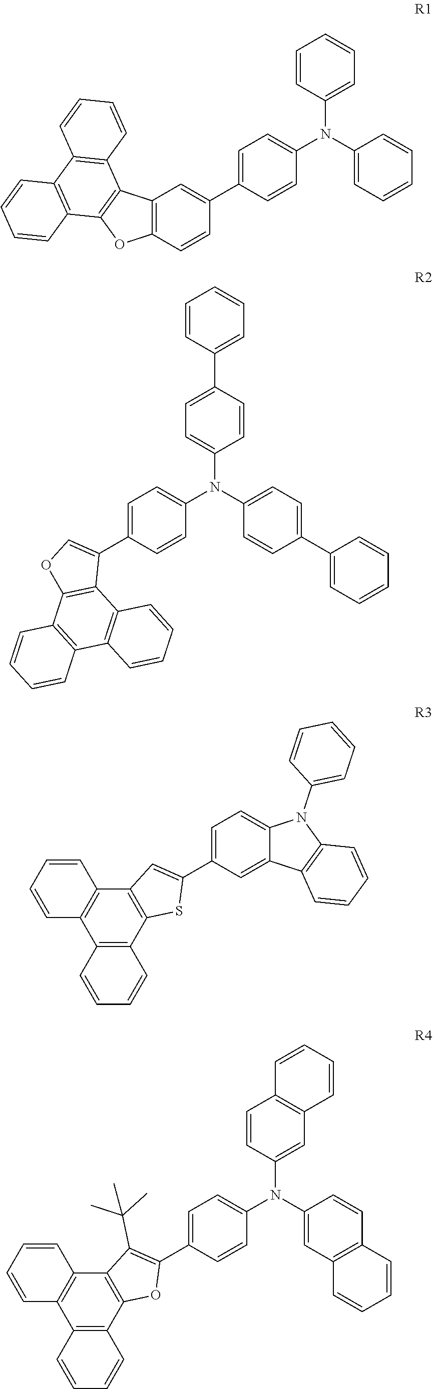 [SMILES: c1ccc(N(c2ccccc2)c2ccc(-c3ccc4oc5c6ccccc6c6ccccc6c5c4c3)cc2)cc1 ; c1ccc(-c2ccc(N(c3ccc(-c4ccccc4)cc3)c3ccc(-c4coc5c6ccccc6c6ccccc6c45)cc3)cc2)cc1 ; c1ccc(-n2c3ccccc3c3cc(-c4cc5c6ccccc6c6ccccc6c5s4)ccc32)cc1 ; CC(C)(C)c1c(-c2ccc(N(c3ccc4ccccc4c3)c3ccc4ccccc4c3)cc2)oc2c3ccccc3c3ccccc3c12]

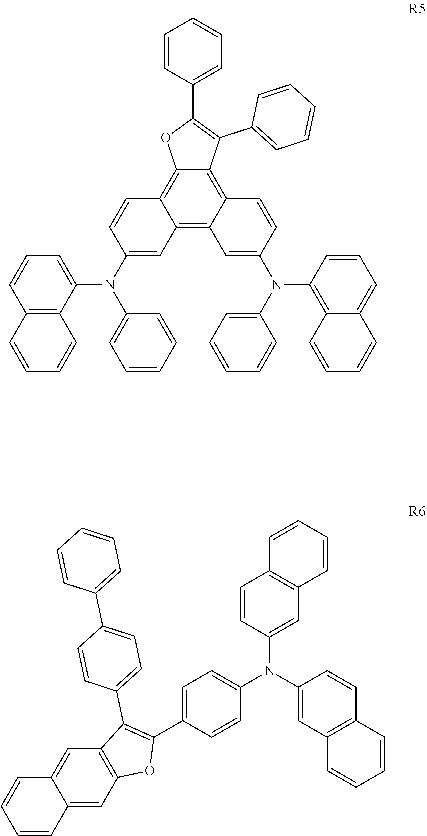 [SMILES: c1ccc(-c2oc3c4ccc(N(c5ccccc5)c5cccc6ccccc56)cc4c4cc(N(c5ccccc5)c5cccc6ccccc56)ccc4c3c2-c2ccccc2)cc1 ; c1ccc(-c2ccc(-c3c(-c4ccc(N(c5ccc6ccccc6c5)c5ccc6ccccc6c5)cc4)oc4cc5ccccc5cc34)cc2)cc1]

The organic electroluminescence devices of the Examples and the Comparative Examples were manufactured by the method as follows. On a glass substrate, ITO of a thickness of about 150 nm was patterned, washed with ultrapure water and treated with UV ozone for about 10 minutes to form a first electrode. After that, 2-TNATA was deposited to a thickness of about 60 nm, and the Example Compound or Comparative Compound was deposited to a thickness of about 30 nm to form a hole transport region. Then, an emission layer was formed using ADN doped with 3% TBP to a thickness of about 25 nm, and on the emission layer, a layer was formed using Alq₃ to a thickness of about 25 nm and a layer was formed using LiF to a thickness of about 1 nm to form an electron transport region. After that, a second electrode was formed using aluminum (Al) to a thickness of about 100 nm. Each layer was formed by a vacuum deposition method.

The emission efficiency of the organic electroluminescence devices according to Examples 1 to 10 and Comparative Examples 1 to 6 are shown in Table 1 below. The emission layer represents values measured at about 10 mA/cm², and half life represents test results at about 1.0 mA/cm².

TABLE 1

| | Hole transport | Voltage | Efficiency | Life (LT50 |
|---|---|---|---|---|
| | layer | (V) | (cd/A) | (h)) |
| Example 1 | Example | 5.6 | 7.0 | 2100 |
| | Compound A2 | | | |
| Example 2 | Example | 5.5 | 7.2 | 2000 |
| | Compound A48 | | | |
| Example 3 | Example | 5.5 | 7.1 | 2150 |
| | Compound A57 | | | |
| Example 4 | Example | 5.4 | 7.5 | 1850 |
| | Compound B22 | | | |
| Example 5 | Example | 5.4 | 7.4 | 1900 |
| | Compound B42 | | | |
| Example 6 | Example | 5.7 | 7.5 | 1950 |
| | Compound B44 | | | |
| Example 7 | Example | 5.6 | 6.9 | 2000 |
| | Compound C11 | | | |
| Example 8 | Example | 5.5 | 6.8 | 2050 |
| | Compound D32 | | | |
| Example 9 | Example | 5.6 | 7.0 | 2050 |
| | Compound E54 | | | |
| Example 10 | Example | 5.7 | 7.2 | 1950 |
| | Compound F51 | | | |
| Comparative | Comparative | 6.4 | 5.3 | 1700 |
| Example 1 | Compound R1 | | | |
| Comparative | Comparative | 6.8 | 4.9 | 1550 |
| Example 2 | Compound R2 | | | |
| Comparative | Comparative | 6.7 | 4.7 | 1500 |
| Example 3 | Compound R3 | | | |
| Comparative | Comparative | 6.5 | 4.8 | 1550 |
| Example 4 | Compound R4 | | | |
| Comparative | Comparative | 6.0 | 5.4 | 1600 |
| Example 5 | Compound R5 | | | |
| Comparative | Comparative | 6.3 | 5.8 | 1750 |
| Example 6 | Compound R6 | | | |

Referring to Table 1, it was confirmed that Examples 1 to 10 achieved a decreased voltage, longer life and higher efficiency when compared with Comparative Examples 1 to 6.

The polycyclic compound according to the inventive concepts introduced a phenanthrofuran or phenanthrothiophene structure, which has excellent resistance to heat and charge to an amine group, and achieved the decrease of a voltage, the increase of life and the increase of efficiency of a device. In addition, since an aryl group or a heteroaryl group with high stability was substituted at a highly reactive α—position and β—position of a furan ring and a thiophene ring of the phenanthrofuran and phenanthrothiophene, respectively, structural stability in a radical state was improved and at the same time, distorted steric structure was maintained due to steric electronic repulsion, and thus, a volume was increased to restrain crystallinity. Accordingly, it is thought that layer quality was improved and hole transport properties were improved to increase device efficiency.

In Examples 1 to 3, the emission life was particularly improved. It is thought that, in the polycyclic compounds of Examples 1 to 3, since an amine group was substituted at an α— position of a furan ring or a thiophene ring, which is included in a phenanthrofuran structure or a phenanthrothiophene structure, the highest occupied molecular orbital (HOMO) of a substituent including the amine group was widely enlarged in the phenanthrofuran structure or the phenanthrothiophene structure and the stability in a radical state was improved.

In Examples 4 to 6, the emission efficiency was particularly improved. It is thought that, in the polycyclic compounds of Examples 4 to 6, since an amine group was substituted at a β—position of a furan ring which was included in the phenanthrofuran structure, the substituent substituted at β—position and the phenanthrofuran ring were distorted, the planarity of an entire molecule was deteriorated, crystallinity was restrained, and thus, hole transport properties were improved, and recombination probability of holes and electrons in an emission layer was improved.

The aryl group substituted at the α—position and β—position of the furan ring or thiophene ring, included in the phenanthrofuran structure or the phenanthrothiophene structure is distorted from the phenanthrofuran structure or the phenanthrothiophene structure in different angles, respectively. That is, the aryl group substituted at the α—position has high planarity with the phenanthrofuran structure or the phenanthrothiophene structure, but the aryl group substituted at the β—position maintains largely distorted steric structure from the phenanthrofuran structure or the phenanthrothiophene structure. Accordingly, the improving properties of the device may be changed according to the substitution position of the amine group.

In Examples 7 to 10, the emission life was particularly improved. It is thought that, in the polycyclic compounds of Examples 7 to 10, an amine group was substituted at the side chain of a phenanthrene ring included in the phenanthrofuran structure of the phenanthrothiophene structure, and the HOMO orbital of a substituent including the amine group was sufficiently enlarged in the phenanthrofuran structure or the phenanthrothiophene structure, and the stability in a radical state was improved.

In Comparative Example 1, the device efficiency was particularly degraded when compared with the Examples. It is thought that since R₁ of Comparative Example 1 had a phenanthrobenzofuran structure, entire planarity was improved, intermolecular interaction was strengthened, and hole transport properties were degraded.

In Comparative Examples 2 to 4, both device efficiency and life were degraded when compared with the Examples. It is thought that, R₂ and R₃ of Comparative Examples 2 and 3 had similar phenanthrofuran structures as the inventive concepts, but highly reactive α— position and β—position of a furan ring were not protected by an aryl group, and stability in a radical state was not good and the decomposition of a material was generated. In addition, in R₄ of Comparative Example 4, an alkyl group was substituted at the β—position of a furan ring, and stability in a radical state was insufficient, and device properties were degraded when compared with the Examples.

It is thought that Comparative Example 5 was a diamine compound and the carrier balance thereof was collapsed, and thus, both the device efficiency and life were degraded when compared with the Examples.

It is thought that Comparative Example 6 was an amine containing a condensed polycyclic structure having a smaller resonance region than the polycyclic compound according to an embodiment of the inventive concepts, and the HOMO orbital enlargement was decreased, and the device life was particularly decreased.

The polycyclic compound according to an embodiment of the inventive concepts is used in a hole transport region and contributes to the achievement of the decrease of a driving voltage, the increase of efficiency and the increase of life of an organic electroluminescence device.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. An organic electroluminescence device, comprising:
a first electrode;
a hole transport region on the first electrode;
an emission layer on the hole transport region;
an electron transport region on the emission layer; and
a second electrode on the electron transport region,
wherein the hole transport region comprises a polycyclic compound represented by Formula 5 or Formula 6: 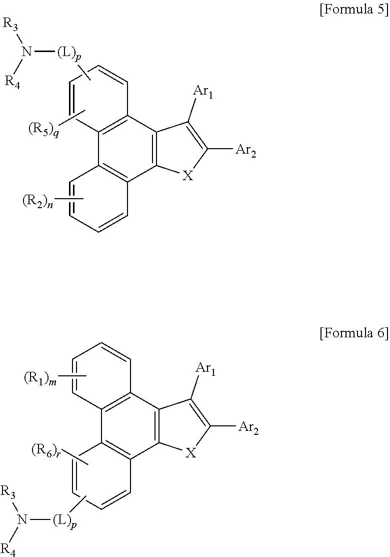
wherein, in Formula 5 and Formula 6, X is 0 or S,
Ar₁ and Ar₂ are each independently a substituted or unsubstituted aryl group of 6 to 30 carbon atoms for forming a ring, or a substituted or unsubstituted heteroaryl group of 2 to 30 carbon atoms for forming a ring,
R₁ and R₂ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring,
“m” and “n” are each independently an integer of 0 to 4,
R₅ and R₆ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring,
“q” and “r” are each independently an integer of 0 to 3,
L is a direct linkage, a substituted or unsubstituted arylene group of 6 to 30 carbon atoms for forming a ring, or a substituted or unsubstituted heteroarylene group of 2 to 30 carbon atoms for forming a ring,
“p” is an integer of 0 to 3, and
R₃ and R₄ are each independently a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, or combined with an adjacent group to form a ring.

2. The organic electroluminescence device of claim 1, wherein L is a substituted or unsubstituted arylene group of 6 to 12 carbon atoms for forming a ring.

3. The organic electroluminescence device of claim 2, wherein L is a substituted or unsubstituted phenylene group.

4. The organic electroluminescence device of claim 1, wherein Ar₁ and Ar₂ are each independently a substituted or unsubstituted aryl group of 6 to 20 carbon atoms for forming a ring.

5. The organic electroluminescence device of claim 1, wherein X is O.

6. The organic electroluminescence device of claim 1, wherein Formula 5 is represented by Formula 7 or Formula 8: 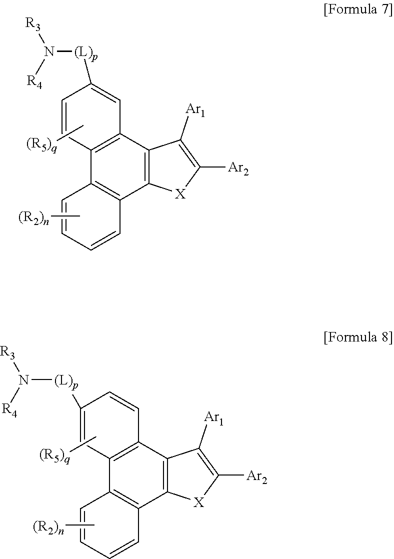
wherein in Formula 7 and Formula 8,
X, Ar₁, Ar₂, R₂ to R₅, L, “n”, “p” and “q” are the same as defined in Formula 5.

7. The organic electroluminescence device of claim 1, wherein Formula 6 is represented by Formula 9 or Formula 10: 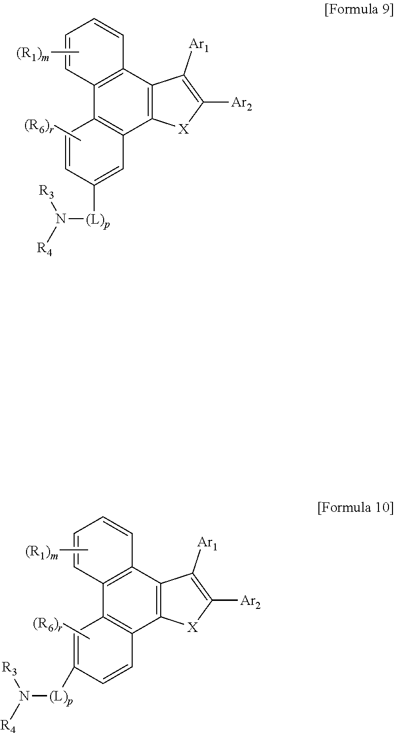
wherein in Formula 9 and Formula 10,
X, Ar₁, Ar₂, R₁, R₃, R₄, R₆, L, “m”, “p” and “r” are the same as defined in Formula 6.

8. The organic electroluminescence device of claim 1, wherein the hole transport region comprises:
a hole injection layer on the first electrode; and
a hole transport layer on the hole injection layer,
wherein the hole transport layer comprises the polycyclic compound represented by Formula 1.

9. The organic electroluminescence device of claim 8, wherein the hole transport region further comprises an electron blocking layer on the hole transport layer.

10. The organic electroluminescence device of claim 1, wherein the polycyclic compound represented by Formula 5 and Formula 6 is any one selected among compounds represented in Compound Group 3 to Compound Group 6:

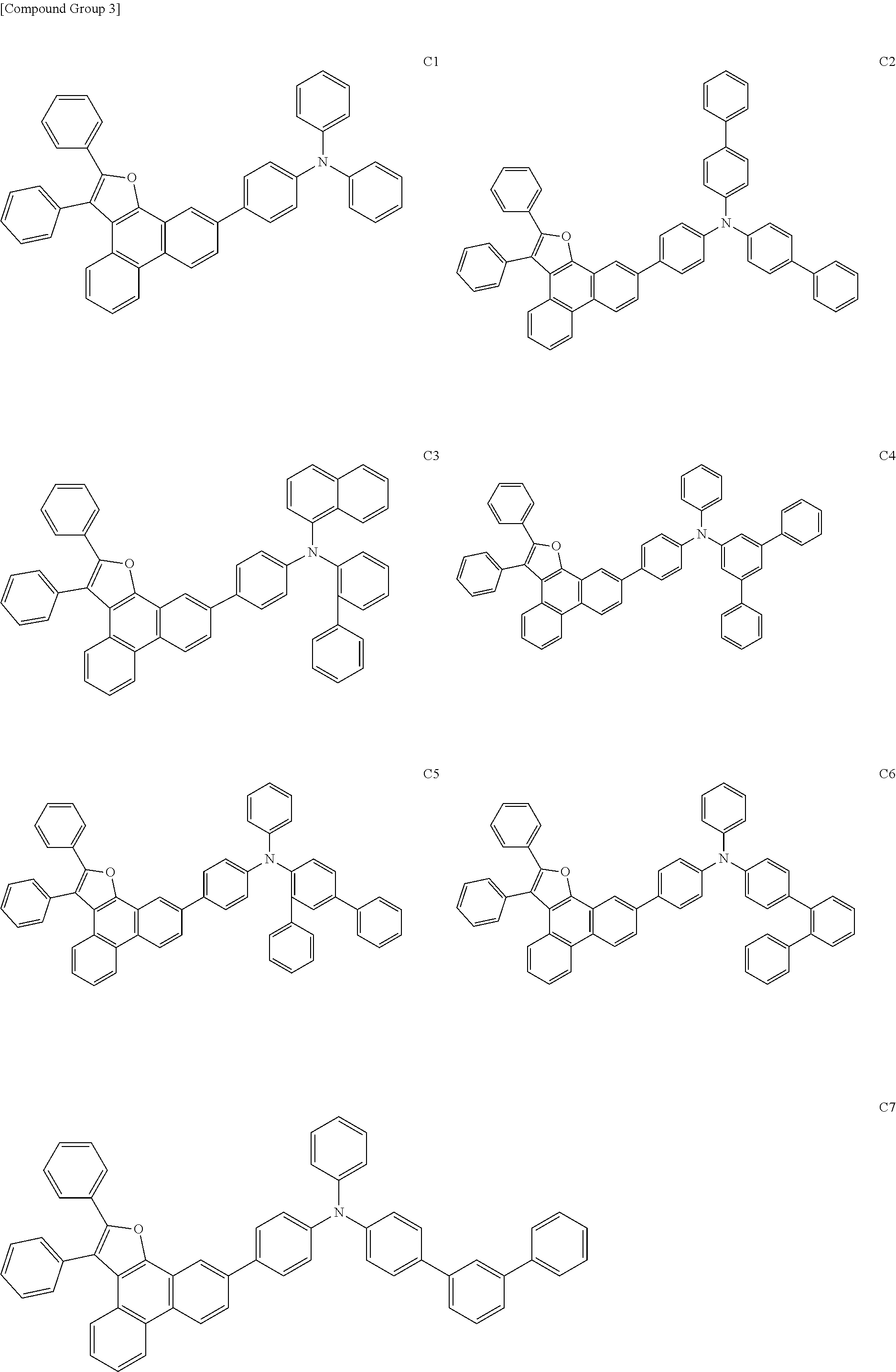

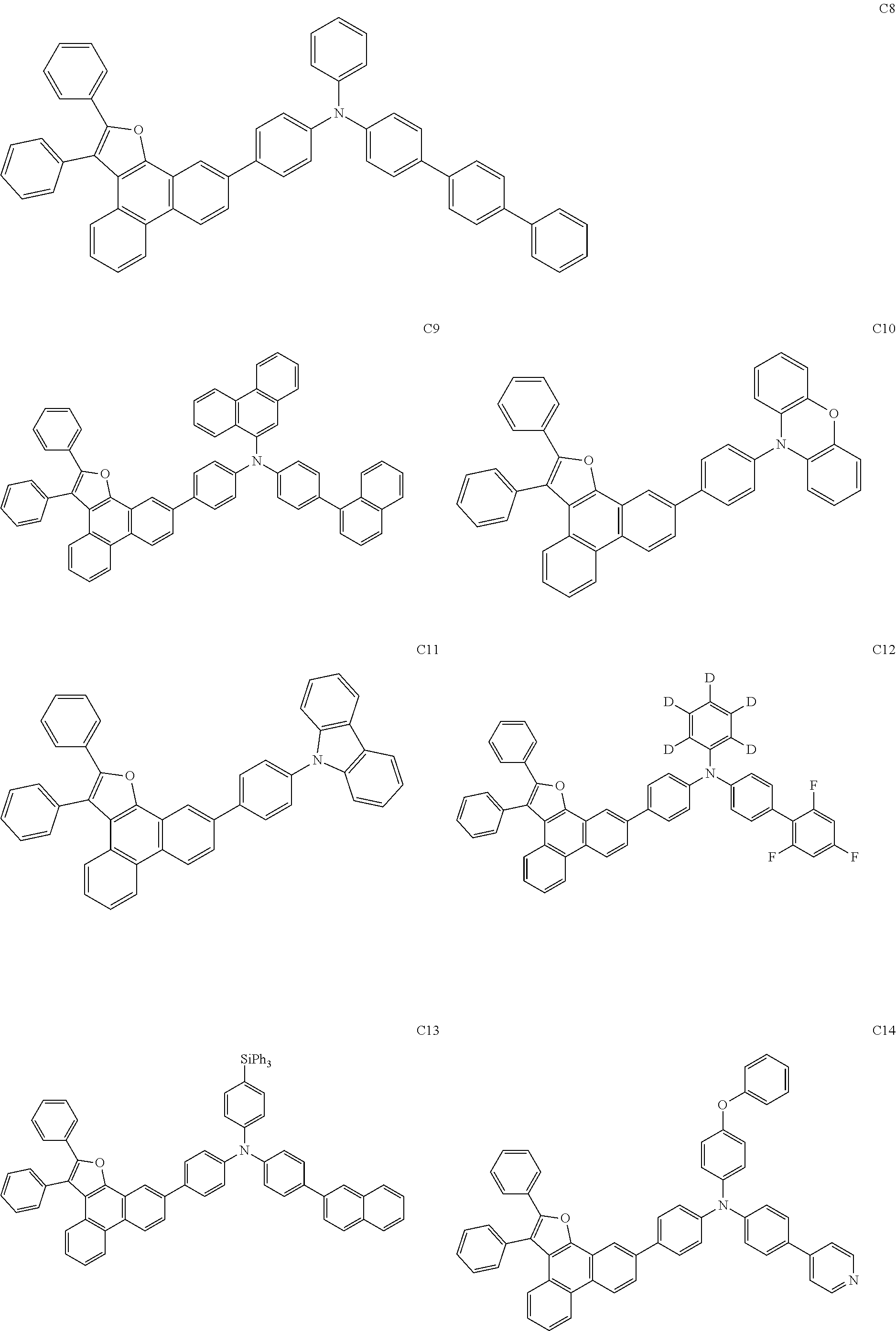

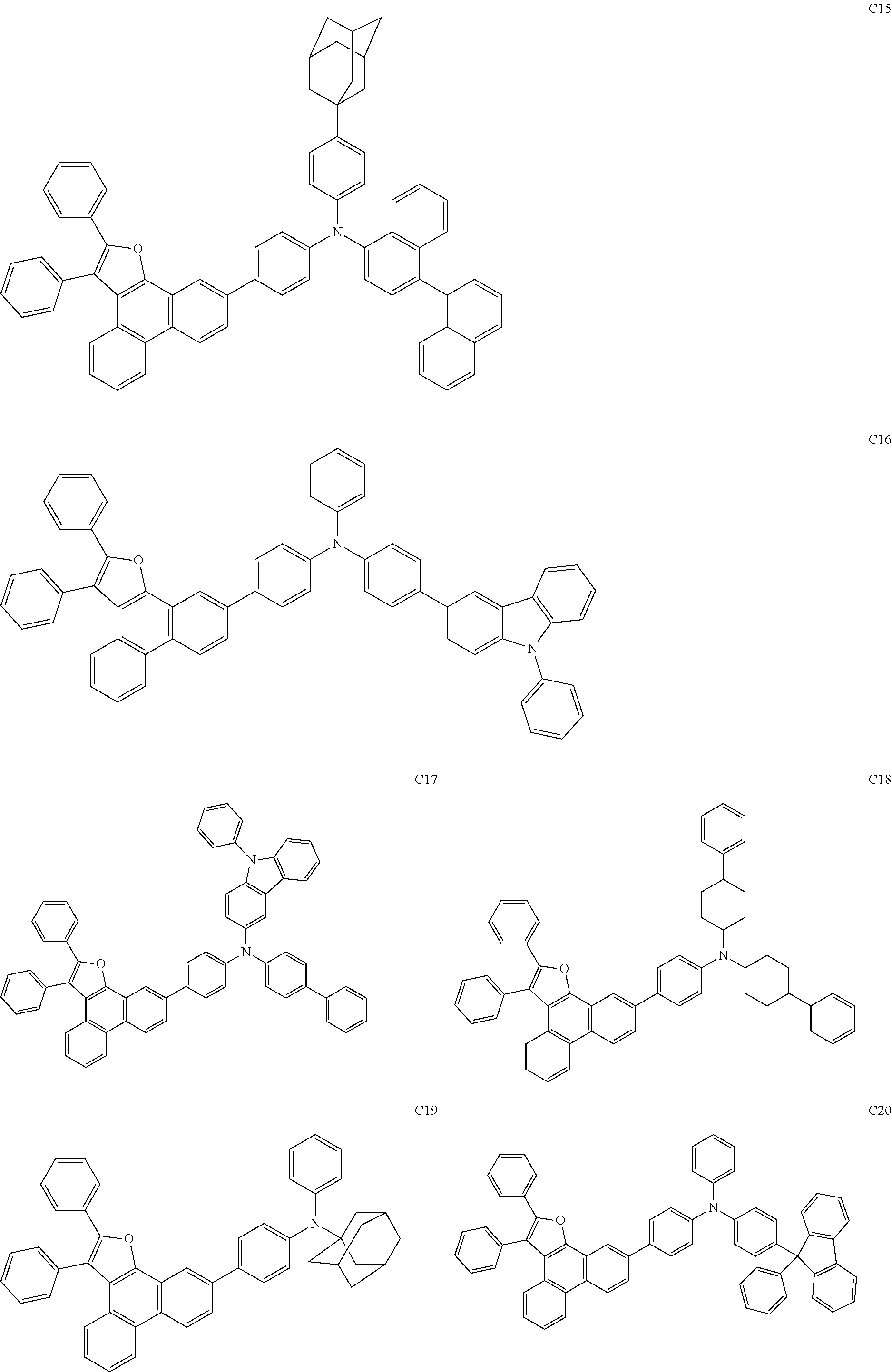

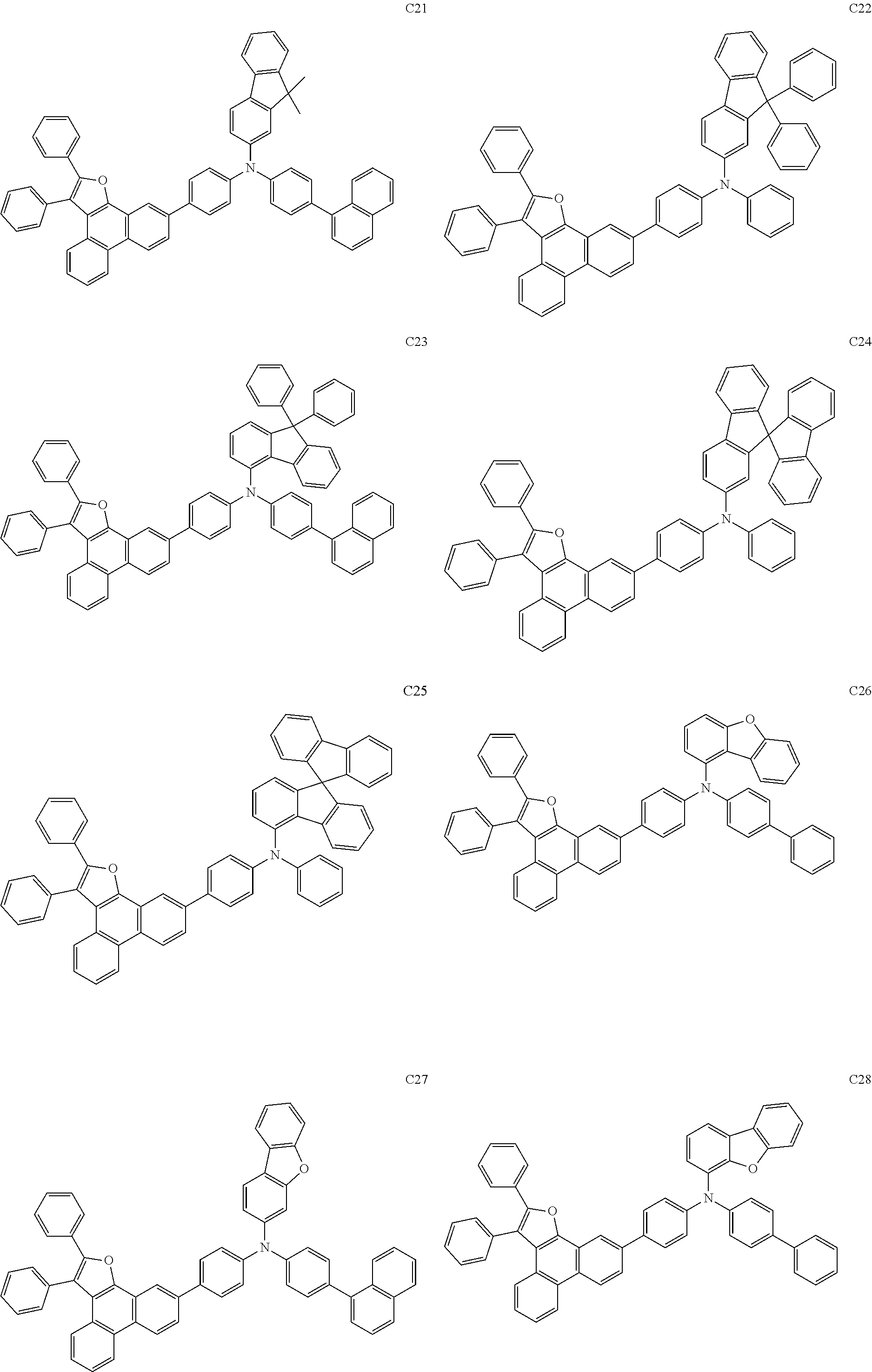

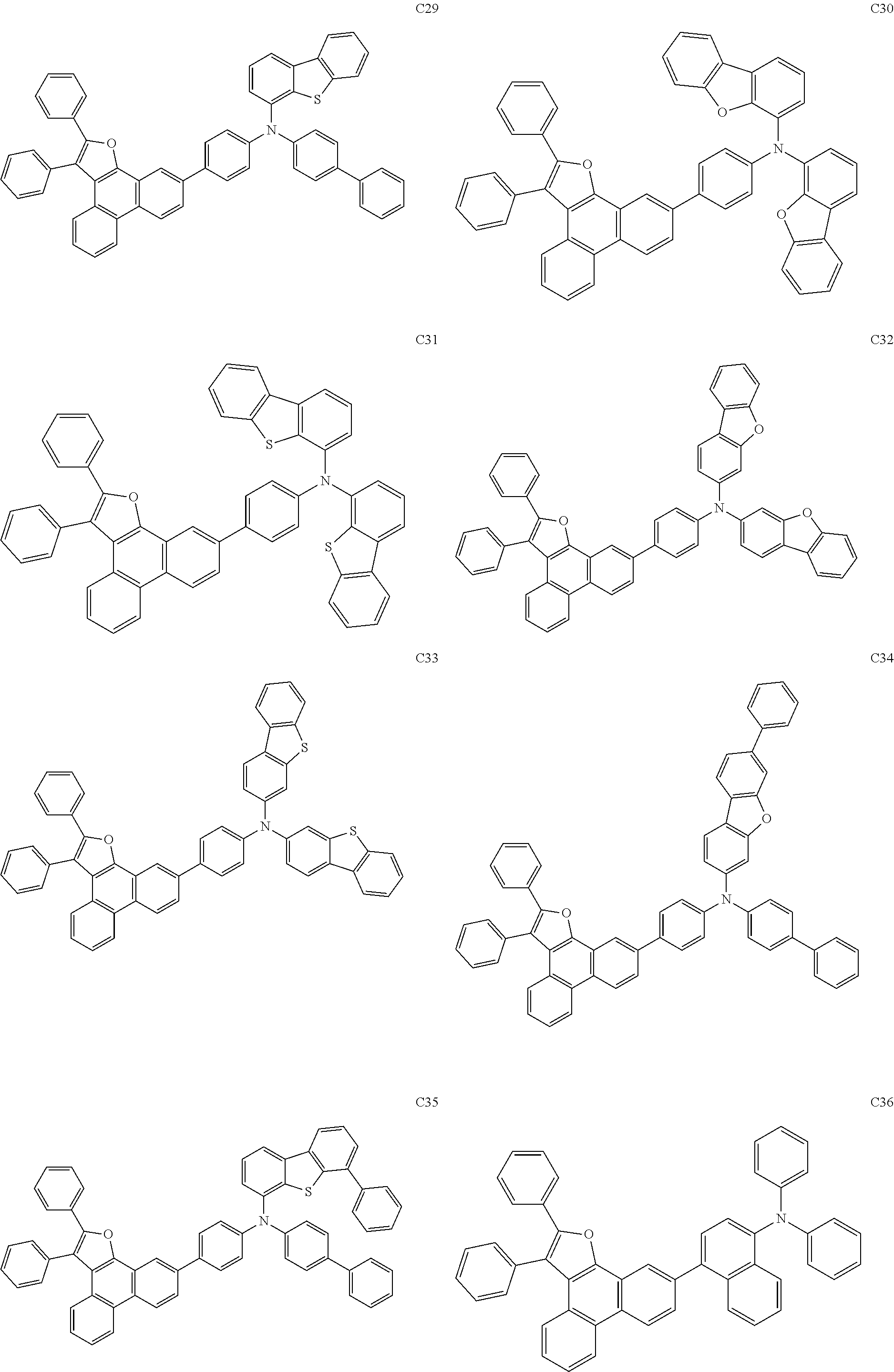

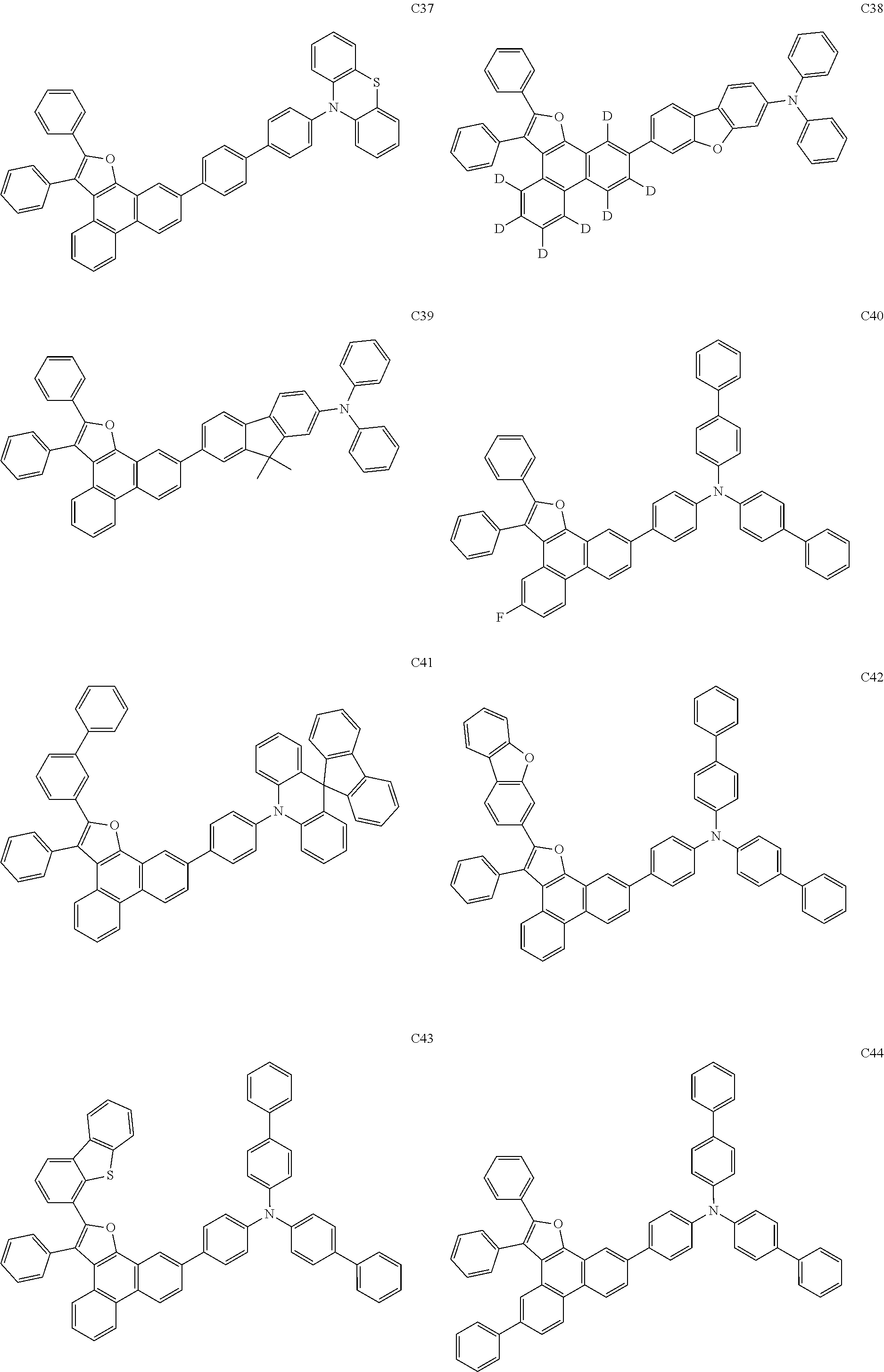

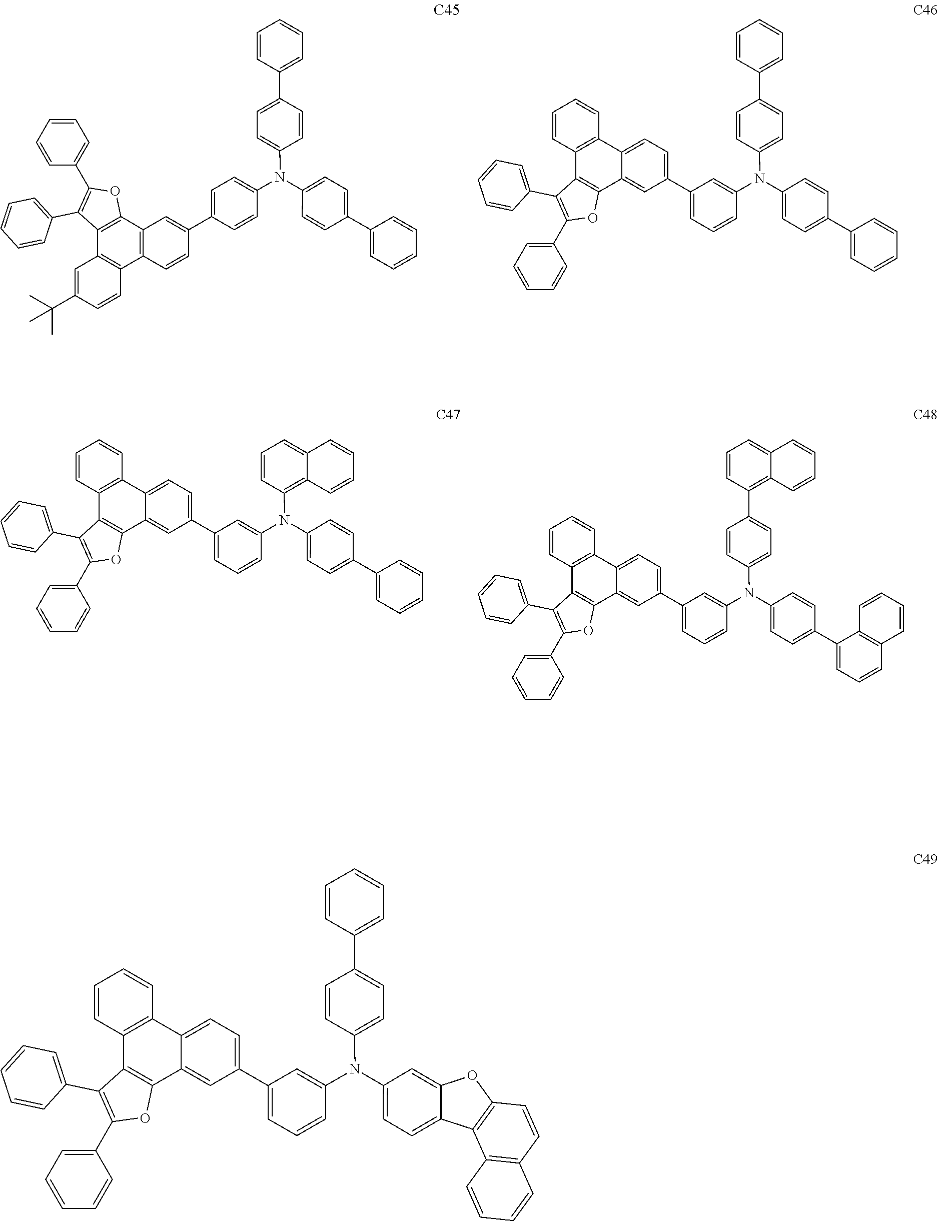

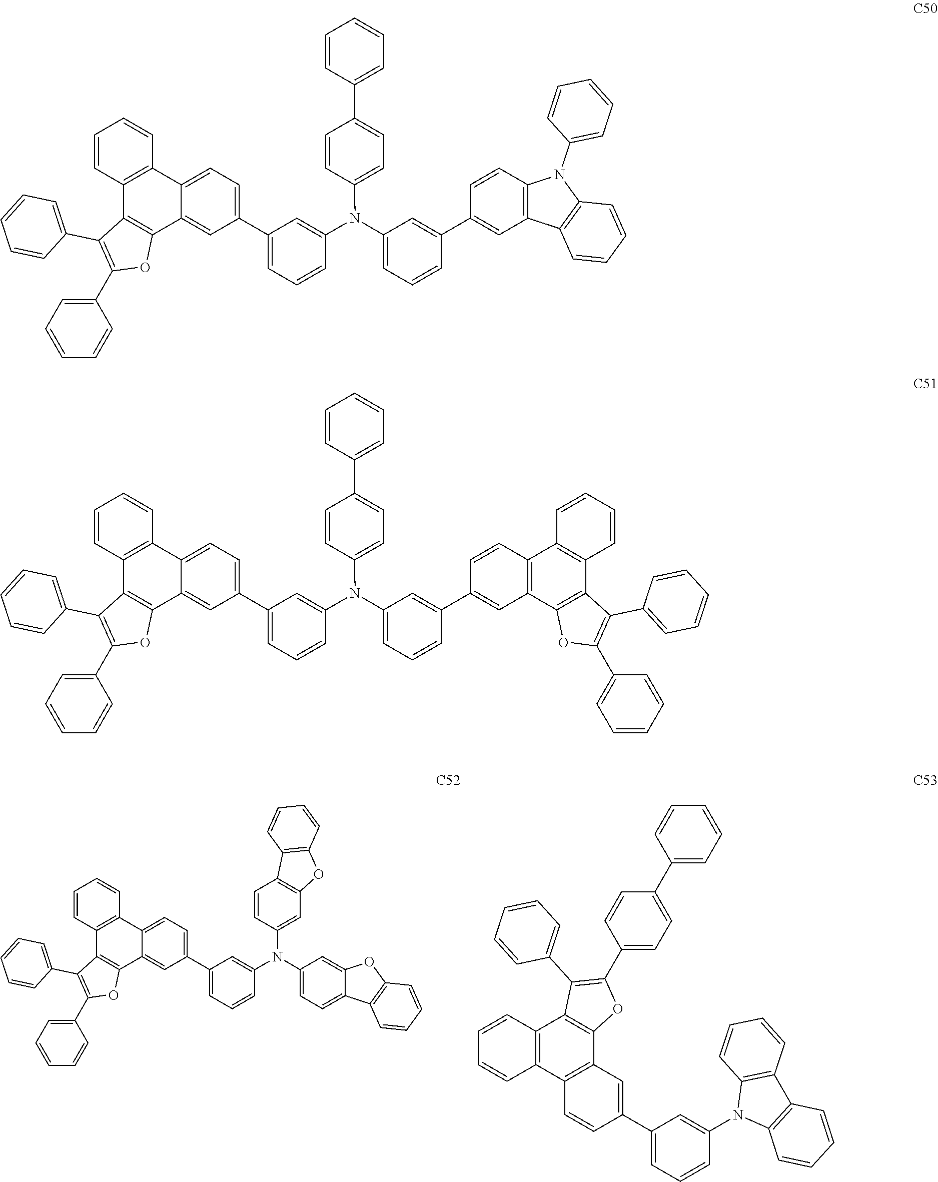 [SMILES: c1ccc(-c2ccc(N(c3cccc(-c4ccc5c6ccccc6c6c(-c7ccccc7)c(-c7ccccc7)oc6c5c4)c3)c3cccc(-c4ccc5c(c4)c4ccccc4n5-c4ccccc4)c3)cc2)cc1 ; c1ccc(-c2ccc(N(c3cccc(-c4ccc5c6ccccc6c6c(-c7ccccc7)c(-c7ccccc7)oc6c5c4)c3)c3cccc(-c4ccc5c6ccccc6c6c(-c7ccccc7)c(-c7ccccc7)oc6c5c4)c3)cc2)cc1 ; c1ccc(-c2oc3c4cc(-c5cccc(N(c6ccc7c(c6)oc6ccccc67)c6ccc7c(c6)oc6ccccc67)c5)ccc4c4ccccc4c3c2-c2ccccc2)cc1 ; c1ccc(-c2ccc(-c3oc4c5cc(-c6cccc(-n7c8ccccc8c8ccccc87)c6)ccc5c5ccccc5c4c3-c3ccccc3)cc2)cc1]

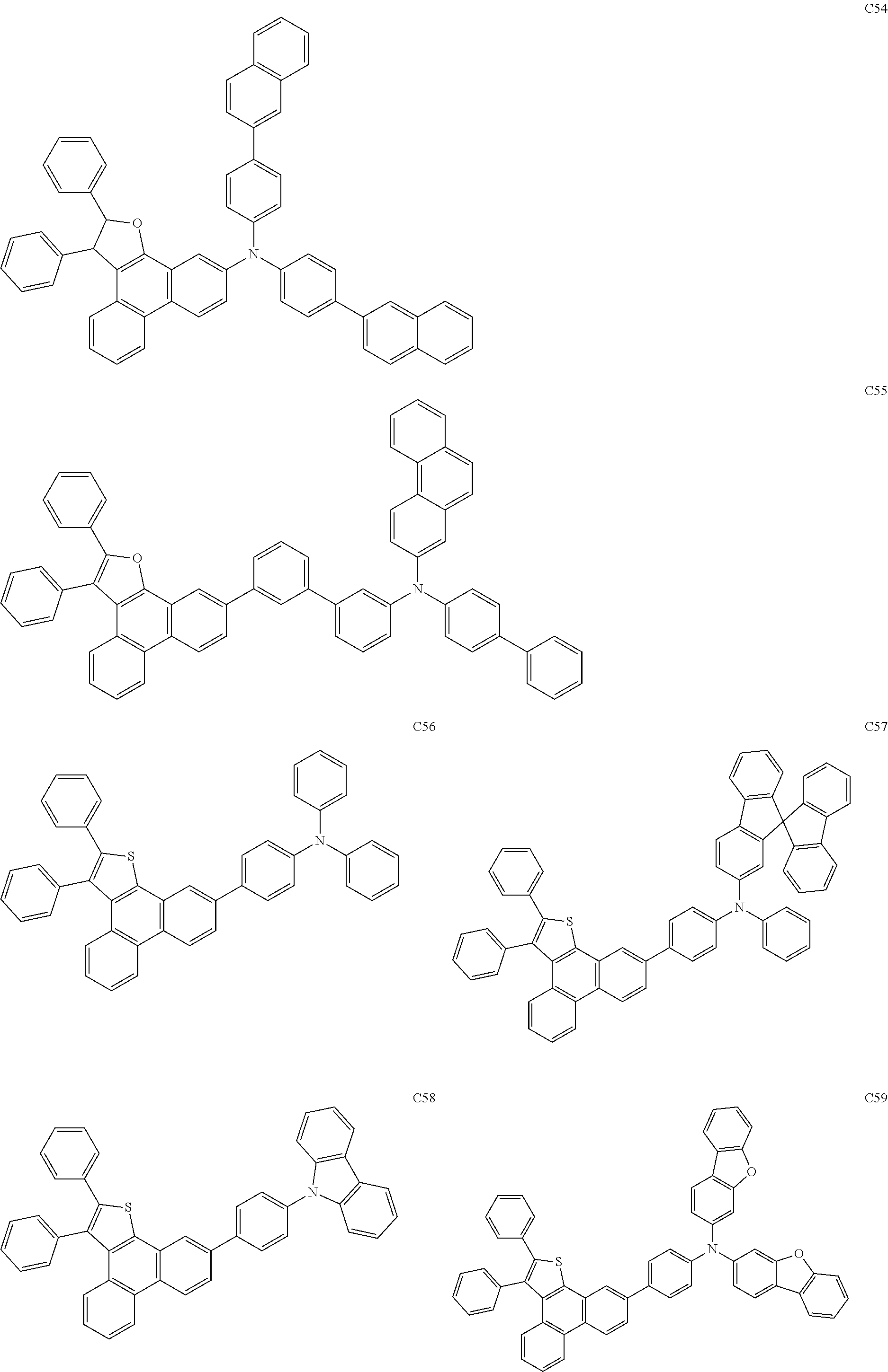

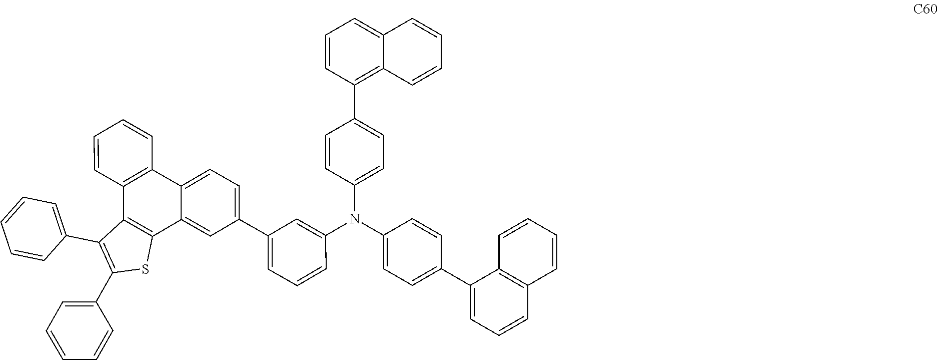 [SMILES: c1ccc(-c2sc3c4cc(-c5cccc(N(c6ccc(-c7cccc8ccccc78)cc6)c6ccc(-c7cccc8ccccc78)cc6)c5)ccc4c4ccccc4c3c2-c2ccccc2)cc1]

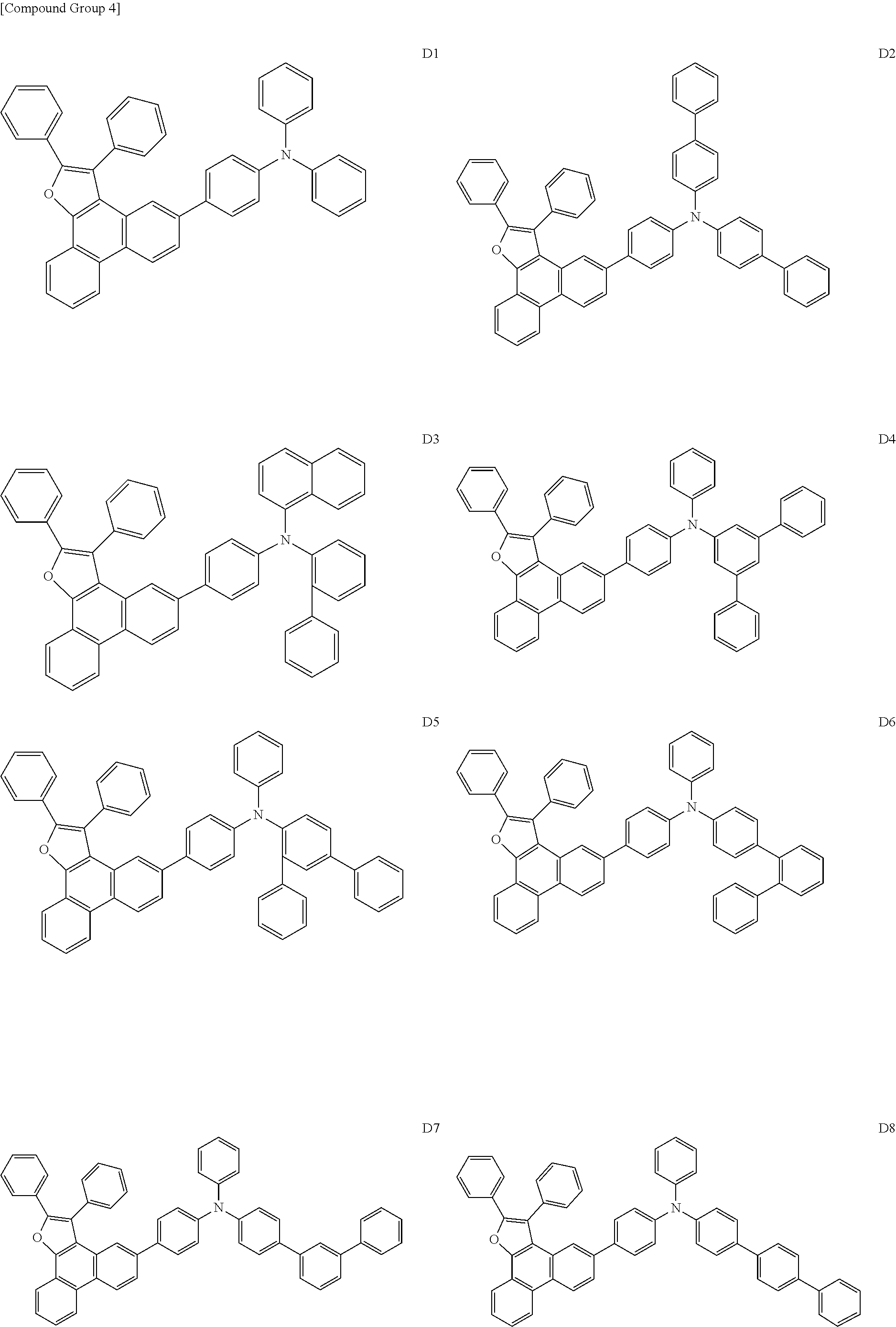

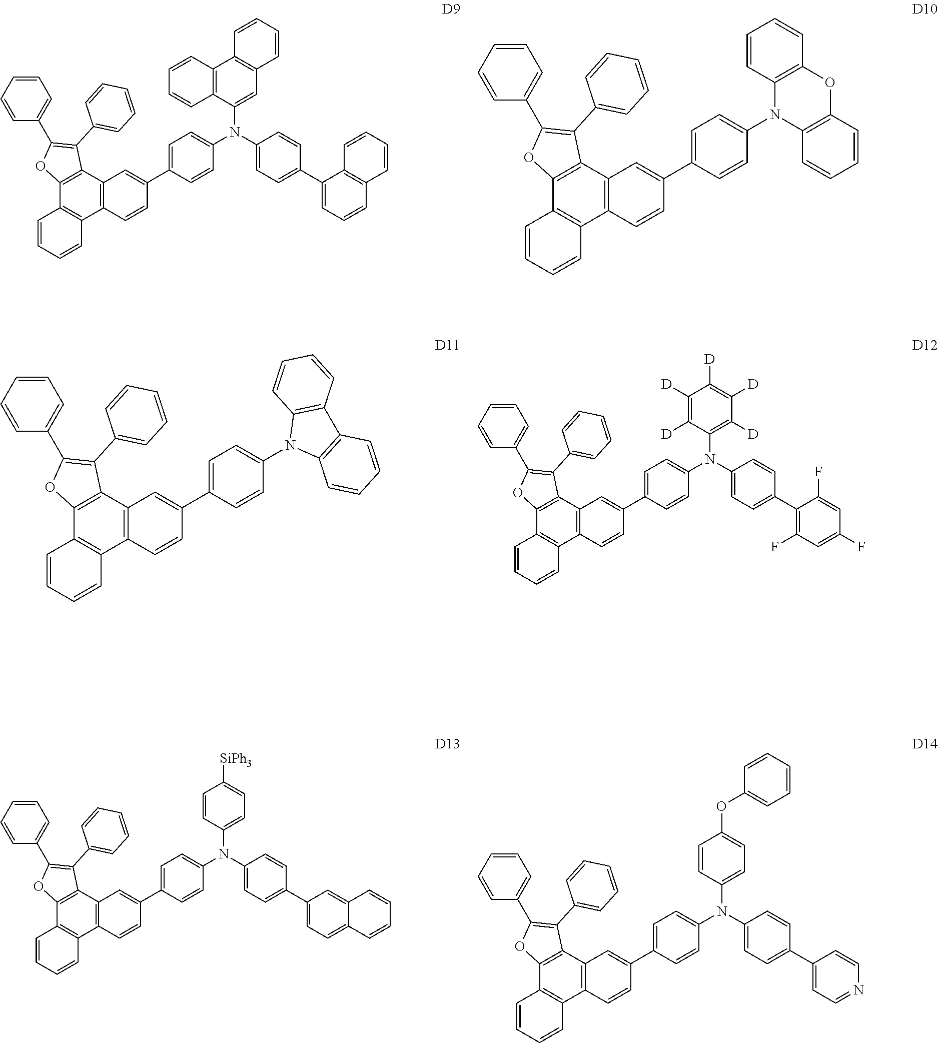

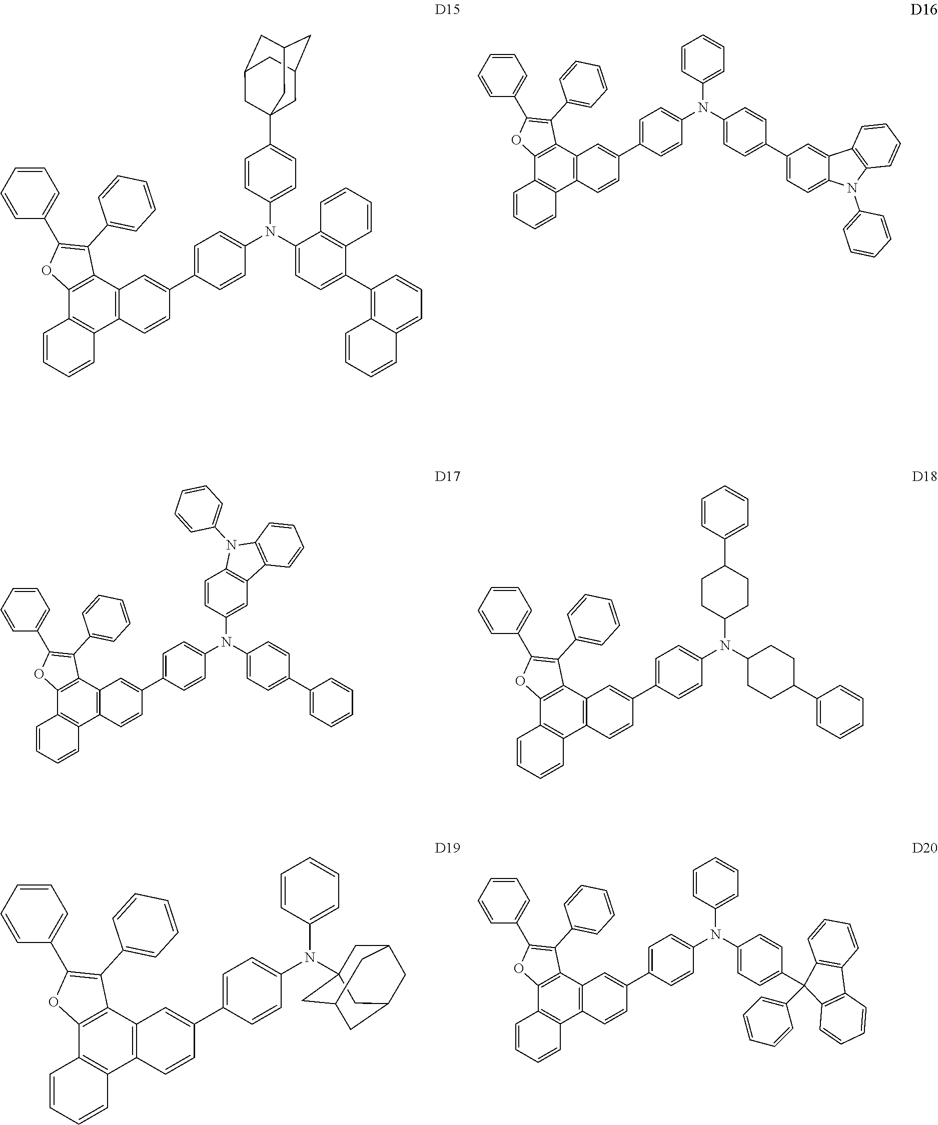

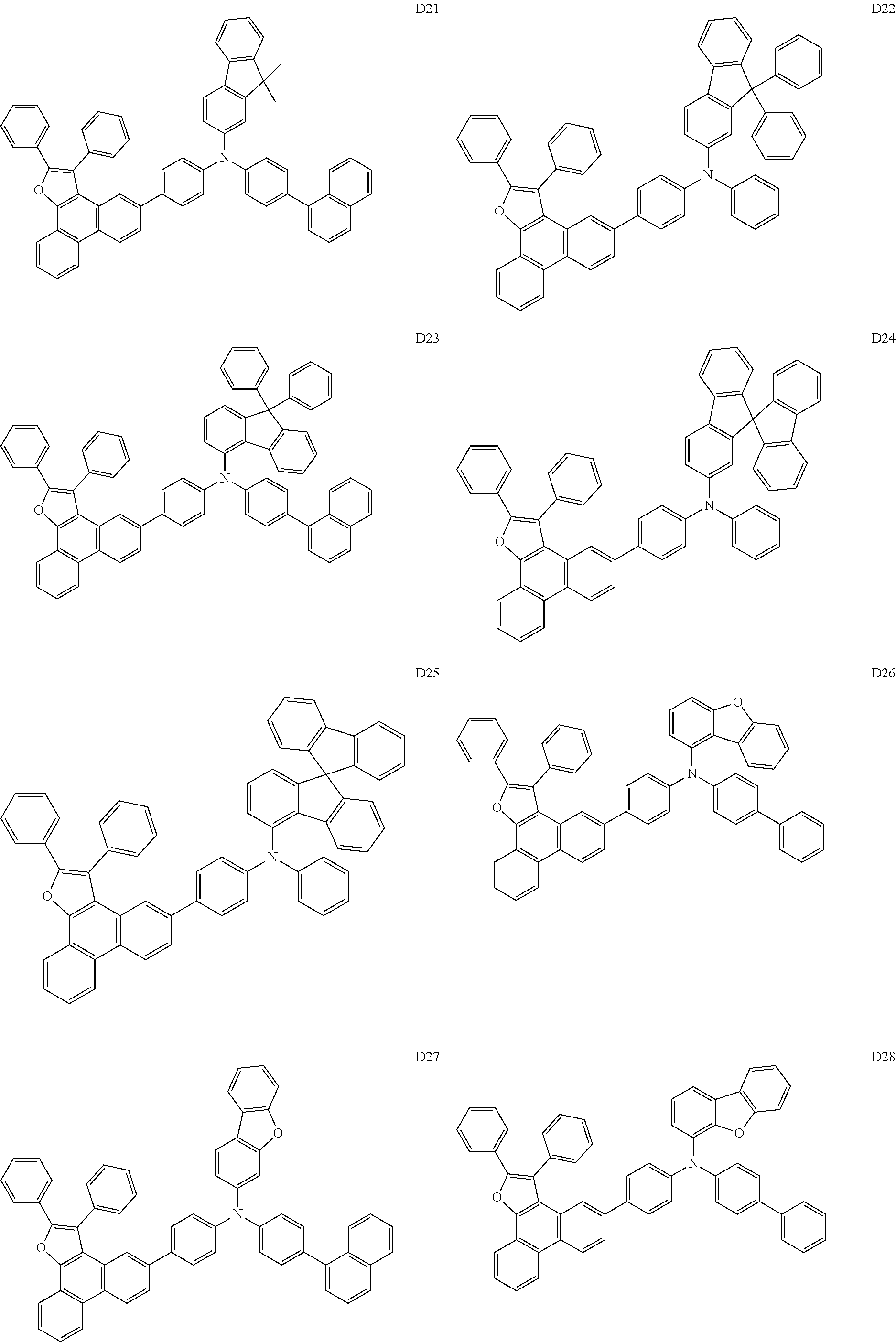

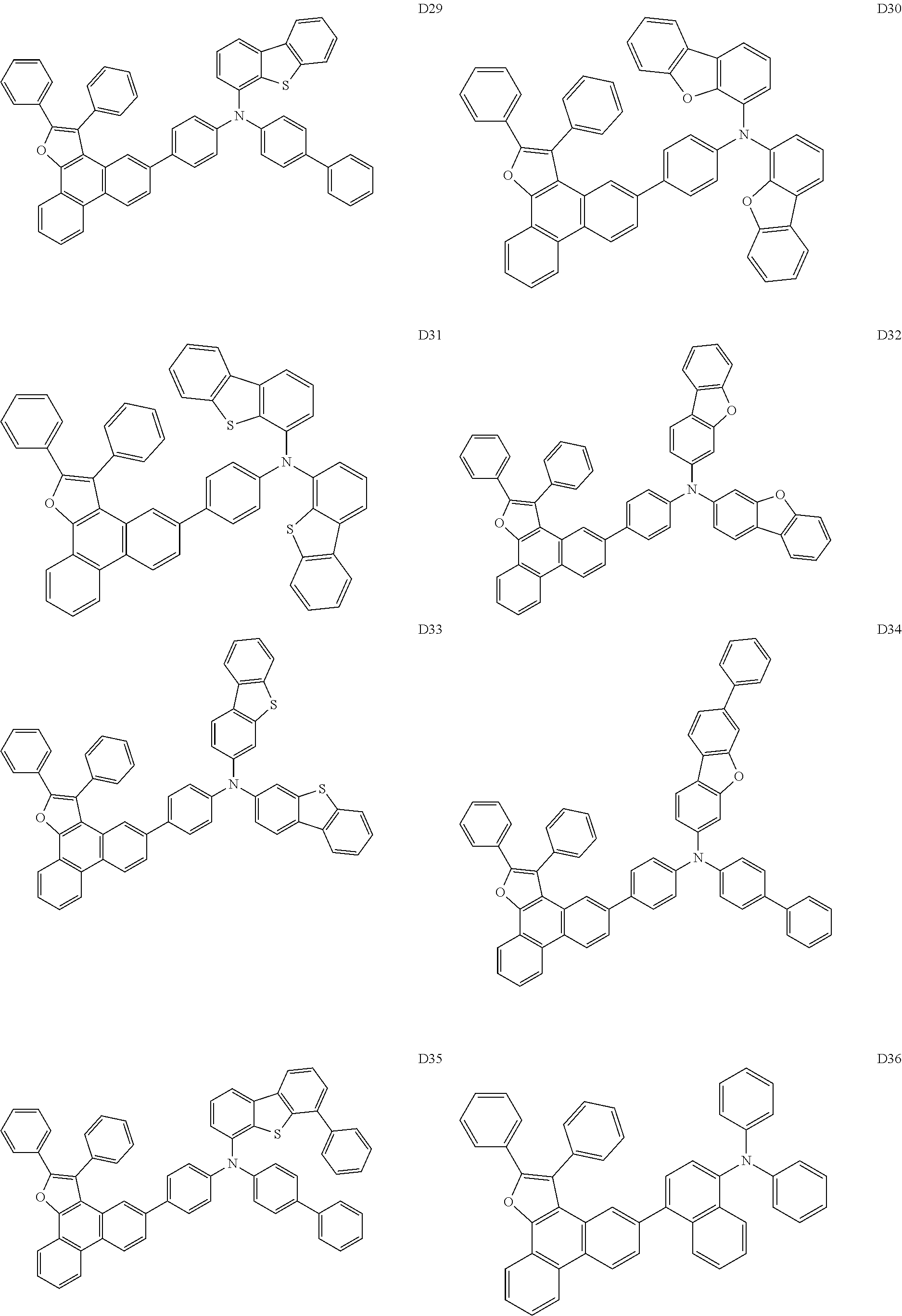

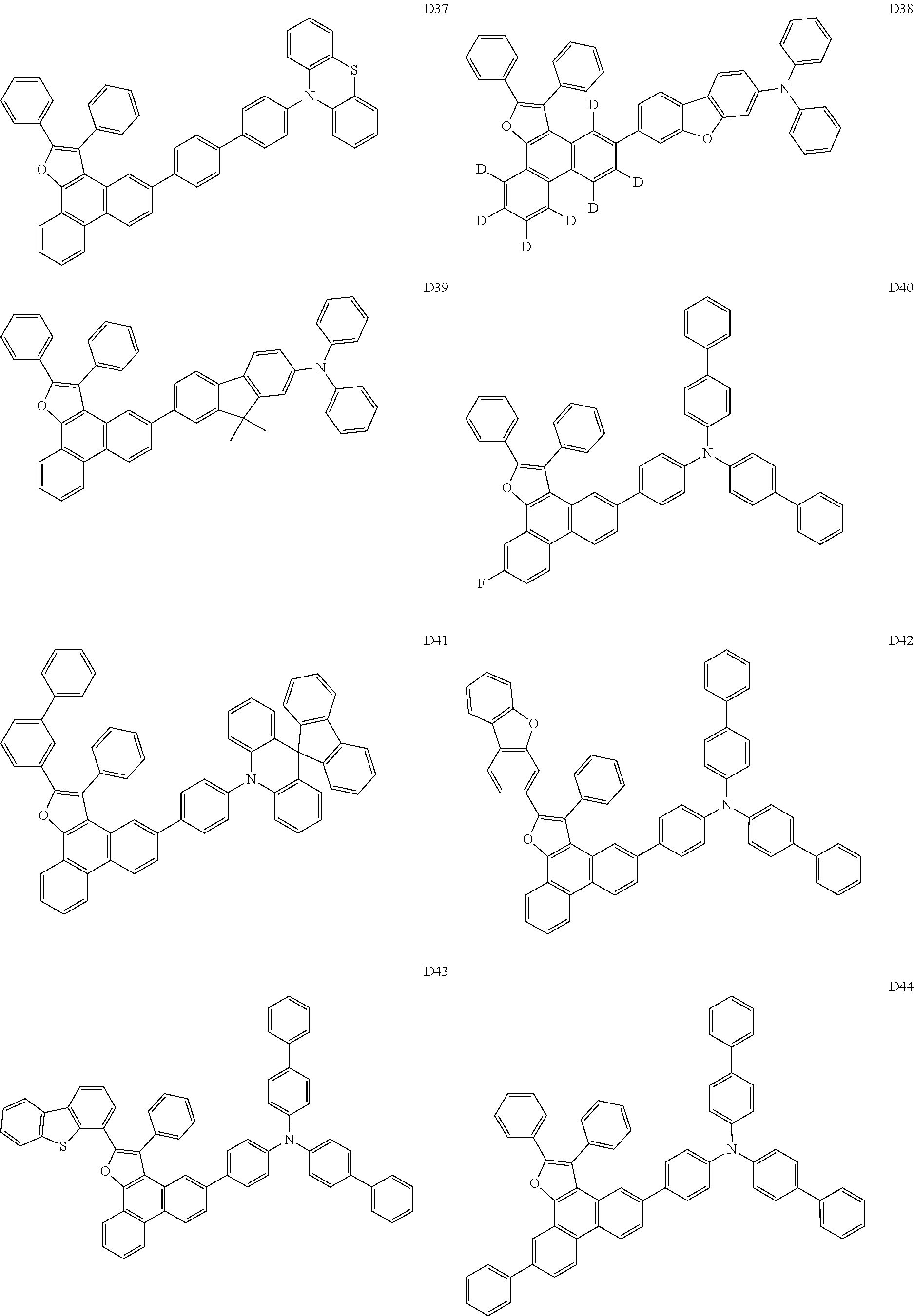

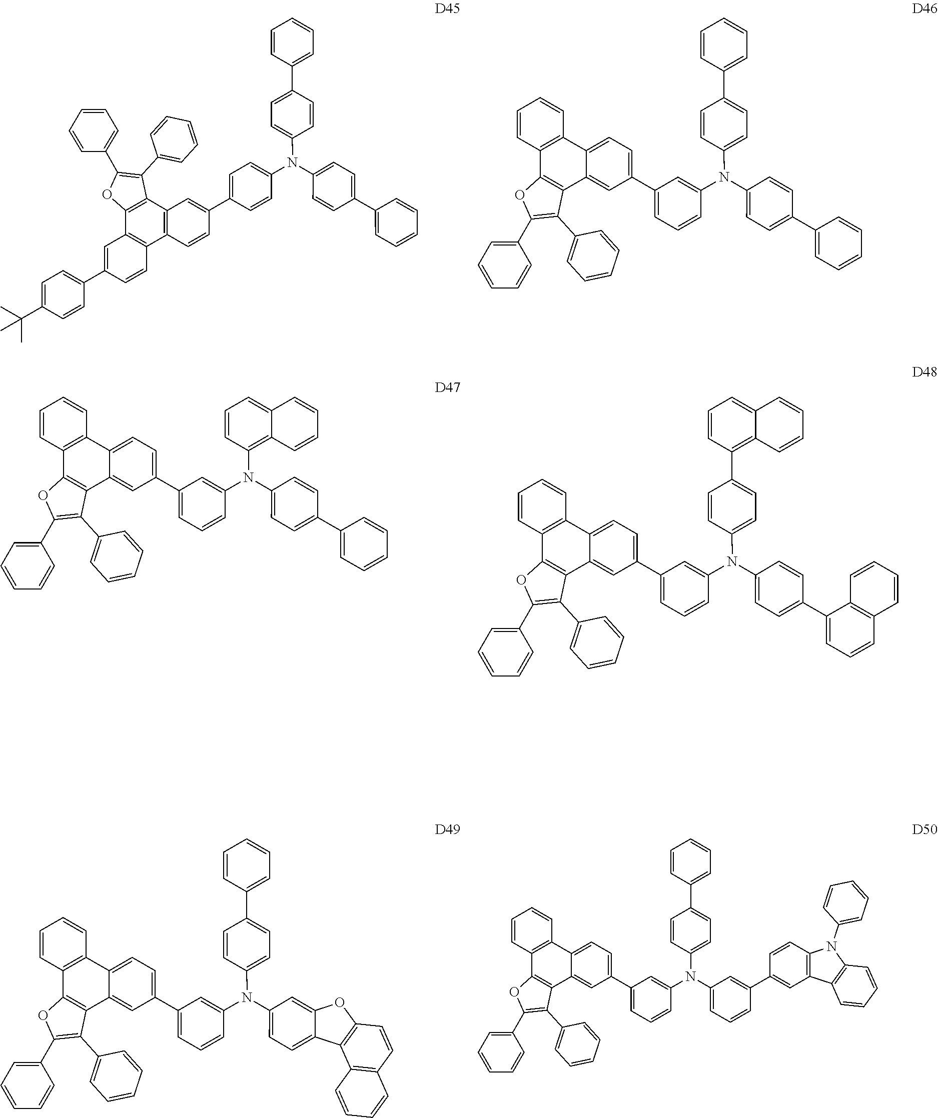

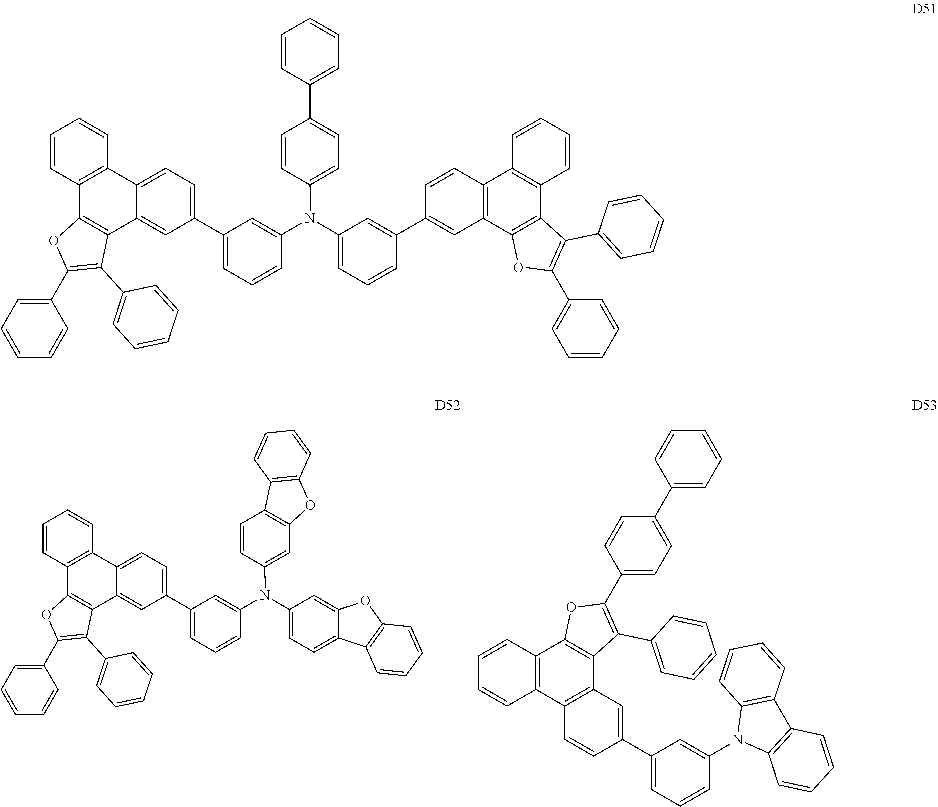 [SMILES: c1ccc(-c2ccc(N(c3cccc(-c4ccc5c6ccccc6c6c(-c7ccccc7)c(-c7ccccc7)oc6c5c4)c3)c3cccc(-c4ccc5c6ccccc6c6oc(-c7ccccc7)c(-c7ccccc7)c6c5c4)c3)cc2)cc1 ; c1ccc(-c2oc3c4ccccc4c4ccc(-c5cccc(N(c6ccc7c(c6)oc6ccccc67)c6ccc7c(c6)oc6ccccc67)c5)cc4c3c2-c2ccccc2)cc1 ; c1ccc(-c2ccc(-c3oc4c5ccccc5c5ccc(-c6cccc(-n7c8ccccc8c8ccccc87)c6)cc5c4c3-c3ccccc3)cc2)cc1]

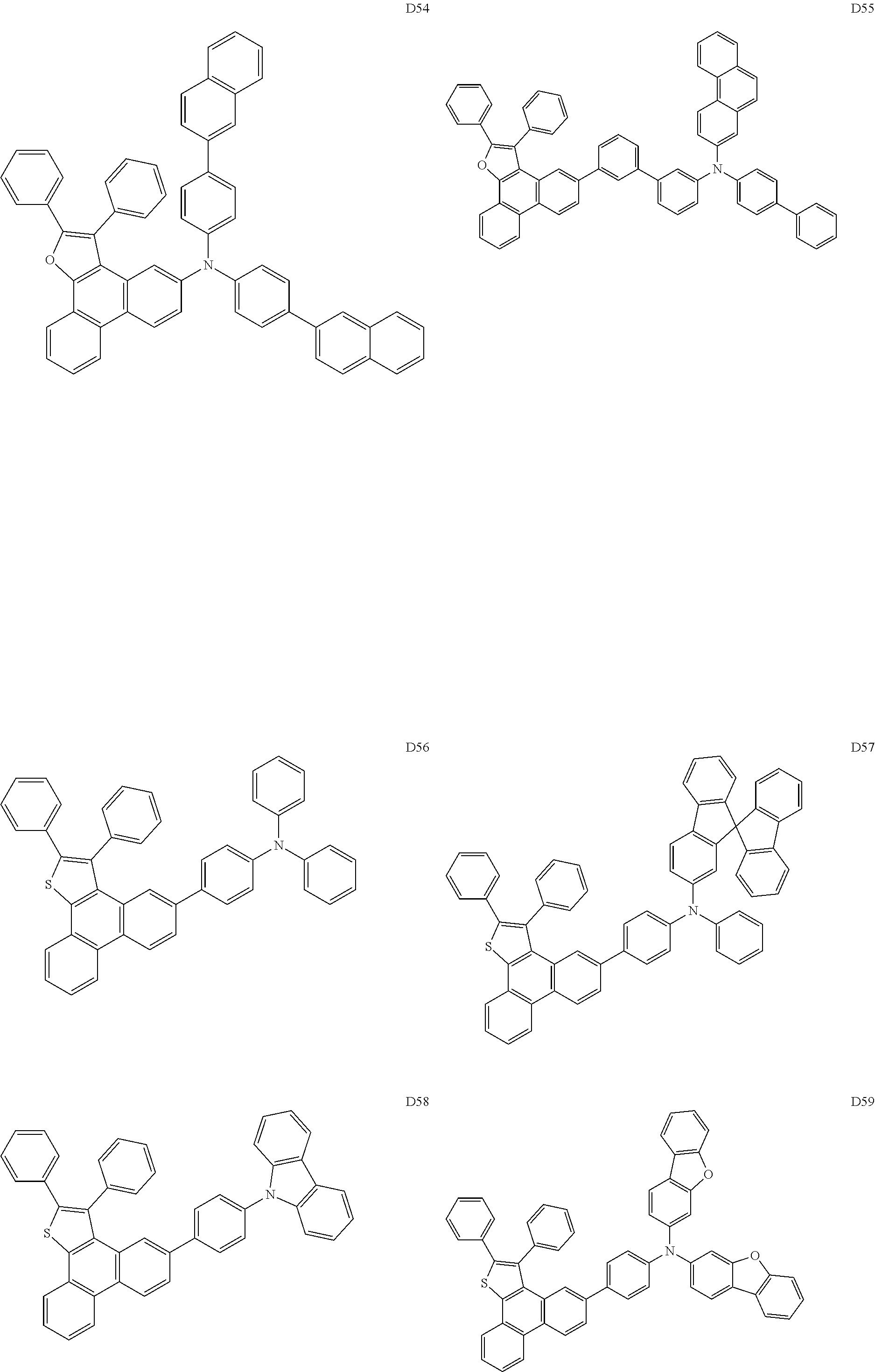

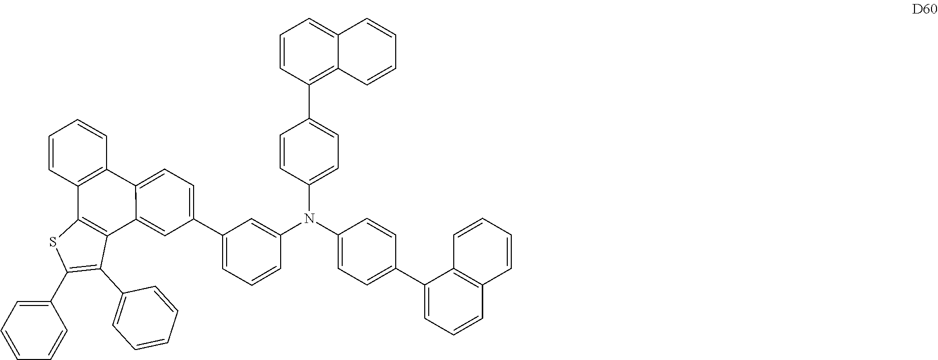 [SMILES: c1ccc(-c2sc3c4ccccc4c4ccc(-c5cccc(N(c6ccc(-c7cccc8ccccc78)cc6)c6ccc(-c7cccc8ccccc78)cc6)c5)cc4c3c2-c2ccccc2)cc1]

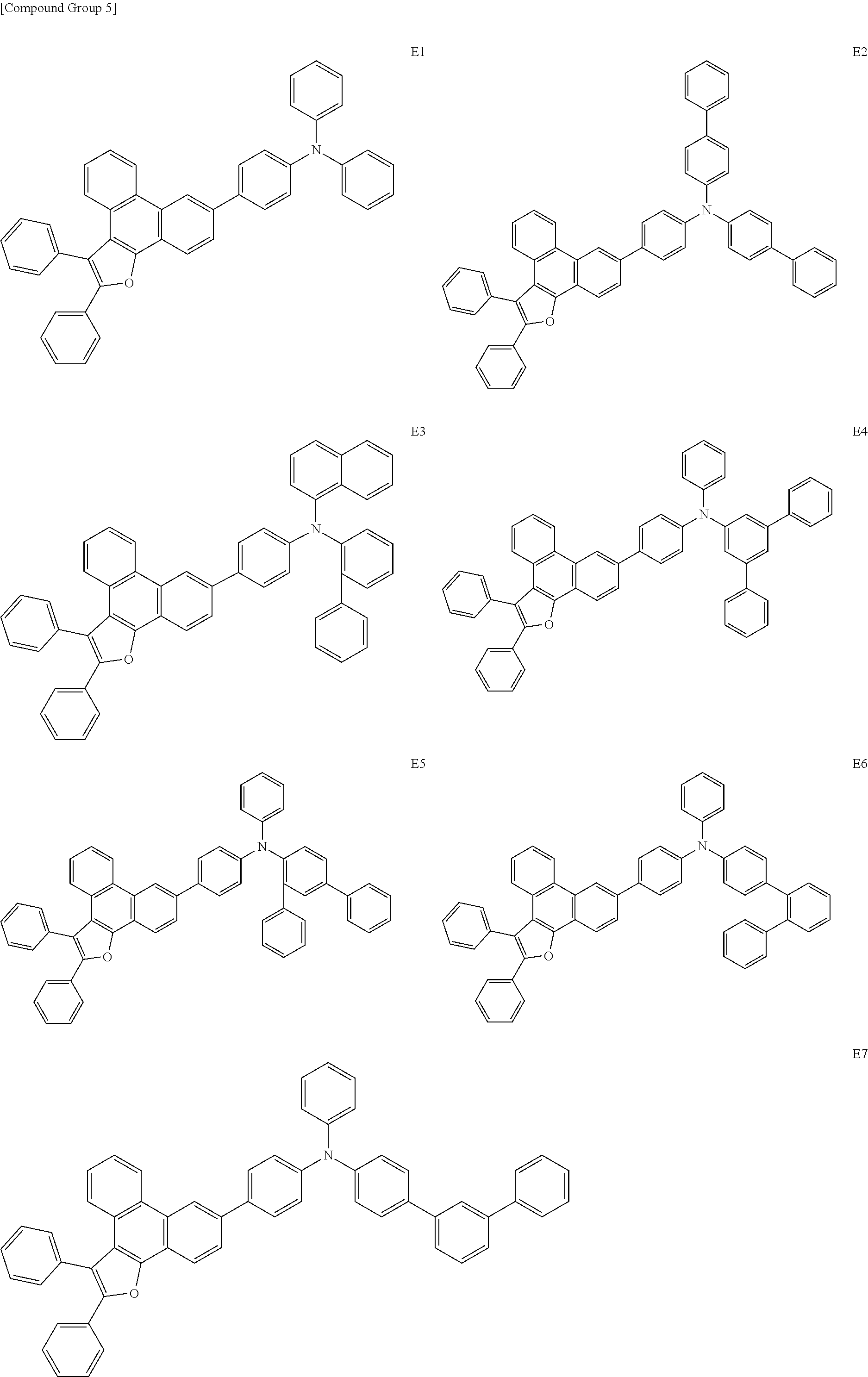

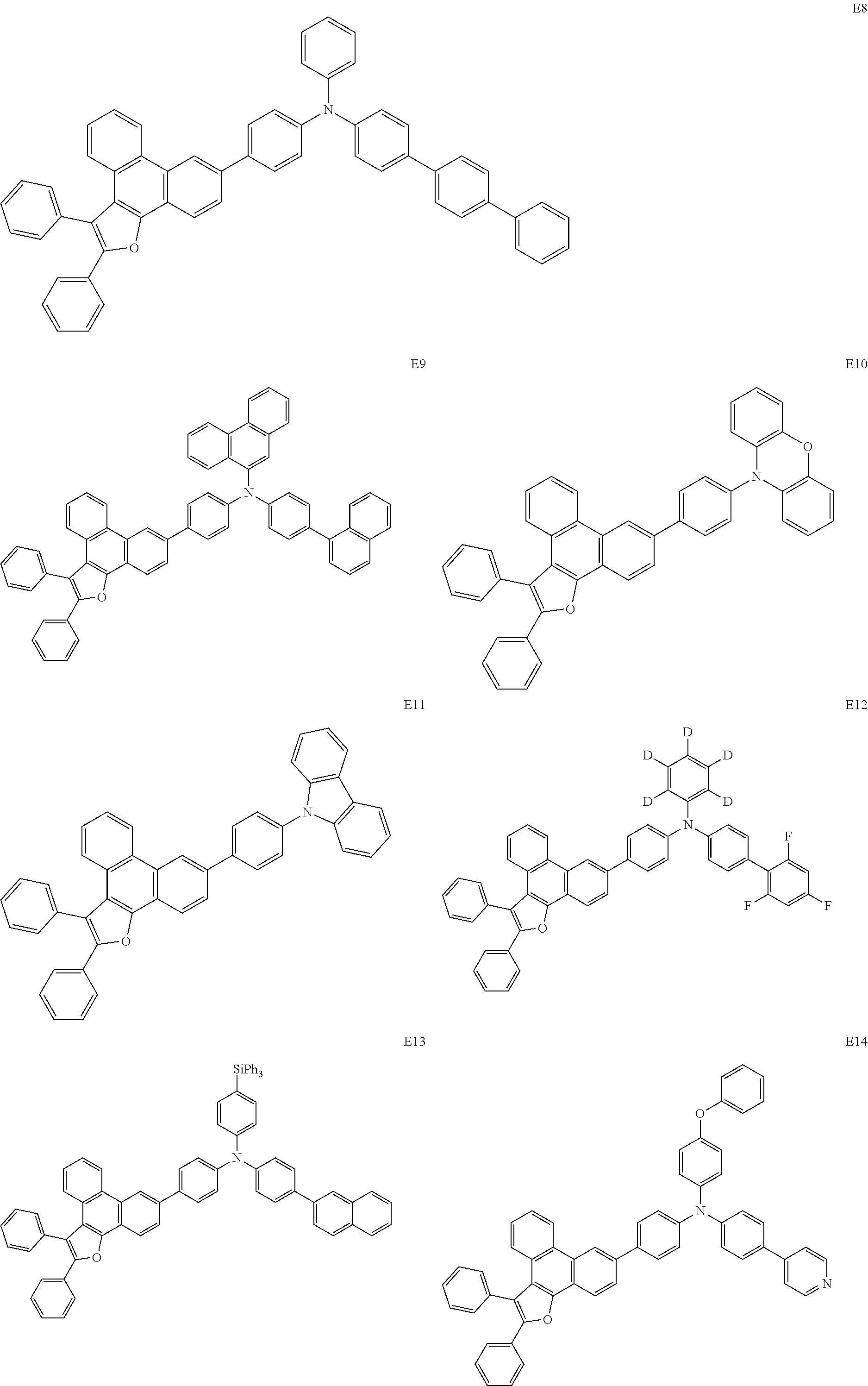

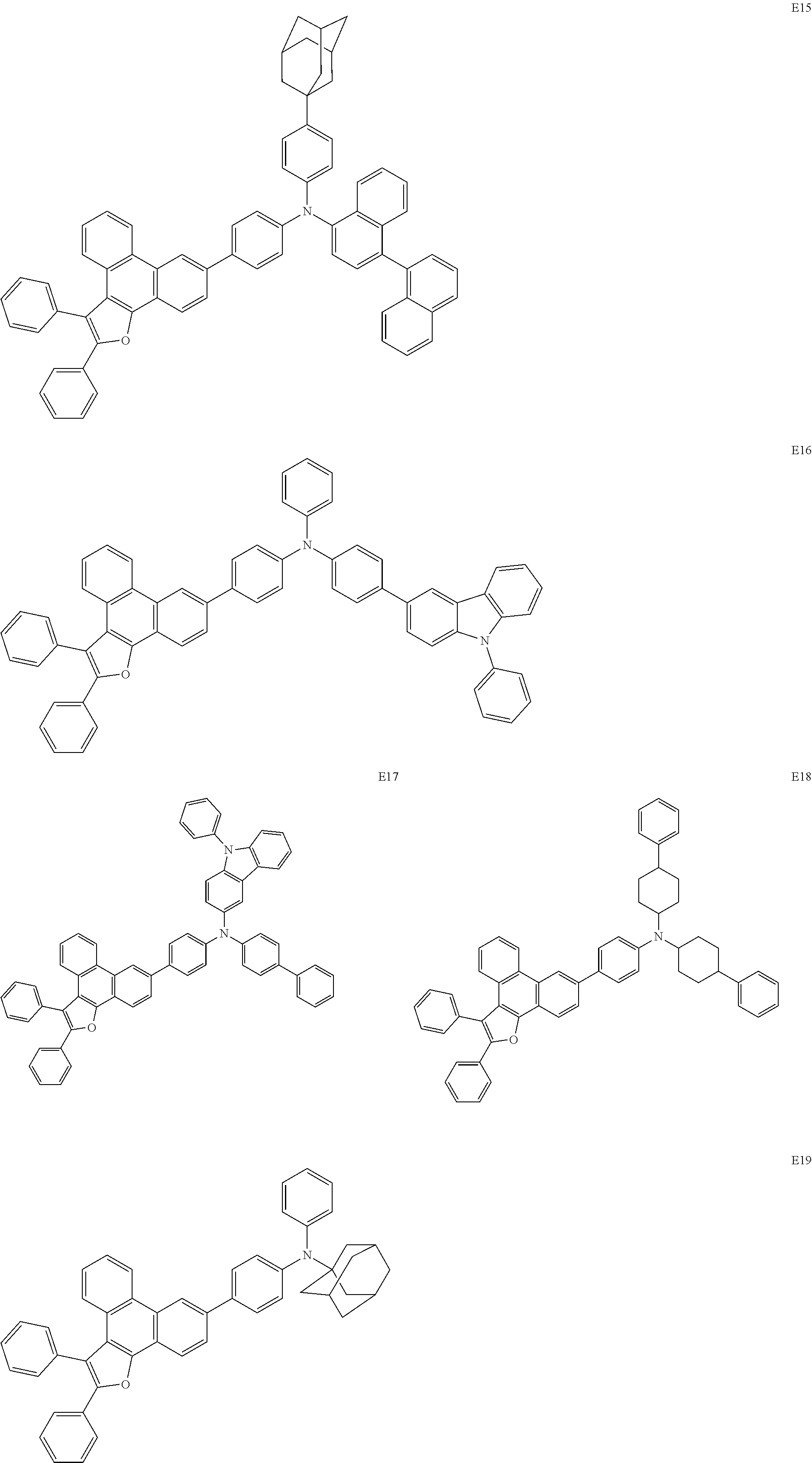

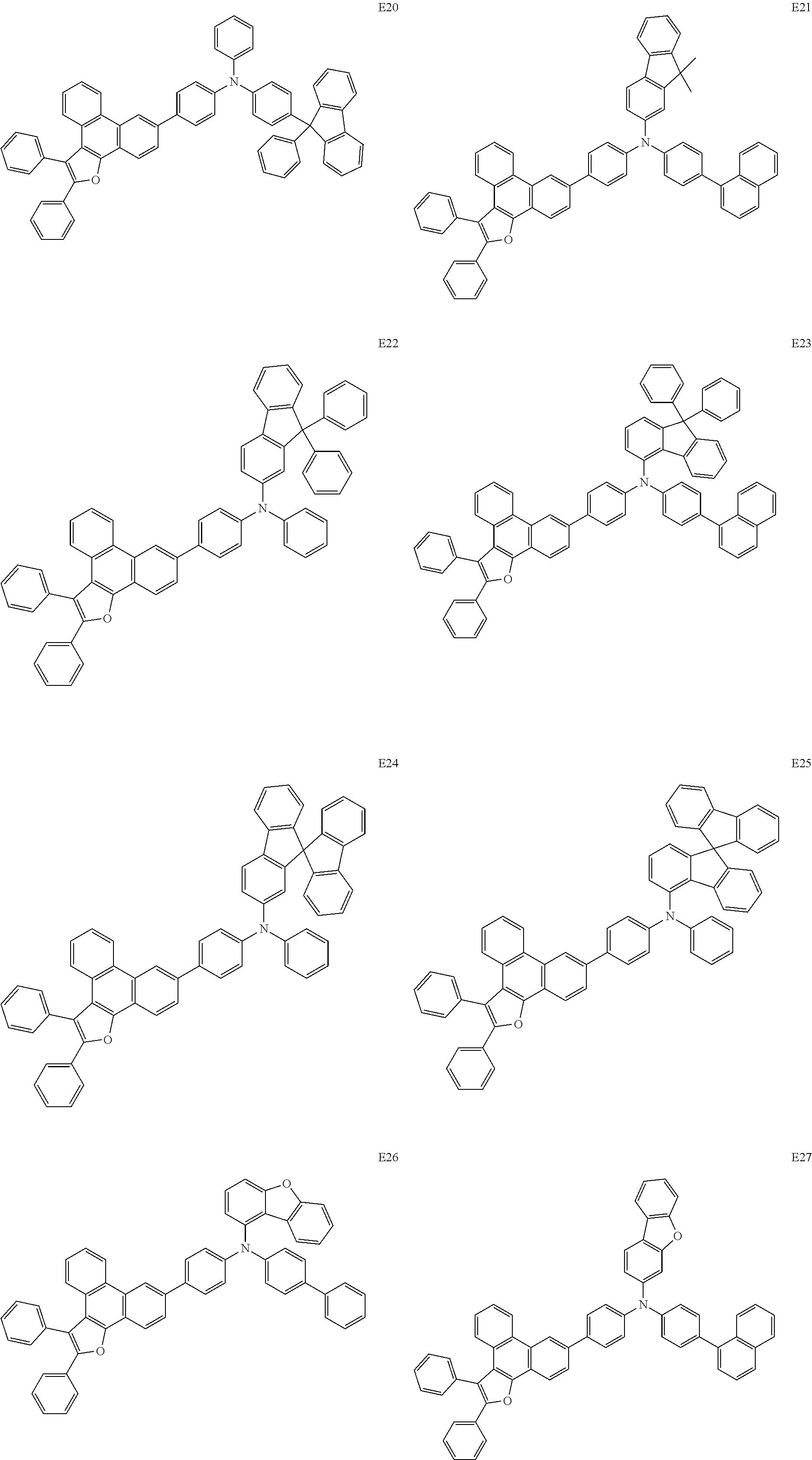

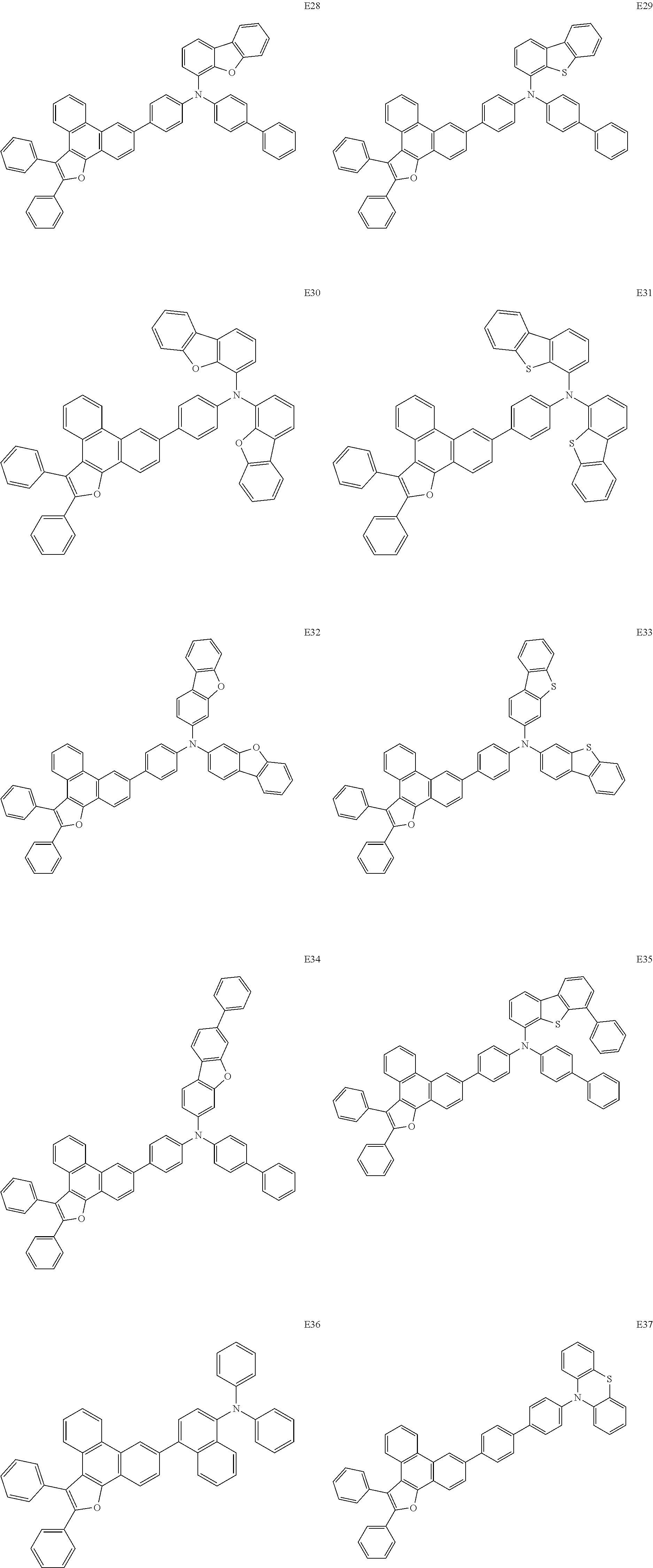

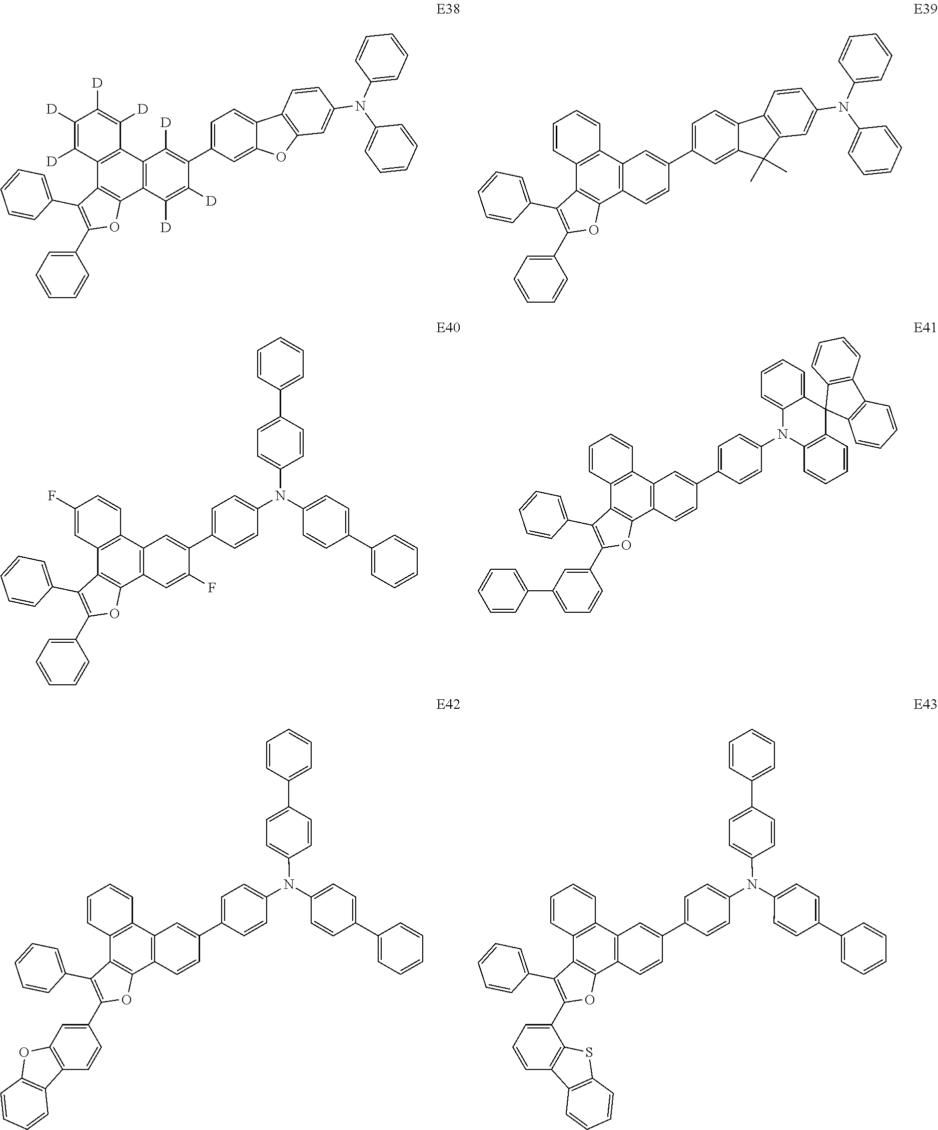

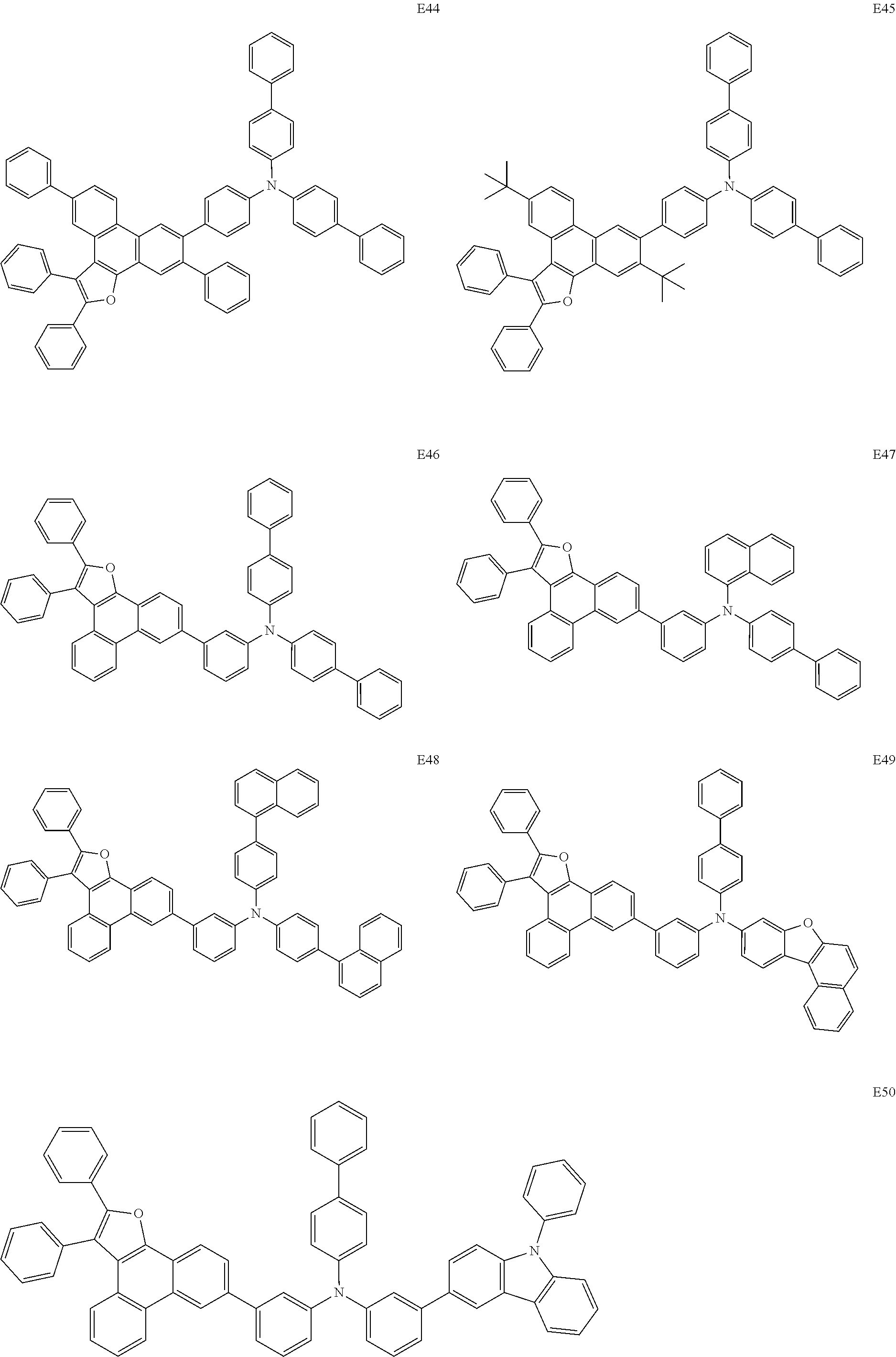

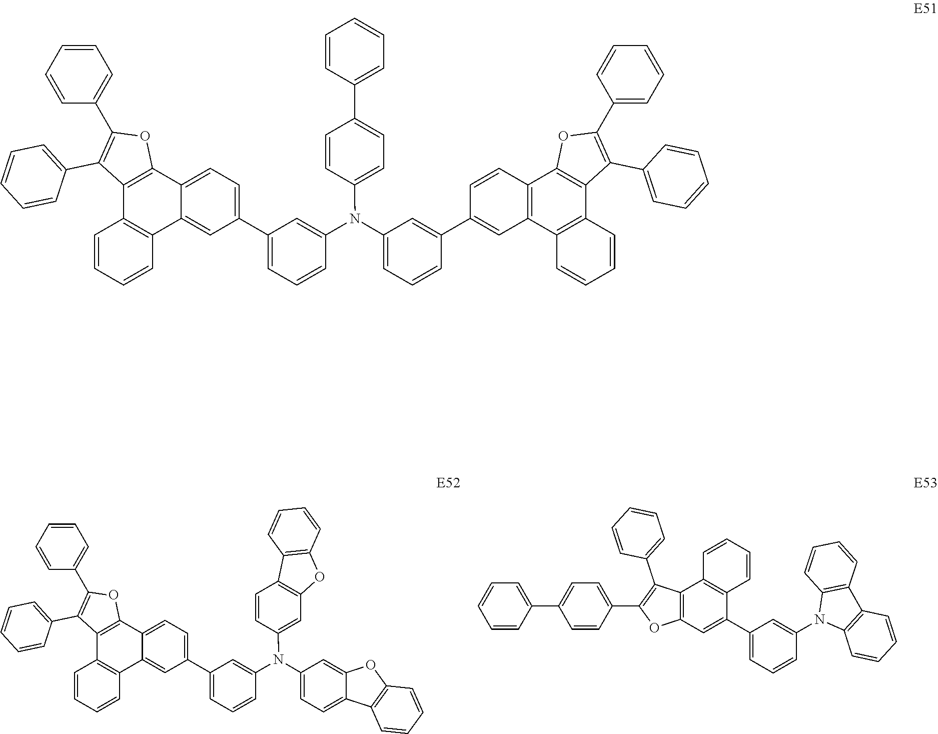 [SMILES: c1ccc(-c2ccc(N(c3cccc(-c4ccc5c(c4)c4ccccc4c4c(-c6ccccc6)c(-c6ccccc6)oc54)c3)c3cccc(-c4ccc5c(c4)c4ccccc4c4c(-c6ccccc6)c(-c6ccccc6)oc54)c3)cc2)cc1 ; c1ccc(-c2oc3c4ccc(-c5cccc(N(c6ccc7c(c6)oc6ccccc67)c6ccc7c(c6)oc6ccccc67)c5)cc4c4ccccc4c3c2-c2ccccc2)cc1 ; c1ccc(-c2ccc(-c3oc4cc(-c5cccc(-n6c7ccccc7c7ccccc76)c5)c5ccccc5c4c3-c3ccccc3)cc2)cc1]

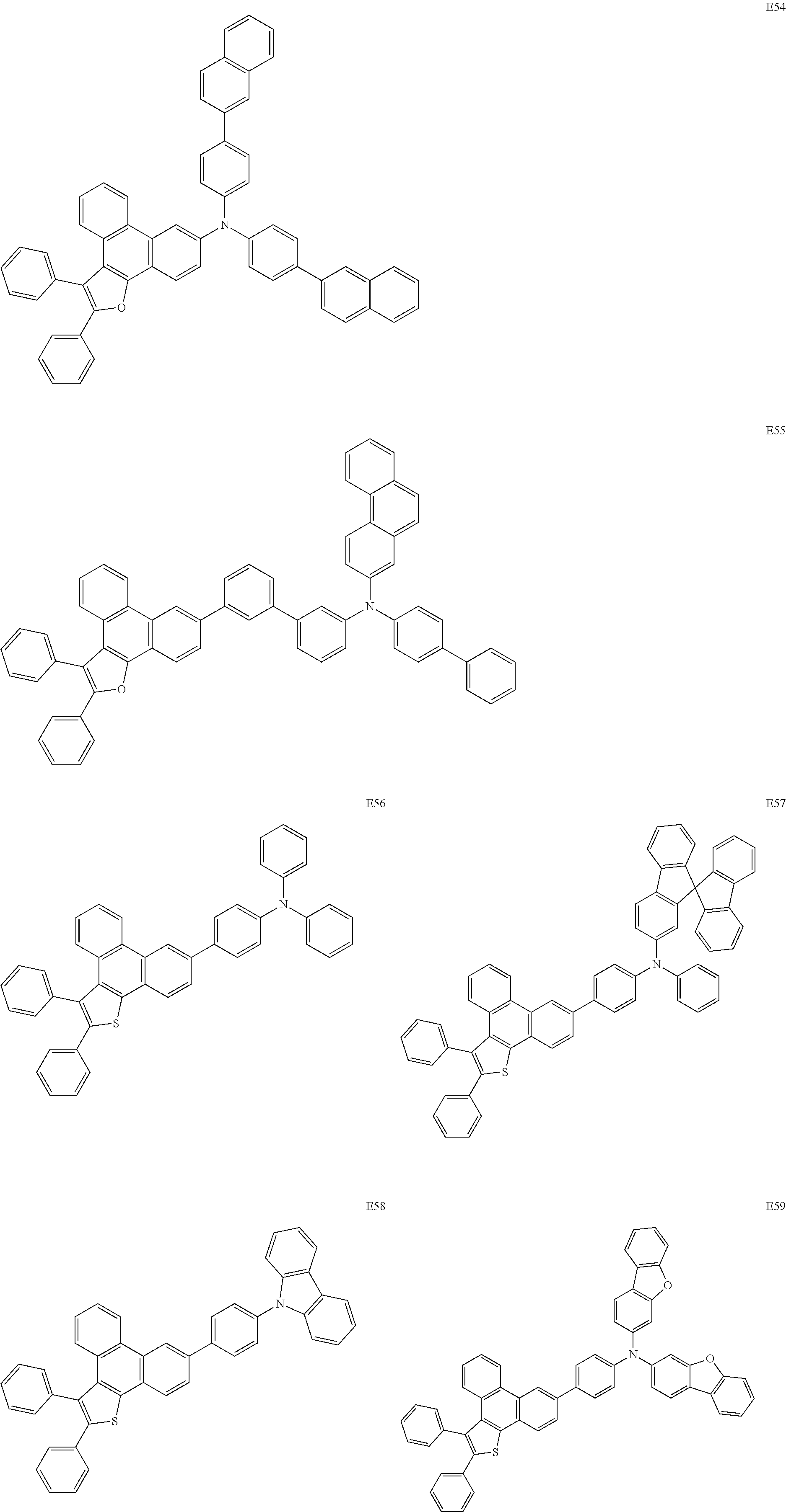

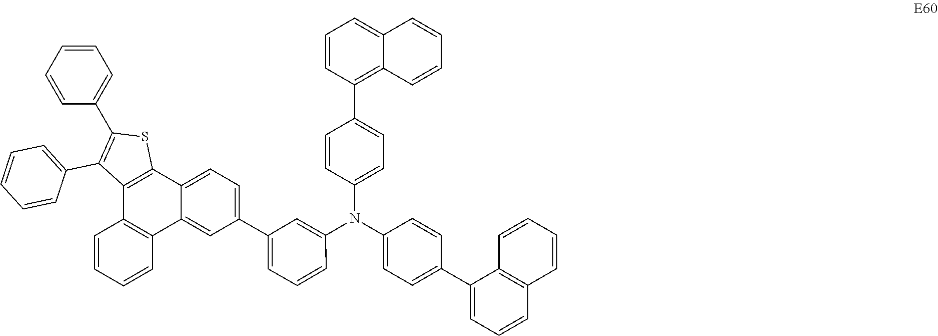 [SMILES: c1ccc(-c2sc3c4ccc(-c5cccc(N(c6ccc(-c7cccc8ccccc78)cc6)c6ccc(-c7cccc8ccccc78)cc6)c5)cc4c4ccccc4c3c2-c2ccccc2)cc1]

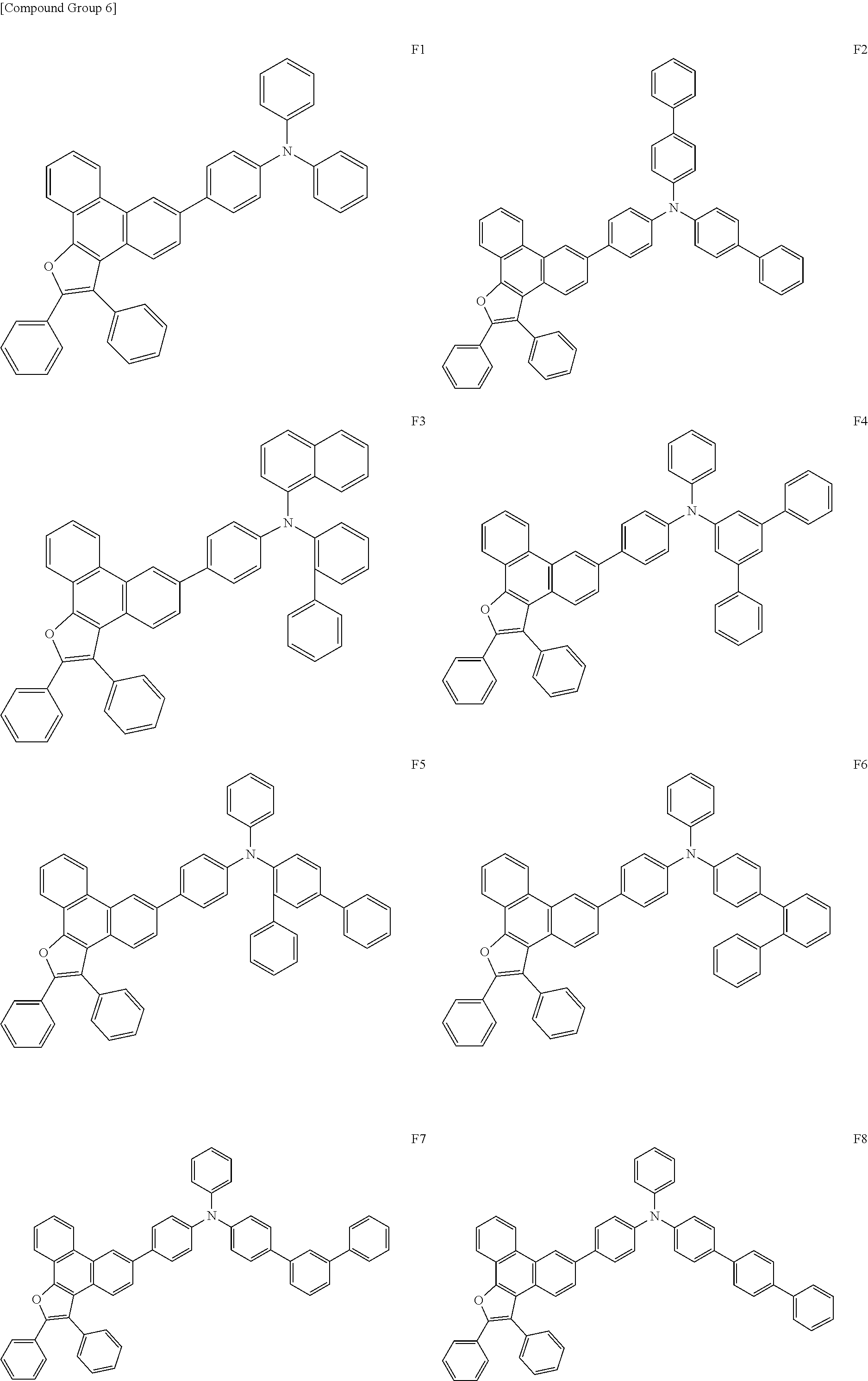

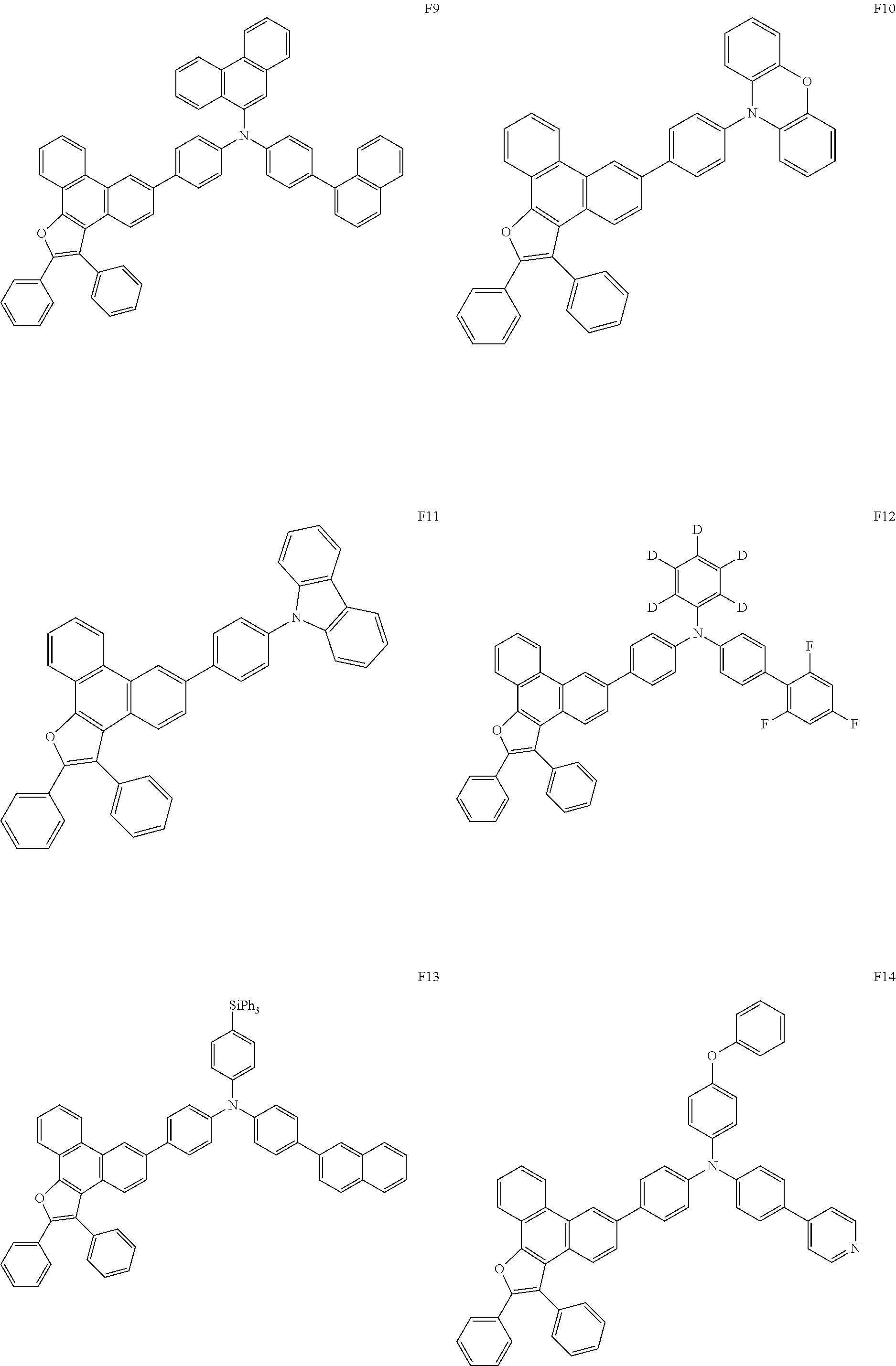

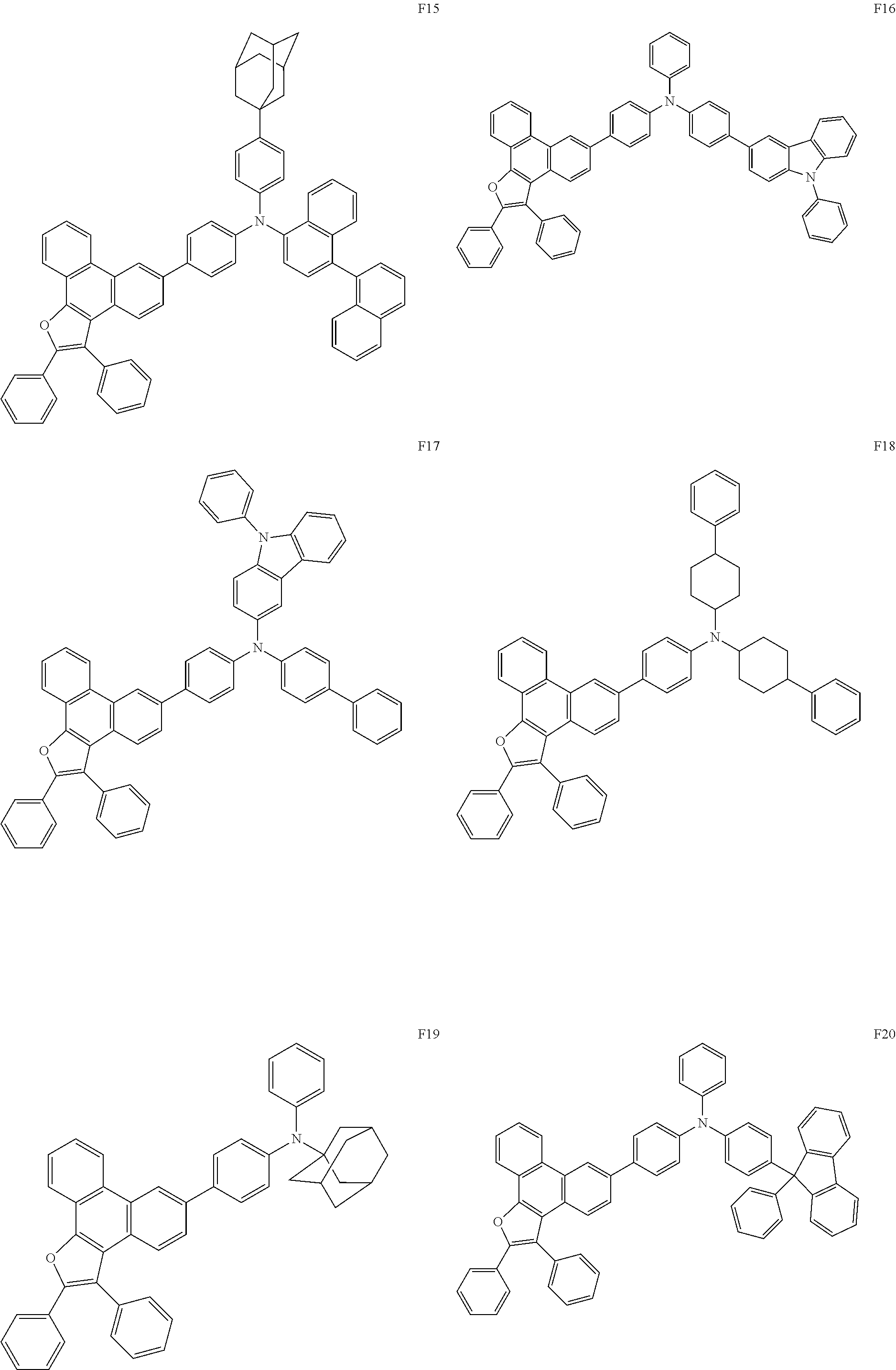

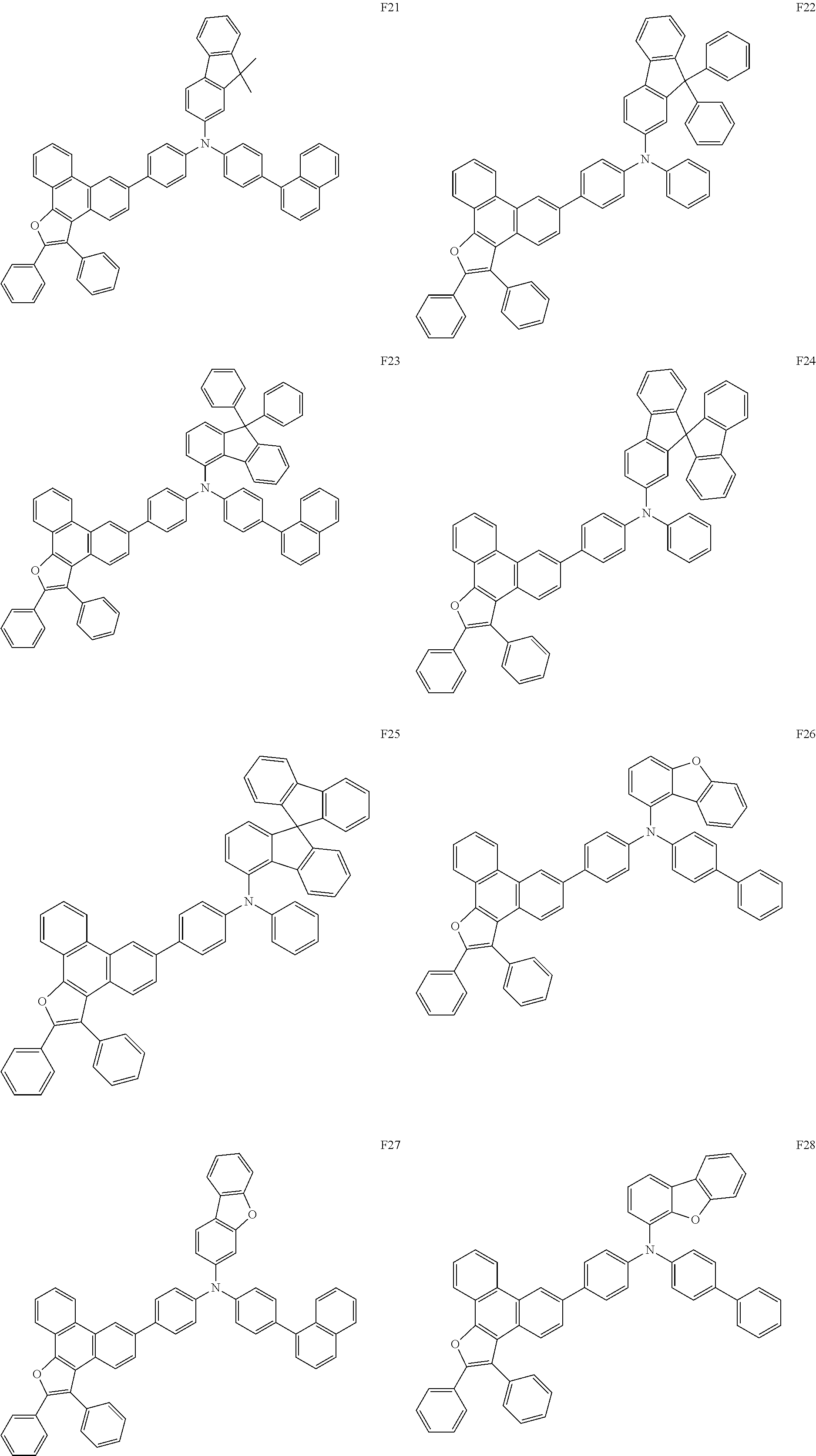

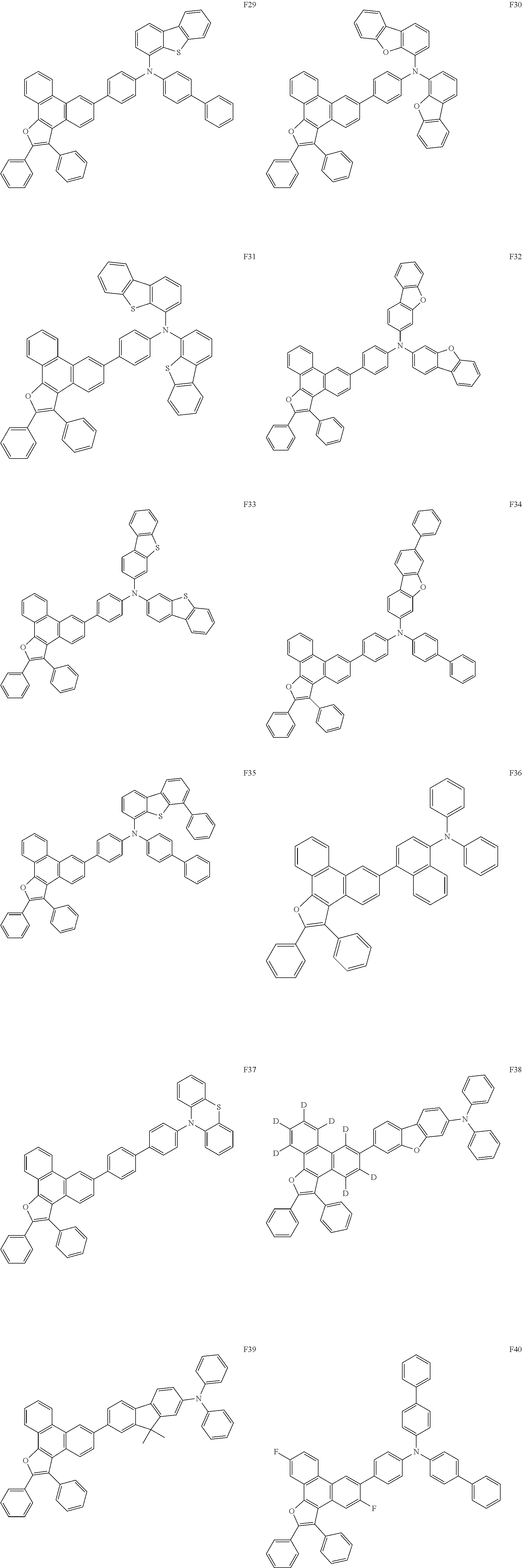

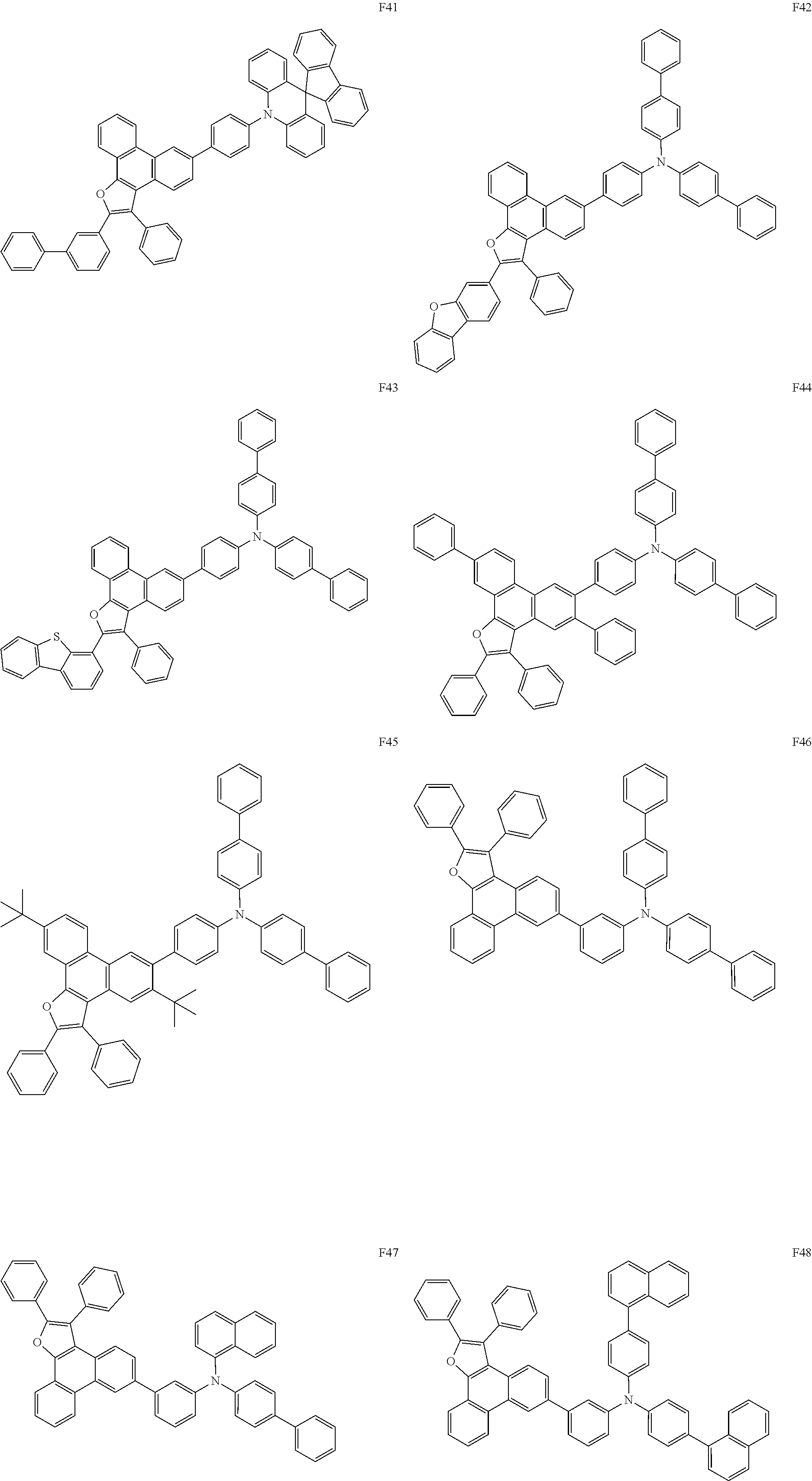

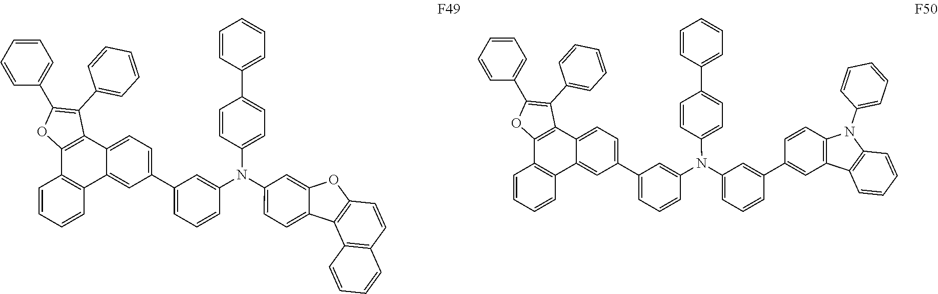 [SMILES: c1ccc(-c2ccc(N(c3cccc(-c4ccc5c(c4)c4ccccc4c4oc(-c6ccccc6)c(-c6ccccc6)c54)c3)c3ccc4c(c3)oc3ccc5ccccc5c34)cc2)cc1 ; c1ccc(-c2ccc(N(c3cccc(-c4ccc5c(c4)c4ccccc4c4oc(-c6ccccc6)c(-c6ccccc6)c54)c3)c3cccc(-c4ccc5c(c4)c4ccccc4n5-c4ccccc4)c3)cc2)cc1]

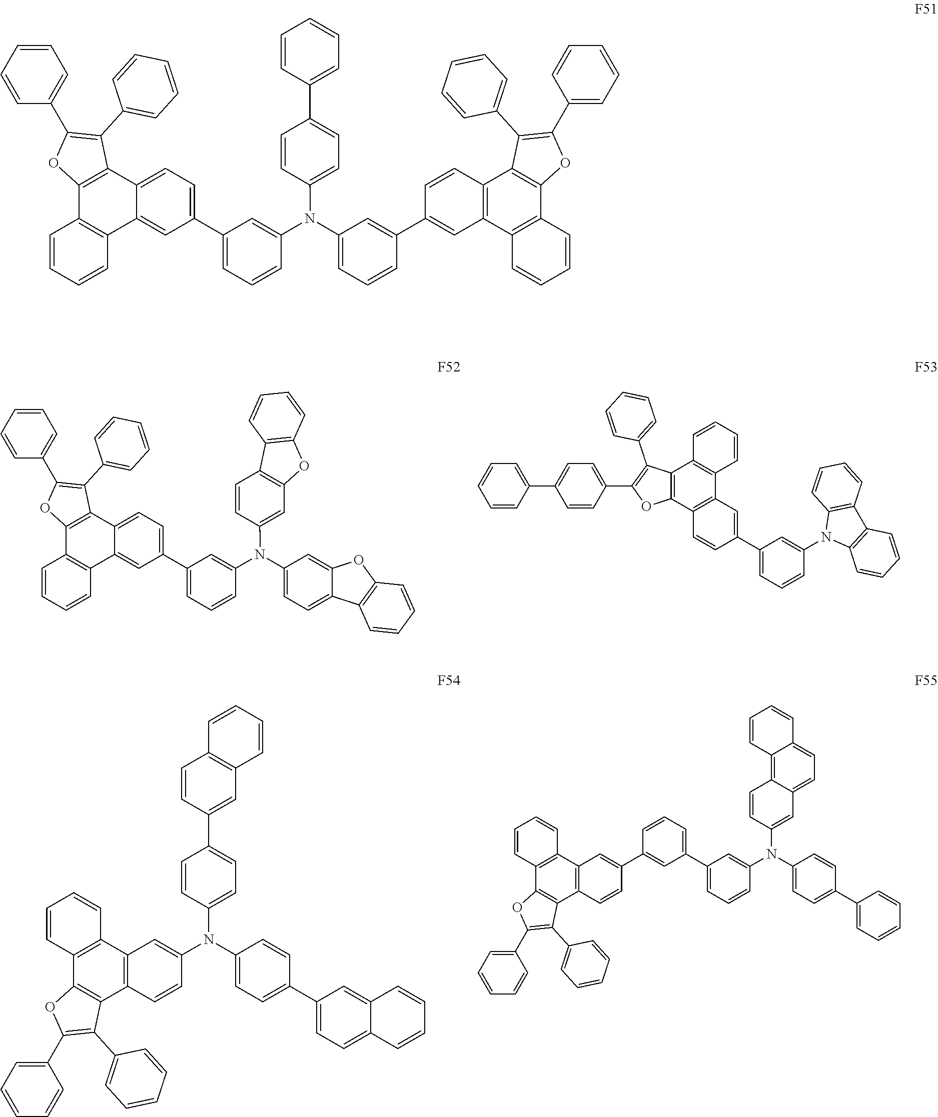

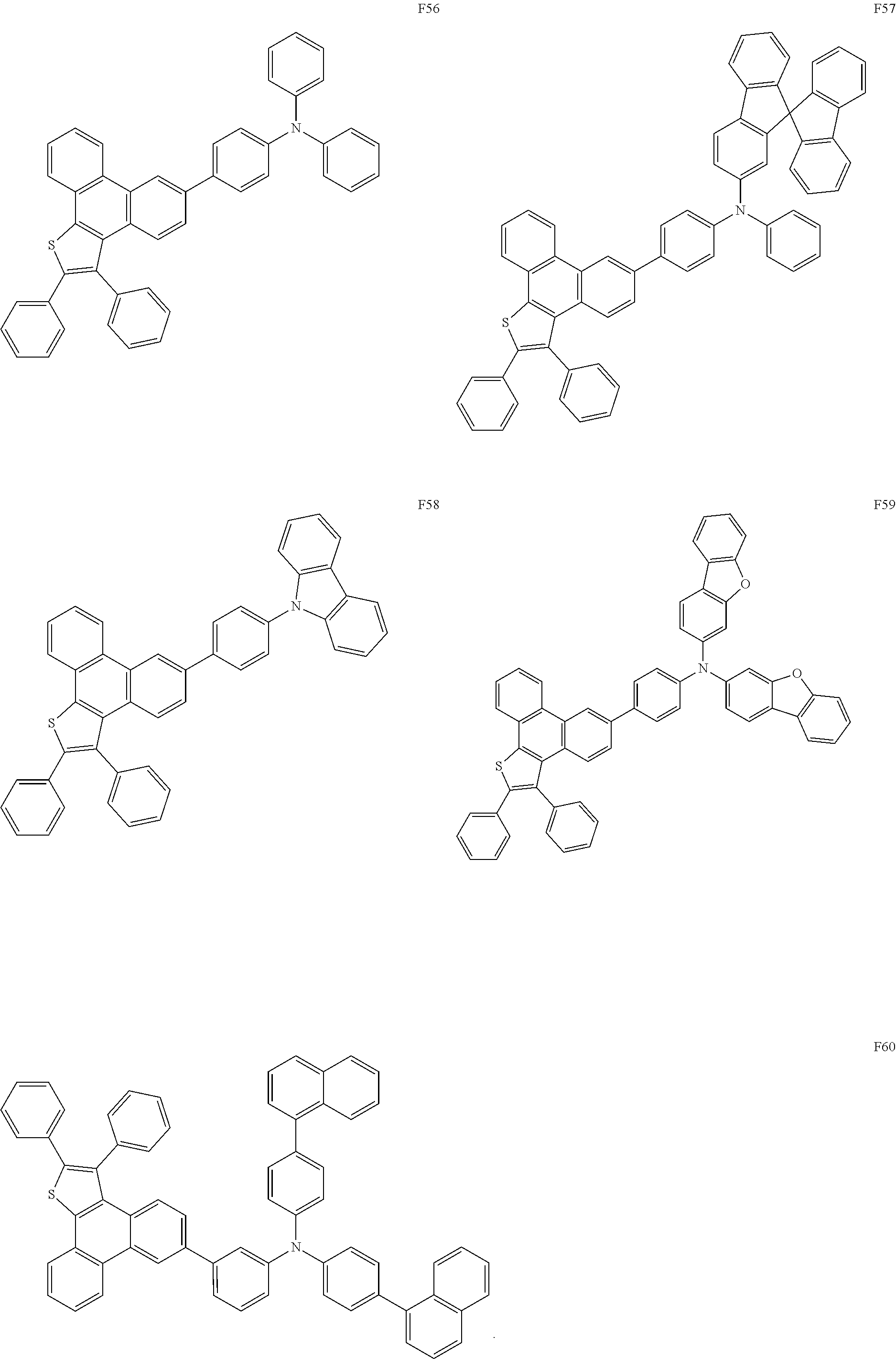 [SMILES: c1ccc(-c2sc3c4ccccc4c4cc(-c5ccc(N(c6ccccc6)c6ccccc6)cc5)ccc4c3c2-c2ccccc2)cc1 ; c1ccc(-c2sc3c4ccccc4c4cc(-c5ccc(N(c6ccccc6)c6ccc7c(c6)C6(c8ccccc8-c8ccccc86)c6ccccc6-7)cc5)ccc4c3c2-c2ccccc2)cc1 ; c1ccc(-c2sc3c4ccccc4c4cc(-c5ccc(-n6c7ccccc7c7ccccc76)cc5)ccc4c3c2-c2ccccc2)cc1 ; c1ccc(-c2sc3c4ccccc4c4cc(-c5ccc(N(c6ccc7c(c6)oc6ccccc67)c6ccc7c(c6)oc6ccccc67)cc5)ccc4c3c2-c2ccccc2)cc1 ; c1ccc(-c2sc3c4ccccc4c4cc(-c5cccc(N(c6ccc(-c7cccc8ccccc78)cc6)c6ccc(-c7cccc8ccccc78)cc6)c5)ccc4c3c2-c2ccccc2)cc1]

11. A polycyclic compound represented by Formula 5 or Formula 6: 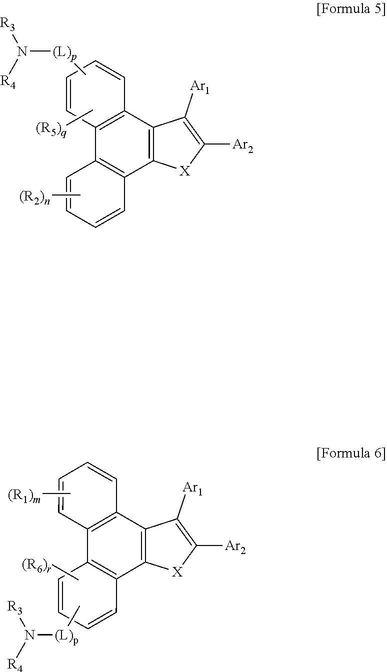
wherein in Formula 5 and Formula 6, X is O or S,
Ar₁ and Ar₂ are each independently a substituted or unsubstituted aryl group of 6 to 30 carbon atoms for forming a ring, or a substituted or unsubstituted heteroaryl group of 2 to 30 carbon atoms for forming a ring,
R₁ and R₂ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring,
“m” and “n” are each independently an integer of 0 to 4,
is R₅ and R₆ are each independently a hydrogen atom, a deuterium atom, a halogen atom, a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring,
“q” and “r” are each independently an integer of 0 to 3,
L is a direct linkage, a substituted or unsubstituted arylene group of 6 to 30 carbon atoms for forming a ring, or a substituted or unsubstituted heteroarylene group of 2 to 30 carbon atoms for forming a ring,
“p” is an integer of 0 to 3, and
R₃ and R₄ are each independently a substituted or unsubstituted alkyl group of 1 to 20 carbon atoms, a substituted or unsubstituted aryl group of 6 to 30 carbon atoms for forming a ring, or a substituted or unsubstituted heteroaryl group of 2 to 30 carbon atoms for forming a ring, or combined with an adjacent group to form a ring.

12. The polycyclic compound of claim 11, wherein L is a substituted or unsubstituted arylene group of 6 to 12 carbon atoms for forming a ring.

13. The polycyclic compound of claim 11, wherein X is O.

14. The polycyclic compound of claim 11, wherein the polycyclic compound represented by Formula 5 and Formula 6 is any one selected among compounds represented in Compound Group 3 to Compound Group 6: 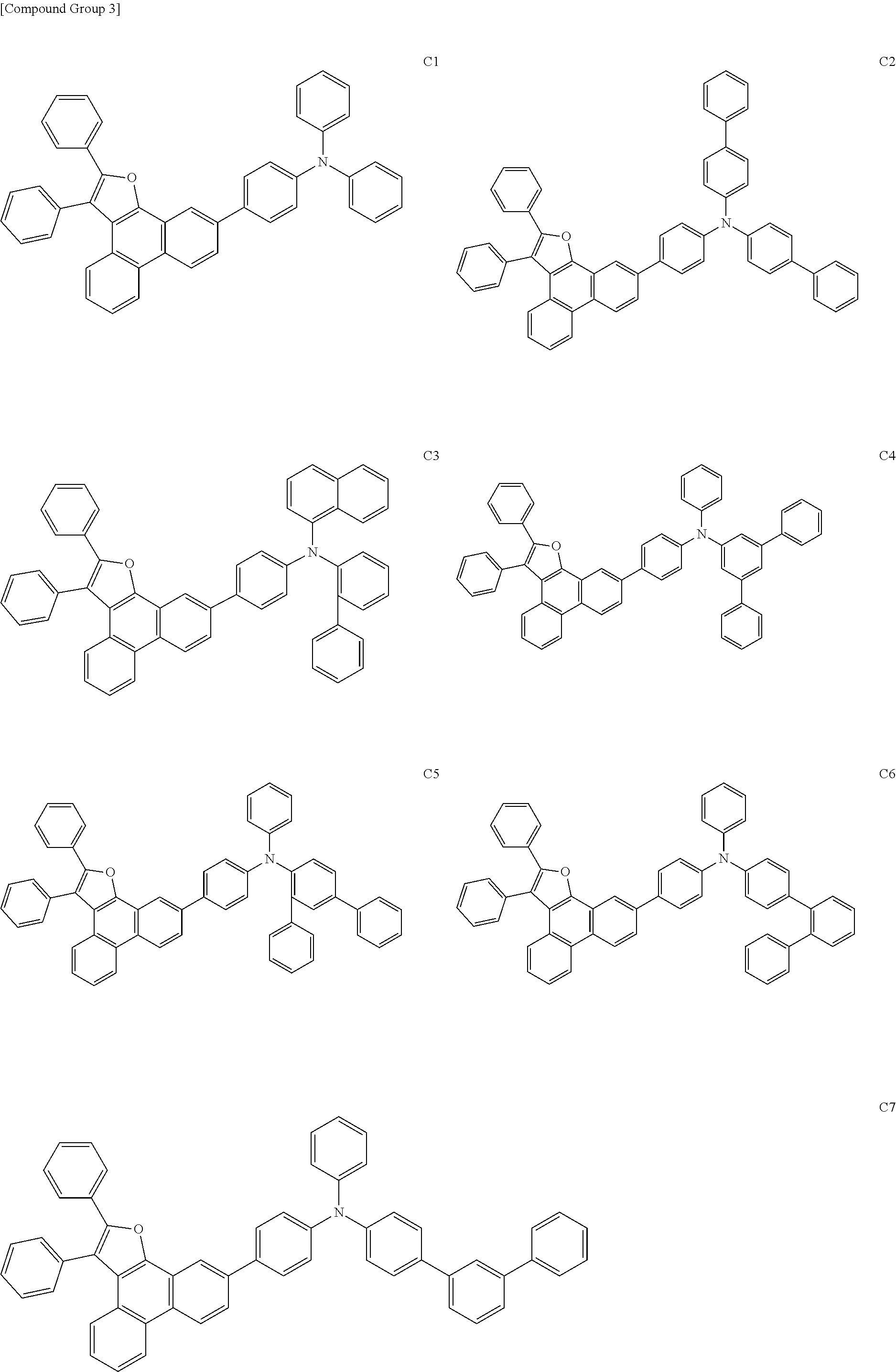 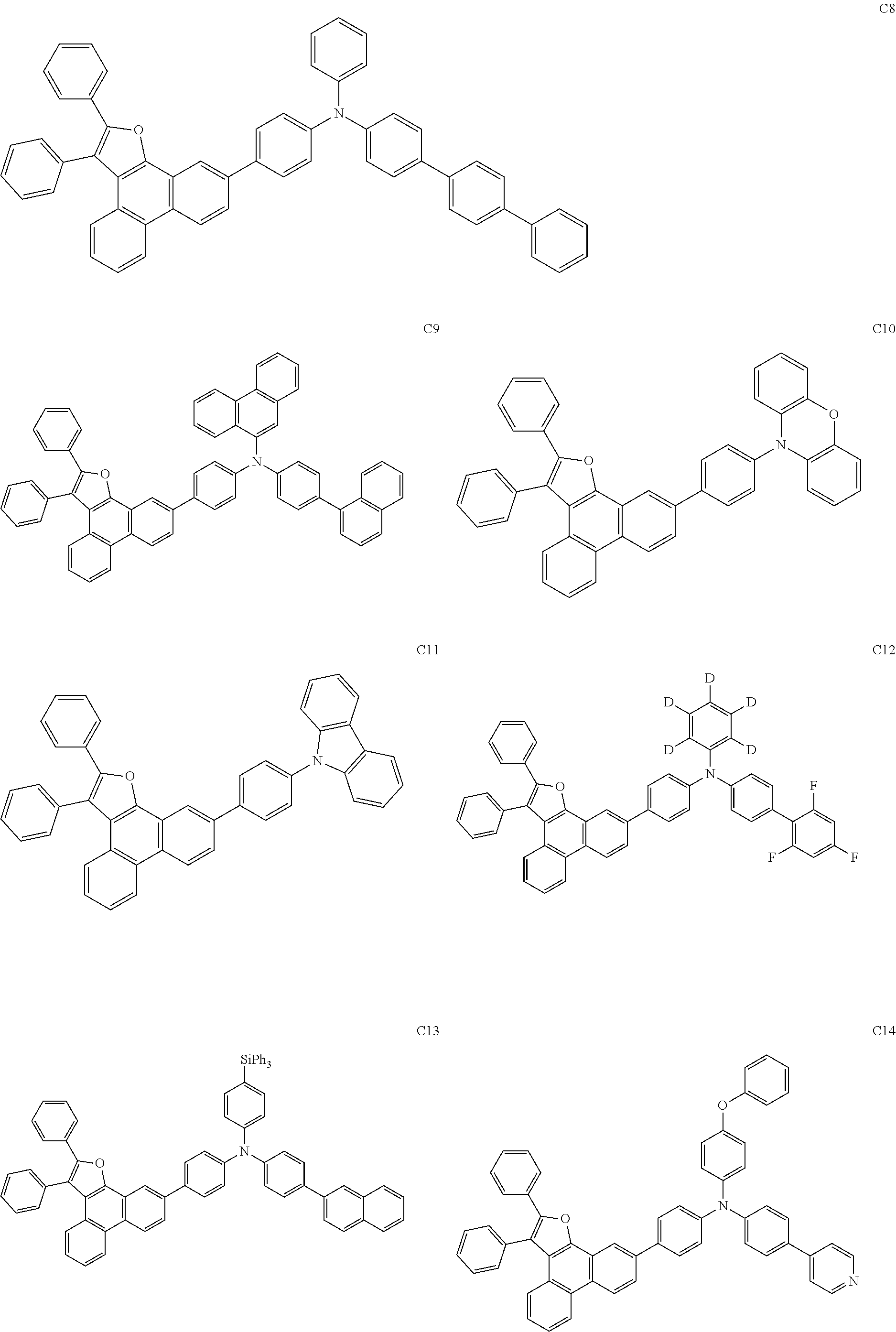 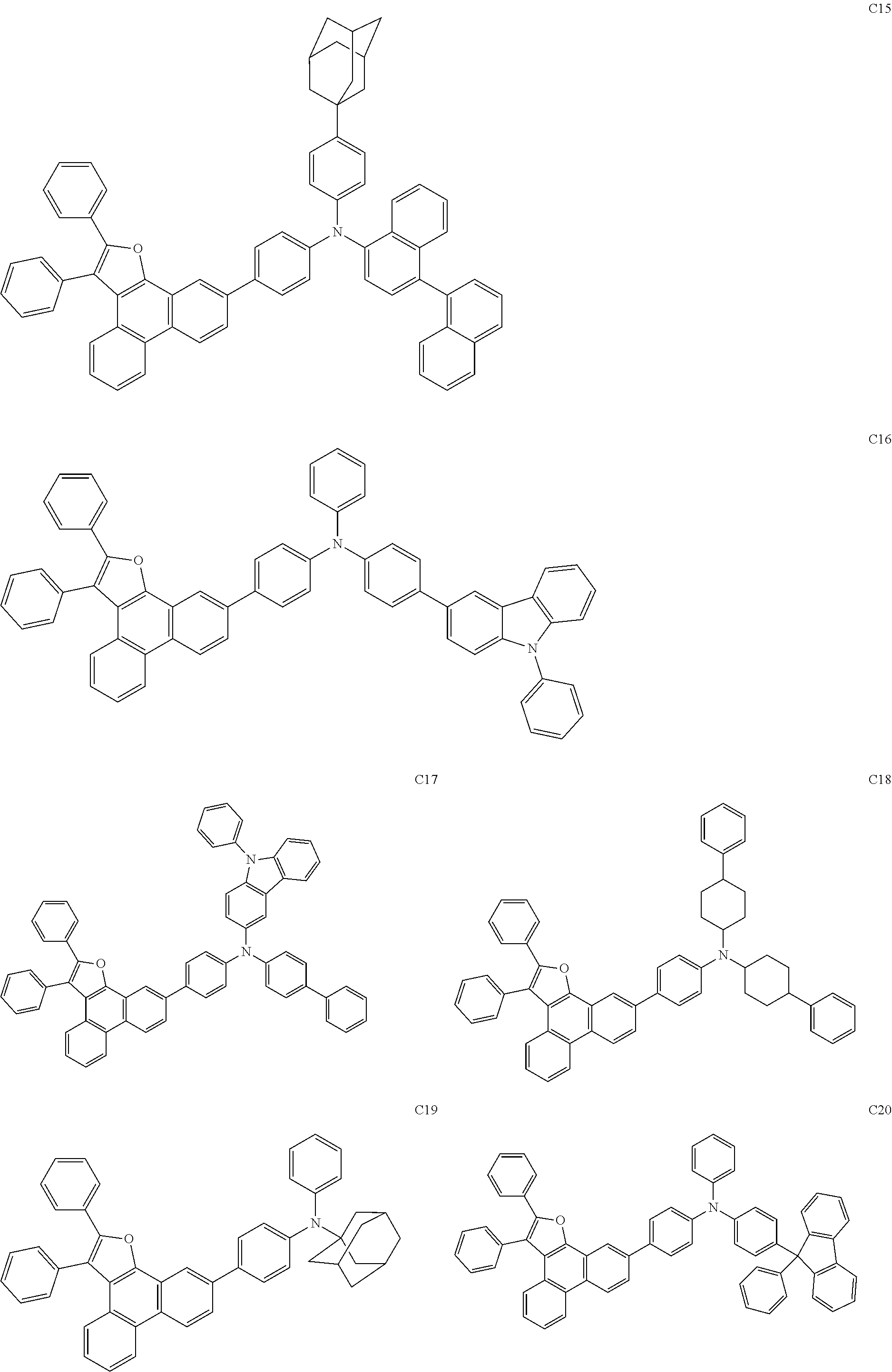 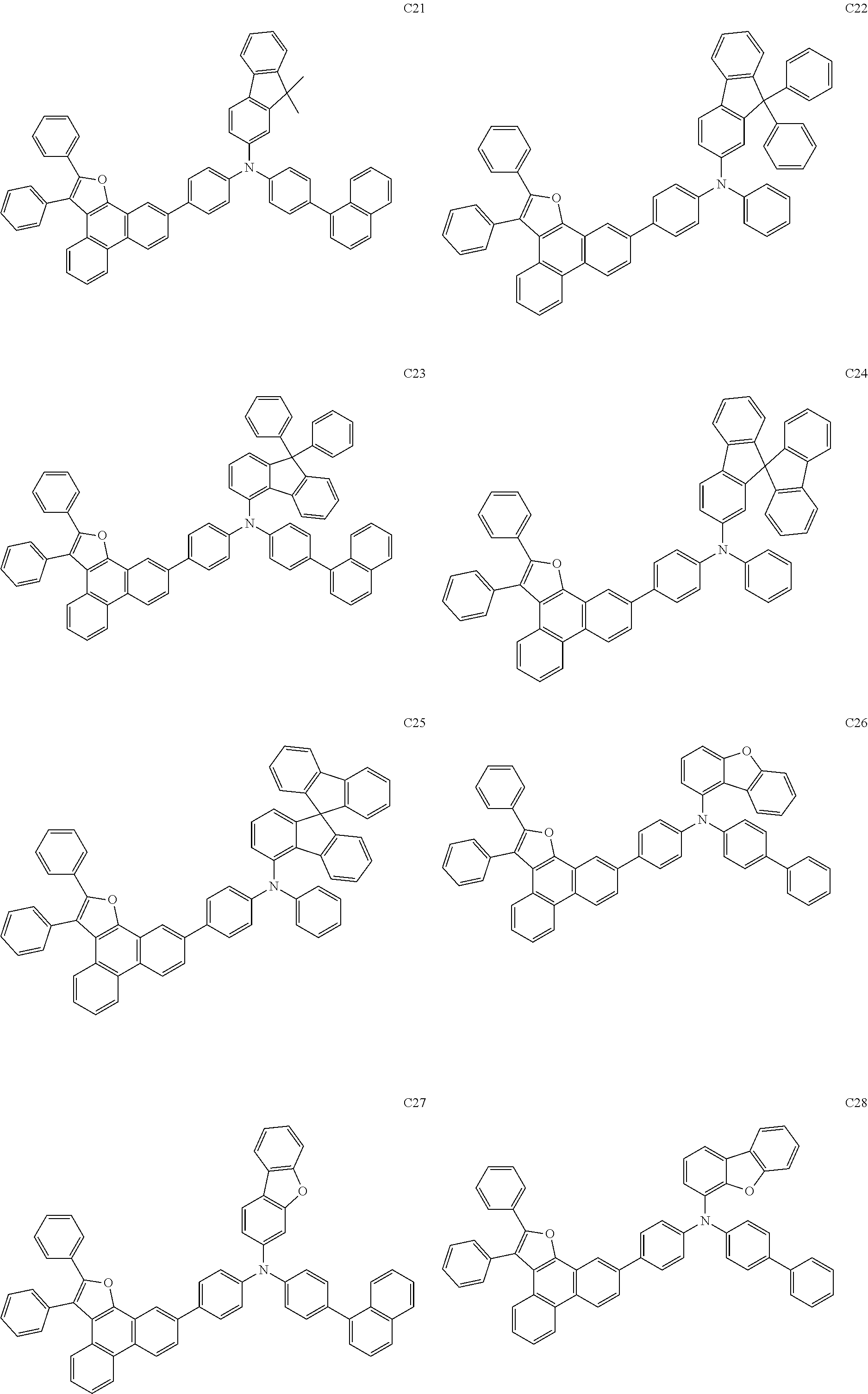 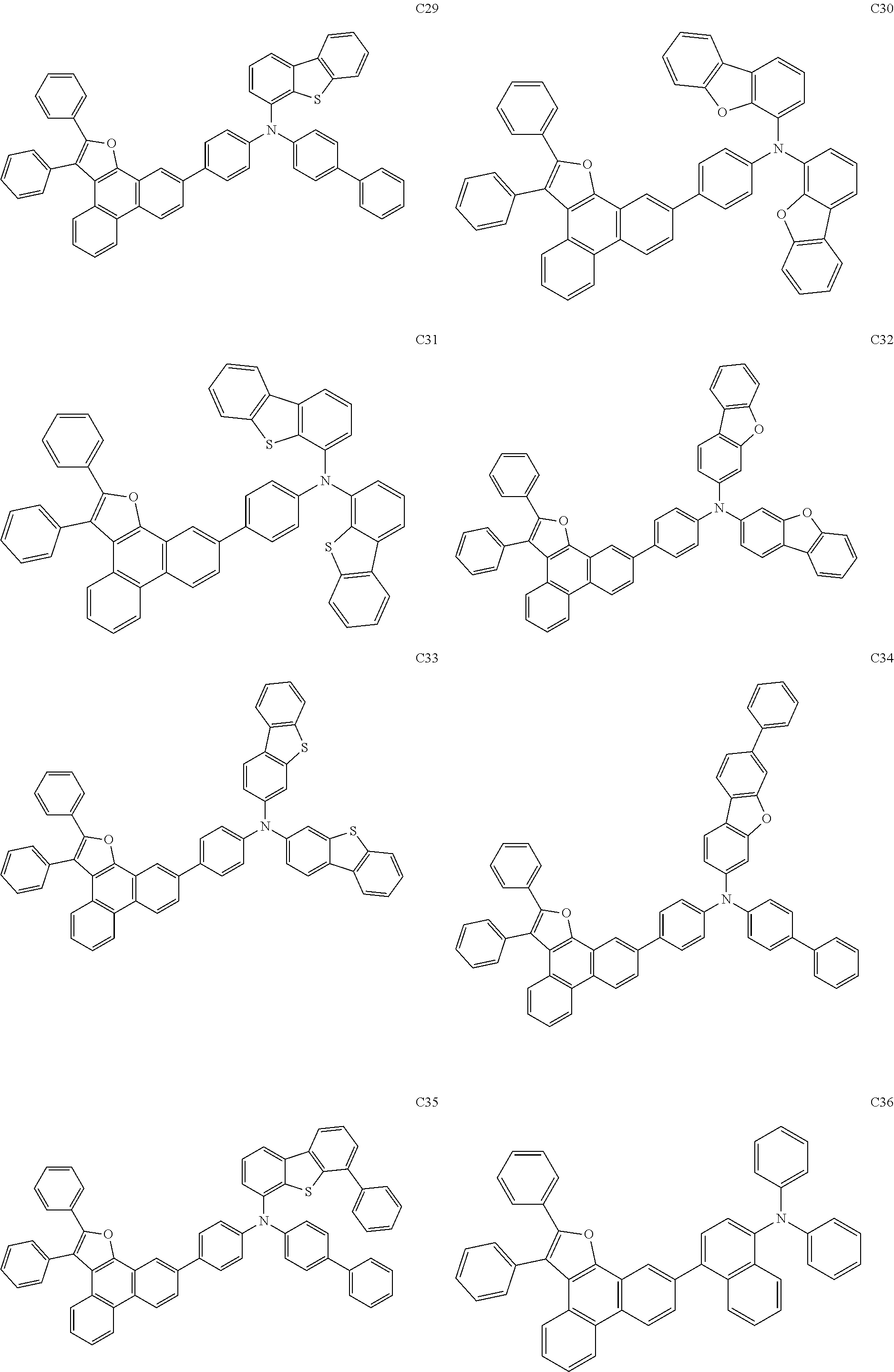 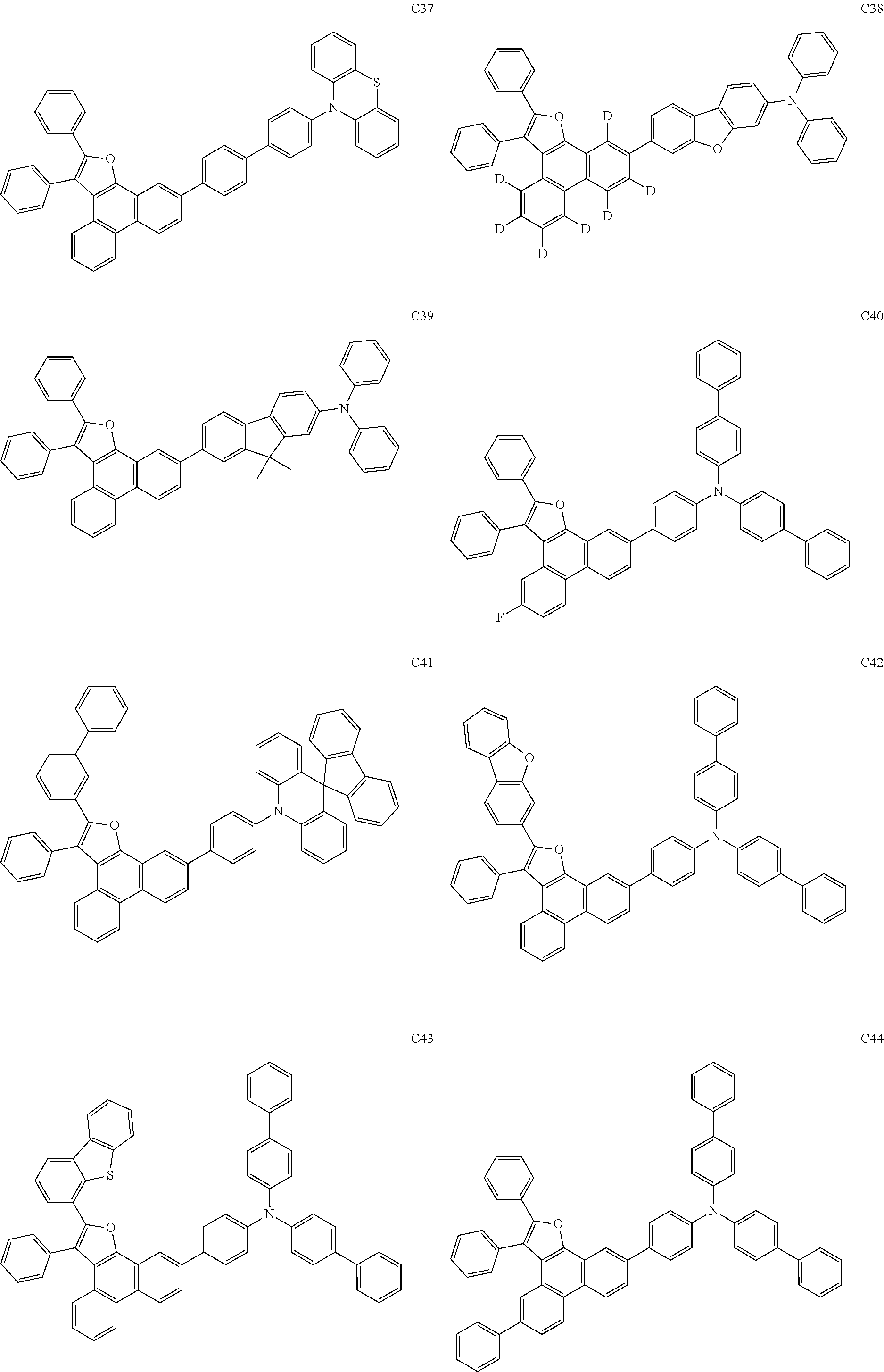 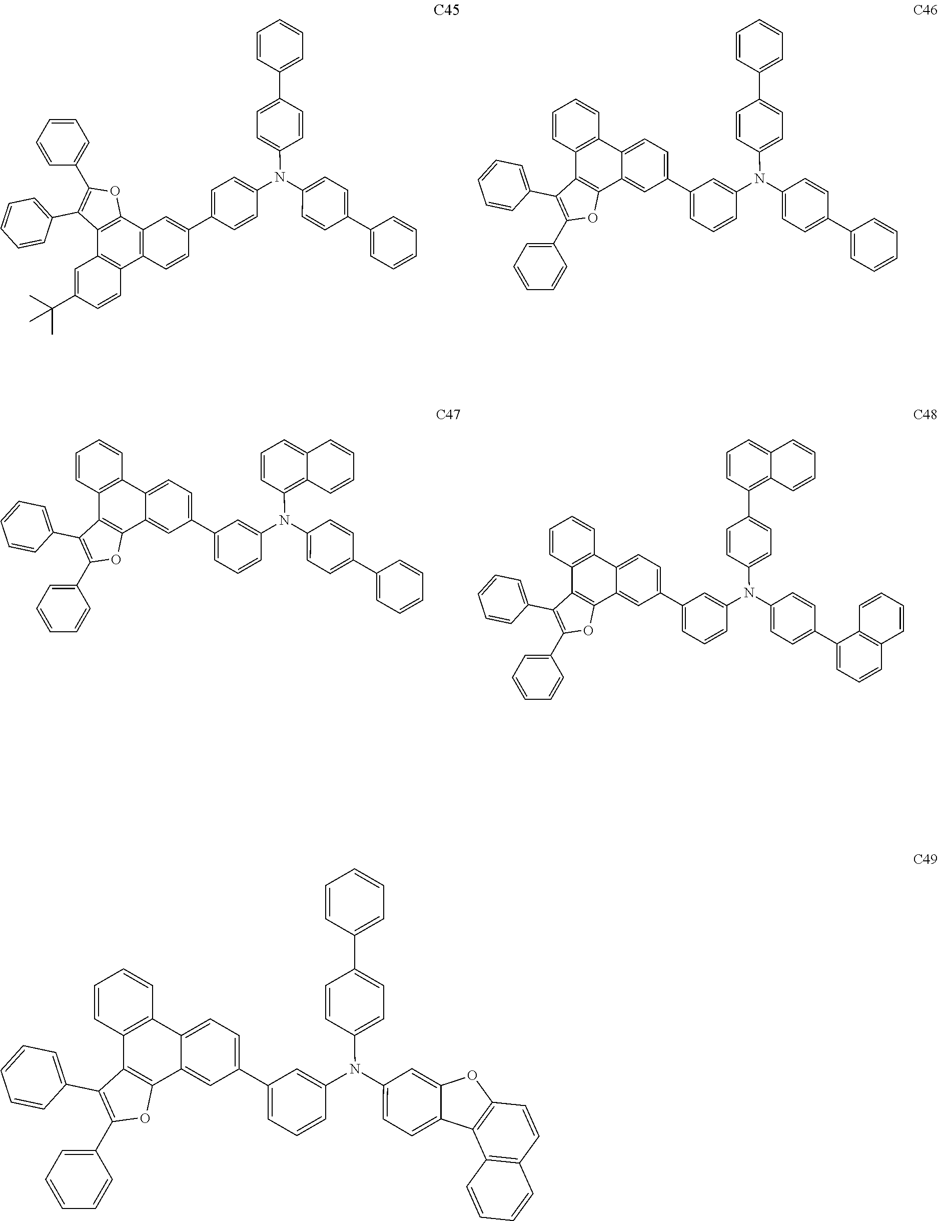 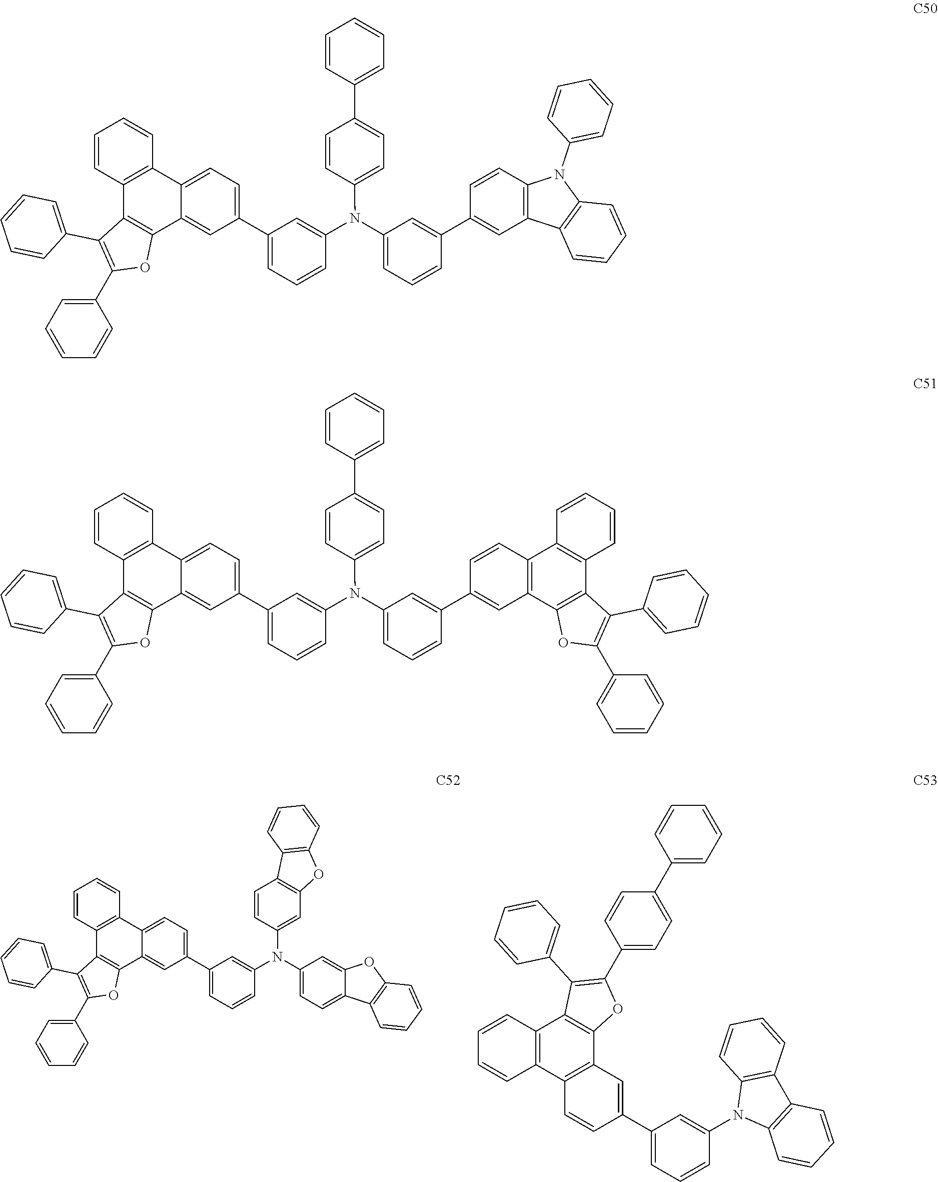 [SMILES: c1ccc(-c2ccc(N(c3cccc(-c4ccc5c6ccccc6c6c(-c7ccccc7)c(-c7ccccc7)oc6c5c4)c3)c3cccc(-c4ccc5c(c4)c4ccccc4n5-c4ccccc4)c3)cc2)cc1 ; c1ccc(-c2ccc(N(c3cccc(-c4ccc5c6ccccc6c6c(-c7ccccc7)c(-c7ccccc7)oc6c5c4)c3)c3cccc(-c4ccc5c6ccccc6c6c(-c7ccccc7)c(-c7ccccc7)oc6c5c4)c3)cc2)cc1 ; c1ccc(-c2oc3c4cc(-c5cccc(N(c6ccc7c(c6)oc6ccccc67)c6ccc7c(c6)oc6ccccc67)c5)ccc4c4ccccc4c3c2-c2ccccc2)cc1 ; c1ccc(-c2ccc(-c3oc4c5cc(-c6cccc(-n7c8ccccc8c8ccccc87)c6)ccc5c5ccccc5c4c3-c3ccccc3)cc2)cc1] 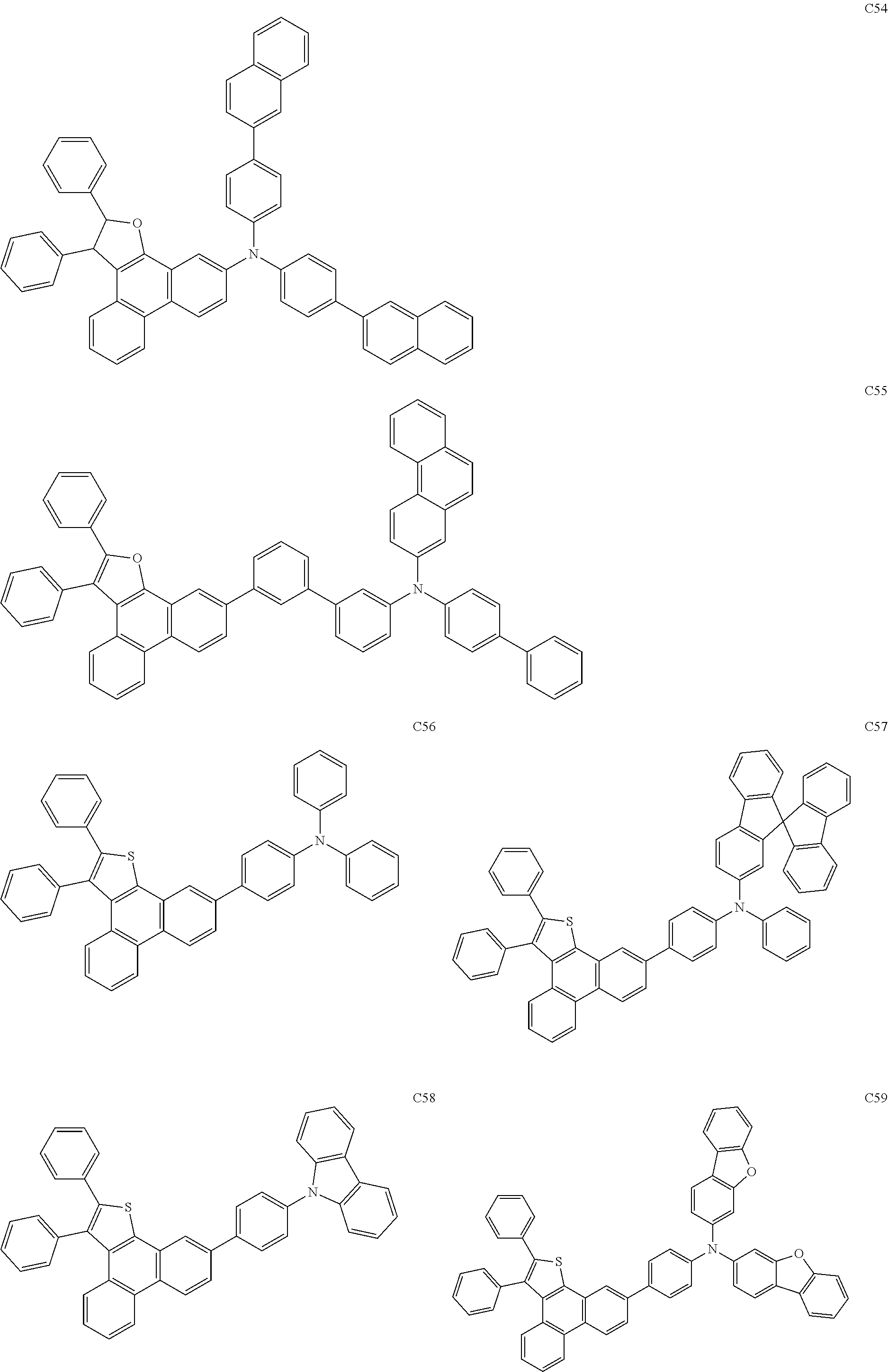 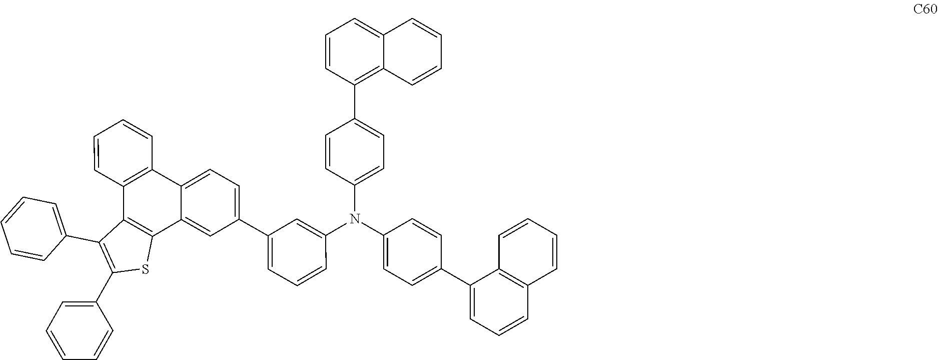 [SMILES: c1ccc(-c2sc3c4cc(-c5cccc(N(c6ccc(-c7cccc8ccccc78)cc6)c6ccc(-c7cccc8ccccc78)cc6)c5)ccc4c4ccccc4c3c2-c2ccccc2)cc1] 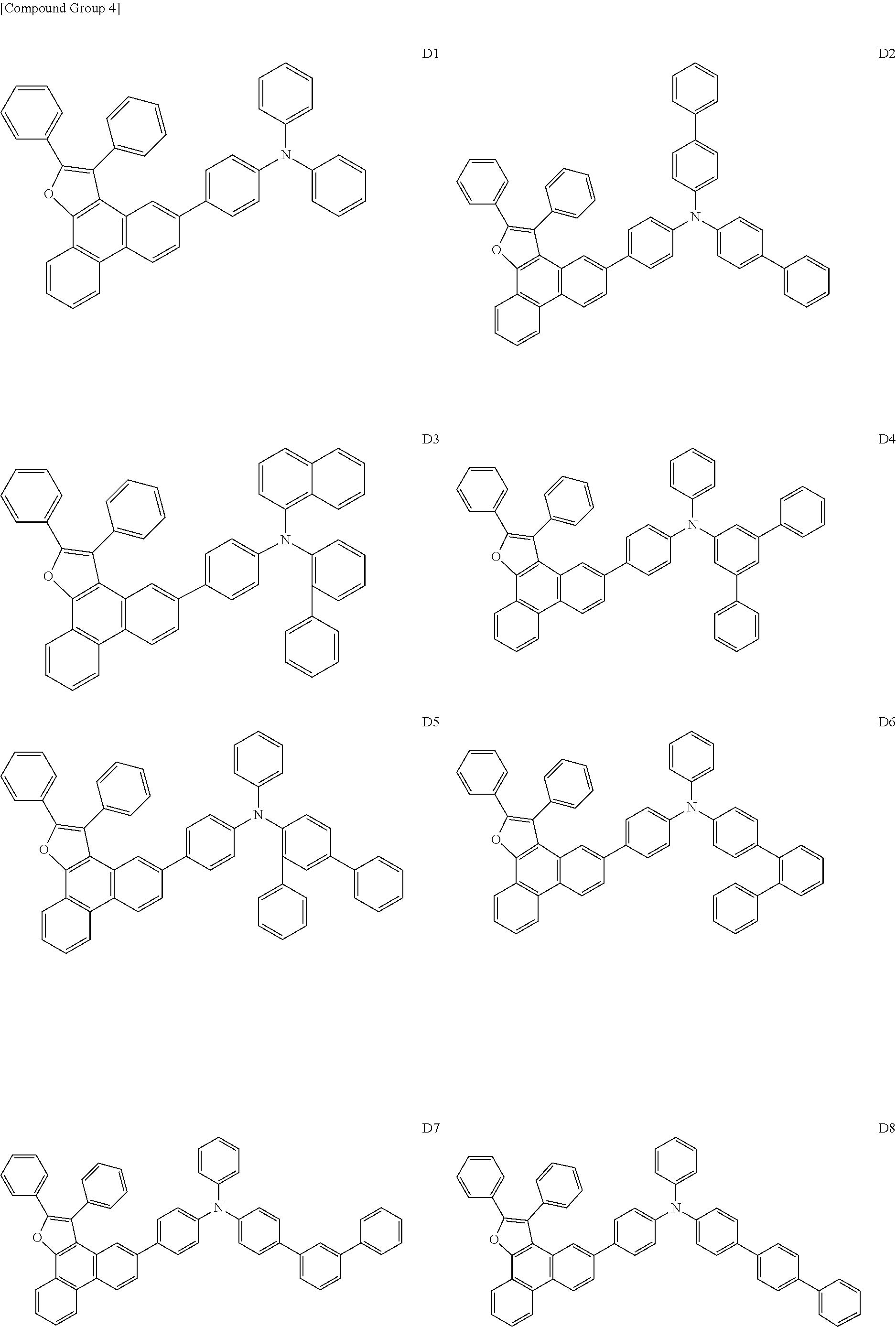 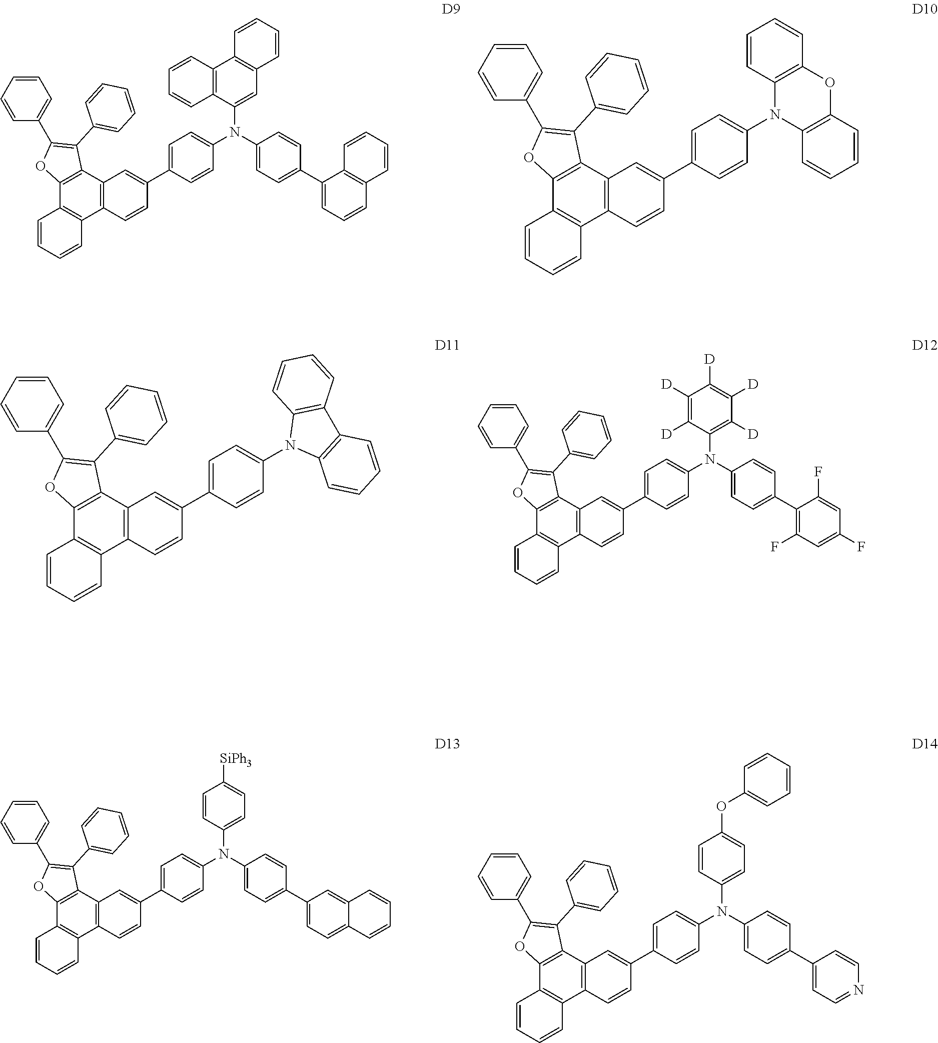 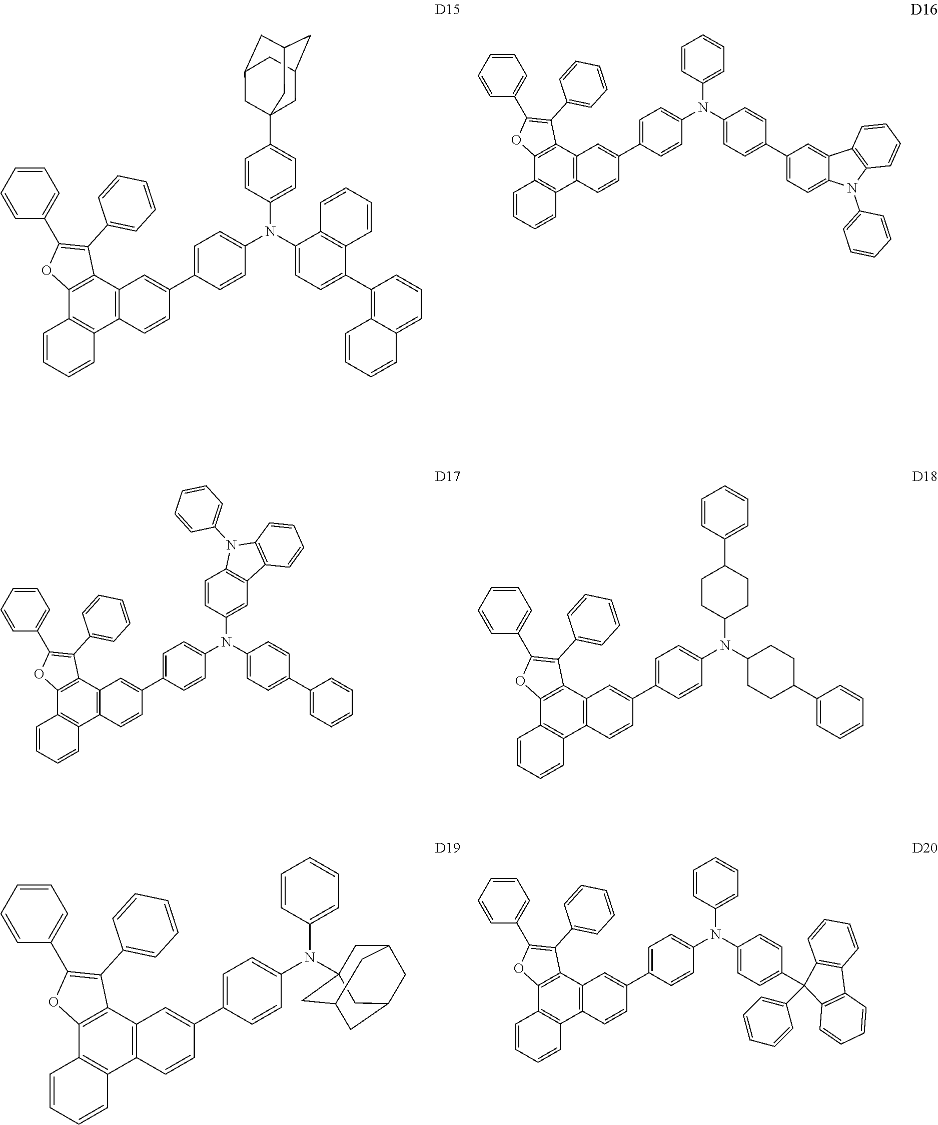 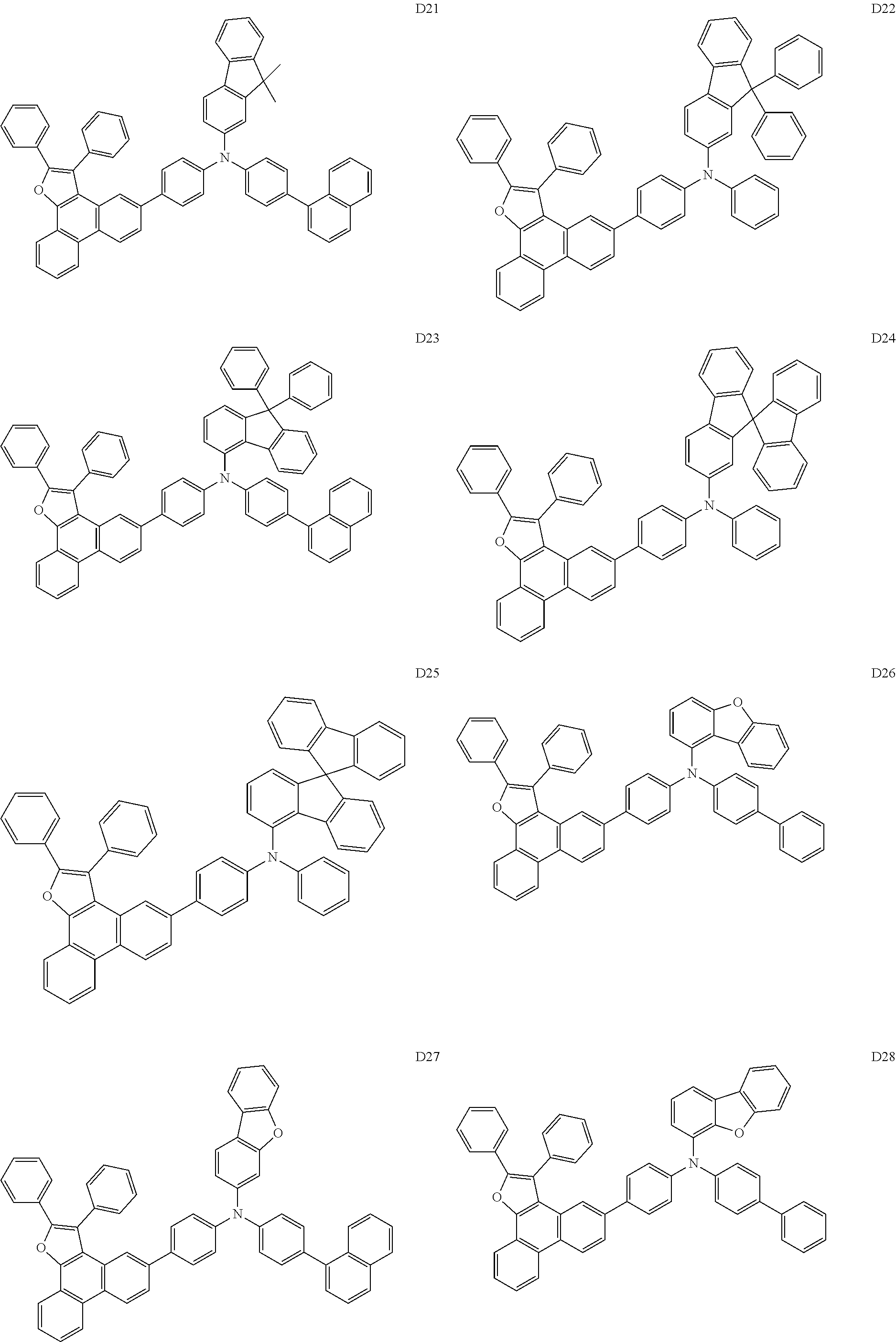 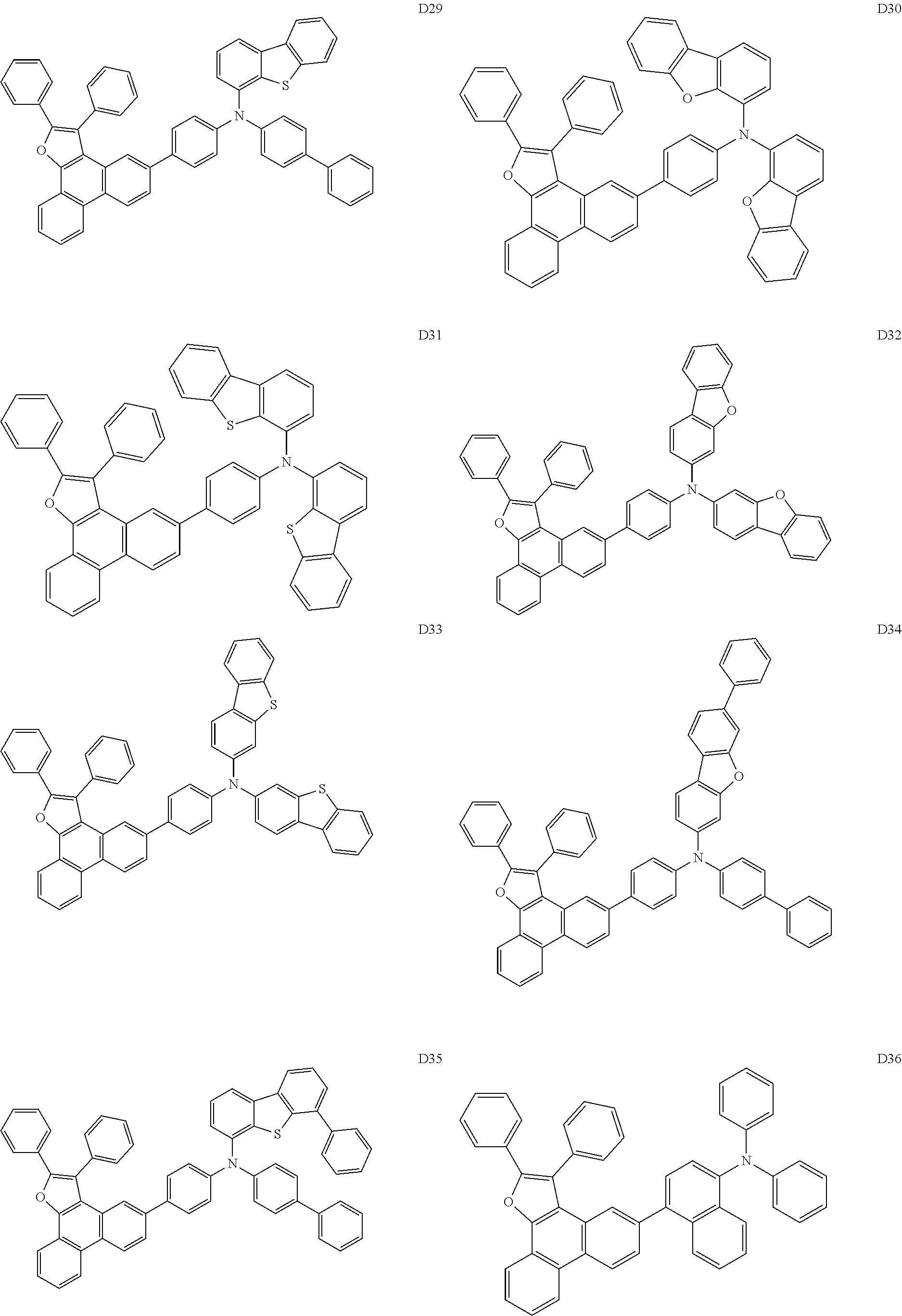 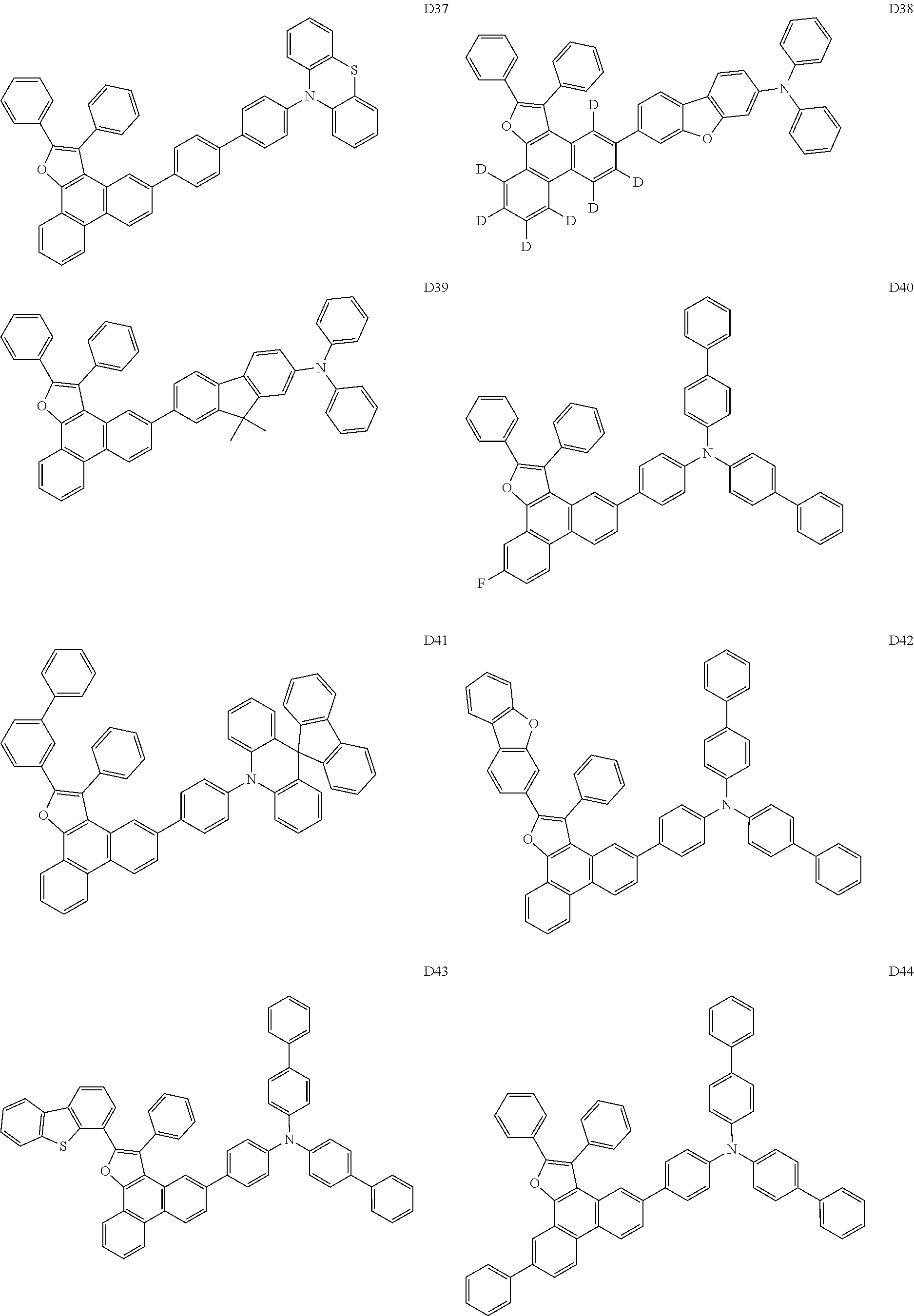 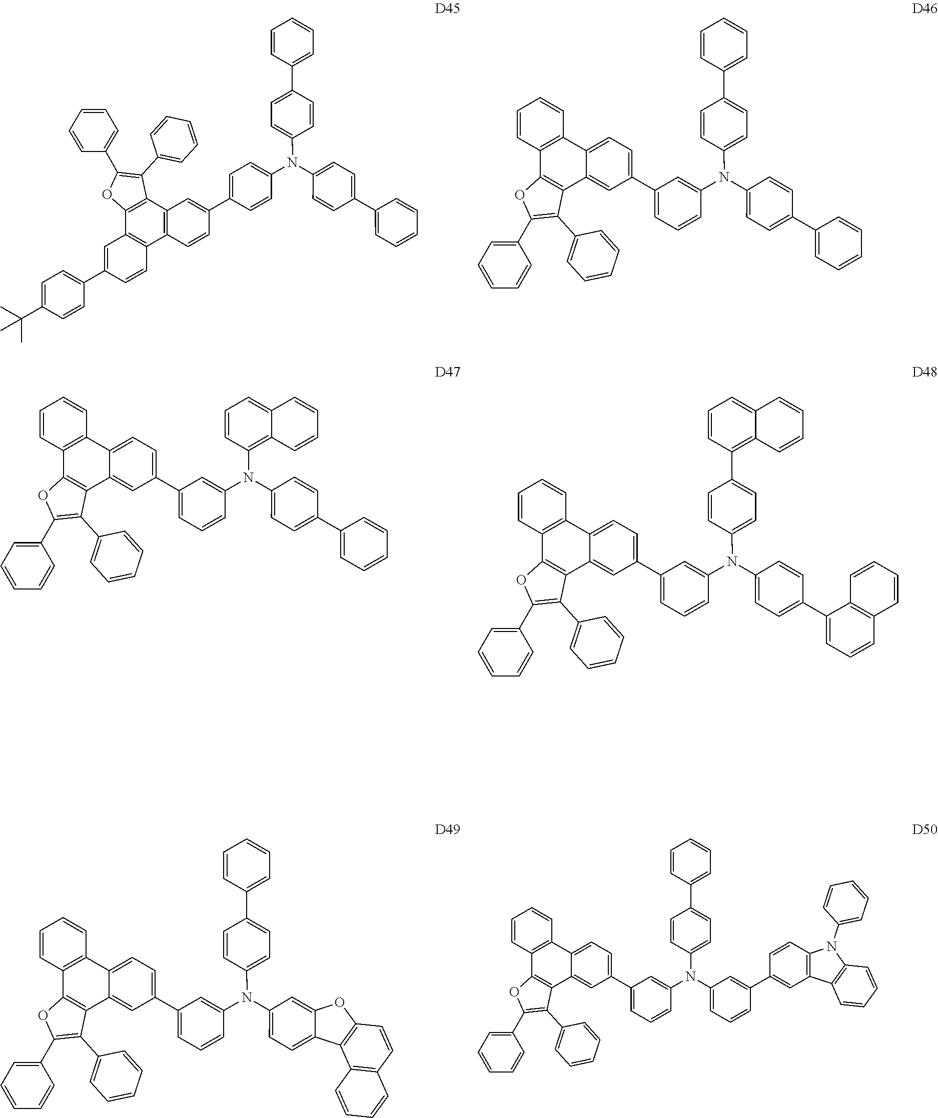 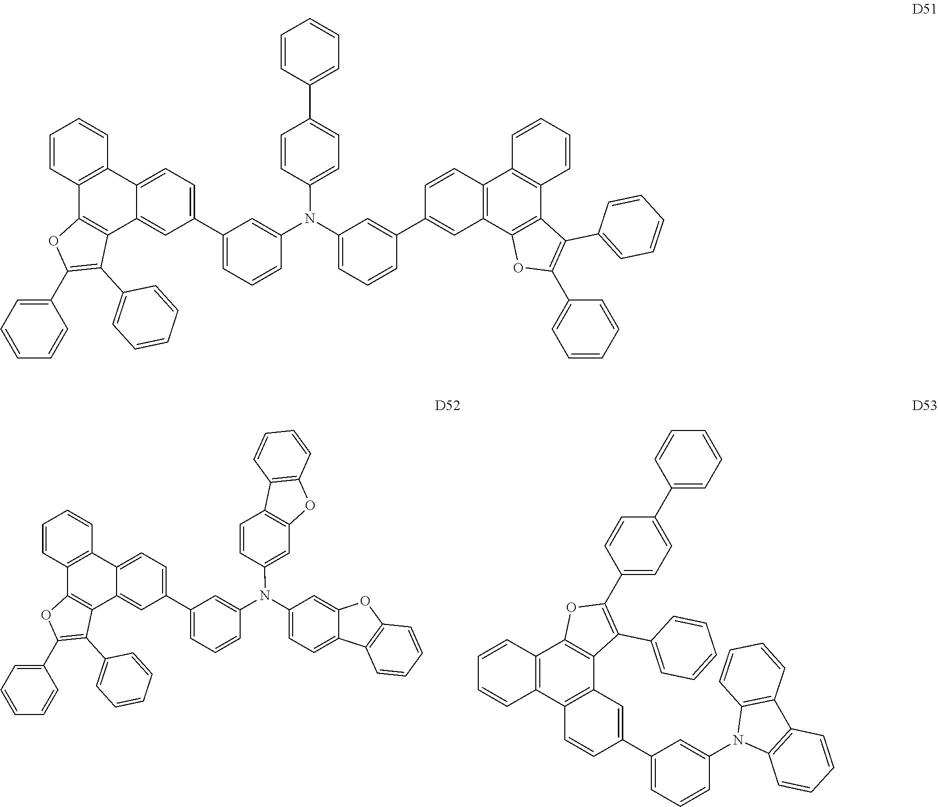 [SMILES: c1ccc(-c2ccc(N(c3cccc(-c4ccc5c6ccccc6c6c(-c7ccccc7)c(-c7ccccc7)oc6c5c4)c3)c3cccc(-c4ccc5c6ccccc6c6oc(-c7ccccc7)c(-c7ccccc7)c6c5c4)c3)cc2)cc1 ; c1ccc(-c2oc3c4ccccc4c4ccc(-c5cccc(N(c6ccc7c(c6)oc6ccccc67)c6ccc7c(c6)oc6ccccc67)c5)cc4c3c2-c2ccccc2)cc1 ; c1ccc(-c2ccc(-c3oc4c5ccccc5c5ccc(-c6cccc(-n7c8ccccc8c8ccccc87)c6)cc5c4c3-c3ccccc3)cc2)cc1] 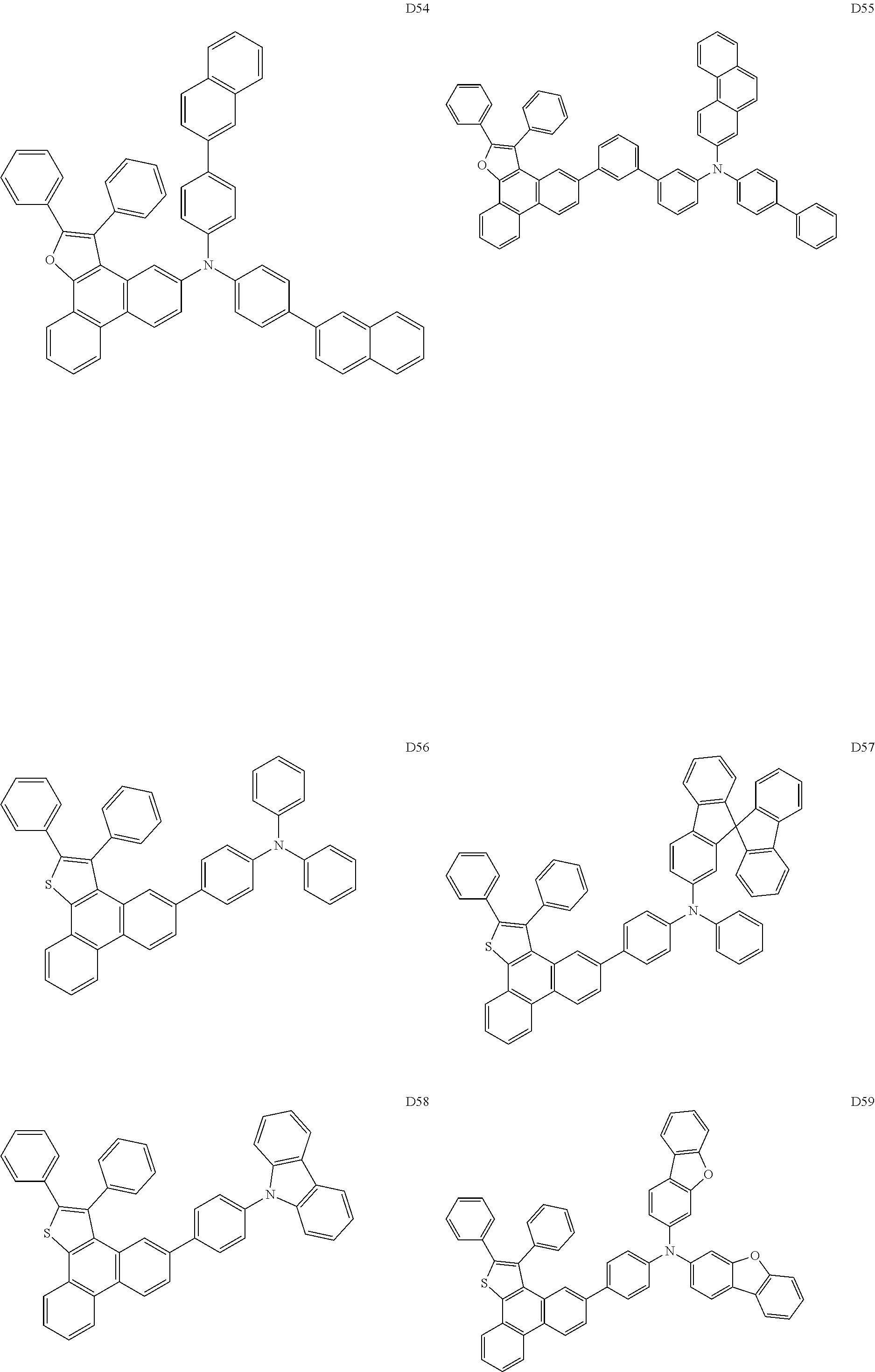 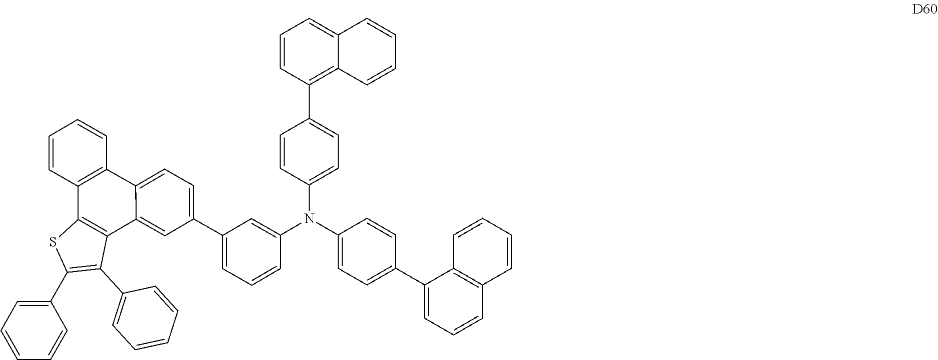 [SMILES: c1ccc(-c2sc3c4ccccc4c4ccc(-c5cccc(N(c6ccc(-c7cccc8ccccc78)cc6)c6ccc(-c7cccc8ccccc78)cc6)c5)cc4c3c2-c2ccccc2)cc1] 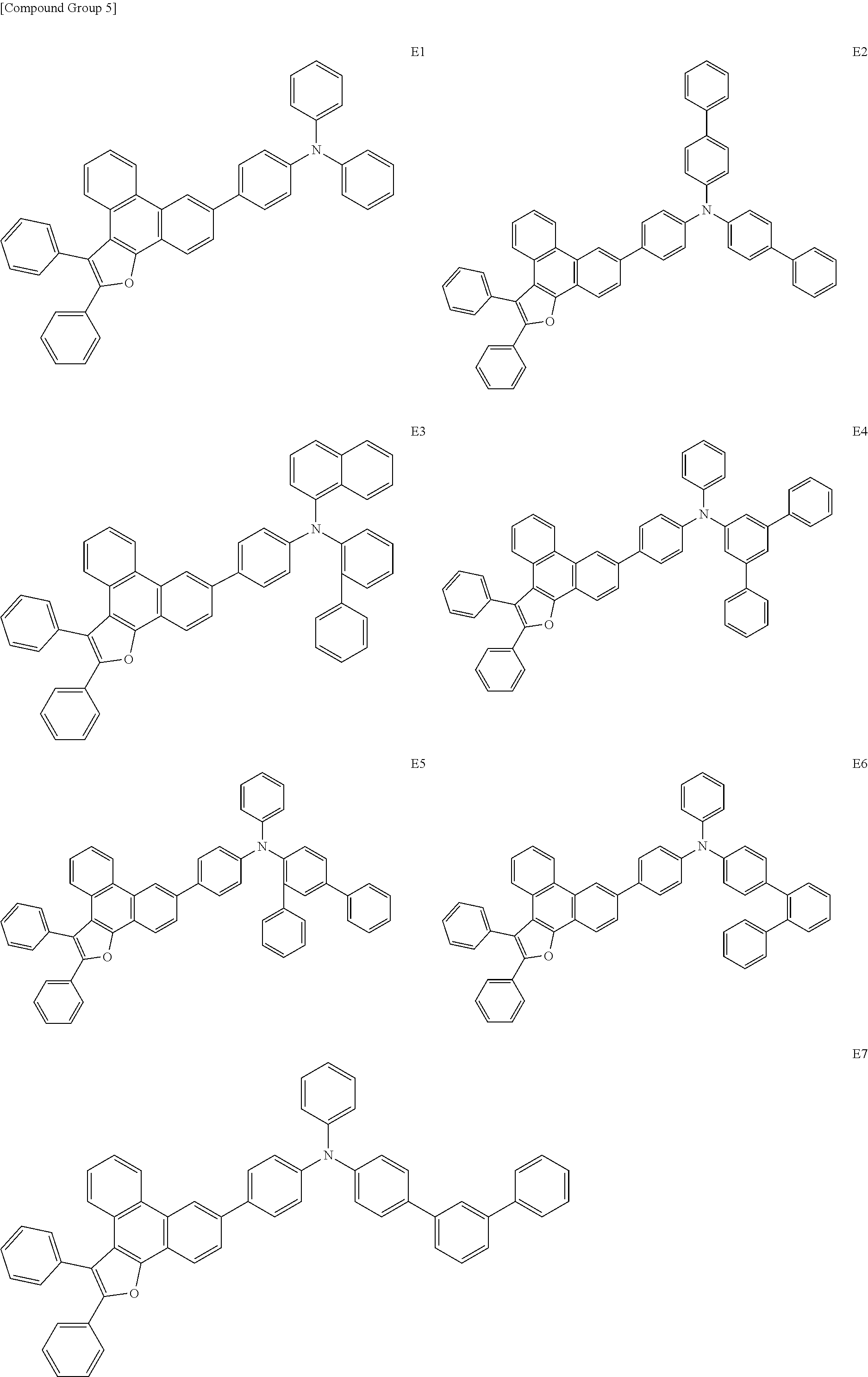 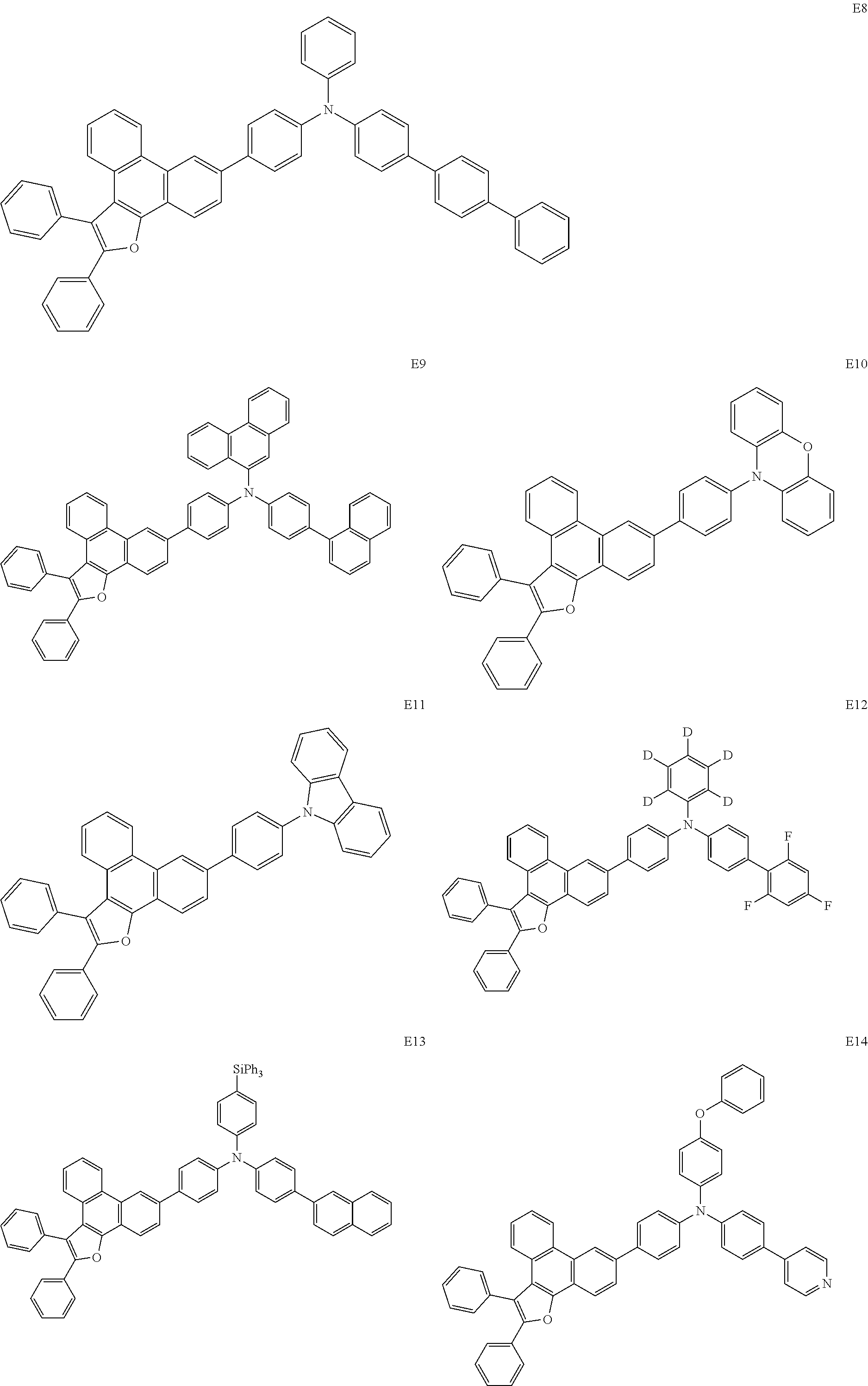 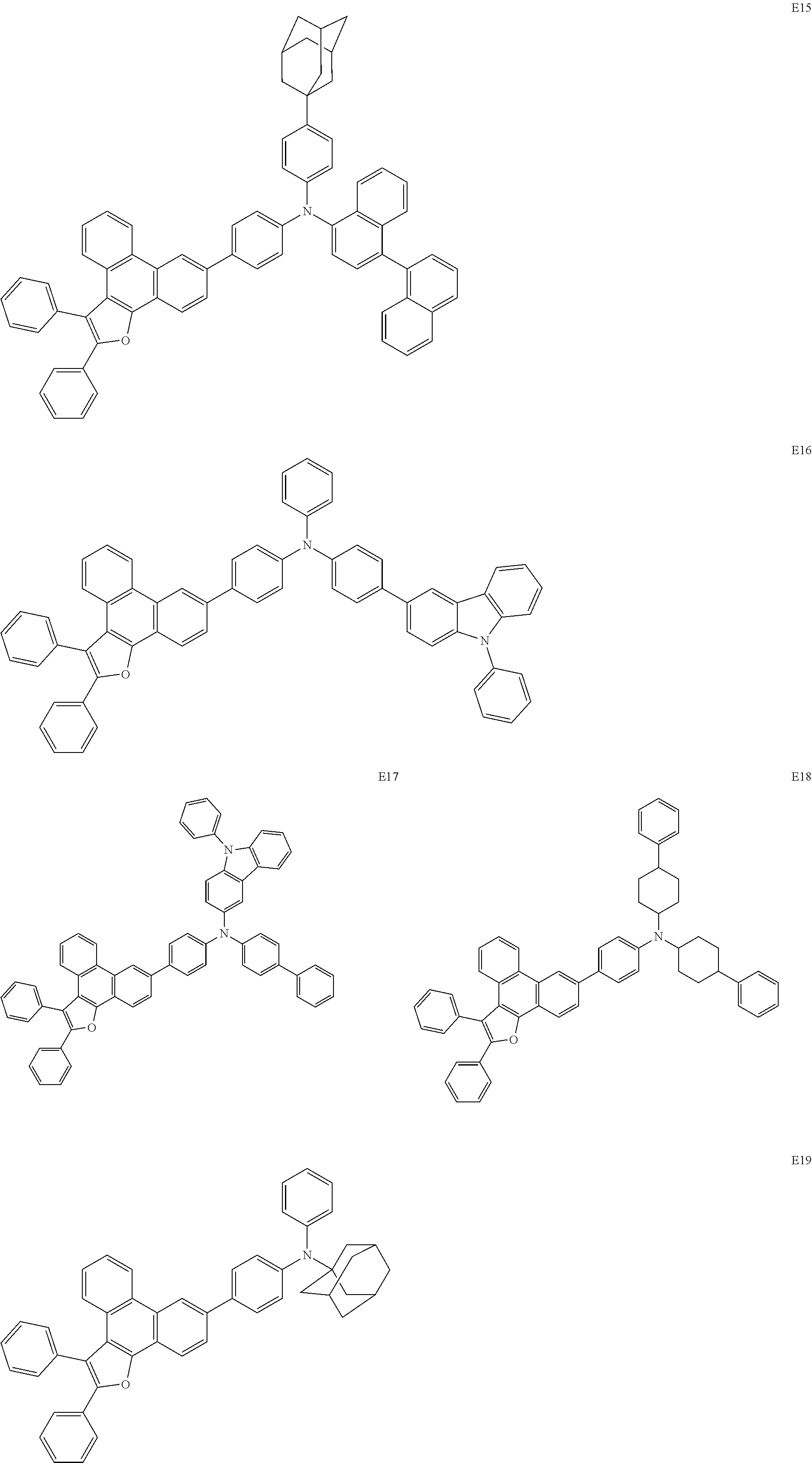 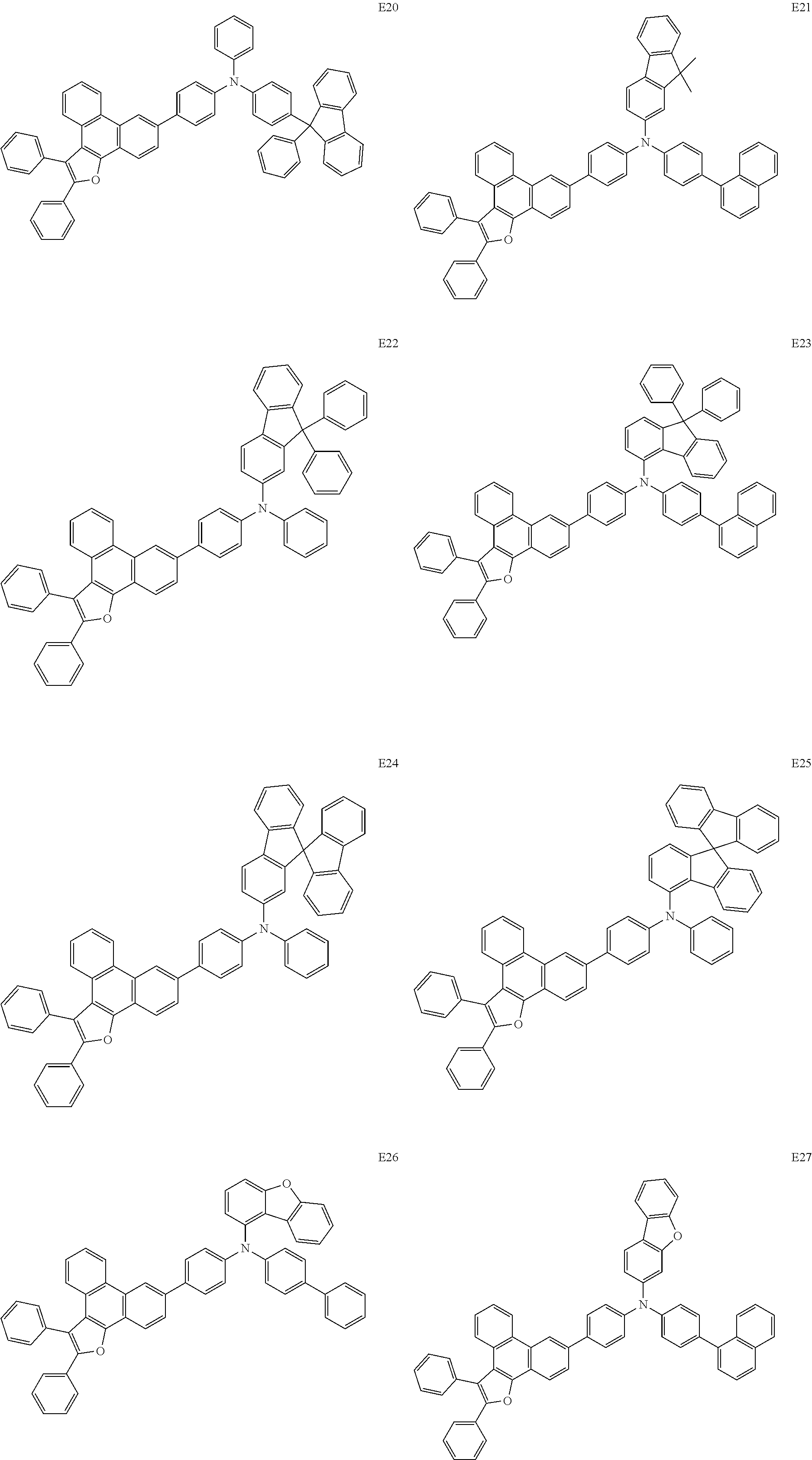 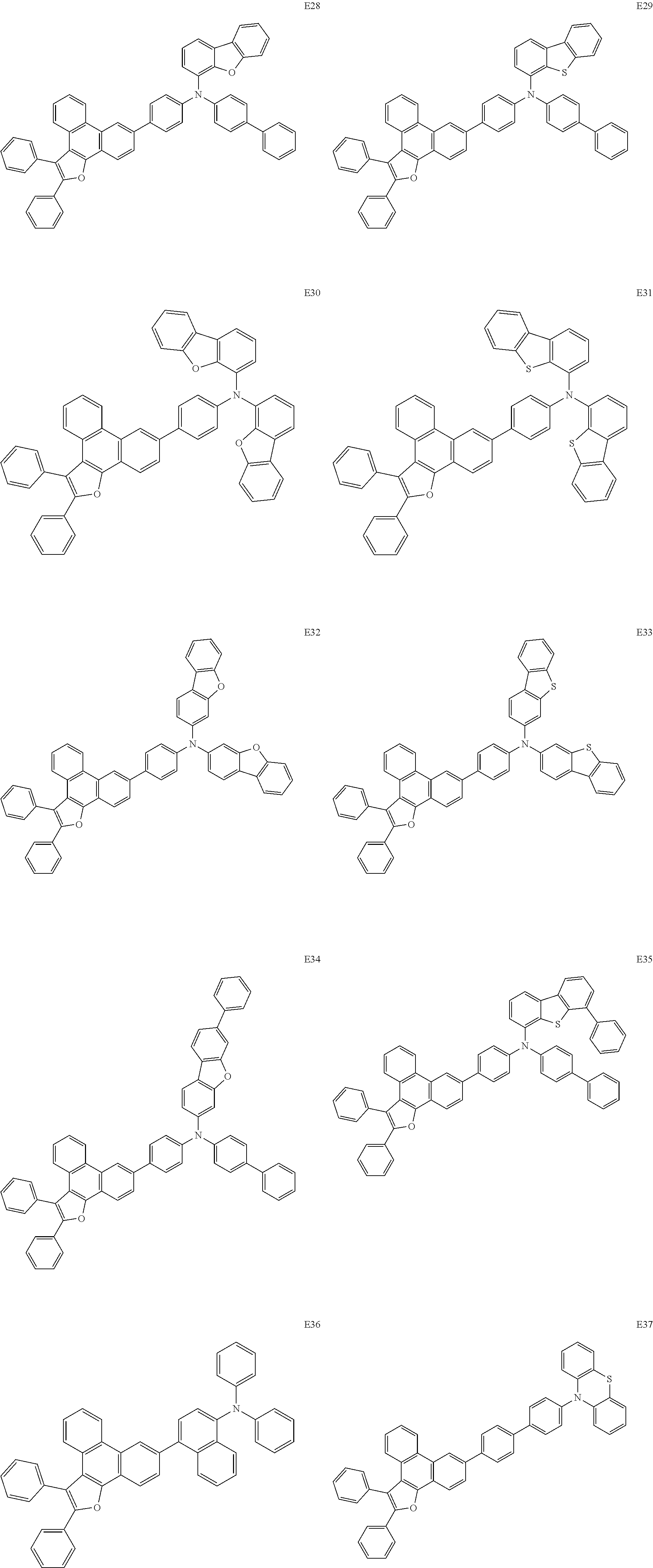 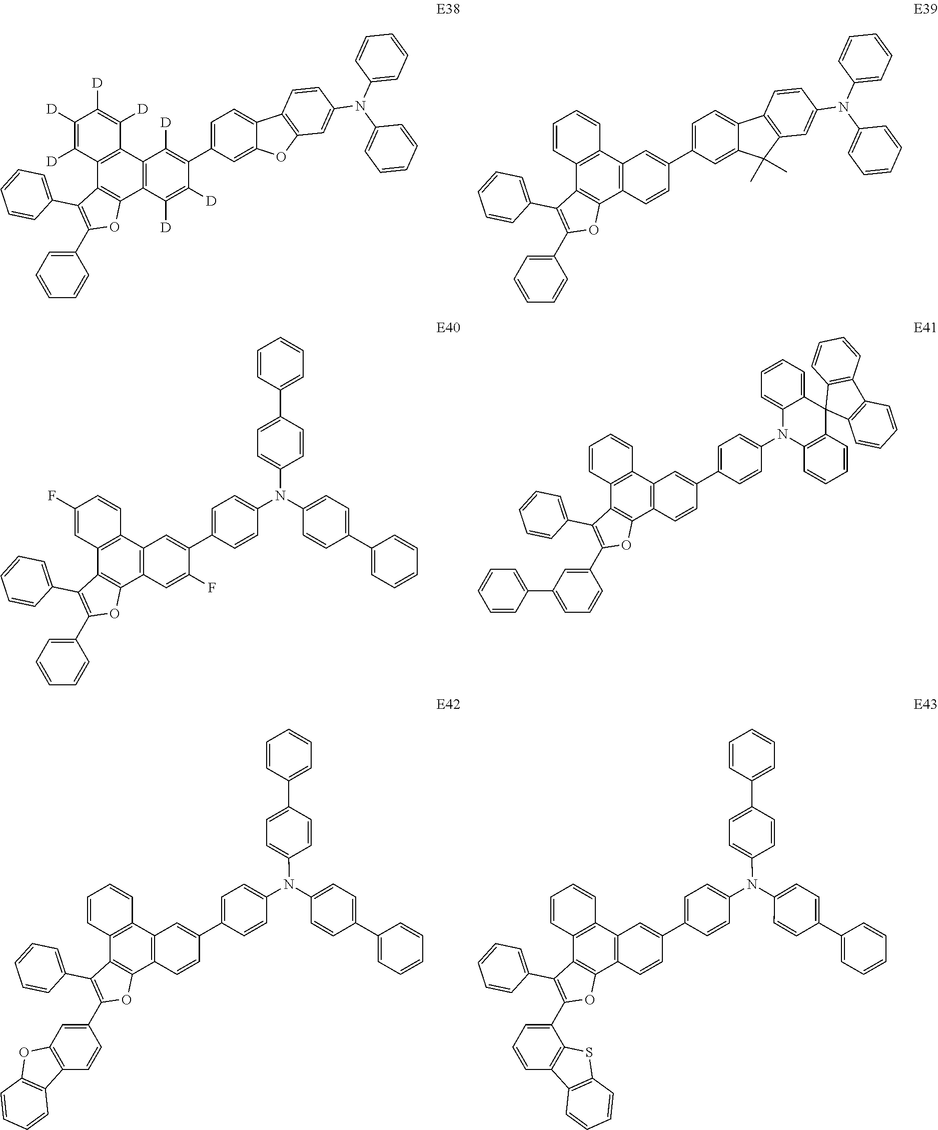 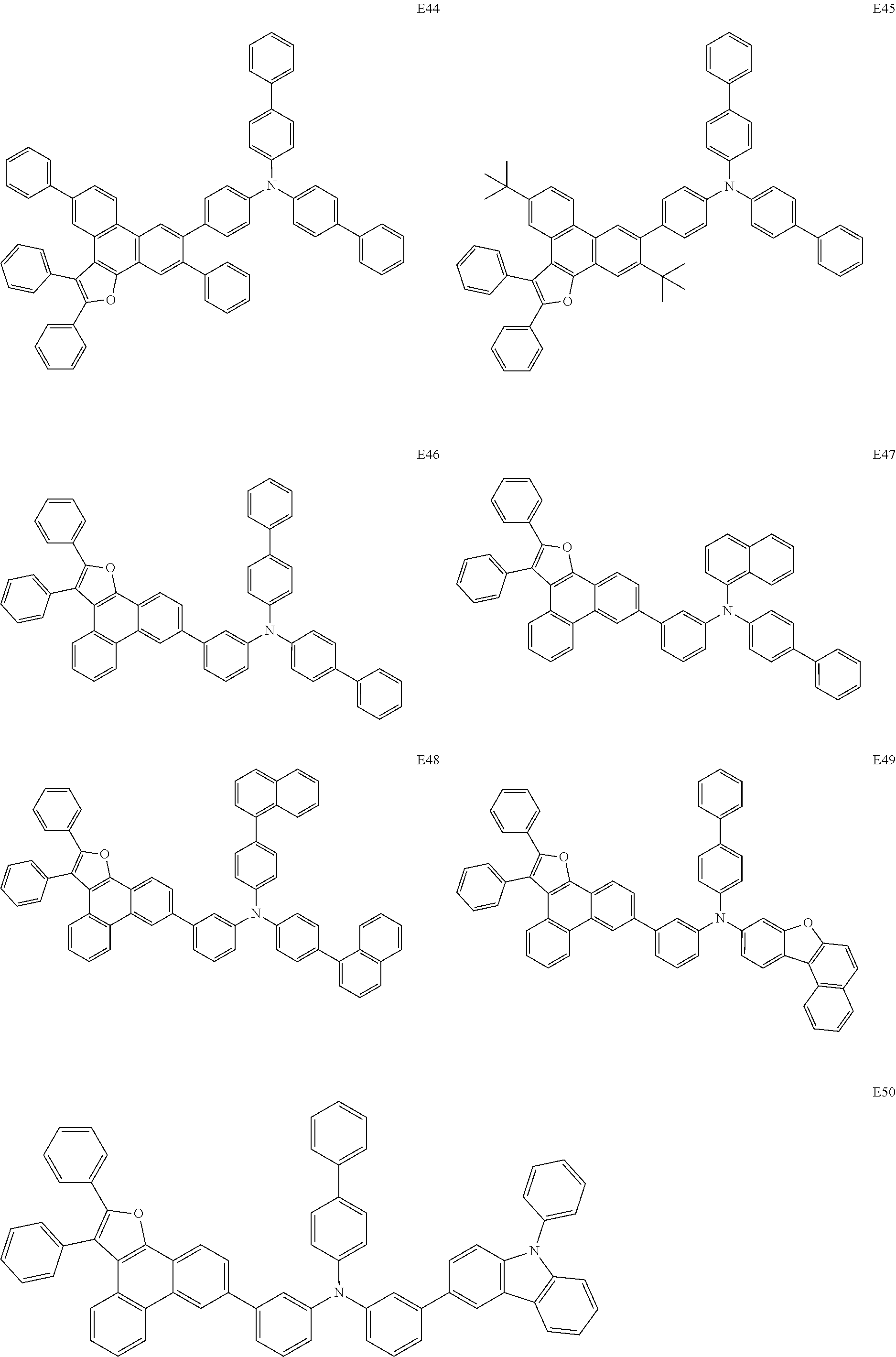 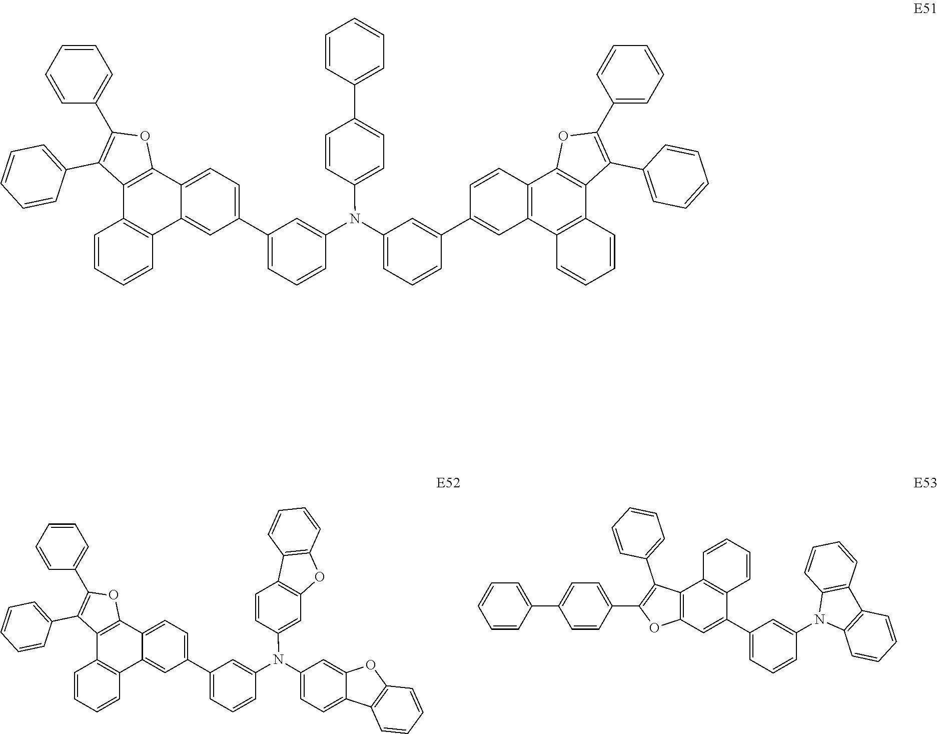 [SMILES: c1ccc(-c2ccc(N(c3cccc(-c4ccc5c(c4)c4ccccc4c4c(-c6ccccc6)c(-c6ccccc6)oc54)c3)c3cccc(-c4ccc5c(c4)c4ccccc4c4c(-c6ccccc6)c(-c6ccccc6)oc54)c3)cc2)cc1 ; c1ccc(-c2oc3c4ccc(-c5cccc(N(c6ccc7c(c6)oc6ccccc67)c6ccc7c(c6)oc6ccccc67)c5)cc4c4ccccc4c3c2-c2ccccc2)cc1 ; c1ccc(-c2ccc(-c3oc4cc(-c5cccc(-n6c7ccccc7c7ccccc76)c5)c5ccccc5c4c3-c3ccccc3)cc2)cc1] 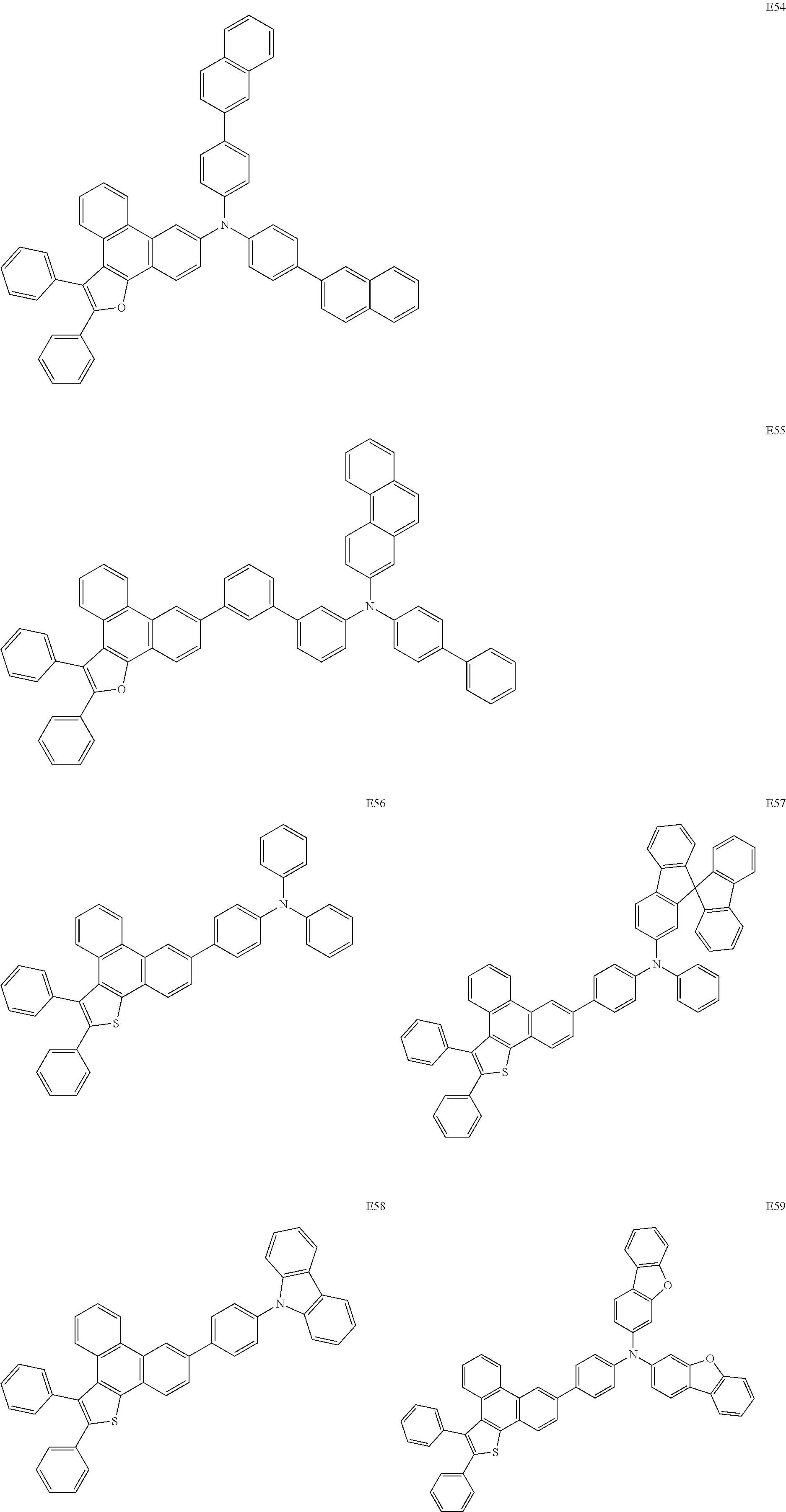 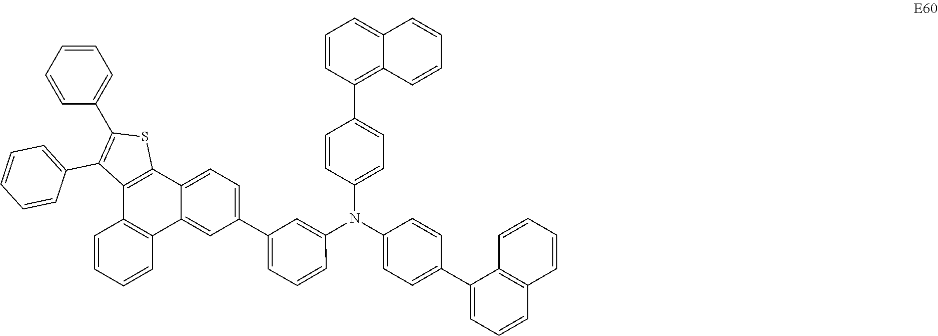 [SMILES: c1ccc(-c2sc3c4ccc(-c5cccc(N(c6ccc(-c7cccc8ccccc78)cc6)c6ccc(-c7cccc8ccccc78)cc6)c5)cc4c4ccccc4c3c2-c2ccccc2)cc1] 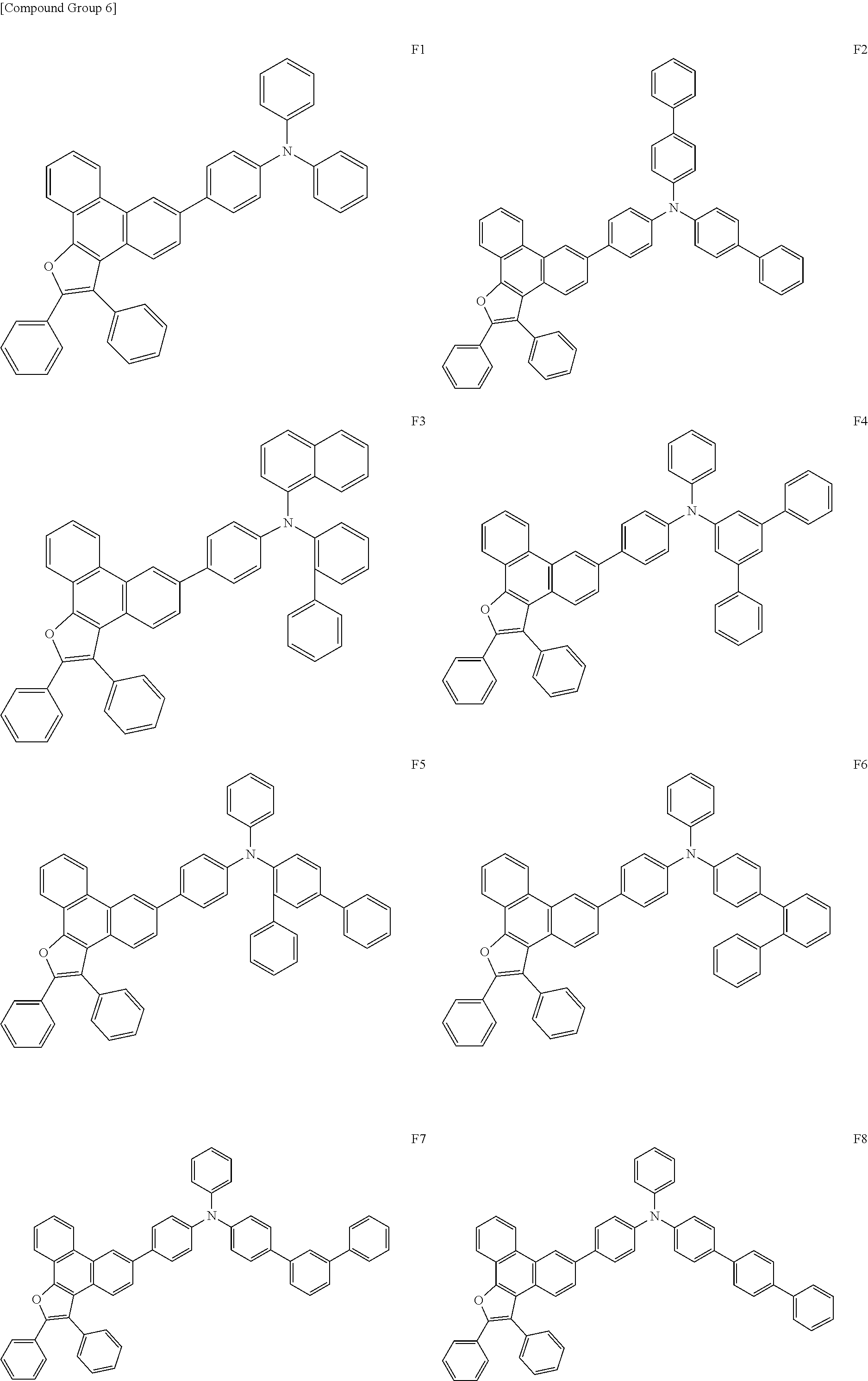 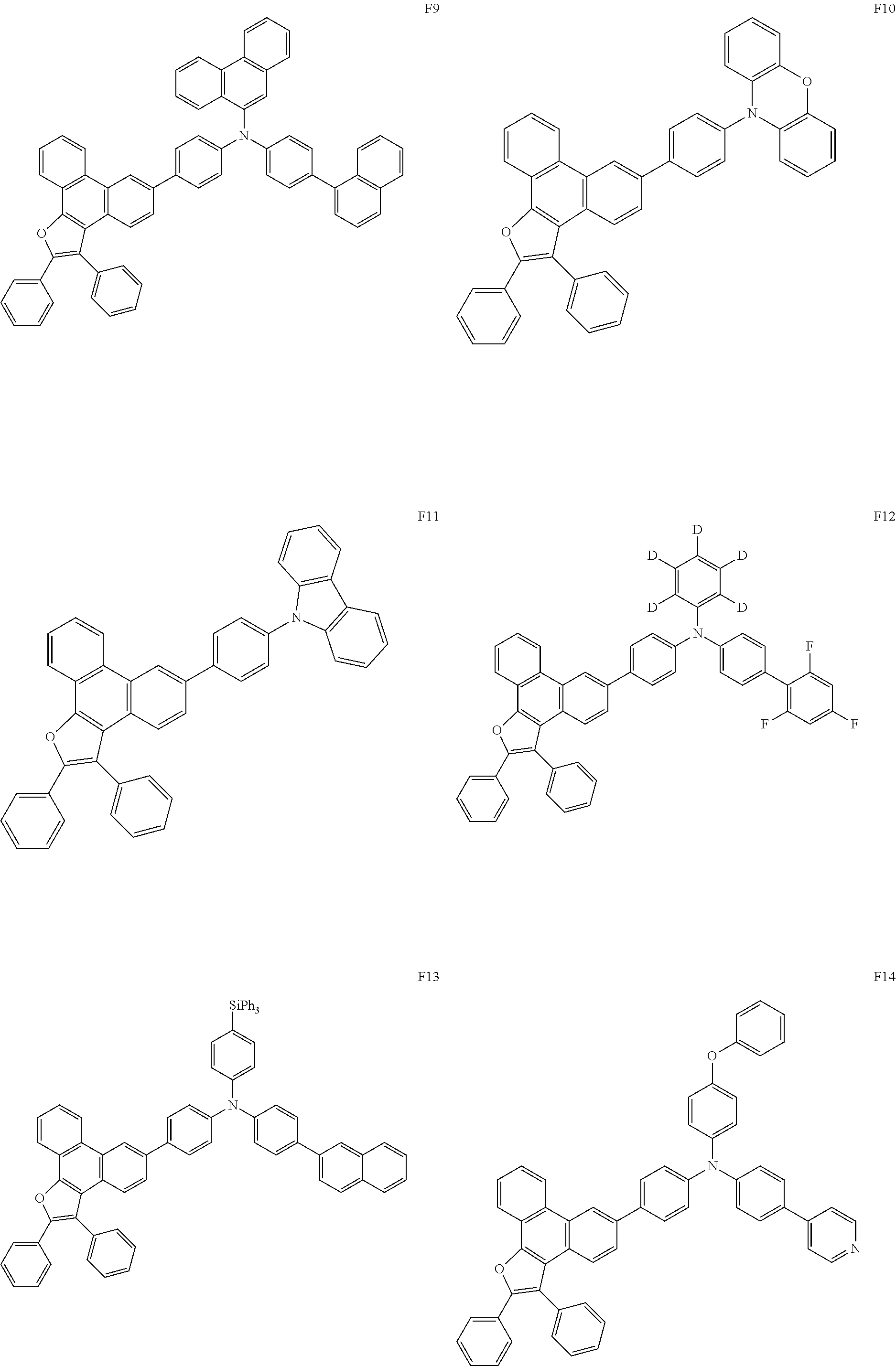 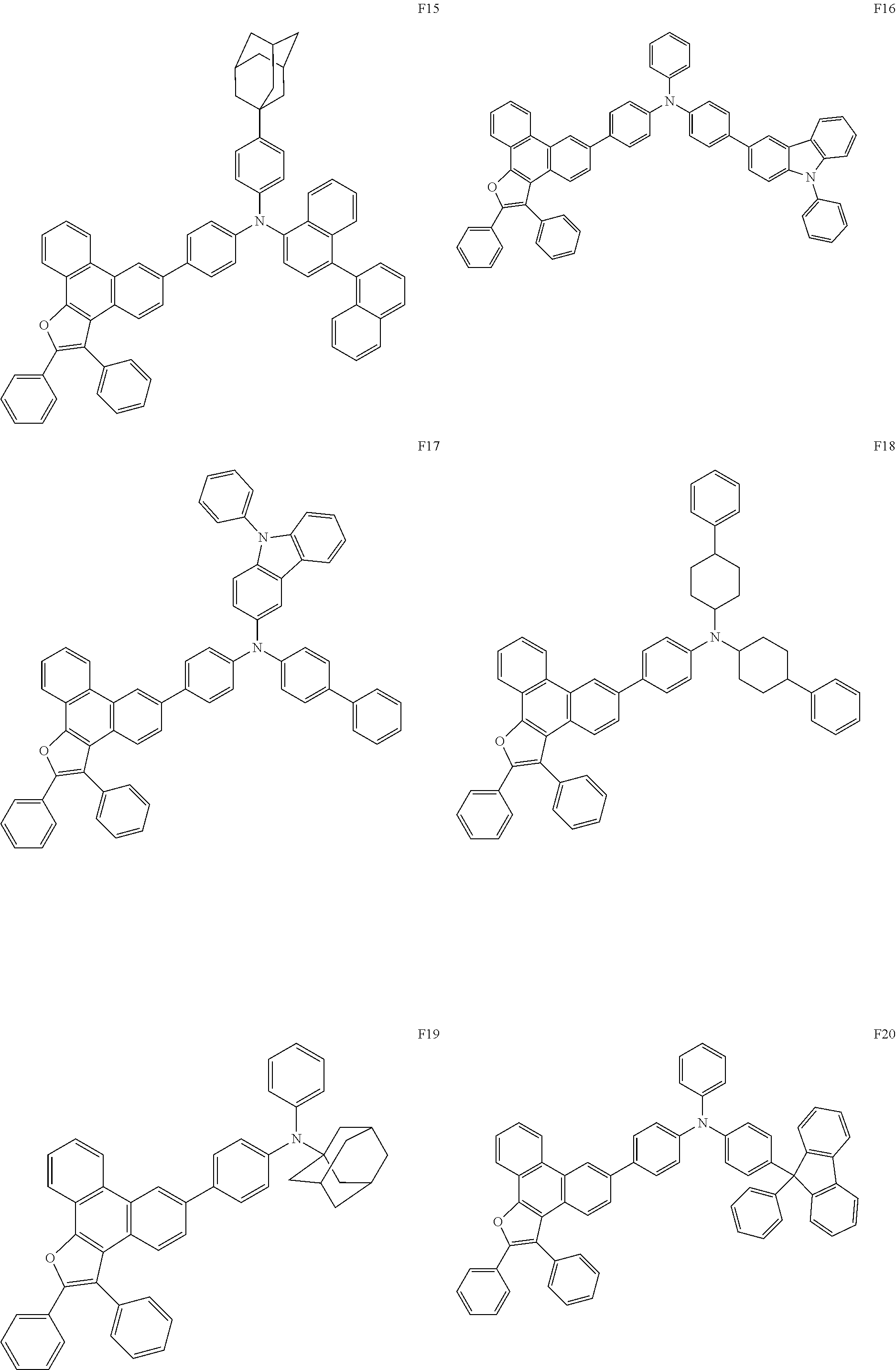 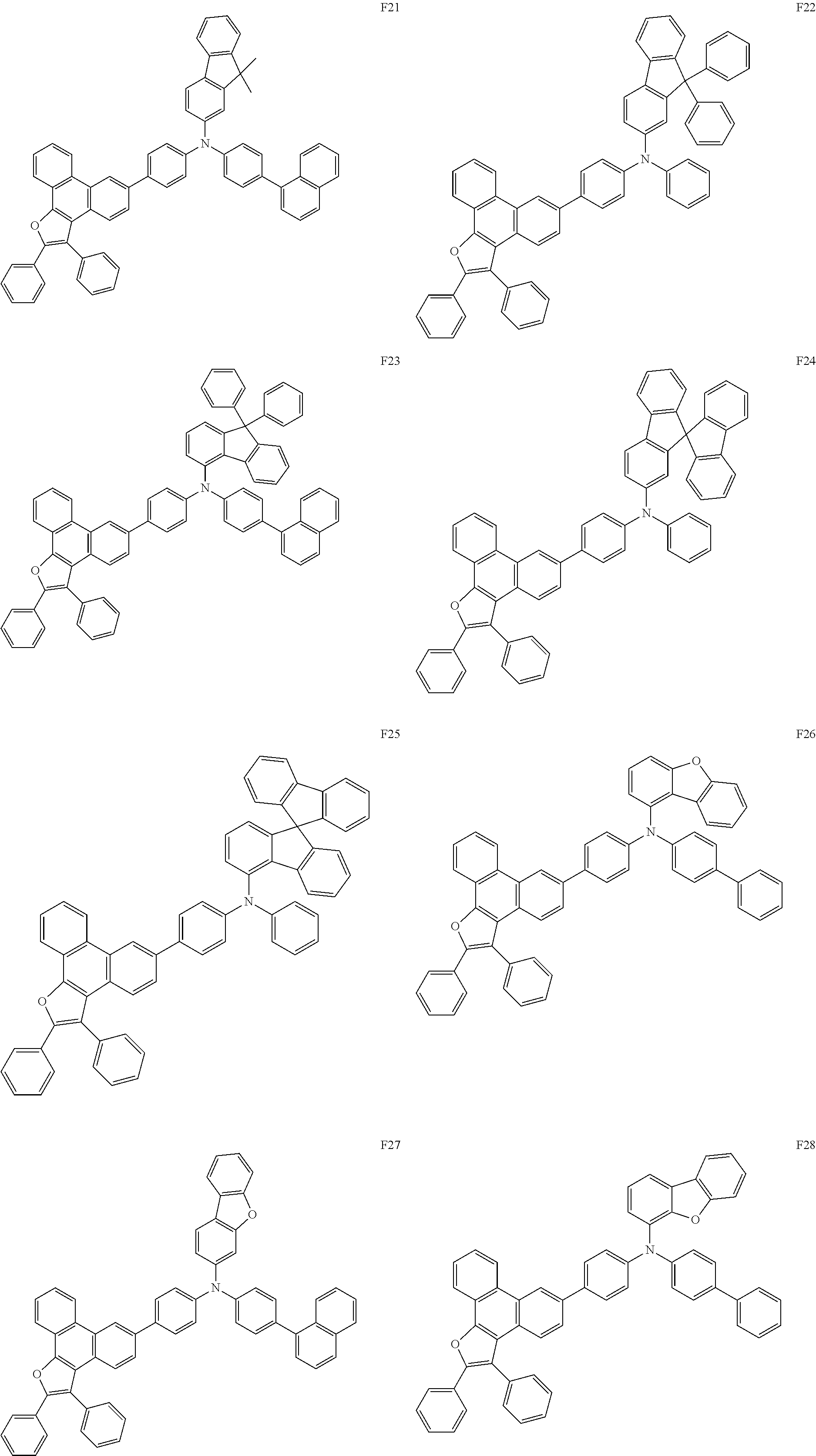 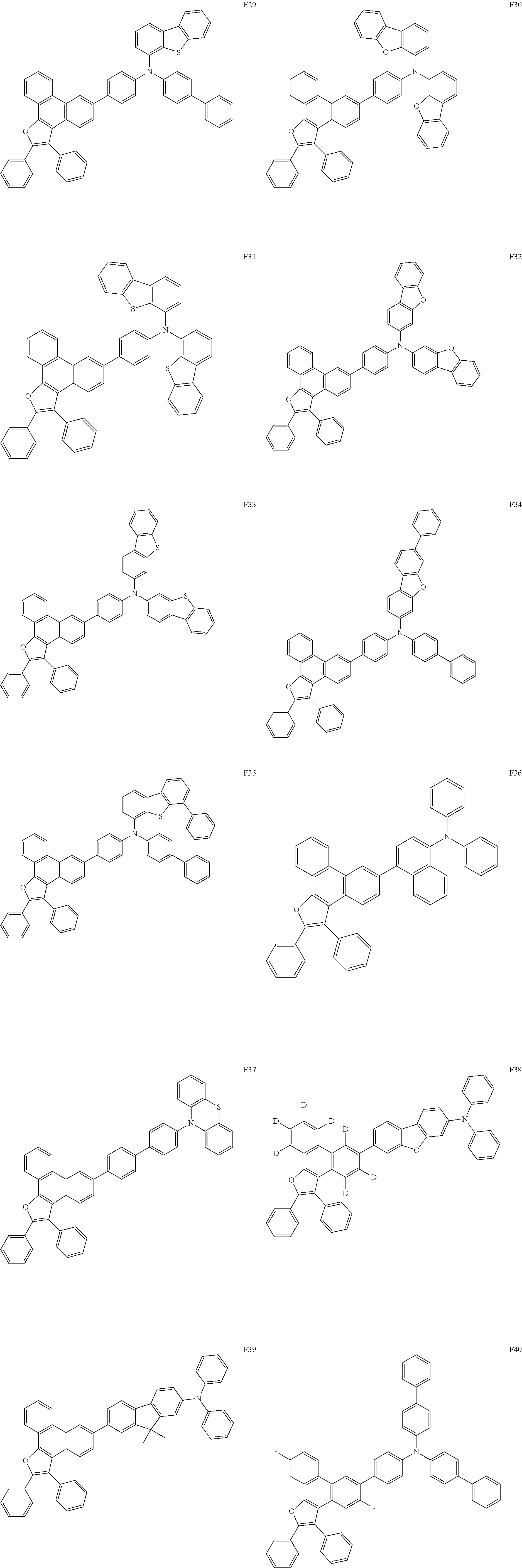 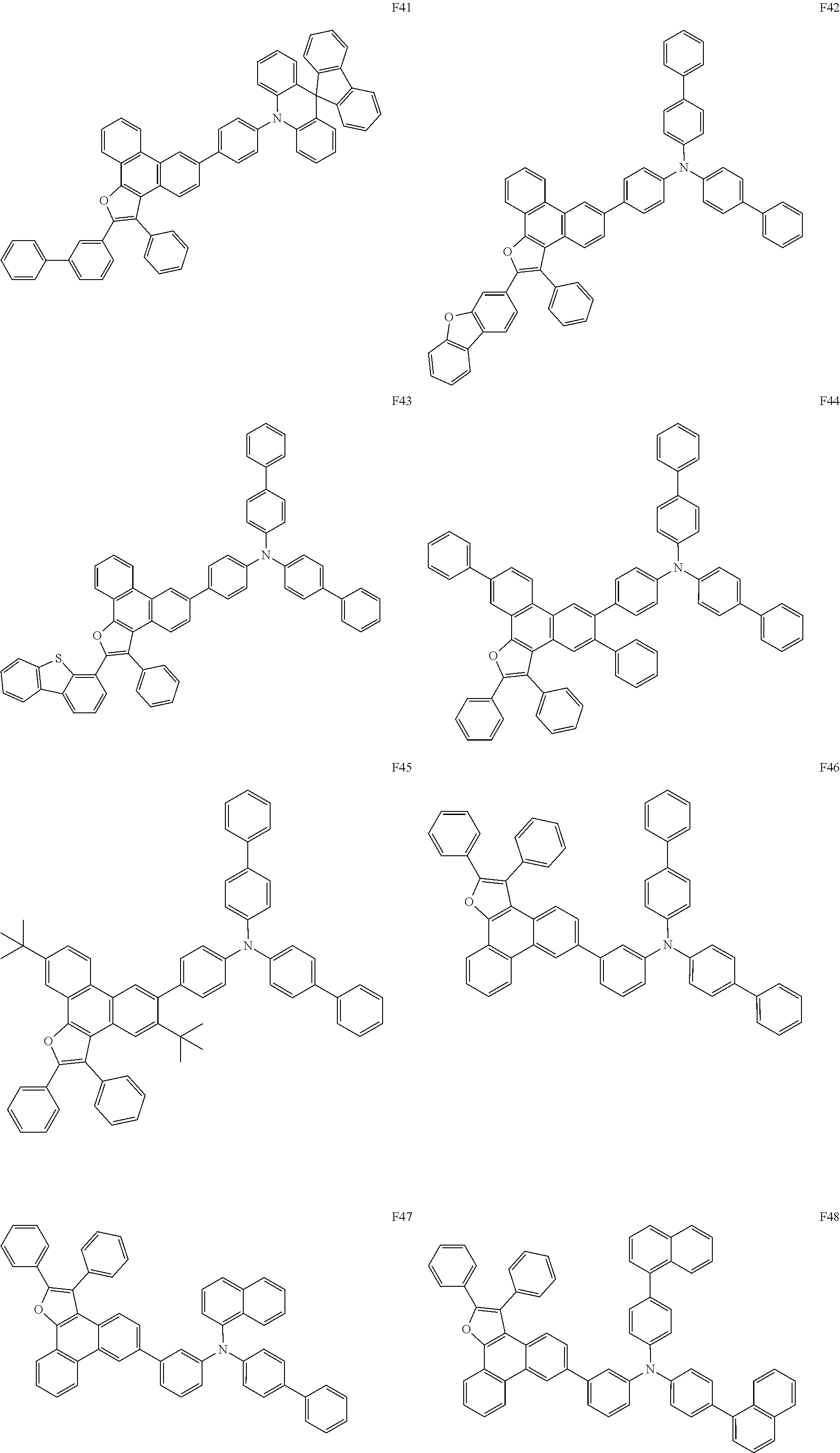 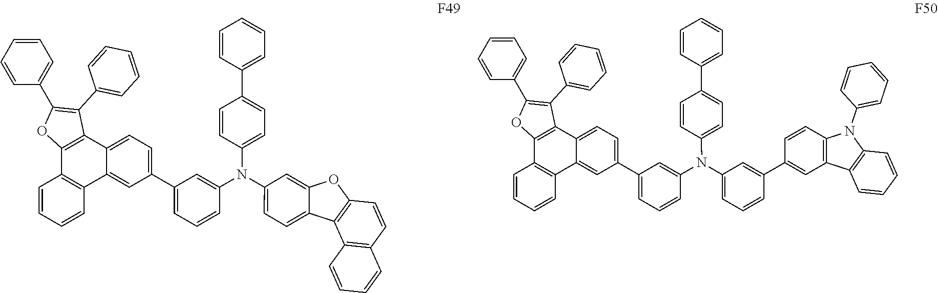 [SMILES: c1ccc(-c2ccc(N(c3cccc(-c4ccc5c(c4)c4ccccc4c4oc(-c6ccccc6)c(-c6ccccc6)c54)c3)c3ccc4c(c3)oc3ccc5ccccc5c34)cc2)cc1 ; c1ccc(-c2ccc(N(c3cccc(-c4ccc5c(c4)c4ccccc4c4oc(-c6ccccc6)c(-c6ccccc6)c54)c3)c3cccc(-c4ccc5c(c4)c4ccccc4n5-c4ccccc4)c3)cc2)cc1] 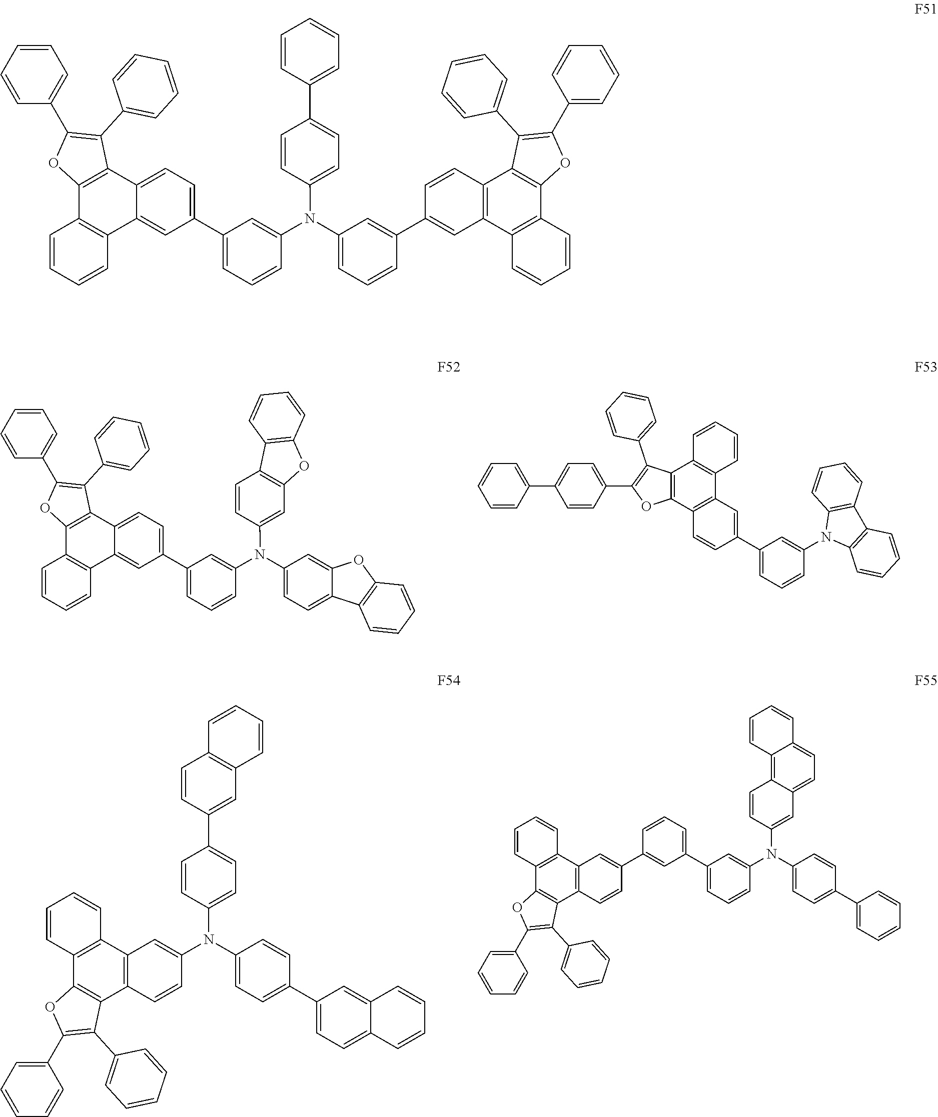 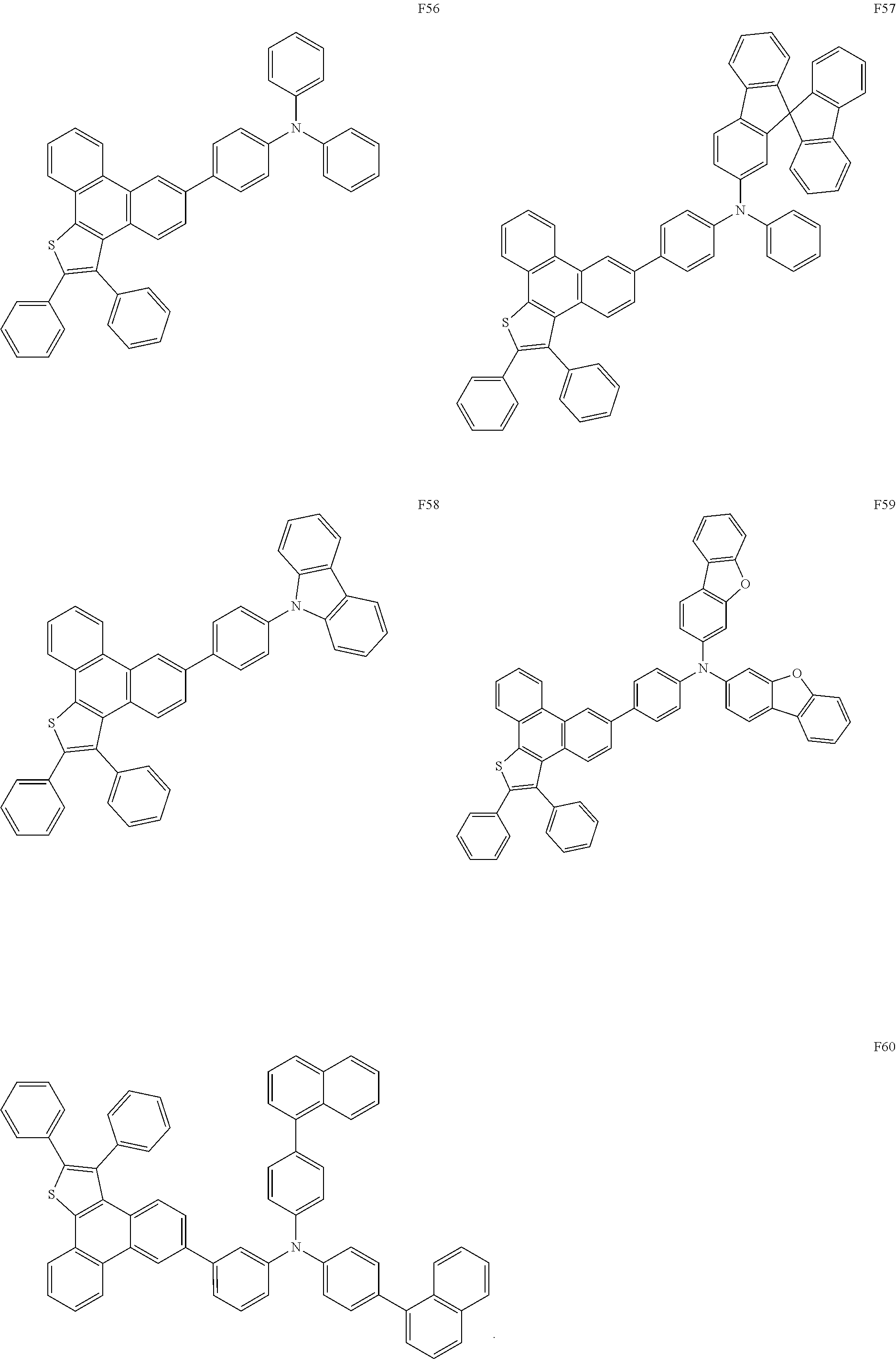 [SMILES: c1ccc(-c2sc3c4ccccc4c4cc(-c5ccc(N(c6ccccc6)c6ccccc6)cc5)ccc4c3c2-c2ccccc2)cc1 ; c1ccc(-c2sc3c4ccccc4c4cc(-c5ccc(N(c6ccccc6)c6ccc7c(c6)C6(c8ccccc8-c8ccccc86)c6ccccc6-7)cc5)ccc4c3c2-c2ccccc2)cc1 ; c1ccc(-c2sc3c4ccccc4c4cc(-c5ccc(-n6c7ccccc7c7ccccc76)cc5)ccc4c3c2-c2ccccc2)cc1 ; c1ccc(-c2sc3c4ccccc4c4cc(-c5ccc(N(c6ccc7c(c6)oc6ccccc67)c6ccc7c(c6)oc6ccccc67)cc5)ccc4c3c2-c2ccccc2)cc1 ; c1ccc(-c2sc3c4ccccc4c4cc(-c5cccc(N(c6ccc(-c7cccc8ccccc78)cc6)c6ccc(-c7cccc8ccccc78)cc6)c5)ccc4c3c2-c2ccccc2)cc1]
